(19) United States
(12) Patent Application Publication
(10) Pub. No.: US 2003/0065039 A1
(43) Pub. Date: Apr. 3, 2003

(54) Biologically active 1,3-bis-aromatic-prop-2-en-1-ones, 1,3-bis-aromatic-propan-1-ones, and 1,3-bis-aromatic-prop-2-yn-1-ones

(75) Inventors: Arsalan Kharazmi, Hellerup (DK); Soren Brogger Christensen, Nivaa (DK); Simon Feldbaek Nielsen, Herlev (DK)

Correspondence Address: DIKE, BRONSTEIN, ROBERTS AND CUSHMAN,, INTELLECTUAL PROPERTY PRACTICE GROUP, EDWARDS & ANGELL, LLP., P.O. BOX 9169, BOSTON, MA, 02209, US

(73) Assignee: Statens Serum Institute, Copenhagen K (DK)

(21) Appl. No.: 10/062,208

(22) Filed: Jan. 31, 2002

Related U.S. Application Data
(62) Division of application No. 09/462,125, filed on Dec. 27, 1999.
Application No. 09/462,125 is a 371 of international application No. PCT/DK98/00283, filed on Jun. 26, 1998.

(30) Foreign Application Priority Data
Jun. 26, 1997 (DK) 0762/97

(51) Int. Cl. A61K 31/12

(52) U.S. Cl. 514/678

(57) ABSTRACT

The invention relates to the use of 1,3-bis-aromatic-prop-2-en-1-ones (chalcones), 1,3-bis-aromatic-propan-1-ones (dihydrochalcones), and 1,3-bis-aromatic-prop-2-yn-1-ones for the preparation of pharmaceutical compositions for the treatment or prophylaxis of a number of serious diseases including i) conditions relating to harmful effects of inflammatory cytokines, ii) conditions involving infection by Helicobacter species, iii) conditions involving infection by viruses, iv) neoplastic disorders, and v) conditions caused by microorganisms or parasites. The invention also relates to novel chalcones and dihydrochalcones (especially alkoxy substituted variants) having advantageous substitution patterns with respect to their effect as drug substances, and to methods of preparing them, as well as to pharmaceutical compositions comprising the novel chalcones. Moreover, the present invention relates to a method for the isolation of Leishmania fumarate reductase, QSAR methodologies for selecting potent compounds for the above-mentioned purposes.

### FIELD OF THE INVENTION

The present invention relates to the use of 1,3-bis-aromatic-prop-2-en-1-ones, 1,3-bis-aromatic-propan-1-ones, and 1,3-bis-aromatic-prop-2-yn-1-ones (in short bis-aromatic compounds) for the preparation of pharmaceutical compositions for the treatment or prophylaxis of a number of serious diseases including i) conditions relating to harmful effects of inflammatory cytokines, ii) conditions involving infection by Helicobacter species, iii) conditions involving infection by viruses, iv) neoplastic disorders, and v) conditions caused by microorganisms or parasites. The invention also relates to novel chalcones and dihydrochalcones having advantageous substitution patterns with respect to their effect as drug substances, and to methods of preparing them, as well as to pharmaceutical compositions comprising the novel chalcones.

### BACKGROUND OF THE INVENTION

WO 93/17671 and WO 95/06628 which are assigned to the same applicant as the present application, describe the use of particular classes of oxygenated chalcones for the treatment or prophylaxis of conditions caused by microorganisms or parasites, in particular protozoa such as Leishmania, Plasmodia, and Coccidia such as Eimeria, and intracellular bacteria including Legionella and Mycobacteria.

### BRIEF DESCRIPTION OF THE FIGURES

FIG. 1. The minimal inhibitory concentrations (MIC) as given in gg/ml of licochalcone A, PH74 and PH98 are shown against 20 different strains of Helicobacter pylori.

FIG. 2. The minimal inhibitory concentrations (MIC) as given in μg/ml of PH104, PH105, PH135 and PH136 are shown against 20 different strains of Helicobacter pylori.

FIG. 3. A: Separation of concentrated sample on Hiload superdex 200 column. B: Separation of fraction 1 from Hiload column on MonoQ column.

FIG. 4. SDS-Page of the purification of a fumarate reductase from Leishmania donovani. A and D: Molecular weight marks. B: Concentrated extract of membrane associated proteins. C: Approximately 90% pure fumarate reductase (peak 21 from MonoQ column). The electrophoresis was performed with a 15% gel on a Bio-Rad mini protein II apparatus. The gel was stained with colloidal comassie.

FIGS. 5-17. Effect of various oxygenated chalcones on the activity of fumarate reductase and other enzymes of the mitochondrial electron transport chain.

FIG. 5. Effect of licochalcone A on Leishmania major fumarate reductase. Digitonin treated promastigotes was incubated with licochalcone A at 28° C. 30 min, and the remaining enzyme activity was measured. Data are mean ±SEM from 6 different experiments.

FIG. 6. Effect of licochalcone A on Leishmania major fumarate reductase. KCL-solubilised fumarate reductase was incubated with licochalcone A at 28° C. 30 min, and the remaining enzyme activity was measured. Data are mean ±SEM from 6 different experiments.

FIG. 7. Effect of SBC-35ma on Leishmania major fumarate reductase. KCL-solubilized fumarate reductase was incubated with licochalcone A at 28° C. 30 min, and the remaining enzyme activity was measured. Data are mean ±SEM from 6 different experiments.

FIG. 8. Effect of SBC-24ma on Leishmania major fumarate reductase. KCL-solubilized fumarate reductase was incubated with SBC24ma at 28° C. 30 min, and the remaining enzyme activity was measured. Data are mean ±SEM from 6 different experiments.

FIG. 9. Fumarate reductase activity.

FIG. 10. Licochalocone A on SDH of L. major promastigotes (membrane rich fraction)

FIG. 11. Effect of licochalcone A on Leishmania major NADH dehhydrogenase. Membrane rich fraction of parasite was incubated with licochalcone A at 28° C. 30 min, and the remaining enzyme activity was measured. Data are mean ±SEM from 6 different experiments.

FIG. 12. Effect of licochalcone A on Leishmania major succinate-cytochrome c reductase. Membrane rich fraction of parasite was incubated with licochalcone A at 28° C. for 5 min, the remaining enzyme activity was measured. Data are mean ±SEM from 4 different experiments.

FIG. 13. Effect of licochalcone A on Leishmania major NADH-cycochrome c reductase. Membrane rich fraction of parasite was incubated with licochalcone A at 28° C. for 5 min, the remaining enzyme activity was measured. Data are mean ±SEM from 4 different experiments.

FIG. 14. Effect of licochalcone A on human MNC succinate dehydrogenase. Membrane rich fraction of parasite was incubated with licochalcone A at 28° C. for 5 min, the remaining enzyme activity was measured. Data are mean ±SEM from 4 different experiments.

FIG. 15. Effect of licochalcone A on human MNC NADH dehydrogenase. Membrane rich fraction of parasite was incubated with licochalcone A at 28° C. for 5 min, the remaining enzyme activity was measured. Data are mean ±SEM from 4 different experiments.

FIG. 16. Effect of licochalcone A on J774 cells succinate dehydrogenase. Membrane rich fraction of parasite was incubated with licochalcone A at 28° C. for 5 min, the remaining enzyme activity was measured. Data are mean ±SEM from 4 different experiments.

FIG. 17. Effect of licochalcone A on J774 cells NADH dehydrogenase. Membrane rich fraction of parasite was incubated with licochalcone A at 28° C. for 5 min, the remaining enzyme activity was measured. Data are mean ±SEM from 4 different experiments.

FIG. 18. Effects of oral administratoin of PH135 (A) and PH 104(B) on LPS-induced TNF-α production in the plasma of BALB/c mice. ∇1, PH135 50 mg/kg, 1 h before, the same time, and 1 h after LPS injection. □II, PH104, 50 mg/kg, 1 h before, the same time, and 1 hr after LPS injection. ▾III, PH135 150 mg/kg at the same time as LPS injection. ▪IV, PH104 150 mg/kg at the same time as LPS injection. O V, control.

FIG. 19. Effect of intraperitoneal administration of PH135 and PH104 on LPS-induced TNF-α production in the plasma of BALB/c mice.

FIG. 20. Effect of oral administration of SBC-24mbc on LPS-induced septic shock in BALB/c mice. SBC-24mbc was suspended in 0.2% Na-CMC and 0.01% tween 80 and was given to mice by oral in a dose of 50 mg/kg at 1 h before, 2 h after, 8 h after, and 24 h after LPS injection.

FIG. 21. Effect of oral administration of SBC-24mbc on LPS-induced septic shock in BALB/c mice. SBC-24mbc was suspended in 0.2% Na-CMC and 0.01% tween 80 and was given to mice by oral in a dose of 50 mg/kg. 1.2 h before, same time, and 2 h after LPS injection, II. 1 h before, 2 h after, and 4 h after LPS-injection; III. 1 h before and 2 h after LPS-injection IV. Same time and 2 h after LPS-injection. V. 1 h and 2 h after LPS-injection.

FIG. 22. Effect of i.p. administration of SBC-24mbc on LPS-induced septic shock in BALB/c mice. SBC-24mbc was dissolved in 100 μl of DMSO and then suspended in sterile pyrogen-free salin and was given to mice by i.p. route in the doses of 30 mg and 10 mg/kg/time, at 1 h before, same time and 2 h after LPS-injection. Control 1 received 200 μl of 10% DMSO in sterile pyrogen-free salin. Control 2 received 200 μl of sterile pyrogen-free salin.

FIG. 23. Dose response study of SBC-24mbc and chlorpromazine (CPZ) by i.p. route on LPS-induced septic shock in BALBlc mice. SBC-24mbc was dissolved in 100 μl of DMSO and then suspended in sterile pyrogen-free salin and was given to mice by i.p. route in the doses of 10 mg, 5 mg, and 2.5 mg/kg/time, at 1 h before, same time, and 2 h after LPS-injection. Control received 200 μl of 10% DMSO in sterile pyrogen-free salin. CPZ was dissolved in sterile pyrogen-free salin and was given to mice by i.p. route in the dose of 4 mg/kg, at the same time as LPS-injection.

FIG. 24. Effect of oral administration of PH135 and PH104 on LPS-induced septic shock in BALB/c mice. PH135 and PH104 were micronized in 0.2% Na-CMC and 0.01% Tween 80 and were given to mice by oral administration in a dose of 50 mg/kg at 1 h before, the same time, and 1 h after LPS injection.

FIG. 25. Effect of oral administration of PH135 and PH104 on LPS-induced septic shock in BALB/c mice. PH135 and PH104 were micronized in 0.2% Na-CMC and 0.01% Tween 80 and were given to mice by oral administration in a dose of 16.6 mg/kg at 1 h before, the same time, and 1 h after LPS injection.

FIG. 26. Effect of oral administration of licochalcone A on protection of C57 BL/6 mice from cerebral malaria infected with P. berghei K173 strain. Mice received 50 mg/kg of licochalcone A once daily over 5 days.

FIG. 27. The result of PCA The values of the new variables PC₁ and PC₂ for all substituents are shown in the diagram.

FIG. 28. Observed and predicted antileishmanial activities of the selected chalcones. The compounds included in the model are designated with O, and validation set is marked with ▾.

FIG. 29. The size of the coefficients for each of the five variables in eight of the ten positions, which can be substituted (position 5′ and 6′ only contribute to a minor extent).

FIG. 30: Observed and predicted lymphocyte suppressing activities of the selected chalcones. The chalcones used for calculating the model are depicted by O, and the validation set is marked with ▾.

FIG. 31. The size of the coefficients for each of the five variables in eight of the ten positions, which can be substituted position 5′ and 6′ only contribute to a minor extent).

FIG. 32: Antileishmanial activity. Stereo plot of negative coefficients (0.0005) for the three probes methyl (top) water (middle) and ammonium ion (bottom).

FIG. 33: Antileishmanial activity. Stereo plot of positive coefficients (0.0005) for the three probes methyl (top) and ammonium ion (bottom).

FIG. 34: Lymphocyte suppressing activity. Stereo plot of negative coefficients (0.0005) for the three probes methyl (top) and water (bottom).

FIG. 35: Lymphocyte suppressing activity. Stereo plot of positive coefficients (0.0005) for the three probes methyl (top) water (middle) and ammonium ion (bottom).

FIG. 36: Antiplasmodium activity. Plot of negative coefficients (0.0005) for the methyl probe.

FIG. 37: Antiplasmodium activity. Plot of positive coefficients (0.0005) for the methyl probe.

### DESCRIPTION OF THE INVENTION

The present invention is based on the teachings disclosed in the above-mentioned published international patent applications. Thus, further studies performed by the inventors have shown that several oxygenated chalcones altered the ultrastructure of Leishmania major promastigote and amastigote mitochondria in a concentration-dependent manner without damaging the organelles of macrophages or the phagocytic function of these cells. Studies on the function of the parasite mitochondria showed that Licochalcone A inhibited the respiration of the parasite in a concentration-dependent manner, as shown by inhibition of O₂ consumption and CO₂ production by the parasites. Moreover, licochalcone A inhibited the activity of the parasite mitochondrial dehydrogenase.

The present inventors have now found that certain classes of aromatic compounds (1,3-bis-aromatic-prop-2-en-1-ones, 1,3-bis-aromatic-propan-1-ones, and 1,3-bis-aromatic-prop-2-yn-1-ones, in the following denoted bis-aromatic compounds) have a very broad spectrum of biological activities besides having the activities as described in the above-mentioned two international patent applications. At the same time they are relatively well tolerated by normal mammalian cells, including human cells. In the examples, details are given with respect to the toxicological tests which have been performed. Overall the toxicological tests have demonstrated that none of the compounds tested were positive in Ames test and that non-mutagenic compounds exist amongst the bis-aromatic compounds.

With respect to antileishmanial activity, the target molecule for the compounds appears to be parasite-specific mitochondrial enzyme fumarate reductase. This enzymes does not exist in mammalian cells and, accordingly, substances having an activity against this enzymes are potential drug candidates for diseases or conditions relating to fumarate reductase such as, e.g., Leishmanasis and diseases related to Helicobacter species such as, e.g., gastric ulcer. One aspect of the invention relates to a method for the preparation and purification of leishmania fumarate reductase and the use thereof in a molecular modelling model for predicting potential substances capable of inhibiting the activity of this enzyme.

This finding is novel and provides a unique mechanism of action which has not been described for other anti-protozoal drugs.

As mentioned above, it has been found that the compounds exert activity against Helicobacter pylori and it has been demonstrated that they also have antiviral and/or antineoplastic activities as well as an inhibiting effect against inflammatory cytokines. In the following is given an overview over the biological activities.

Indications

Helicobacter pylori

As mentioned above, the present invention relates to the use of bis-aromatic compounds like chalcones and related compounds for the preparation of a pharmaceutical composition for the treatment or prophylaxis in humans of conditions involving infection by Helicobacter species, such as H. pylori, in the human gastric mucosa, as well as a method of treating such conditions using a chalcone or a related compound defined with the general formula I.

Helicobacter pylori is a microaerophilic spiral shaped organism which is found in the stomach and generally appears to have an exclusive habitat in the human gastrointestinal mucosa. It has been estimated that this bacterium will have infected the gastric mucosa of more than 60% of adult humans by the time they are 60 years old. Moreover, Helicobacter pylori has been implicated as a contributing factor in a number of pathological conditions, including acute (type B) gastritis, gastric and duodenal ulcers, and gastric adenocarcinoma.

Helicobacter pylori has recently been found to posses the enzyme fumarate reductase and it is therefore contemplated that the mechanism of action involved in the activity against different strains of Helicobacter pylori is based on an inhibition of this enzyme by chalcones and related substances.

As described in the examples, several oxygenated chalcones exhibited MIC (minimum inhibiting concentrations) values equal to or less than 16 μg/ml against a large number of H. pylori strains. Furthermore, preliminary experiments have shown that several chalcones inhibit the activity of H. pylori fumarate reductase. H. pylori fumarate reductase is an essential enzyme in H. pylori metabolism when grown under anaerobic or microacrophilic conditions like the gastrointestinal tract of human. Therefore, the potential of using H. pylori fumarate reductase as a target for bis-aromatic compounds like chalcones and related substances makes these compounds unique and important anti-Helicobacter agents.

Thus, the present invention i.a. relates to the use of a bis-aromatic compound for the preparation of a pharmaceutical composition for the treatment or prophylaxis of conditions involving infection by Helicobacter species, wherein the bis-aromatic compound has an MIC value in the Helicobacter pylori Assay of at the most 200 μg/ml, such as at the most, e.g., 100 μg/ml, 75 μg/ml, 50 μg/ml, 40 μg/ml, 30 μg/ml, 20 μg/ml, or 10 μg/ml. In connection with the desirable and useful effects in connection with the treatment or prophylaxis of Helicobacter species induced conditions, it is also important that the compound for the use according to the invention only has minor (preferably no) side effects, thus, it is preferred that the compound, at the MIC in the Helicobacter species Assay, shows a reduction of the thymidine uptake by human lymphocytes in the Lymphocyte Proliferation Assay, as defined herein, using phytomemagglutinin (PHA), of less than 50%, preferably less than 40%, especially less than 30%, in particular less than 20%. In particular, the ratio between the MIC value in the Helicobacter species Assay and the IC₅₀ value in the Lymphocyte Proliferation Assay for the compound in question should be less than 2, such as less than, e.g., 1.5, 1.2., 1.0, 0.9, 0.8, 0.7, 0.6, 0.5, 0.4, 0.3, or 0.2.

The bis-aromatic compounds used according to the invention as well as the novel compounds according to the invention can either be used as the sole active substance or in combination with other drug substances. Especially, it is contemplated that the bis-aromatic compound—where used in the treatment or prophylaxis of conditions involving infection by Helicobacter species—advantageously can be administered in combination with the known anti-Helicobacter combination of amoxycillin, metronidazole and bismuth or, alternatively, the bis-aromatic compound can substitute any one of amoxycillin, metronidazol or bismuth in the combination.

Antiviral Effects

The present invention relates also to the use of chalcones and related substances of the general formula I for the preparation of a pharmaceutical composition for the treatment and/or prophylaxis of a disease caused by a virus in a mammal, including a primate such as a human.

The present invention claims that bis-aromatic compounds like chalcones and related substances can exhibit antiviral effects in infected cells. A very important feature by the chalcones and related substances is that it has been found that they are substantially harmless to mammalian cells in concentrations at which they effectively exert the antiviral effect. This selectivity is very important and surprising.

It is contemplated that the chalcones and related substances are effective against viruses selected from the group consisting of: retrovira such as human immunodeficiency virus (HIV type I and type II; the HIV virus was previously known or referred to as LAV, HTLV-III or ARN); parvovira; papovavira, such as papilloma virus; andenovira; herpes vira such as Epstein-Barr virus, cytomegalovirus, herpes simplex vira (HSV 1 and HSV 2), varicella, herpex zoster virus, hepatitis A, hepatitis B; poxvira such as vaccinia, smallpox, molluscum contagiosum, cowpox, and monkey pox virus; hepadnavira; picornavira such as rhinovira and enterovira; reovira such as rotavirus and orbivirus; arbovira such as toga-, flavi-, bunya, rhabdo-, arena-, and reovira; coronavira; leukaemia, and sarcoma vira; orthomyxovira such as influenza vira; paramyxovira such as mumps virus, measles virus, parainfluenza virus, and RSV; and other unclassified viruses such as lentivira, non-A, non-B hepatitis vira, and viroids.

In the examples are shown the cytotoxicity and antiviral activity of a number of bis-aromatic compounds like chalcones and related substances. Activity against parainfluenza-3 virus, reovirus-1, sindbis virus, coxsackie virus B4, semliki forest virus and cyromegalovirus in human embryonic lung. The results clearly demonstrate that the compounds have antiviral activity and, therefore, the compounds are potential antiviral drug substances.

The present invention, thus, also relates to the use of a bis-aromatic compound for the preparation of a pharmaceutical composition for the treatment or prophylaxis of conditions involving infection by viruses, wherein the bis-aromatic compound has an IC₅₀ value in the Virus plaque formation and/or virus cytophatic Assay of at the most 50 μg/ml, such as at the most, e.g. 40 μg/ml, 30 μg/ml, 20 μg/ml, 10 μg/ml, or 5 μg/ml.

In connection with the desirable and useful effects in connection with the treatment or prophylaxis of conditions caused by viruses, it is also important that the compound for the use according to the invention only has minor (preferably no) side effects, thus, it is preferred that the compound, at the IC₅₀ concentration in the Virus plaque formation and /or virus cytophatic Assay, shows a reduction of the thymidine uptake by human lymphocytes in the Lymphocyte Proliferation Assay, as defined herein, using phytomemagglutinin (PHA), of less than 50%, preferably less than 40%, especially less than 30%, in particular less than 20%. In particular, the ratio between the IC₅₀ value in the Virus Assay and the IC₅₀ value in the Lymphocyte Proliferation Assay for the compound in question should be equal to or less than 1, such as less than, e.g., 0.9, 0.8, 0.7, 0.6, 0.5, 0.4, 0.3, or 0.2.

Neoplastic Disorders

The compound or mixture of compounds for use according to the invention are claimed to be potent antineoplastic candidates.

Thus, the compound or mixture of compounds for use as antineoplastic compounds may be further defined as a bis-aromatic compound like a chalcone or a related substance which, when tested according to a standard test system for testing potential anticancer drugs, demonstrates an antineoplastic effect. Such a standard test could be, e.g., a systematic protocol established by the National Cancer Institute (NCI) involving the testing of a compound against a standard cell line panel containing 60 human tumor cell lines. The protocol and the established statistical means for analysing the results obtained by the standardised testing are well described in the literature, see, e.g., Boyd M. R.: Principles & Practice of Oncology, PPO Updates, Volume 3, No. 10, October 1989 (description of the testing protocol) and Paul, K. D.: Display and Analysis of Patterns of Differential Activity of Drugs Against Human Tumor Cell Lines, Development of Mean Graph and COMPARE Algorithm, Journal of the National Cancer Institute Reports, Vol. 81, No. 14, p. 1088, Jul. 14, 1989 (description of the methods of statistical analysis).

It is contemplated that the bis-aromatic compounds like chalcones and related compounds are effective in the treatment or prevention of neoplastic disorders such as neoplastic disorders selected from the group consisting of epithelial neoplasms and non-epithelial and mixed neoplasms. In the table given below is listed relevant neoplasms based on a histogenetic classification.

TABLE

| Cell or Tissue | | |
|---|---|---|
| Type | Benign | Malignant |
| Epithelial neoplasms | | |
| surface | papilloma | carcinoma, basal-cell |
| | | carcinoma, transitional cell |
| | | carcinoma, papillary |
| | | carcinoma, apudomas, |
| | | nesidiocytoma, clear-cell |
| | | carcinoma, choriocarcinoma, |
| | | and trabecular carcinoma |
| glandular | adenoma | adenocarcinoma |
| | | spheroidal cell carcinoma, |
| | | cystadenocarcinoma, |
| | | papillary adenocarcinoma, |
| | | and mucous or colloid |
| | | carcinoma |
| Non-epithelial and | | |
| mixed neoplasms | | |
| Connective tissues | | |
| adipose | lipoma | liposarcoma |
| fibrous | fibroma | fibrosarcoma |
| cartilage | chondroma | chondrosarcoma |
| bone | osteoma | osteosarcoma |
| smooth muscle | leiomyoma | leiomyosarcoma |
| striped muscle | rhabdomyoma | rhabonyosarcoma |
| mesothelia | | mesothelioma |
| Neuro-ectodermal | — | gliomas, astrocytoma, |
| glial cells | | oligodendroglioma, |
| | | ependymoma and anaplastic |
| | | variants |
| nerve cells | ganglioneuroma | neuroblastoma |
| | | medulloblastoma |
| | | retinoblastoma |
| melanocytes | pigmented naevus | malignant melanoma |
| meninges | meningioma | malignant meningioma |
| nerve sheaths | schwannoma | |
| | | neurofibrosarcoma |
| | neurofibroma | |
| Haemopoietic | | leukaemias |
| and lymphoreticular | | acute leukaemias, monocytic |
| | | leukaemia, myeloblastic |
| | | leukaemia (AML), |
| | | lymphoblastic leukaemia |
| | | (ALL) and chronic |
| | | leukaemia, chronic mycloid |
| | | leukaemia (CML), chronic |
| | | lymphocytic leukaemia |
| | | (CLL), hairy cell leukaemia |
| | | other |
| | | myeloproliferative |
| | | disorders, myelomatosis, |
| | | myelofibrosis |
| | | lymphomas |
| | | Hodgkin's disease, |
| | | non-Hodgkin's lymphomas, |
| | | and histiocytic lymphomas |
| Blood vessels | haemangioma | haemangiosarcoma |
| and | glomangioma | Kaposi's disease |
| lymphatic vessels | lymphangioma | lymphangiosarcoma |
| Germinal and | benign teratoma | malignant teratoma |
| embryonal cells | | dysgerminoma (F) |
| | | serminoma (M) |
| placenta | hydatidiform | choriocarcinoma |
| | mole | |

Based upon the disclosure of the present invention, a person skilled in the art will be able to test the compounds of formula I as outlined. Substances which are considered useful may then be tested for cytotoxic effects in appropriate cell systems such as, e.g., CEM and HL60 cell lines.

In the examples, results are given showing that the compounds tested in a concentration of 5 μg/ml or higher exhibited potent inhibitory activity against two human cell lines CEM and HL60.

This being said, the present invention also relates to the use of a bis-aromatic compound for the preparation of a pharmaceutical composition for the treatment or prophylaxis of neoplastic disorders, wherein the 1,3-bis-aromatic-prop-2-en-1-one, 1,3-bis-aromatic-propan-1-one, or 1,3-bis-aromatic-prop-2-yn-1-one has an IC₅₀ value in the Anti-cancer Assay of at the most 100 μg/ml, such at the most, e.g., 75, 60, 50, 40, 30, 20, or 10 μg/ml.

Cytokine Inhibition

Cytokines have been shown to play an essential role in the regulation of the immune response. Some of them such as interleukin-1 (IL-1), IL-2, IL-4, IL-10, IL-12 and interferon-gamma (IFN-γ) specify whether cell-mediated or humoral immunity will develop against a given antigen or in a given infection. This is reflected in the activation of the T-helper type 1 (Th1) or type 2 (Th2) cells. The outcome of a cytokine-mediated response could be either a protective/beneficial or a pathogenic/harmful effect.

In Summary

TNF-α is a cytokine produced primarily by activated monocytes/macrophages.

TNF-α is synthesised as a pro-protein consisting of 233 amino acids (26 kDa).

The pro-protein is cleaved by a specific metalloprotease called TNF-α-converting enzyme (TACE) to a monomer of 157 amino acids (17 kDa).

It is then processed into a non-covalently bound cone-shaped homotrimer.

TNF-α plays a key role in the cytokine network with regard to the pathogenesis of many infectious and inflammatory diseases.

TNF-α was purified and the gene cloned in the mid 1980s.

TNF-α effects are transmitted via crosslinking of the membrane-bound TNF receptors (TNFRI & TNFRII) on the target cell.

Knock out mice for either TNF-α itself or the TNF-α receptors are shown to resistant to endotoxin shcok or protected from obesity-induced insulin resistance.

The role of inflammatory cytokines such as IL-1, IL-6 and tumor necrosis factor a (TNF-α) in autoimmune diseases, i.e. rheumatoid arthritis, lupus erythematosus and diabetes and in other chronic inflammatory diseases, has been well documented. It has also been shown that these cytokines play an important role in both the protection and in the pathogenesis of certain infectious diseases. Other human diseases associated with TNF-α are

Infections:

Sepsis syndrome

Bacterial meningitis

cerebral malaria

AIDS

Autoimmune Diseases:

Rheumatoid arthritis

Crohn's disease

Multiple sclerosis.

Graft-versus-host disease.

Transplant rejection.

Organ Injury Disorders:

Diabetes.

ARDS.

Myocardial infarction.

Liver failure.

TNF-α has been shown to be a key mediator in the endotoxin-induced toxic shock and plays a key role in the pathogenesis of septic shock. High TNF-α-serum levels have been demonstrated after lipopolysaccharide (LPS) administration to animals and human volunteers, or in septic shock patients (Michie et al. N. Eng. J. Med. 318: 1481, 1981; Waage et al. J. Exp. Med. 169: 333, 1989). Administration of anti-TNF antibodies has been shown to protect against the lethal effect of LPS and live bacteria in a variety of animal models (Beutler et al. Science 229: 869, 1985; Tracey et al. Nature 330: 662, 1987). Anti-TNF antibodies have been shown also to be effective in a variety of animal models of human diseases, including cerebral malaria (Grau et al. Science 237: 210, 1987), bacterial meningitis (Saukkonen et al. J. Exp. Med. 171: 439, 1990), bleomycin lung toxicity (Piquet et al. J. Exp. Med. 170: 655, 1989), liver ischemia/reperfusion damage (Colletti et al. J. Clin. Invest. 85: 1936, 1990), and graft-versus-host reaction (Shalaby et al. Transplantation 47: 1057, 1989), indicating an involvement of TNF in the pathogenesis of these diseases.

Therefore, TNF might be an important target for pharmacological action, and inhibitors of its synthesis or bioactivity might be useful in the therapy of these diseases.

Recently there has been a great deal of interest in developing drugs which can inhibit production of certain cytokines such as IL-1α, IL-6 and TNF-α, and preventing the harmful effect of these inflammatory cytokines. Anti-TNF antibodies have generated a lot of interest and at present several clinical studies are underway to assess the beneficial effect of these antibodies. The commonly used antiinflammatory compounds such as glucocorticoids and particularly non-steroid antiinflammatory drugs are also being considered as potential anti-TNF drugs. However, there are a number of problems with these drugs both in terms of their efficiency and their side-effects. For example it has been shown that some animal models of endotoxic shock are resistant to glucocorticoid therapy (Bone et al. N. Eng. J. Med. 317: 653, 1987). Tenidap, an antirheumatic agent, has been shown to be effective against TNF-α, but because of its toxicity further development of this drug has been abandoned.

Because of the scope of the problems associated with IL-1α, IL-6 and TNF-α, and the lack of efficient drugs against these inflammatory cytokines, there is a great need for the development of new drugs against the inflammatory cytokines.

We have now found that 1,3-bis-aromatic-prop-2-en-1-ones, 1,3-bis-aromatic-propan-1-ones, and 1,3-bis-aromatic-prop-2-yn-1-ones are capable of inhibiting the activity of inflammatory cytokines. A 100% protection in mice in an LPS-induced septic shock model at doses of 5 mg/kg has been found. In contrast, Batt et al. (J. Med. Chem. 36: 1434, 1993) describing 2′-substituted chalcone derivatives have only observed a 50% protection with a dose of 600 mg/kg.

Furthermore, the bis-aromatic compounds studied have no toxic effect on human peripheral blood leukocytes and lymphocytes at concentrations of up to 100 μg/ml (300 μM).

Thus, a substance like a bis-aromatic compound capable of affecting a inflammatory cytokine is a potential drug candidate for the treatment or prophylaxis of all the diseases or conditions mentioned above; in more general terms, these diseases or conditions encompass immune diseases, including infectious diseases, allergic diseases, auto-immune diseases, immune defects, and graft-related or transplantation related diseases. The inflammatory response in all these diseases plays an important pathogenic role and, therefore, a substance affecting inflammatory cytokines is also a potential drug candidate for the treatment and/or prevention of inflammatory diseases in general, including auto-immune diseases.

In addition, mechanical, traumatical, thermal, chemical and toxic impacts on tissue may cause inflammatory reaction independent of the immune system.

The above-mentioned diseases or disorders relevant in the present context include inflammatory rheumatic diseases, degenerative rheumatic diseases, metabolic diseases of the bones, rheumatic manifestations of general medical diseases and hereditary connective tissue disorders.

More specifically, relevant diseases or conditions are arthritic disorders, including infectious arthritis, osteoarthritis, rheumatoid arthritis and other rheumatoid diseases such as Juvenile Arthritis, Systemic Lupus Erythematosis, Sjögren's Syndrome, Progressive Systemic Sclerosis, Polymyositis, Dermatomyositis, Ankylosing Spondilitis, Reiter's Syndrome, Reynaud's Syndrome, Psoriatic Arthritis, Relapsing Polychondritis, Relapsing Panniculitis, Crohn's Disease, Ulcerative Colitis, Hereditary Complement Deficiencies, Collagen Vascular Diseases, rheumatological manifestations associated with bacterial and viral endocarditis or myocarditis and other rheumatological manifestations such as anaemia of chronic disorders, mixed connective tissue disease, vasculitis, polyarthritiis nodosa, nephritis, fibrotic syndroms, sarcoidose, diabetes mellitus, and diseases of the thyreoidea, including goitre.

Other relevant diseases and groups of disorders wherein inflammatory cytokines may play an important role also include the wide range of diseases well known in the art to be associated with The Major Histocompability Complex such as juvenile diabetes, Reiter's syndrome, gluten-sensitive enteropathy.

Among the allergic disorders wherein inflammatory cytokines may be involved are asthma bronchiale and atopic dermatitis.

Also virus induced neoplastic disorders such as Burkitt's lymfhoma are believed to be associated with cytokine interaction.

In many other diseases such as, e.g., in dissiminated sclerosis, cytokines are believed to play an important pathogenic role, and accordingly, such diseases may be prevented or treated by use of substances capable of regulating cytokine production and function.

In order to illustrate an aspect which the present invention also intends to cover, the Cytokine Inhibition Assay described herein has been used to determine the useful effect of the compounds described herein. Thus, the present invention also relates to the use of a 1,3-bis-aromatic-prop-2-en-1-one, 1,3-bis-aromatic-propan-1-one, or 1,3-bis-aromatic-prop-2-yn-1-one for the preparation of a pharmaceutical composition for the treatment or prophylaxis of conditions relating to harmful effects of inflammatory cytokines, wherein the 1,3-bis-aromatic-prop-2-en-1-one, 1,3-bis-aromatic-propan-1-one, or 1,3-bis-aromatic-prop-2-yn-1-one has an IC₅₀ value in the Cytokine inhibition Assay of at the most 100 μg/ml, such at the most, e.g., 75, 60, 50, 40, 30, 20, or 10.

In connection with the desirable and useful effects in connection with the treatment or prophylaxis of harmful effects of inflammatory cytokines, it is also important that the compound for the use according to the invention only has minor (preferably no) side effects, thus, it is preferred that the compound, at the IC₅₀ concentration in the Cytokine inhibition Assay, shows a reduction of the thymidine uptake by human lymphocytes in the Lymphocyte Proliferation Assay, as defined herein, using phytomemagglutinin (PHA), of less than 50%, preferably less than 40%, especially less than 30%, in particular less than 20%. In particular, the ratio between the IC₅₀ value in the Cytokine inhibition Assay and the IC₅₀ value in the Lymphocyte Proliferation Assay for the compound in question should be equal to or less than 1, such as less than, e.g., 0.9, 0.8, 0.7, 0.6, 0.5, 0.4, 0.3, or 0.2.

Compounds

In the present context, the term “hydrocarbyl” is intended to mean a group which in itself comprises carbon and hydrogen atoms exclusively. Typically, hydrocarbyl groups mentioned herein comprise 1-12 carbon atoms (C₁₋₁₂), and more specifically 1-6 carbon atoms (C-₁₋₆). Hydrocarbyl groups may be straight-chained, branched or cyclic in their basis structure, when ignoring any substituents. It should be understood that hydrocarbyl groups may also consist of, e.g. a straight-chained part and a cyclic part as, e.g. in a cyclohexyl-ethyl group. Hydrocarbyl groups within the meaning of the present invention may be saturated or may contain one or more unsaturated bonds selected from double bonds and triple bonds, thus, hydrocarbyl groups may be based on alkanes, alkenes, alkadienes, alkatrienes, alkynes, etc.

Illustrative examples of C₁₋₁₂ hydrocarbyl are methyl, ethyl, propyl, isopropyl, cyclopropyl, butyl, tertbutyl, secbutyl, isobutyl, cyclobutyl, 1-methylbutyl, 1,1-dimethylpropyl, pentyl, cyclopentyl, hexyl, cyclohexyl, 3-methylbutyl, dodecyl, vinyl, prop-2-enyl (allyl), butenyl, pentenyl, 3-methylbut-2-enyl, 1,1-dimethyl-prop-2-enyl, 1-methylpentyl, 1-ethylbutyl, hexenyl, heptenyl, octenyl, decaenyl, butadienyl, pentadienyl, hexadienyl, heptadienyl, hexatrienyl, heptatrienyl, octatrienyl, among which methyl, ethyl, propyl, isopropyl, butyl, isobutyl, secbutyl, tertbutyl, pentyl, 1-methylbutyl, 1,1-dimethylpropyl, hexyl, 1-methylpentyl, 1-ethylbutyl, cyclopentyl, cyclohexyl, prop-2-enyl, 1,1-dimethyl-prop-2-enyl, 3-methylbutyl, and 3-methylbut-2-enyl are especially relevant examples.

It should also be understood that the various possible stereoisomers of the groups mentioned above are within the meaning of the individual terms and examples. As an illustrative example, “1-methyl-butyl” exists in both the (R) and the (S) form, thus, both (R)-1-methyl-butyl and (S)-1-methyl-butyl is covered by the term “1-methyl-butyl”. This is of course especially relevant because various examples from the literature show that even the minor changes in the stereochemical configuration of a given substituent may alter the effect dramatically.

As mentioned above, hydrocarbyl may be substituted with one or more substituents, preferably substituents selected from hydroxy, carboxy, halogen such as fluoro, chloro, bromo or iodo, amino, and amino which is optionally alkylated with one or two C₁₋₆ alkyl groups (i.e. —NH(C₁₋₆-alkyl) and —N(C₁₋₆-alkyl)₂);

The term “halogen” used in the description and claims is intended to coer fluoro, chloro, bromo and iodo.

The term “alkyl” is used in its normal meaning, cf. IUPAC. Examples of “C₁₋₆-alkyl” are methyl, ethyl, propyl, isopropyl, pentyl, cyclopentyl, hexyl, cyclohexyl, and examples of “C₁₋₄-alkyl” are methyl, ethyl, propyl, isopropyl, cyclopropyl, butyl, isobutyl, tertbutyl, cyclobutyl. Analogously, “C₁₋₃-alkyl” covers methyl, ethyl, propyl, isopropyl, cyclopropyl.

The term “alkoxy” is intended to mean “alkyl-oxy”, and the term “acyl” is intended to mean “alkyl-carbonyl”.

As described above, the present invention is based on the finding that various bis-aromatic compounds possess highly interesting biological effects. Especially promising compounds are bis-aromatic compounds of the general formula I

(WA)ₘ—Ar¹—CO—V—Ar²—(AW)ₙ I

wherein V is either —CRR—CRR—, —CR═CR— or —C≡C. Thus, it is clear that the general formula comprises 1,3-bis-aromatic propan-1-ones, 1,3-bis-aromatic prop-2-en-1-ones, 1,3-bis-aromatic propyn-1-ones, as well as α- and/or β-substituted analogues thereof. When V is —CR═CR—, it may either be in the cis (Z) or the trans (E) form, of which the trans form is preferred. It is preferred that the group V is selected from —CHR—CHR—, —CR═CH—, —CH═CR—, —CH₂—CHR—, and —CHR—CH₂—.

As mentioned above, the compounds may be substituted in the α- and/or β-position relative to the keto group; thus, the substituents R may independently designate hydrogen, cyano, nitro, nitroso, amino, and halogen such as fluoro, chloro, bromo, or iodo, R_{H}, and AW, among which hydrogen, C₁₋₃ alkyl, cyano, and halogen such as fluoro, chloro, bromo, or iodo are the most preferred. Although the unsubstituted variants are the most common, it is contemplated that bis-aromatic compounds in which one or both of the α- and/or β-position(s) R is/are substituted with e.g. methyl or ethyl are of great value with respect to the relevant activity and selectivity/tolerability.

The introduction of such substituent has previously been difficult to accomplish, however, the present invention also provides methods (see Experimental) for the introduction of substituents in the α- and, β-position. Thus, these novel and highly interesting methods are to be considered a further aspect of the present invention (see further below).

The groups Ar¹ and Ar² in the general formula I each designate an aromate selected from phenyl and 5- or 6-membered unsaturated heterocyclic rings containing one, two or three heteroatoms selected from oxygen, sulphur, and nitrogen, such as furanyl, thiophenyl, pyrrolyl, imidazolyl, isoxazolyl, oxazolyl, thiazolyl, pyrazolyl, pyridinyl, or pyrimidinyl, etc. Typically, at least one of the aromates Ar¹ and Ar² is optionally substituted phenyl, and preferably both of Ar¹ and Ar² are optionally substituted phenyl, whereby the compound is either a chalcone (V═—CR═CR—), a dihydrochalcone (V═—CRR—CRR—), or a dehydrochalcone (V═—C≡C—). Preferred compounds are compounds of the chalcone and the dihydrochalcone types.

Apart from the important substitution with the groups AW as defined below in connection with formula I, the aromate may carry other substituents which either will not to any substantial extent detract from the useful effect and selectivity of the bis-aromatic compounds, or will enhance these properties or relevant properties related to the use and utility of the bis-aromatic compounds, e.g., their solubility (such as when the bis-aromatic compounds carry a nitrogen-containing basic group or a carboxyl group which can form water-soluble salts with pharmaceutically acceptable counter ions). Thus, the aromates may be substituted with one or more substituents selected from halogen; cyano; nitro; nitroso; carboxy and C₁₋₁₂, preferably C₁₋₆, straight, branched or cyclic aliphatic hydrocarbyl which may be saturated or may contain one or more unsaturated bonds selected from double bonds and triple bonds, which hydrocarbyl may be substituted with one or more substituents selected from hydroxy, carboxy halogen such as fluoro, chloro, bromo or iodo, amino, and amino which is optionally alkylated with one or two C₁₋₆ alkyl groups; preferably such optional further substituents on the aromates Ar¹ and Ar² are selected from halogen; cyano; nitro; and C₁₋₆, straight, branched or cyclic aliphatic hydrocarbyl which may be saturated or may contain a double bond, which hydrocarbyl may be substituted with one or more substituents selected from hydroxy, halogen such as fluoro, chloro, bromo or iodo, amino, and amino which is optionally alkylated with one or two C₁₋₆ alkyl groups. Especially suitable examples of such groups are methyl, ethyl, propyl, isopropyl, butyl, isobutyl, secbutyl, tertbutyl, pentyl, 1-methylbutyl, 1,1-dimethylpropyl, hexyl, 1-methylpentyl, 1-ethylbutyl, cyclopentyl, cyclohexyl, prop-2-enyl, 1,1-dimethyl-prop-2-enyl, 3-methylbutyl, and 3-methylbut-2-enyl.

According to the general formula I each of the two aromates may carry one or more further substituents AW. It is believed that the compound should carry at least one substituent AW (i.e. m and n are not both zero). The substituents independently designate a group AR_{H}, AH, or a group AZ, wherein each A independently is selected from —O—, —S—, —NH—, or —NR_{H}—, preferably from —O— and —NR_{H}—, R_{H} designates C₁₋₆ straight, branched or cyclic aliphatic hydrocarbyl which may be saturated or may contain one or more unsaturated bonds selected from double bonds and triple bonds, which hydrocarbyl may be substituted with one or more substituents selected from hydroxy, halogen such as fluoro, chloro, bromo or iodo, amino, and amino which is optionally alkylated with one or two C₁₋₆ alkyl groups, and Z designates (when the compound is a prodrug) a masking group which is readily decomposed under conditions prevailing in the animal body to liberate a group AH, in which A is as defined above.

Among the bis-aromatic compounds of the general formula I, the preferred ones are generally those in which at least one A is —O—, preferably each A is O, mainly because of their expected excellent properties with respect to activity and selectivity/tolerability. However, it is well known that the oxygen atom in the form of oxy in many biologically active compounds may, with greater or lesser retention of, and indeed in certain cases with enhancement of, the biological activity, be replaced with bioisosteric groups, such as —S—, —NH—, and —NR_{H}— as mentioned above.

With respect to the group(s) R_{H} above, it is preferred that these independently designate C₁₋₆ straight, branched, or cyclic aliphatic hydrocarbyl which may be saturated or may contain a double bond, which hydrocarbyl may be substituted with one or more substituents selected from hydroxy, halogen such as fluoro, chloro, bromo or iodo, amino, and amino which is optionally alkylated with one or two C₁₋₆ alkyl groups, preferably R_{H} designates C₁₋₆ straight, branched, or cyclic aliphatic hydrocarbyl which may be saturated or may contain a double bond. Especially relevant examples of the group R_{H} are methyl, ethyl, propyl, isopropyl, butyl, isobutyl, secbutyl, tertbutyl, pentyl, 1-methylbutyl, 1,1-dimethylpropyl, hexyl, 1-methylpentyl, 1-ethylbutyl, cyclopentyl, cyclohexyl, prop-2-enyl, 1,1-dimethyl-prop-2-enyl, 3-methylbutyl, and 3-methylbut-2-enyl.

The symbol m designates the number of further substituents AW on the aromate Ar¹ and m is 0, 1, 2, 3 or 4 i.e. aromate in the 1-position relative to the keto group carries 0-4 subtituents of the type AW in addition to any of the substituents defined above. Preferably, the number of substituents in that particular aromate (which preferably is phenyl) is 1, 2, 3, or 4, in particular 2, 3, or 4. Analogously, the symbol n designates the number of further substituents AW on the aromate Ar², i.e. the aromate in the 3-position relative to the keto group, and is 0, 1, 2 or 3, preferably 1, 2, or 3, in particular 2 or 3. Furthermore, it is preferred that the sum n+m is at least 2, preferably at least 3, in particular at least 4.

In the cases where a substituent is present in the two aromates, which preferably are phenyl, it is believed to be relevant that at least one of the groups AW is in a position in Ar¹ most remote relative to and/or next to the position through which Ar¹ is bound to the carbonyl group, and that at least one of the groups AW is in a position in Ar² most remote relative to and/or next to the position through which Ar² is bound to V.

Alternatively, it may also be relevant to consider compounds where the aromates are phenyl, and where a substituent is present in the 3-position of Ar¹.

The prodrugs used according to the invention are, e.g., compounds of the general formula I, II or IIa in which Z is a group which is readily decomposed under conditions prevailing in the animal body to liberate the group AH. As an important example, when A is O such as is the case in important compounds used according to the invention, it is preferred that Z is a group which is readily decomposed under conditions prevailing in the animal body to liberate the group OH.

The establishment of prodrug forms suitable in connection with particular substituents in drugs is based upon the fact that certain types of groups will tend to be decomposed in the animal body in accordance with various decomposition pathways. Thus, among the specific prodrug groups (A)-(F)

—CO—R″ (A)

—CON(CH₃)₂ (B)

—CR*R**—O—R″ (C)

—CR*R**—O—CO—R″ (D)

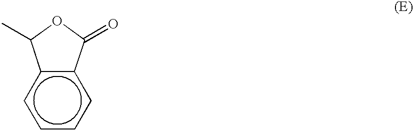 —CO—R* (F)

wherein

R* and R** each independently designate hydrogen or C₁₋₃ alkyl, R″ designates C₁₋₆ alkyl or an optionally substituted aromate Ar¹ or Ar² as defined above and in claim 15,

the groups (A), (D), (E), and (F) are groups which will be decomposed by esterases to result in the corresponding free group such as the hydroxy group. The group (B) will be subjected to removal of one of the methyl groups in the liver, and the group thus formed will be relatively readily decomposable in plasma. The oxy-containing groups (C) are groups which are relatively labile under acidic conditions and, as such, are adapted to be decomposed in the human body, that is, in macrophages. Quite generally, the prodrug group Z will be one which prevents the active molecule from being converted, in the liver, to a form which, from a practical point of view, will be inactive and quickly will be eliminated from the animal body, such as the forms where free phenolic OH groups are sulphated in the liver or are coupled to glucuronic acid in the liver. Preferred prodrug groups Z are pivaloyl, pivaloyloxymethyl, N,N-dimethylcarbamoyl, and C₁₋₆ acyl.

Based upon the above-mentioned general preference for substituents AW which contain —O— (but taking into consideration that the oxygen atom could be replaced with the a bioisosteric group), this substituent could be called “an oxy-functional substituent”. While it is presumed that the activity of the oxy-functional substituent is related to the substituent in the “free” form, that is, to hydroxy when A is —O—, to thiolo when A is —S—, and to amino or monoalkylamino when A is —NH— or —NR_{H}—, very interesting results obtained with bis-aromatic compounds of the formula I where AW is alkenyloxy raise the intriguing question whether the active form in theses cases is the alkenyloxy-substituted form, or whether the alkenyloxy group is converted to a hydroxy group, maybe in the environment where the compound is expressing its biological action, before the bis-aromatic compound exerts it action. As will be understood, this possibility is covered by the definition R_{H} above, while the definition of Z is adapted to represent “prodrug” forms which, in accordance with well known principles used in the construction of suitable administration embodiments of chemical compounds containing, e.g., free hydro groups as substituents on aromatic rings, will be decomposed in the animal body to result in the corresponding compound in which Z is hydrogen.

More specific compounds for the uses according to the present invention are defined in the claims 11-56 and 57-289. Thus, these claims represent preferred embodiments of the uses according to the invention.

Among the compounds defined in the claims related to the various uses, i.e. claims 11-56, it is believed that some are novel as such. Thus, the present invention also relates to novel compounds which are highly interesting with respect to their uses for the treatment of a number of diseases.

It should be understood that the above definitions also apply to the novel bis-aromatic compounds, i.e. the novel chalcones and the novel dihydrochalcones, according to the invention. Thus, the present invention also relates to the compounds defined in any of the claims 57-289.

Furthermore, the present invention also relates to the general use of the novel chalcones and dihydrochalcones as drug substances, as well as to pharmaceutical compositions comprising such novel compounds.

Synthesis

Synthesis of Bis-aromatic Compounds

As mentioned above, a number of the compounds of the general formula I are known, whereas many of the compounds of the general formula I are novel compounds. The known compounds may be isolated or synthesised in accordance with methods known from the literature or methods analogous thereto. The novel compounds may, likewise, be produced by methods known per se or methods which are analogous to such methods. Examples of excellent methods for preparing compounds of the 1,3-bis-aromatic-prop-2-enone or the 1,3-bis-aromatic-prop-2-ynone types are given in the following. Further examples of methods for the preparation of the compound used according to the present invention are described in WO 95/06628 and WO 93/17671 and in the references cited therein.

Compounds of the general formula I in which V is —CH═CH— are prepared by reacting a ketone (an acetophenone in the case where Ar¹ is phenyl)

(WA)ₘ—Ar¹—CO—CH₃

with an aldehyde (a benzaldehyde in the case where Ar² is phenyl)

HCO—Ar²—(Aw)ₙ

wherein W, A, m, n, Ar¹, Ar² are as defined above.

This reaction, which is a condensation reaction, is suitably carried out under acid or base catalysed conditions. A review of such processes may be found in Nielsen, A. T., Houlihahn, W. J., Org. React. 16, 1968, p 1-444. In particular the method described by Wattanasin, S. and Murphy, S., Synthesis (1980) 647 has been found to be very successful. The reaction may suitably be carried out in protic organic solvents, such as lower alcohols (e.g. methanol, ethanol, or tert.butanol), or lower carboxylic acids (formic, glacial acetic, or propionic acid), or in aprotic organic solvents such as ethers (e.g. tetrahydrofuran, dioxane, or diethyl ether), liquid amides (e.g. dimethylformamide or hexamethylphosphordiamide), dimethylsulfoxide, or hydrocarbons (e.g. toluene or benzene), or mixtures of such solvents. When carrying out the reaction under base catalysed conditions, the catalyst may be selected from sodium, lithium, potassium, barium, calcium, magnesium, aluminum, ammonium, or quaternary ammonium hydroxides, lower alkoxides (e.g. methoxides, ethoxides, tert.butoxides), carbonates, borates, oxides, hydrides, or amides of lower secondary amines (e.g. diisopropyl amides or methylphenyl amides). Primary aromatic amines such as aniline, free secondary amines such as dimethyl amine, diethyl amine, piperidine, or pyrrolidine as well as basic ion exchange resins may also be used.

Acid catalysts may be selected from hydrogen chloride, hydrogen bromide, hydrogen iodide, sulfuric acid, sulfonic acids (such as paratoluenesulfonic or methanesulfonic acid), lower carboxylic acids (such as formic, acetic or propionic acid), lower halogenated carboxylic acids (such as trifluoroacetic acid), Lewis acids (such as BF₃, POCl₃, PCl₅, or FeCl₃), or acid ion exchange resins.

A drawback of the base catalysed condensation is the poor yield obtained if the aromatic ring in which the ketone or the aldehyde or both is substituted with one or more hydroxy groups. This drawback can be overcome by masking the phenolic group as described by T. Hidetsugu et al. European patent application 0370 461 (1989). Deprotection is easily performed by mineral acids such as hydrochloric acid.

The reaction may be carried out at temperatures in the range of 0-100° C., typically at room temperature. Reaction times may be from 30 min. to 24 hours.

Compounds of the general formula I in which V is —C≡C— may be prepared by reacting an activated derivative of a carboxylic acid of the general formula

(WA)ₘ—Ar²—COOH

with an ethyne derivative

H—C≡C—Ar²—(AW)ₙ

wherein W, A, m, n, Ar¹, and Ar² are as defined above. Reactions of this type are described by Tohda, Y., Sonogashihara, K., Haghara, N., Synthesis 1977, p 777-778. It is contemplated that the activated derivative of the carboxylic acid may be an activated ester, an anhydride or, preferably, an acid halogenide, in particular the acid chloride. The reaction is normally carried out using the catalysts described by Tohda, Y. et al. cited above, namely copper(I)iodide/triphenylphosphine-palladium dichloride. The reaction is suitably carried out in triethylamine, a mixture of triethylamine and pyridine or triethylamine and toluene under a dry inert atmosphere such as nitrogen or argon. The reaction is generally carried out at reduced temperature such as in the range from −80° C. to room temperature, the reaction time typically being from 30 minutes to 6 hours.

In the above reactions, it may be preferred or necessary to protect various sensitive or reactive groups present in the starting materials so as to prevent said groups from interfering with the reactions. Such protection may be carried out in a well-known manner, e.g. as described in “Protective Groups in Organic Chemistry” by Theodora Green. For example, in the reaction between the activated acid derivative and the acetylene derivative, a hydroxy group on Ar¹ and/or Ar² may be protected in the form of the methoxymethyl ether, N,N-dimethylcarbamoyl ester, or allyl ether. The protecting group may be removed after the reaction in a manner known per se.

Compounds of the general formula I in which V is —CH₂—CH₂— can be prepared by hydrogenation of the corresponding α, β-unsaturated compound where V is —CH═CH— as it has been described by the inventors in Nielsen, S. F. et al. Tetrahedron, 53, pp 5573-5580 (1997) and in the examples. Further possible synthetic routes for the preparation of the saturated variants are described in “Advanced Organic Chemistry” by Jerry March, 3^{rd} ed. (especially chapter 15, pages 691-700) and references cited therein. Thus, it is possible to obtain a large variety of compounds of the 1,3-bis-aromatic-propan-1-one type from the corresponding prop-2-en-1-ones.

Synthesis of α- and/or β-substituted bis-aromatic compounds

A number of methods for the preparation of β-alkyl substituted and β-alkyl substituted chalcones have been published. However, these methods have certain drawbacks either with respect to the scope of the possible resulting compound, the use of not generally applicable reaction conditions, or with respect to yields and purity of the products.

Thus, published methods for preparing β-methylchalcones include cycloaddition of diazomethane to chalcones followed by pyrolysis (C. B Rao et al. Ind. J. Chem. 1986, 25B, 400-403), self-condensation of acetophenones catalysed by various catalysts to give β-methylchalcones with identical substitution pattern at the two aromatic rings (L. J. Mazza et al. Synthesis 1980, 41-44; N. O. Calloway et al. J. Am. Chem. Soc. 1937, 59, 809; R. E. Lyle et al. J. Am. Chem. Soc. 1953, 75, 5959-5961; H. Alper et al. J. Org. Chem. 1976, 41, 806-808), palladium assisted condensation of substituted α-1,2-propadienylbenzenemethanols to halogenated benzenes (I. Shimuzu et al. J. Org. Chem. 1985, 50, 537-539), and conjugate addition of lithium dimethylcuprate to give a dihydrochalcone, which is selenylated with phenylselenyl bromide followed by oxidation and elimination of phenylselenoxide (A. Pelter et al. Tetrahedron 1979, 35, 531-533). In principle the latter method can be used for introduction of alkyl groups larger than methyl. However, certain limitations exist in connection with these methods. α-alkylated chalcones are, e.g., obtained by condensation of an arylketone with a benzaldehyde. In general, however, the yields are poor especially when larger arylketones are used as starting material. A number of catalysts including alkali hydroxides (T. Széll J. Prakt. Chem. 1962, 17, 346-348), piperidinium acetate (M. L. Edwards et al. J. Med. Chem. 1990, 33, 1948-1954), and hydrochloric acid (R. D. Abell J. Chem. Soc. 1953, 2834-2836) have not increased the yields. A further drawback is the difficult availability of the arylketones used as starting materials.

The present invention now provides general and straightforward methods for the preparation of α- and β-hydrocarbyl substituted chalcones as well as the dihydro analogues thereof. Thus, α- and β-hydrocarbyl substituents are introduced by a simple route, resulting in high yields from readily available starting materials. For specific embodiments see claims 306 and 311 and the claims dependent thereon.

The key starting material in the novel method for the preparation of β-hydrocarbyl substituted chalcones is the compound of the general formula 2 wherein Ar¹ and Ar² are as defined above, i.e aromates, preferably phenyl. The compound 2 can easily be synthesised in high yields from the compounds 1 by conjugated addition of a cyanide such as hydrogen cyanide, potassium cyanide, or sodium cyanide, preferably sodium cyanide by methods known per se, e.g. as described in “Advanced Organic Chemistry” by Jerry March, 3^{rd} ed. (especially chapter 15, Reaction 5-25) and references cited therein. It is noted that the compounds of the general formula 1 can be synthesised by methods described elsewhere herein.

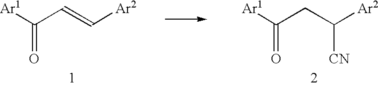

The compound of the general formula 3, wherein R_{β} may be selected from C₁₋₆ straight, branched, and cyclic aliphatic hydrocarbyl which may be saturated or may contain one or more unsaturated bonds selected from double bonds and triple bonds, which hydrocarbyl may be substituted with one or more substituents selected from C₁₋₆ alkoxy, hydroxy, halogen such as fluoro, chloro, bromo, or iodo, amino, amino which is optionally substituted with one or two C₁₋₆ alkyl groups, preferably C₁₋₆ straight, branched, or cyclic alkyl such as methyl, ethyl, propyl, isopropyl, butyl, isobutyl, cyclopentyl, and cyclohexyl, may be prepared by treatment of the compound 2 with a strong base such as NaH or a basic salt of a dialkylamine, e.g. lithium diisopropylamide, followed by addition of a hydrocarbyl halide corresponding to the hydrocarbyl group R_{β}. Examples of hydrocarbyl halides are hydrocarbyl fluoride, hydrocarbyl chloride, hydrocarbyl bromide, hydrocarbyl iodide, among which the bromide and iodide seem to be the most relevant.

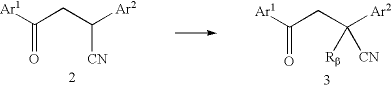

The reaction is typically performed in an anhydrous aprotic solvent such as in dimethylformamide, lower aliphatic ketones like acetone and butanone, aliphatic ethers like tetrahydrofuran or diethylether, at a temperature between −100 and 0° C., preferably between −100 and −50° C., and in the case of tetrahydrofuran preferably between −90 and −70° C. such as around −78° C. A relatively low reaction temperature is preferred in order to suppress by-product formation. Dry tetrahydrofuran seems to be especially suited as a solvent as in addition to non-reactivity toward the strong base and the halide it remains soluble at −78° C.

The compound of the general formula 4 may be obtained by elimination of HCN from the β,β-disubstituted species 3. The elimination conditions may be established by addition of a strong base such as NaH or a basic salt of a dialkylamine, e.g. lithium diisopropylamide, to compound 3. Addition of a strong base may be done either with or without prior isolation and optionally also purification of the compound 3, with due consideration, of course, to the solvent selected. Good results have been obtained with isolation of the compound 3.

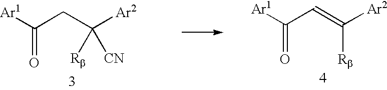

The elimination reaction is typically performed in an inert organic solvent such as benzene, toluene, o-, m-, or p-xylene at a temperature between room temperature and the boiling point of the organic solvent, preferably at the boiling point of the organic solvent or at a temperature of 50-120° C.

With the above method it is possible to prepare β-alkyl substituted chalcones. Furthermore, the β-hydrocarbyl substituted dihydrochalcone analogues thereof may be prepared from the corresponding chalcone by the methods for hydrogenation described herein.

The present invention also provides a novel method for the preparation of α-hydrocarbyl substituted chalcones. The key starting material in this method is the compound of the general formula 5, wherein Ar¹ and Ar² are as described above, and where X₂ is selected from cyano and halogen such as fluoro, chloro, bromo, or iodo. In the same situation where X₂ cyano, the compound 5 is identical compound 2 and can be prepared as described above, and where X₂ is halogen, the compound 5 may be prepared from 1 by regioselective hydrohalogenation methods known per se, see e.g. “Advanced Organic Chemistry” by Jerry March, 3^{rd} ed. (especially Chapter 15, reaction 5-1).

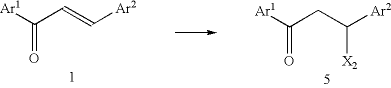

According to the invention, the compound 6, wherein R is selected from C₁₋₆ straight, branched, an cyclic alkyl, may be prepared by reacting 5 with a trialkylsilyl halide such as terbutyldimethylsilyl chloride, in the presence of a strong base, e.g. NaH.

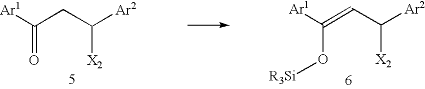

A preferred silyl enol-ether is the tertbutyldimethyl enol ether since this leads to stable compounds which can be isolated and purified. The silylation reaction is performed in an anhydrous aprotic solvent such as dimethylformamide, lower aliphatic ketones like acetone, butanone, aliphatic ethers like tetrahydrofuran or diethylether, preferably tetrahydrofuran, at a temperature between 0° C. and the boiling point of the solvent, preferably at room temperature.

The α, β-substituted compound 7, wherein R_{α} is selected from C₁₋₆ straight, branched, and cyclic aliphatic hydrocarbyl which may be saturated or may contain one or more unsaturated bonds selected from double bonds and triple bonds, which hydrocarbyl may be substituted with one or more substituents selected from C₁₋₆ alkoxy, hydroxy, halogen such as fluoro, chloro, bromo, or iodo, amino, amino which is optionally substituted with one or two C₁₋₆ alkyl groups, preferably C₁₋₆ straight, branched, or cyclic alkyl such as methyl, ethyl, propyl, isopropyl, butyl, isobutyl, cyclopentyl, and cyclohexyl, may be obtained by treatment of the silyloxy compound 6 with a hydrocarbyl halide such as hydrocarbyl fluoride, hydrocarbyl chloride, hydrocarbyl bromide, hydrocarbyl iodide, preferably the bromide or the iodide, wherein the hydrocarbyl halide corresponds to the hydrocarbyl group R_{α} as defined above. The alkylation reaction is performed in the presence of a dried fluoride-donating agent such as HF, LiF, KF, RbF, CsF, preferably CsF (which can be almost completely dried), or a fluoride salt of mono-, di-, tri-, or tetraalkylammonium such as (C₂H₅)₃NHF and (C₄H₉)₄NF.

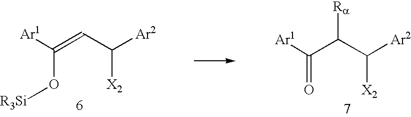

The reaction is performed in an anhydrous aprotic solvent as described for the reaction between the compound 5 and the trialkylsilyl halide at a temperature between 0° C. and the boiling point of the solvent, preferably at room temperature. Preferably dimethylformamide is used. Although isolation of compound 6 is normally performed, it is envisaged that the reaction may be accomplished without isolation of 6 since the solvent systems preferred in the silylation and in the addition steps are both aprotic.

Finally, as for the β-substituted analogues, the compound 8 may be prepared by subjecting the compound 7 to elimination conditions whereby HX₂, e.g. HCN, is eliminated. The elimination conditions may comprise addition of a strong base such as NaH, a basic salt of a dialkylamine like lithium diisopropylamide, or an alkoxide like sodium ethoxide, to compound 7 either with or without isolation and optionally purification of the compound 7.

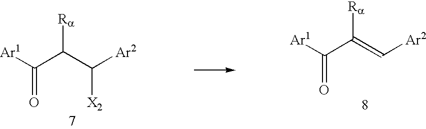

As above, the reaction is performed in an inert organic solvent such as benzene, toluene, o-, m-, or p-xylene at a temperature between room temperature and the boiling point of the organic solvent, preferably at the boiling point of the organic solvent.

Thus, with these novel methods at hand, it is possible to prepare α- as well as β-hydrocarbyl substituted chalcones, and also, as mentioned above, the corresponding dihydrochalcones. It is furthermore contemplated that sequential combination of the two methods (e.g. by exclusion of the final elimination step in the first sequence) may give rise to a further interesting group of compounds, namely the α, β-dihydrocarbyl substituted chalcones and the corresponding dihydrochalcones.

It should be understood that depending on the substituents present in the two aromatic rings, Ar¹ and Ar², it may be preferred or even necessary to protect various sensitive or reactive groups, e.g. free hydroxy groups and amines such as primary and secondary amines, present in the compounds 1-8 so as to prevent said groups from interfering with the reactions. Such protection may be carried out in a well-known manner, e.g. as described in “Protective groups in Organic Chemistry” by Theodora Green.

As it is clear from the above, key starting materials for the preparation of compound used according to the invention and the novel compounds are, on the one hand, aromatic methyl ketones of the formula (WA)ₘ—Ar¹—CO—CH₃ (an acetophenone in the case where Ar¹ is phenyl) and aromatic aldehydes of the formula HCO—Ar²—(AW)ₙ (a benzaldehyde in the case where Ar² is phenyl) which are used in one of the possible routes for the preparation of 1,3-bis-aromatic-prop-2-en-1-ones, and, on the other hand, aromatic carboxylic acids of the formula (WA)ₘ—Ar¹—COOH (benzoic acids where Ar¹ is phenyl) and ethyne derivatives of the formula H—C≡C—Ar²—(AW)ₙ preparation of 1,3-bis-aromatic-prop-2-yn-1-ones. (As mentioned above, the preparation of 1,3-bis-aromatic-propan-1-ones may be accomplished via the corresponding 1,3-bis-aromatic-prop-2-en-1-one).

This being said, it will be clear for the person skilled in the art that the chemical literature provides an almost unlimited source for possible starting materials for the preparation of the compounds discussed herein, especially in the cases where the aromates Ar¹ and Ar² are phenyl since the benzene chemistry is one of the most developed within the field of organic chemistry. In connection herewith it is noted that the ethyne derivatives mentioned above can be easily prepared from the corresponding aromatic aldehydes, cf. also the Examples. Especially interesting compounds discussed herein are polyalkoxylated chalcones and derivatives hereof. It is clear that starting materials for such compounds may be obtained by the alkylation of the corresponding polyhydroxylated compounds. Thus, again an almost unlimited number of readily accessible starting materials are either commercially available or may be readily synthesised.

Formulation of Pharmaceutical Compositions

The administration route of the aromatic compound as defined above, such as the bisaromatic α, β-unsaturated ketones of the general formula I, may be of any suitable route which leads to a concentration in the blood corresponding to a therapeutic concentration. Thus, e.g., the following administration routes may be applicable although the invention is not limited thereto: the oral route, the parenteral route, the cutaneous route, the nasal route, the rectal route, the vaginal route and the ocular route. It should be clear to a person skilled in the art that the administration route is dependant on the compound in question, particularly, the choice of administration route depends on the physico-chemical properties of the compound together with the age and weight of the patient and on the particular disease and the severity of the same.

The aromatic compounds as defined above, such as the bis-aromatic α, β-unsaturated ketones or derivatives thereof, may be contained in any appropriate amount in a pharmaceutical composition, and are generally contained in an amount of about 1-95% by weight of the total weight of the composition. The composition may be presented in a dosage form which is suitable for the oral, parenteral, rectal, cutaneous, nasal, vaginal and/or ocular administration route. Thus, the composition may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, aerosols and in other suitable form.

The pharmaceutical compositions may be formulated according to conventional pharmaceutical practice, see, e.g., “Remington's Pharmaceutical Sciences” and “Encyclopedia of Pharmaceutical Technology”, edited by Swarbrick, J. & J. C. Boylan, Marcel Dekker, Inc., New York, 1988.

Pharmaceutical compositions according to the present invention may be formulated to release the active compound substantially immediately upon administration or at any substantially predetermined time or time period after administration. The latter type of compositions are generally known as controlled release formulations.

In the present context, the term “controlled release formulations” embraces

i) formulations which create a substantially constant concentration of the drug within the body over an extended period of time,

ii) formulations which after a predetermined lag time create a substantially constant concentration of the drug within the body over an extended period of time,

iii) formulations which sustain drug action during a predetermined time period by maintaining a relatively, constant, effective drug level in the body with concomitant minimization of undesirable side effects associated with fluctuations in the plasma level of the active drug substance (sawtooth kinetic pattern),

iv) formulations which attempt to localize drug action by, e.g., spatial placement of a controlled release composition adjacent to or in the diseased tissue or organ,

v) formulations which attempt to target drug action by using carriers or chemical derivatives to deliver the drug to a particular target cell type.

Controlled release formulations may also be denoted “sustained release”, “prolonged release”, “programmed release”, “time release”, “rate-controlled” and/or “targeted release” formulations.

Controlled release pharmaceutical compositions may be presented in any suitable dosage forms, especially in dosage forms intended for oral, parenteral, cutaneous nasal, rectal, vaginal and/or ocular administration.

Administration of the aromatic compounds defined above, such as bis-aromatic α, β-unsaturated ketones in form of a controlled release formulation is especially preferred in such cases where the compound in question

i) has a narrow therapeutic index [i.e. the difference between the plasma concentration leading to harmful side effects or toxic reactions and the plasma concentration leading to a therapeutic effect is small; in general, the therapeutic index, TI, is defined as the ratio of median lethal dose (LD₅₀) to median effective dose (ED₅₀)],

ii) has a narrow absorption window in the gastrointestinal tract. In such cases, it is important that the intact dose of the aromatic compound reaches the site of absorption in order to avoid a substantially uniform distribution of the compound administered in the whole gastrointestinal tract,

iii) has a very short biological half-live so that frequent dosing during a day is required in order to sustain the plasma level at a therapeutic level,

iv) in cases where it is desirable to enable preparation of a pharmaceutical composition intended for use only once or twice daily or even less frequent with the purpose of reducing patient compliance problems,

v) in cases where it is desirable to avoid peak concentrations in the plasma so that harmful side or toxic effects related to such high concentrations can be substantially reduced, or

vi) in cases where it is desirable to avoid fluctuations in plasma concentration of the compound administered (in order to even out any peak and valley concentration).

In general, two basically different strategies can be applied in order to obtain a controlled release formulation in which the rate of release outweighs the rate of metabolism of the compound in question.

In the first strategy, the principle aims at changing the properties of the active drug substance by converting the substance into a masked form. The compounds of the above formulae in which Z is one of the groups (A)-(E) are representatives of this strategy.

In the second strategy, controlled release is obtained by appropriate selection of various formulation parameters and ingredients, including, e.g. various types of controlled release compositions and coatings (formulation-method).

As mentioned above, the first strategy comprises use of the prodrug principle, i.e. converting the active drug substance into a per se inactive derivative which, upon administration to the organism, within the body of the organism by an enzymatic or non-enzymatic process releases the active drug substance so that the drug substance can exert its therapeutic effect. By proper choice of the prodrug it is possible to obtain a prodrug which releases the active drug substance with a controlled rate so that it thereby is possible to extend the effect of the drug in the body.

The other strategy comprises the use of the active drug substance per se and then formulate the active drug substance together with appropriate excipients into a pharmaceutical composition which upon administration of the composition to the organism releases the active substance in a controlled manner. Examples include single or multiple unit tablet or capsule compositions, oil solutions, suspensions, emulsions, microcapsules, microspheres, nanoparticles, liposomes, delivery devices such as those intended for oral, parenteral, cutaneous, nasal, vaginal or ocular use.

It will be appreciated that a combination of the above-mentioned two methods can be used in controlled release compositions, comprising the aromatic compounds defined above, such as bis-aromatic α, β-unsaturated ketones, according to the invention, e.g., by using a prodrug of the compound in question and then formulating according to the principles mentioned above.

In the present context every pharmaceutical composition is an actual drug delivery system, since upon administration it presents the active drug substance to the body of the organism.

Solid Dosage Forms for Oral Use

Formulations for oral use include tablets which contain the active ingredient in admixture with non-toxic pharmaceutically acceptable excipients. These excipients may be, for example,

inert diluents or fillers, such as sucrose, sorbitol, sugar, mannitol, microcrystalline cellulose, starches including potato starch, calcium carbonate, sodium chloride, lactose, calcium phosphate, calcium sulfate or sodium phosphate;

granulating and disintegrating agents, for example, cellulose derivatives including microcrystalline cellulose, starches including potato starch, croscarmellose sodium, alginates or alginic acid;

binding agents, for example, sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone or polyethylene glycol; and

lubricating agents, including glidants and antiadhesives, for example, magnesium stearate, zinc stearate, stearic acid, silicas, hydrogenated vegetable oils or talc.

Other pharmaceutically acceptable excipients can be colorants, flavouring agents, plasticizers, humectants, buffering agents etc.

The tablets may be uncoated or they may be coated by known techniques, optionally to delay disintegration and absorption in the gastrointestinal tract and thereby providing a sustained action over a longer period. The coating may be adapted to release the active drug substance in a predetermined pattern, e.g; in order to achieve a controlled release formulation (see below) or it may be adapted not to release the active drug substance until after passage of the stomach (enteric coating). The coating may be a sugar coating, a film coating (e.g. based on hydroxypropyl methylcellulose, methylcellulose, methyl hydroxyethylcellulose, hydroxypropylcellulose, carboxymethylcellulose, acrylate copolymers (Eudragit E®), polyethylene glycols and/or polyvinylpyrrolidone) or an enteric coating (e.g. based on methacrylic acid copolymer (Eudragit® L and S), cellulose acetate phthalate, hydroxypropyl methylcellulose phthalate, hydroxypropyl methylcellulose acetate succinate, polyvinyl acetate phthalate, shellac and/or ethylcellulose).

Furthermore, a time delay material such as, e.g., glyceryl monostearate or glyceryl distearate may be employed.

In addition, the solid tablet compositions as mentioned above may be provided with a coating adapted to protect the composition from unwanted chemical changes, e.g. chemical degradation, prior to the release of the active drug substance.

The coating may be applied on the solid dosage form in a similar manner as that described in “Aqueous film coating” by James A. Seitz in “Encyclopedia of Pharmaceutical Technology”, Vol 1, pp.337-349 edited by Swarbrick, J. & J. C. Boylan, Marcel Dekker, Inc., New York, 1988.

Formulations for oral use may also be presented as chewing tablets, or as hard gelatin capsules wherein the active ingredient is mixed with an inert solid diluent, for example, potato starch, lactose, microcrystalline cellulose, calcium carbonate, calcium phosphate or kaolin, or as soft gelatin capsules wherein the active ingredient is mixed with water or an oil medium, for example, peanut oil, liquid paraffin, or olive oil.

Powders and granulates may be prepared using the ingredients mentioned above under tablets and capsules in a conventional manner using, e.g., a mixer, a fluid bed apparatus or a spray drying equipment.

Controlled Release Oral Dosage Forms

Controlled release compositions for oral use may, e.g., be constructed to release the active drug substance by controlling the dissolution and/or the diffusion of the active drug substance.

Dissolution or diffusion controlled release can be achieved by appropriate coating of a tablet, capsule, pellet or granulate formulation of the aromatic compounds defined above, such as the bis-aromatic α, β-unsaturated ketones, or by incorporating the compound in question in, e.g., an appropriate matrix.

A controlled release coating may comprise one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3-butylene glycol, ethylene glycol methacrylate and/or polyethylene glycols.

In a controlled release matrix formulation of the aromatic compounds defined above, such as the bis-aromatic α, β-unsaturated ketones, the matrix material may comprise, e.g., hydrated metylcellulose, carnauba wax and stearyl alcohol, carbopol 934, silicone, glyceryl tristearate, methyl acrylate-methyl methacrylate, polyvinyl chloride, polyethylene and/or halogenated fluorocarbon.

A controlled release composition of the aromatic compound defined above, such as the bis-aromatic α, β-unsaturated ketones, may also be in the form of a buoyant tablet or capsule, i.e. a tablet or capsule which upon oral administration floats on top of the gastric content for a certain period of time. A buoyant tablet formulation of the compound in question can be prepared by granulating a mixture of the drug, excipients and 20-75% w/w of hydrocolloids, such as hydroxyethylcellulose, hydroxypropylcellulose and hydroxypropylmethylcellulose. The obtained granules can then be compressed into tablets. On contact with the gastric juice, the tablet can form a substantially water-impermeable gel barrier around its surface. This gel barrier takes part in maintaining a density of less than one, thereby allowing the tablet to remain buoyant in the gastric juice.

Fluid Liquid Compositions

Powders, dispersible powders or granules suitable for preparation of an aqueous suspension by addition of water are also convenient dosage forms. Formulation as a suspension provides the active ingredient in admixture with a dispersing or wetting agent, suspending agent and one or more preservatives.

Suitable dispersing or wetting agents are, for example, naturally-occurring phosphatides, as e.g. lecithin, or condensation products of ethylene oxide with e.g. a fatty acid, a long chain aliphatic alcohol or a partial ester derived from fatty acids and a hexitol or a hexitol anhydrides, for example, polyoxyethylene stearate, polyoxyethylene sorbitol monooleate, polyoxyethylene sorbitan monooleate etc.

Suitable suspending agents are, for example, sodium carboxymethylcellulose, methylcellulose, sodium alginate etc.

Parenteral Compositions

The pharmaceutical composition may also be administered parenterally by injection, infusion or implantation (intravenous, intramuscular, intraarticular, subcutaneous or the like) in dosage forms, formulations or e.g. suitable delivery devices or implants containing conventional, non-toxic pharmaceutically acceptable carriers and adjuvants.

The formulation and preparation of such compositions is well-known to those skilled in the art of pharmaceutical formulation. Specific formulations can be found in the textbook entitled “Remington's Pharmaceutical Sciences”.

Compositions for parenteral use may be presented in unit dosage forms, e.g. in ampoules, or in vials containing several doses and in which a suitable preservative may be added (see below). The composition may be in form of a solution, a suspension, an emulsion, an infusion device or a delivery device for implantation or it may be presented as a dry powder to be reconstituted with water or another suitable vehicle before use. Apart from the active drug substance, the compositions may comprise suitable parenterally acceptable carriers and/or excipients or the active drug substance may be incorporated into microspheres, microcapsules, nanoparticles, liposomes or the like for controlled release. Furthermore, the composition may, in addition, conveniently comprise suspending, solubilising, stabilising, pH-adjusting agents and/or dispersing agents.

As indicated above, the pharmaceutical compositions according to the invention may comprise the active drug substances in the form of a sterile injection. To prepare such a composition, the suitable active drug substances are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl or n-propyl p-hydroxybenzoate. In cases where the aromatic compound defined above, such as the bis-aromatic α, β-unsaturated ketone, is only sparingly or slightly soluble in water, a dissolution enhancing or solubilising agent can be added or the solvent may apart from water comprise 10-60% w/w of propylene glycol or the like.

Controlled Release Parenteral Compositions

As mentioned above under the heading parenteral compositions, controlled release parenteral compositions may be in form of aqueous suspensions, microspheres, microcapsules, magnetic microspheres, oil solutions, oil suspensions, emulsions or the active drug substance may be incorporated in biocompatible carrier(s), liposomes, nanoparticles, implants or infusion devices.

Materials for use in the preparation of microspheres and/or microcapsules are, e.g., biodegradable/bioerodible polymers such as polyglactin, poly-(isobutyl cyanoacrylate), poly(2-hydroxyethyl-L-glutamine) and poly(lactic acid).

Biocompatible carriers which may be used when formulating a controlled release parenteral formulation are, e.g., carbohydrates such as dextrans, proteins such as albumin, lipoproteins or antibodies.

Materials for use in implants are, e.g., non-biodegradable as, e.g., polydimethylsiloxane, or biodegradable such as, e.g., poly(caprolactone), poly(lactic acid), poly(glycolic acid) or poly(ortho esters).

Rectal Compositions

For rectal application, suitable dosage forms for a composition include suppositories (emulsion or suspension type), and rectal gelatin capsules (solutions or suspensions). In a typical suppository formulation, the active drug compounds are combined with an appropriate pharmaceutically acceptable suppository base such as cocoa butter, esterified fatty acids, glycerinated gelatin, and various water-soluble or dispersible bases like polyethylene glycols and polyoxyethylene sorbitan fatty acid esters. Various additives like, e.g., enhancers or surfactants may be incorporated.

Nasal Compositions

For nasal application, typical dosage forms include nasal sprays and aerosols for inhalation. In a typically nasal formulation, the active ingredients are dissolved or dispersed in a suitable vehicle. The pharmaceutically acceptable vehicles and excipients and optionally other pharmaceutically acceptable materials present in the composition such as diluents, enhancers, flavouring agents, preservatives etc. are all selected in accordance with conventional pharmaceutical practice in a manner understood by the persons skilled in the art of formulating pharmaceuticals.

Percutaneous and Topical Compositions

The pharmaceutical compositions may also be administered topically on the skin for percutaneous absorption in dosage forms or formulations containing conventionally non-toxic pharmaceutical acceptable carriers and excipients including microspheres and liposomes. The formulations include creams, ointments, lotions, liniments, gels, hydrogels, solutions, suspensions, sticks, sprays, pastes, plasters and other kinds of transdermal drug delivery systems. The pharmaceutically acceptable carriers or excipients may include emulsifying agents, antioxidants, buffering agents, preservatives, humectants, penetration enhancers, chelating agents, gelforming agents, ointment bases, perfumes and skin protective agents.

Examples of emulsifying agents are naturally occurring gums, e.g. gum acacia or gum tragacanth, naturally occurring phosphatides, e.g. soybean lecithin and sorbitan monooleate derivatives.

Examples of antioxidants are butylated hydroxy anisole (BHA), ascorbic acid and derivatives thereof, tocopherol and derivatives thereof, butylated hydroxy anisole and cysteine.

Examples of preservatives are parabens, such as methyl or propyl p-hydroxybenzoate and benzalkonium chloride.

Examples of humectants are glycerin, propylene glycol, sorbitol and urea.

Examples of penetration enhancers are propylene glycol, DMSO, triethanolamine, N,N-dimethylacetamide, N,N-dimethylformamide, 2-pyrrolidone and derivatives thereof, tetrahydrofurfuryl alcohol and Azone®.

Examples of chelating agents are sodium EDTA, citric acid and phosphoric acid.

Examples of gel forming agents are Carbopol, cellulose derivatives, bentonite, alginates, gelatin and polyvinylpyrrolidone.

Examples of ointment bases are beeswax, paraffin, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

The pharmaceutical compositions mentioned above for topical administration on the skin may also be used in connection with topical administration onto or close to the infected parts of the body which is to be treated. The compositions may be any suitable medicated mass adapted for direct application or for introduction into relevant orifice(s) of the body, e.g. the rectal, urethral, vaginal or oral orifices. The compositions may simply be applied directly onto the infected part, e.g. the mucosa. In certain cases it might be applied by means of special drug delivery devices such as dressings or alternatively plasters, pads, sponges, strips or other forms of suitable flexible material.

Controlled Release Percutaneous and Topical Compositions

In general, four different approaches are applicable in order to provide rate control over the release and transdermal permeation of a drug compound. These approaches are: membrane-moderated systems, adhesive diffusion-controlled systems, matrix dispersion-type systems and microreservoir systems. It is appreciated that a controlled release percutaneous and/or topical composition may be obtained by using a suitable mixture of the above-mentioned approaches.

In a membrane-moderated system, the active drug substance is present in a reservoir which is totally encapsulated in a shallow compartment molded from a drug-impermeable laminate, such as a metallic plastic laminate, and a rate-controlling polymeric membrane such as a microporous or a non-porous polymeric membrane, e.g., ethylene-vinyl acetate copolymer. The active drug substance is only permitted to be released through the ratecontrolling polymeric membrane. In the drug reservoir, the active drug substance may either be dispersed in a solid polymer matrix or suspended in an unleachable, viscous liquid medium such as silicone fluid. On the external surface of the polymeric membrane, a thin layer of an adhesive polymer is applied to achieve an intimate contact of the transdermal system with the skin surface. The adhesive polymer is preferably a polymer which is hypoallergenic and compatible with the active drug substance.

In an adhesive diffusion-controlled system, a reservoir of the active drug substance is formed by directly dispersing the active drug substance in an adhesive polymer and then—by, e.g., solvent casting—spreading the adhesive containing the active drug substance onto a flat sheet of substantially drug-impermeable metallic plastic backing to form a thin drug reservoir layer.

A matrix dispersion-type system is characterized in that a reservoir of the active drug substance is formed by substantially homogeneously dispersing the active drug substance in a hydrophilic or lipophilic polymer matrix and then, the drug-containing polymer is molded into disc with a substantially well-defined surface area and controlled thickness. The adhesive polymer is spread along the circumference to form a strip of adhesive around the disc.

A microreservoir system may be considered as a combination of the reservoir and matrix dispersion type systems. In this case, the reservoir of the active substance is formed by first suspending the drug solids in an aqueous solution of water-soluble polymer and then dispersing the drug suspension in a lipophilic polymer to form a multiplicity of unleachable, microscopic spheres of drug reservoirs.

Compositions for Administration to the Eye

Formulations for administration to the eye may be presented in the form of eye drops, lotions, ointments or delivery devices. Typically, the composition comprises the active drug substance in combination with pharmaceutically inert vehicles or the active drug substance is incorporated in a suitable carrier system. Pharmaceutically inert vehicles and/or excipients for the preparation of eye drops include, e.g., buffering agents such as boric acid or borates, pH adjusting agents to obtain optimal stability or solubility of the active drug substance, tonicity adjusting agents such as sodium chloride or borates, viscosity adjusting agents such as hydroxypropyl cellulose, methylcellulose, polyvinylpyrrolidone, polyvinyl alcohols or polyacrylamide, oily vehicle such as vehicles comprising arachis oil, castor oil and/or mineral oil. Emulsions and suspensions of the active drug substance may also be presented in form of eye drops. In these cases, the composition may furthermore comprise stabilizing, dispersing, wetting, emulsifying and/or suspending agents. Eye lotions and eye ointments may comprise pharmaceutically acceptable carriers and/or excipients such as those used in an eye drop composition or in other relevant topical composition such as, e.g., ointments, creams and lotions.

In general, an aqueous eye drop composition may be prepared by dissolving the active drug substance (or preferably a water-soluble salt or prodrug thereof) in sterile water in a specific concentration, optionally adjusting pH to a suitable pH by adding an appropriate amount of an appropriate buffer solution or hydrochloric acid or sodium hydroxide, optionally adding a preservative such as phenethanol, optionally adding a viscosity increasing agent such as methylcellulose, and subject the resulting solution to filtration followed by sterilization e.g. by autoclaving or by membrane filtration.

The formulation and preparation of the above-mentioned compositions are well-known to those skilled in the art of pharmaceutical formulation. Specific formulations can be found in “Remington's Pharmaceutical Sciences”.

Addition to Animal Feed and Fish Water

As mentioned above, the aromatic compounds defined above, such as the bis-aromatic α, β-unsaturated ketones or derivatives thereof, may be most valuable for controlling parasites in e.g. cattle, birds and fish. This may be carried out, e.g. by adding the compound in question to the feed or the drinking water of the animals, or when the animals to be treated are fish, the compound in question may also be added to the fish water.

Dosages

The bis-aromatic α, β-ketones are preferably administered in an amount of about 0.1-50 mg per kg body weight per day, such as about 0.5-25 mg per kg body weight per day. The compound in question may be administered orally in the form of tablets, capsules, elixirs or syrups, or rectally in the form of suppositories. Parenteral administration of the aromatic compound defined above, such as the bis-aromatic α, β-unsaturated ketone, is suitably performed in the form of saline solutions of the ketones (or salts thereof) or with the compound incorporated into liposomes or in oil emulsions and cubic structures in oil. In cases where the compound in itself is not sufficiently soluble to be dissolved, an acid addition salt of a basic compound of the formula I (that is, a compound of the formula I in which either an aromatic ring or a substituent contains a basic nitrogen atom) can be used, or a solubiliser such as ethanol can be applied.

Oral administration. For compositions adapted for oral administration for systemic use, the dosage is normally 2 mg to 1 g per dose administered 1-4 times daily for 1 week to 12 months depending on the disease to be treated.

The dosage for oral administration for the treatment of parasitic diseases is normally 1 mg to 1 g per dose administered 1-2 times daily for 1-4 weeks, in particular the treatment of malaria is to be continued for 1-2 weeks whereas the treatment of leishmaniasis will normally be carried out for 3-4 weeks.

The dosage for oral administration for the treatment of bacterial diseases is normally 1 mg to 1 g per dose administered 1-4 times daily for 1 week to 12 months; in particular, the treatment of tuberculosis will normally be carried out for 6-12 months.

The dosage for oral administration of the composition in order to prevent diseases is normally 1 mg to 75 mg per kg body weight per day. The dosage may be administered once or twice daily for a period starting 1 week before the exposure to the disease until 4 weeks after the exposure.

Rectal administration. For compositions adapted for rectal use for preventing diseases, a somewhat higher amount of aromatic compounds, such as bis-aromatic α, β-unsaturated ketones or derivatives thereof is usually preferred, i.e. from approximately 1 mg to 100 mg per kg body weight per day.

Parenteral administration. For parenteral administration, a dose of about 0.1 mg to about 50 mg per kg body weight per day is convenient. For intravenous administration a dose of about 0.1 mg to about 20 mg per kg body weight per day administered for 1 day to 3 months is convenient. For intraarticular administration a dose of about 0.1 mg to about 20 mg per kg body weight per day is usually preferable. For parenteral administration in general, a solution in an aqueous medium of 0.5-2% or more of the active ingredients may be employed.

Percutaneous administration. For topical administration on the skin, a dose of about 1 mg to about 5 g administered 1-10 times daily for 1 week to 12 months is usually preferable.

Transmission control. As mentioned above, the use of the aromatic compounds defined above, such as the bis-aromatic α, β-unsaturated ketones, or derivatives thereof in controlling parasites in their vectors is an interesting and promising aspect of the present invention. The principle is to destroy the parasites in their vectors, thereby preventing transmission of the disease. The data presented herein demonstrate clearly that the promastigote stage, the same form of the parasite which is present in the sandfly vector, of the L. major and L. donovani parasite, is killed by the bis-aromatic α, β-unsaturated ketones or derivatives thereof. For example, spraying endemic areas for malaria or leishmania or other protozoal diseases transmitted by their respective vectors will be an attractive means of controlling such important parasitic diseases.

For the compounds mentioned above containing a double bond, the corresponding compounds in which the bond is a triple bond such as discussed in connection with the general formula I are also very interesting and should be considered correspondingly disclosed herein in connection with each and every structural formula shown herein and each and every compound named herein.

In many cases, it will be preferred to administer the compound defined herein together with another antiparasitic, antimycotic or antibiotic drug, thereby reducing the risk of development of resistance against the conventional drugs, and reducing the amount of each of the drugs to be administered, thus reducing the risk of side effects caused by the conventional drugs. Important aspects of this is the use of the compound against Leishmania, where the compound I is combined with another antileishmanial drug, or the antimalarial use of the compound I where the compound I is used together with another antimalarial drug.

As examples of another antileishmanial drug to be combined with the compounds defined herein may be mentioned pentavalent antimony-sodium gluconate. As examples of other antimalarial drugs to be combined with the compounds defined herein may be mentioned chloroquine and derivatives thereof, quinine, proguanil, cycloguanil, mefloquine, pyrimethamine and artemisinin. As an example of an additional antibiotic drug to be combined with the compounds defined herein may be mentioned an antituberculous drug such as isoniazide, ethambutol, pyrazinamide, and rifampicin or anti-Helicobacter drugs such as bismuth, metranidazole and amoxicillin. As examples of additional antimycotic drugs to be combined with the compounds defined herein may be mentioned amphotericin B, muconarcidol, griseofulvin, and miconazol. As examples of additional antibabesial drugs to be combined with the compounds defined herein may be mentioned quinuronium sulfate, pentamidine isethionate, imidocarb or diminazene. As examples of additional anticoccidial drugs to be combined with the compounds defined herein may be mentioned fulfonamides, amprocid and coccidiostatic agents such as inomycins, in particular monensin and salinomycin.

As examples of additional drugs against fish parasites to be combined with the compounds defined herein may be mentioned benzimidazol and formaldehyde.

One general advantage of the compounds defined herein are their broad-spectered character, which makes it possible to use the compounds as sole medication in cases where the host to be treated is infected with, or suspected to be infected with, more than one of the bacteria and parasites discussed herein, or to use them as supplements to known antibacterial agents and antiparasitic agents in order to reduce the dose of the conventional antibiotics or antiparasitic agents, thus reducing the risk of side effects, in addition to the above-mentioned advantages with respect to reduction of drug resistance development.

In particular for prophylaxis, the broad-spectered character of the compounds of the general formula I is of great advantage, and may be further augmented by combination with more than one antibacterial or antiparasitic agent, such as combination both with another antileishmanial agent and another antimalarial agent. It is justified to presume that also the other aromatic compounds defined herein will show the same valuable broad-spectered character.

Experimental Design for Selection of Lead Compounds (QSAR Analysis)

One way of selecting compounds which have a high likelihood of possessing valuable biological activities (which can be confirmed by actual testing) within one or more of the indication areas covered by the present invention is to take advantage of the Quantitative Structure Activity Relationship (QSAR) analysis methodology (see, e.g., Hellberg, S. A, Multivariate Approach to QSAR, 1986, Ph.D. Thesis, University of Umeå).

As appears from the experimental data reported herein, it has been found that the QSAR analysis methodology is excellently suited for the types of compounds used according to the present invention, especially for the group of chalcones ((E)-1,3-diphenyl-prop-2-en-1-ones), for the group of dihydrochalcones (1,3-diphenyl-propan-1-one), and for the group of dehydrochalcones (1,3-diphenyl-prop-2-yn-1-one) used according to the present invention. The QSAR model is equally applicable for all variants of the compounds defined herein, also, e.g., for the α, β-substituted variants of the compounds of the general formula I.

Thus, the influence of the substitution pattern on the two aromatic nuclei of the compounds used according to the invention can be elucidated by synthesis of chalcones covering substituents having different properties for variables such as hydrophobic parameters, e.g. lipophilicity (π) and partition coefficient (log P), electronic parameters, e.g. ionisation constants (pKₐ), charge, and Hammet values (σ) such as the electron distribution in the two aromatic rings (σₘ and σₚ), and steric parameters, e.g. molecular refractivity (MR) and Van der Waals volume, and combinations hereof, testing the activity of the compounds in a relevant assay, and performing a QSAR analysis on the data thus obtained. (see, e.g., Skagerberg, B., Bonelli, D., Cruciani, G., Ebert, C., Principal Properties for Aromatic Substituents. A Multivariate Approach for Design in QSAR. Quant. Struct.-Act. Relat., 1989, 8, 32-38.). An optimum way of selecting compounds for the QSAR analysis is to use a statistical design based on the so-called Principal Component Analysis (PCA) for reduction of the number of variables to be processed. The principal components which summarise the information in the selected variables are linear combinations of the selected variables, possibly multiplied by a “loading” factor. The principal components may be determined using suitable chemical/statistical software, such as, e.g., SIMCA P 2.1™, SIMCA Umetri AB, Umeå Sweden.

For each principal component, a statistical design affording optimum variation of the variables is preferably used. Such an optimum design uses variables in each position that can be substituted resulting in at least a high and a low value of the principal component. As an example of variables may be mentioned the variable lipophilicity (π) which can be relatively broadly represented by, e.g., including compounds having a hydroxy group and a tert-butyl group, respectively, as a substituent in a specific position.

The number of principal components and the number of places where substitution can be performed determine the necessary number of individual compounds to be included in the analysis. Thus, in the compounds defined herein which are chalcones, there are, in principle, 12 possible substitution sites, which means that the number of compounds should preferably be at least 2 to the power of principal components multiplied by 12. Thus, where the number of principal components is 2, the number of compounds to be included in the QSAR should preferably be at least 48. In the compounds defined herein which are 1,3-diphenylpropenynes, there are, in principle, 10 positions that may be substituted, and the number of compounds to scan this spectre of possibilities in the QSAR will preferably be at least 40, assuming that the number of principal components is two also here. Finally, in the compounds defined herein which are 1,3-diphenylpropanones, there are, in principle, 14 positions that might be substituted, which means that the number of compounds to be included in the QSAR should preferably be at least 56, still presuming that there are two principal components. When smaller numbers of substituent positions are to be analysed, the preferred minimum number of compounds included can be smaller in accordance with the calculations described above. While the above explanation with respect to the number of compounds to be included in the QSAR analysis represent the so-called complete factor design approach, it is also possible to use smaller numbers of compounds according to the so-called fractional factor design approach or the so-called D-optimal design. Considerations with respect to the selection of compounds for the establishment of a set of compound are presented in the references mentioned above as well as in references describing the complete factor design approach (see, e.g., Austel, V., A manual method for systematic drug design. Eur. J. Med. Chem, 1982, 17, 9-16, and Eriksson, L., Johansson, E., Tutorial: Multivariate design and modelling in QSAR., Chemom. Intell. Lab. Sys. 1996, 34, 1-9), the fractional factor design approach (see, e.g. Austel, ibid.), and the D-optimal design approach (see, e.g., Eriksson et al. ibid. and Baroni, M, Blementi, S., Cruciani, G., Kettaneh-Wold, N., D-Optimal Designs in QSAR., Quant. Struct.-Act. Relat., 1993, 12, 225-231).

The QSAR analysis is based on the set of compound selected according to the statistical design described above. For each of the compounds, the relevant biological activity is determined, typically in vitro. It is preferred that each of the compounds in the set actually possesses a measurable biological activity in order to obtain the most precise prediction of the biological activity for compounds outside the set of compounds. It is also preferred that the range of biological activity represented by the set of compounds is a broad as possible. Thus, these considerations may necessitate exchange of some of the compounds which were suitable according to the statistical design described above. Ease of synthesis is another factor which should be taken into consideration in the selection of the compounds.

The results of the QSAR analysis are given as predicted IC₅₀ values and as a representation indicating sites in which increase of the variable in question, such as lipophilicity or hydrophilicity, will increase or decrease, respectively, the affinity to the target involved in the biological effect in question (whether the target molecule is known or not). In the so-called 2D QSAR, this representation is an indication of the range of values to be aimed at in each particular site. In the so-called 3D QSAR, the presentation is a stereo presentation showing regions in which increase of the variable in question will increase or decrease, respectively, the affinity to the target. Based on these representations, the person skilled in the art can select, for each position, substituents which will have properties increasing or decreasing the IC₅₀ value. The selection may typically be performed using data available for the relevant variable in standard chemical tables or databases. (See Example 9). Thus, as most known possible substituents appear in published tables or databases with their relevant data within the various types of variables, the method points to a number of appropriate “suggested” substituents in each site, which means that the stereographic representation can be said inter alia to represent, to the person skilled in the art, a relatively well-defined class of suggested compounds of increased or decreased (as the case may be) biological effect with respect to the target in question. Based upon this “suggestion”, the person skilled in the art can construct one or more compounds and subject these constructed compounds to the QSAR analysis. Those constructed compounds which are predicted by the QSAR analysis to have a desired low IC₅₀ with respect to the desired activity against the disease-related target can then be synthesised and tested for their actual activity.

Representative compounds thus synthesised and tested and found to have improved actual activity can then, if desired, be included in the panel of compounds to constitute the base of a new QSAR analysis, and in this iterative manner, better and better compounds—with respect to the desired activity in question—can be identified.

On the other hand, also a desirable low activity in a particular assay can be a desired property of a compound, such as, e.g., indicating low toxicity. In the present case, it has been found that the QSAR analysis lends itself excellently to the prediction of IC₅₀ values for the activity of the compounds defined herein in the Lymphocyte Proliferation assay.

This means that for the compounds defined herein, the QSAR analysis is an excellent tool for predicting the relevant biological activity of subclasses of the compounds. This is most valuable in the screening for lead compounds. Even though the synthesis of the compounds defined herein is relatively simple and efficient, it is important that the synthesis can be limited to a smaller number of compounds which can then be used as a basis for guiding, through the QSAR analysis, the skilled person towards compounds having a high likelihood of showing more valuable properties. Once attractive candidate compounds have been identified using the QSAR analysis, it is easy to synthesise and test the compound.

As will be understood on the basis of the above, the QSAR analysis can be used to predict one or several properties of compounds. Thus, one valuable utilisation of the QSAR analysis would be to predict both the biological activity of compounds against the target to be controlled and (in another QSAR analysis) the biological activity against the human system itself, in other words, the tolerability or toxicity data of compounds. All of the above-discussed assays relating to targets to be controlled (in connection with Leishmania, Malaria, Helicobacter pylori, inflammatory cytokines, virus, and cancer) can be used in connection with QSAR analysis. In addition to the lymphocyte test mentioned above, also other toxicity tests the results of which can be indicated by concentration values such as IC₅₀ values can be used in connection with QSAR analysis, including, e.g., the Ames mutagenicity test.

The value of the parameter Q² indicates the predictivity of the particular QSAR model in the particular context. Q² is defined as one minus the summation of the second power of the difference between the measured and the predicted IC₅₀ values of the panel of compounds divided by the summation of the second power of the difference between the measured IC₅₀ values and the mean IC₅₀ value of the compounds included. It is preferred that Q² is at least 0.5, preferably at least 0.6 and more preferably at least 0.7 or, even more preferably, at least 0.8.

Thus, based on the well-known routines and criteria, the person skilled in the art will be able to establish a set of compounds of the classes defined herein for use in the QSAR model and to use the QSAR model for the purposes discussed above.

As indicated above, it is preferred that the set of basis compound (or simply “a set of compounds”) is designed taking into consideration both hydrophobic, electronic, and steric parameters, e.g. lipophilicity (π), Hammet values (σ) such as the electron distribution in the two aromatic rings (σₘ and σₚ), and molecular refractivity (MR).

An excellent example of a set of 24 chalcones which represent the variables lipophilicity (π), the electron distribution in the two aromatic rings (σₘ and σₚ), molecular refractivity (MR), and Van der Waals volume is a set comprising

(1) 2′-butoxy-3,5-dimethoxychalcone

(2) 2′-dimethylamino-3,5-dimethoxychalcone

(3) 2′-nitro-3,5-dimethoxychalcone

(4) 2′-fluoro-3,5-dimethoxychalcone

(5) 3′-butoxy-2,4-dimethoxychalcone

(6) 3′-dimethylamino-3,5-dimethoxychalcone

(7) 3′-nitro-2,4-dimethoxychalcone

(8) 3′-fluoro-3,5-dimethoxychalcone

(9) 4′-cyclohexyl-3,5-dimethoxychalcone

(10) 4′-dimethylamino-3,5-dimethoxychalcone

(11) 4′-nitro-3,5-dimethoxychalcone

(12) 4′-fluorochalcone

(13) 2-butoxy-2′,3′,4′-trimethoxychalcone

(14) 2-dimethylamino-2′,3′,4′-trimethoxychalcone

(15) 2-nitro-2′,3′,4′-trimethoxychalcone

(16) 2-flouro-2,3′,4′-trimethoxychalcone

(17) 3-butoxy-2′,3′,4′-trimethoxychalcone

(18) 3-dimethylamino-2′,3′,4′-trimethoxychalcone

(19) 3-nitro-2′, 3′,4′-trimethoxychalcone

(20) 3-flouro-2′,3′,4′-trimethoxychalcone

(21) 4′-fluoro-4-phenoxychalcone

(22) 4-dimethylamino-2′,3′,4′-trimethoxychalcone

(23) 4-cyano-2′,3′,4′-trimethoxychalcone

(24) 4,4′-dinitrochalcone

which have all been or can be prepared according to the methods described herein.

In the cases where, e.g., the biological activity of dihydro-chalcones is to be predicted, a corresponding set of dihydro analogues, e.g. the dihydro analogues of the compounds (1)-(24), may be prepared by the method described herein and used in the QSAR Model, and in the case where α- and/or β-substituted chalcones are relevant, a variant of the set of compounds, e.g. the compounds (1)-(24), where either further α- and/or β-substituted compounds or α- and/or β-substituted analogues of the compounds of the set are prepared and included in the QSAR Model.

Thus, in an aspect of the present invention, the compounds according to the invention or the compounds to be used according to the invention are compounds which, according to QSAR analysis, are predicted to have an IC₅₀ below a certain defined value with respect to the disease target to be controlled, and/or, according to QSAR analysis, are predicted to have an IC₅₀ above a certain defined value with respect to assays for testing the toxicity of the compounds. Another aspect of the invention relates to a method for selecting bis-aromatic compounds which have an IC₅₀ below a certain defined value with respect to the disease target to be controlled, and/or, according to QSAR analysis, are predicted to have an IC₅₀ above a certain defined value with respect to assays for testing the toxicity of the compounds, the method comprising establishing a panel of compounds of the molecular type in question and having a variability of variables suitable for the QSAR analysis, including for the establishment of Principal Components, testing the panel of compounds in the relevant assay, subjecting the compounds with their thus established assay data to QSAR analysis, constructing compounds which, according to the output of the QSAR analysis, would be likely to have an improved activity in the assay in question, synthesising and testing the compounds constructed and optionally including one or more of the thus synthesised and tested compounds with their data in a further QSAR analysis, etc.

In this way, by optionally repeated QSAR-based prediction, synthesis, testing, and incorporation of new compounds in the QSAR model, the skilled person will, based on the disclosure herein, and based on the suitability to QSAR analysis demonstrated herein for relevant compounds, be able to identify classes of or individual compounds according to the invention which have a superior biological effect and a low toxicity.

The QSAR analysis described above is based on the biological effects of the bis-aromatic compounds discussed herein. Another approach is the receptor-based method (the drug design method) described in the following. Since the present invention also provides a method for the isolation and purification of fumarate reductase, it is believed that knowledge about this specific receptor, in combination with the drug design method described below, can be used for identification of novel bis-aromatic compound, which have superior pharmacological profiles In order to design such a drug it is of great value that the motif of binding between the fumarate reductase and a known ligand (e.g. a natural substrate or a bis-aromatic compound known to inhibit fumarate reductase-ligand interaction) is known in detail, in order to develop the method to effectively identify compounds capable of blocking this binding.

The bis-aromatic compound can be any compound which has one of the above mentioned effects on the interaction between fumarate reductases and known ligands and thereby action of the fumarate reductases. Especially interesting bis-aromatic compounds are those which are likely to interact with the substrate binding part of the fumarate reductase, but interaction with other sites in the fumarate reductases may also cause the desired inhibitory effect. This can be the result of direct steric blocking of the normal binding between the substrate and the fumarate reductase, but it may also be an effect of a conformational change in the fumarate reductase. A method of identifying bis-aromatic compounds to be used in the method of the invention is disclosed below.

The interaction between the bis-aromatic compound and the fumarate reductase may be a covalent as well as a non-covalent binding to the fumarate reductase by the bis-aromatic compound.

It will be understood that the above-described methods comprising administration of bis-aromatic compounds in treating and/or preventing diseases are dependent on the identification or de novo design of bis-aromatic compounds which are capable of exerting effects which will lead to inhibition of the binding between substrates and fumarate reductase. It is further important that these bis-aromatic compounds will have a high chance of being therapeutically active.

Thus, an aspect of the invention relates to a method for identifying a potentially therapeutically useful bis-aromatic compound capable of interacting with an fumarate reductase, thereby inhibiting binding between substrate and fumarate reductase, the method comprising at least one of the following steps:

1) testing a candidate bis-aromatic compound in an assay in which the possible inhibition by the bis-aromatic compound of the interaction between fumarate reductase and known ligand is determined by

a) adding the bis-aromatic compound to a system comprising fumarate reductase or an analogue thereof in an immobilized form and the known ligand in a solubilized form and determining the change in binding between the known ligand and fumarate reductase or analogue thereof caused by the addition of the bis-aromatic compound, or

b) adding the bis-aromatic compound to a system comprising the known ligand in an immobilized form and fumarate reductase or an analogue thereof in a solubilized form and determining the change in binding between the known ligand and the fumarate reductase or analogue thereof caused by the addition of the bis-aromatic compound, or

c) adding the bis-aromatic compound to a system comprising the known ligand as well as fumarate reductase or an analogue thereof in solubilized form and determining the change in binding between known ligand and fumarate reductase or analogue thereof caused by the addition of the bis-aromatic compound, or

d) adding the bis-aromatic compound to a system comprising known ligand as well as—fumarate reductase or an analogue thereof in solubilized form and measuring the change in binding energy caused by the addition of the bis-aromatic compound, and identifying the bis-aromatic compound as potentially therapeutically useful if a significant change in the binding energy between the known ligand and fumarate reductase or analogue thereof is observed,

and identifying the bis-aromatic compound as potentially therapeutically useful if a significant change in the binding or binding energy between the known ligand and fumarate reductase or analogue thereof is observed;

2) testing a candidate bis-aromatic compound in an assay in which the possible prevention, inhibition or enhancement of the interaction between fumarate reductase and the known ligand is determined by

adding the bis-aromatic compound to a system comprising living parasites followed by determination of the growth rate of the parasites or bacteria, a reduction in growth rate compared to a corresponding system wherein the bis-aromatic compound has not been added being indicative of inhibition of the binding between fumarate reductase and a known ligand;

or adding the substance in an assay determining the activity of the enzyme;

and identifying the bis-aromatic compound as potentially therapeutically useful if a reduction in growth rate or inhibition of enzyme activity is observed after the addition of the bis-aromatic compound; and

3) administering, to an experimental animal, a bis-aromatic compound which has been established in vitro to inhibit the interaction between a fumarate reductase and a given known ligand, the experimental animal being inoculated with parasites or bacteria before, simultaneously with or after the administration of the bis-aromatic compound, and electing as a bis-aromatic compound suitably capable of interacting with a fumarate reductase, a bis-aromatic compound preventing and/or curing and/or alleviate disease caused by the parasites and bacteria.

The term “an analogue of a fumarate reductase” denotes any compound which have the ability of binding at least one known ligand of the fumarate reductase in a manner corresponding to the binding between fumarate reductase and bis-aromatic compound. Such an analogue of the fumarate reductase can be a truncated form of the intact fumarate reductase or it can be a modified form of the fumarate reductase which may e.g. be coupled to a probe, marker or another moiety. Finally, the analogue of the fumarate reductase can be an isolated, but partially of fully functional, active site of the fumarate reductase or a synthetic compound which mimics such an active site.

The immobilisation mentioned above may be simple non-covalent binding to an adhering surface or a host or receptor molecule such as an antibody, or covalent binding to a spacer molecule such as a polymer or a peptide.

In the above mentioned step 1a), the known ligand being bound to fumarate reductase or an analogue thereof can be detected in a number of ways, e.g. by the known ligand being labelled, or by means of a labelled agent (such as an antibody) capable of reacting with the known ligand, or by means of a refractive index based determination of the extend of binding, such as the Pharmacia BiaCore® assay.

Accordingly, in step 1b) fumarate reductase or the analogue thereof being bound to the known ligand may be detected by fumarate reductase or the analogue thereof being labelled, by means of a labelled agent (e.g. antibody) capable of reacting with fumarate reductase or the analogue thereof, or by means of a refractive index based determination of the extend of binding, such as the Pharmacia BiaCore® assay.

In step 1c) fumarate reductase or the analogue thereof being bound to the known ligand may be detected by separation of ligand-fumarate reductase complexes (e.g. by ultracentrifugation, ultrafiltration, liquid chromatography, such as size exclusion chromatography, or electrophoresis).

The determination of binding energy in step Ic) is preferably performed in a microcalorimetric system using the well-known technique of microcalorimetry.

The above-indicated steps serve 3 purposes. The types of assays in step 1) are intended to shed light over the ability of the candidate bis-aromatic compound of interacting with the fumarate reductase. In the instances wherein labelled, ligands, bis-aromatic compounds, fumarate reductases or antibodies are used, the label could be a radioactive label, a fluorescent or light absorbing label, an enzyme such as horse-radish peroxidase, a ligand such as biotin, or any other conventional labelling system known to the person skilled in the art. The detection of the labelled compound is then dependent on the choice of label: radioactivity may be measured in a liquid-scintillation counter, a gamma counter, or any other convenient detection system for radioactivity, enzyme-labels are detected by the presence or absence of a specific substrate for the enzyme (optical density assessment, chemical reactivity of the remaining substrate or of the product etc.), fluorescent labels may be detected by fluorescence microscopy or simple measurement of the fluorescent emission, light-absorbing labels may be detected by measurement of absorbtion of light of a characteristic wavelength, and biotin may be detected by its binding to streptavidin.

The separation of high molecular complexes by ultracentrifugation or ultrafiltration in 1) may be detected by one of the components of the complex being labelled as described above; it is thus possible to detect the ratio between bound and unbound known ligand, but the detection step may also rely on the binding of antibodies to one of the components of the complex, and the subsequent detection of this antibody. Any conventional chromatographic technique may be employed (HPLC, FPLC, size exclusion, etc) The separation by electrophoresis may e.g. be performed by capillary electrophoresis.

The assay(s) in step 2) relate to the effects of the candidate bis-aromatic compound on parasitic activity in vitro. The demonstration of a reduction in growth rate of the parasites of course cannot be contributed to the effect of interaction with fumarate reductases only, but a demonstration of this kind should provide a good estimate of the potential therapeutical usefulness of such a bis-aromatic compound.

The determination of growth rate may be performed by thymidine uptake in leishmania parasites or CFU in H. pylori, ATP, or by any other convenient detection system known to the person skilled in the art.

The animal study in step 3) is performed in order to demonstrate the potential therapeutic usefulness of the candidate bis-aromatic compound in vivo. Further, such animal studies should also establish the a priori values regarding effective dosage and toxicity before the candidate bis-aromatic compound finally is tested in human beings in controlled clinical trials. The animal studies should also provide information regarding the convenient formulation of the bis-aromatic compound in a pharmaceutical preparation as well as the preferred route of administration, as it is possible to obtain, from the animal model, data for absorbtion of the bis-aromatic compound as well as data for the metabolism and excretion of the bis-aromatic compound. The experimental animal is preferably a mouse, a rat, a cat, a dog, a monkey, a horse, a cow, a pig, or a chicken.

The term “suitably capable of interacting with an fumarate reductase” is intended to indicate that a bis-aromatic compound, apart from being capable of interacting with a fumarate reductase, is also capable of exerting effects in an in vivo system, i.e. that the bis-aromatic compound in addition to its binding capability also exhibits compatibility with a biological system, i.a. a patient.

Although the above-indicated in vivo studies, especially the experiments in animal models, are the best indicators of the potential therapeutical usefulness of a bis-aromatic compound in the inhibition of binding between a fumarate reductase and a known ligand such as the natural substrate, it should not be forgotten that the in vitro assays outlined above serve as important leads when developing compounds with a therapeutical potential. If one relied only on in vivo assays, it is very likely that compounds which in fact exhibit the desired effect on the fumarate reductase-substrate interaction would be screened out by the in vivo assays, because these compounds could lack e.g. the ability to penetrate biological membranes. When using the in vitro assays, a much greater chance of finding a lead compound is maintained.

The evaluation of the effect of a bis-aromatic compound tested in the in vitro assays described herein (cf. in this connection especially the examples) depends on a number of factors. It will be understood by the skilled person that a small molecule could be added in rather high molar concentrations in order to exert an effect on the fumarate reductase/ligand interaction (and even then the small molecule may still be an interesting lead compound), whereas larger molecules may exert marked effects even in rather low molar concentrations. In general, when any in vitro assay described herein is regarded as having a positive result when testing a candidate bis-aromatic compound (i.e. that the bis-aromatic compound tested shows a “significant” effect), the following condition should be fulfilled: The compound should exert a significant effect on ligand/fumarate reductase interaction (or on an interaction in an equivalent system which correlates well to ligand/fumarate reductase interaction), the significant effect being one which with no doubt can be attributed to the interaction between the bis-aromatic compound and the fumarate reductase and which is not an unspecific interaction between the fumarate reductase and the bis-aromatic compound (due to e.g. radical changes in the physical and chemical environment when the bis-aromatic compound is added). One way of excluding unspecific interactions as the reason for the exerted effect is to use at least one control which is a chemically comparable bis-aromatic compound (with respect to molecular mass, charge/polarity and gross 3-dimensional conformation (globular, fibrillar etc.). If the control does not result in substantially the same effect in the assay as the bis-aromatic compound, it can be concluded that the bis-aromatic compound must be regarded as an assay-positive bis-aromatic compound.

The assays described in the examples are all good examples of assay types, which could serve as the test system in the above-described method of the invention.

It will be understood that the above-indicated method for identifying a potentially therapeutically useful bis-aromatic compound is dependent on the actual presence of the bis-aromatic compound. Normally, it is necessary to either purify or synthesize the candidate bis-aromatic compound before it is subjected to the above-mentioned method. However, since many such candidate bis-aromatic compounds are likely to be tested before a bis-aromatic compound which is suitably capable of interacting with a fumarate reductase will be identified, it is of interest to identify such bis-aromatic compounds before they are subjected to the method above, thereby diminishing the resources spent on purification and/or synthesis steps.

Drug Modelling

Hence, the invention also relates to a method for identifying and/or designing a bis-aromatic compound, X, capable of interacting with a fumarate reductase, e.g. binding to the fumarate reductase, with a predicted binding energy equal to or better than a predetermined threshold value, the method comprising

1) selecting a bis-aromatic compound, A, which could potentially interact with a site in the fumarate reductase, and providing a 3-dimensional structural representation thereof,

2) predicting the binding free energy between the bis-aromatic compound A and the site in the fumarate reductase,

3) if the predicted binding free energy between the bis-aromatic compound A and the site in the fumarate reductase is equal to or better than the predetermined threshold value, then identifying the bis-aromatic compound A as the bis-aromatic compound X,

4) if the predicted binding free energy between the bis-aromatic compound A and the site in the fumarate reductase is not equal to or better than a predetermined threshold value, then modifying the 3-dimensional structural representation and predicting the binding free energy between the thus modified bis-aromatic compound, B, and the site in the fumarate reductase, and

5) repeating step 4 until the predicted binding free energy determined between the resulting bis-aromatic compound, X, and the site in the fumarate reductase is equal to or better than the predetermined threshold value.

It is possible to expand the above-mentioned method with two further steps, wherein the actual binding free energy is determined, in order to establish that the experimental binding free energy is in fact better than the predetermined threshold value. By performing the following two steps

6) providing a sample of the chemical bis-aromatic compound X and a sample of the fumarate reductase and measuring the binding free energy between the chemical bis-aromatic compound X and the fumarate reductase (e.g. by microcalometry as mentioned above), and establishing that the measured binding free energy between the chemical bis-aromatic compound X and the fumarate reductase is equal to or better than the predetermined threshold value, and optionally

7) subjecting the bis-aromatic compound X to the method mentioned above for identifying a bis-aromatic compound suitably capable of interacting with a fumarate reductase, in order to verify that the bis-aromatic compound X is a potentially therapeutically useful bis-aromatic compound capable of interacting with a fumarate reductase,

it is thus verified that the binding free energy between the candidate bis-aromatic compound and the fumarate reductase actually is better than the predetermined threshold value. Step 7) further establishes that the candidate bis-aromatic compound stands good chances of being therapeutically useful.

The phrase “predicting the binding free energy” is meant to imply that the binding free energy is determined by calculation rather than by performing experimental work determining the actual binding free energy. One (theoretical) way of predicting binding free energy is by performing free energy perturbation (FEP) calculations on the interacting bis-aromatic compounds, but because of the vast amount of calculations such an approach would have as a result it is preferred that the empirical approximative method described below is employed.

The term “better than” is intended to mean that the binding free energy has a value which is higher than the binding free energy which has been chosen as the threshold value, meaning that the ΔG is numerically higher than the threshold value selected. Or in other words: The term is intended to mean that the binding between the bis-aromatic compound and the fumarate reductase is more favourable energetically than the situation were the bis-aromatic compound and the fumarate reductase are suspended independently in solution.

In order to predict the binding energy in the above-indicated method, according to the invention it is especially preferred to use the following method:

Structure Based Method

Assessing the average energy difference, <ΔV^{el}_{X-s}>, defined as <V^{el}_{X-s}>_{B}−<V^{el}_{X-s}>_{A}, between the contribution from polar interactions to the potential energy between the chemical bis-aromatic compound X and its surroundings (denoted s) in two states, one state (A) being where the chemical bis-aromatic compound is surrounded by solvent, the other state (3) being where the chemical bis-aromatic compound, bound to a fumarate reductase or an analogue thereof, is surrounded by solvent,

assessing the average energy difference, <ΔV^{vdw}_{X-s}>, defined as <ΔV^{vdw}_{X-s}>_{B}−V^{vdw}_{X-s}>_{A}, between the contribution from non-polar interactions to the potential energy between the chemical bis-aromatic compound X and its surroundings (denoted s) in two states, one state (A) being where the chemical bis-aromatic compound is surrounded by solvent, the other state (B) being where the chemical bis-aromatic compound, bound to a fumarate reductase or an analogue thereof, is surrounded by solvent, and

calculating the absolute binding free energy as an adjusted combination of the two above-mentioned average energy differences.

In the mathematical equations herein, the symbol < >means molecular dynamics average. The index X-s means compound-solvent (or compound-surrounding), the letter “X” denoting the chemical bis-aromatic compound X. Normally the bis-aromatic compound X will function as an inhibitor of the binding between the fumarate reductase and known ligands, but as discussed herein, it is also a possibility that the compound or drug will affect the fumarate reductase in such a way that the binding between known ligands and the fumarate reductase is enhanced. The superscript “el” designates the polar or electrostatic energy, while the superscript “vdw” indicates “van der Waals”, another designation for the non-polar interactions. The symbol Δ indicates that the quantity in state A is subtracted from the quantity in state B.

In the present context the term “an analogue of a fumarate reductase” should be understood, in a broad sense, any compound which mimics (with respect to binding characteristics) an interesting part of a fumarate reductase (e.g. the substrate binding part(s)), and the interaction of which with a chemical bis-aromatic compound or a group or plurality of chemical bis-aromatic compounds, e.g. drug candidates, is to be studied. Thus, the analogue may simply be any other chemical compound regarded as capable of interacting with the chemical bis-aromatic compound in a manner which mimics the binding between the fumarate reductase and a known substrate in vivo, but most often the analogue will be a relatively large molecule, in other words a macromolecule such as a protein or an oligonucleotide, which is relatively large compared to the chemical bis-aromatic compound; although the chemical bis-aromatic compound interacting with the analogue, of course, in itself be a macromolecule. In the present context, fumarate reductase or analogue thereof is preferably the fumarate reductase or an analogue thereof which exhibits at least one interesting binding characteristic relevant for the assembly of pili.

The basis for the above-indicated approach for determining the binding free energy is explained in the following:

As a starting point is taken the linear response approximation for electrostatic forces which for polar solutions as a result yields quadratic free energy functions in response to the development of charges. This is, e.g., the familiar result from Marcus's theory of electron transfer reactions (Marcus, 1964). For a system with two states, A and B, given by two potential energy functions V_{A} and V_{B} one obtains, within the approximation of harmonic free energy functions of equal curvature, the relationship (see Lee et al., 1992 and references therein):

λ=<V_{B}−V_{A}>_{A}−ΔG_{AB}=<V_{A}−V_{B}>_{B}+ΔG_{AB} (a)

where ΔG_{AB} is the free energy difference between B and A, λ the corresponding reorganisation energy and < >ᵢ denotes an average evaluated near the minimum of the potential i. Thus,

ΔG_{AB}≡½(<ΔV>_{A}+<ΔV>_{B}) (b)

where ΔV now denotes the energy difference V_{B}−V_{A}. If the hydration of a single ion is considered, this can be shown to give ΔG^{el}ₛₒₗ=½<V^{el}_{X-s}>, i.e. that the electrostatic contribution to the solvation energy equals half of the corresponding ion-solvent interaction energy (Warshel and Russell, 1984; Roux et al., 1990). Returning now to the binding problem, this result may be exploited in the following manner: For each solvation process, i.e. solvation of the bis-aromatic compound in water and inside the protein, two states are considered where the first has the bis-aromatic compound in vacuum and a non-polar cavity (given, e.g., by Lennard-Jones potential) already made in the given environment. The second state corresponds to the intact bis-aromatic compound surrounded by water or the solvated protein. The linear response approximation will then again give that

ΔG^{el}_{bind}≡½<V^{el}_{X-s}>, where V^{el}_{X-s} is the solute-solvent electrostatic term. Hence, the electrostatic contribution to the binding free energy can be approximated by ΔG^{el}_{bind}≡½<V^{el}_{X-s}>(where the Δ now refers to the difference between protein and water) and thus obtained from two MD simulations of the solvated bis-aromatic compound and of the bis-aromatic compound-protein complex.

The validity of the linear response results in the case of ionic solvation has been confirmed, e.g., in the study by Roux et al. (1990). Some additional calculations were also performed on simple systems that corroborate the approximation of equation b. These tests were carried out by comparing the free energy obtained from FEP/MD simulations of charging Na⁺ and Ca²⁺ ions in a spherical water system (Åqvist, 1990) with the corresponding <V^{el}_{X-s}>from 75 ps MD trajectories. This yielded factors relating <V^{el}_{X-s}>to ΔG^{el}ₛₒₗ of 0.49 for Na⁺ and 0.52 for Ca²⁺, both values being close to the predicted result of ½. A similar test on the charging of a methanol molecule, given by the OPLS potential (Jorgensen, 1986) in water gave a Δ G^{el}ₛₒₗ/<V^{el}_{X-s}>ratio of 0.43.

A crucial question is how to account for the contribution of non-polar interactions and hydrophobic effects to the free energy of binding which was termed ΔG^{vdw}_{bind}. In the ideal case, it should be possible to estimate this contribution from the non-polar (or van der Waals) interaction energies. The liquid theories of Chandler and coworkers (Chandler et al., 1983; Pratt and Chandler, 1977) have been successfully used to analyse hydrophobic effects and to calculate free energies of transfer for some non-polar molecules (Pratt and Chandler, 1977), but no analytical treatment of that kind seems possible for solvation in an inhomogeneous environment such as a protein's active site. However, it has been noted that the experimental free energy of solvation for various hydrocarbon compounds, such as n-alkanes, depends approximately linearly on the length of the carbon chain both in their own liquids as well as in water (Ben-Naim and Marcus, 1984). MD simulations of n-alkanes solvated in water and in a non-polar van der Waals solvent have been carried out, which indicate that also the average solute-solvent interaction energies vary approximately linearly with the number of carbons in the chain (the relationships being different in different solvents, of course). It thus seem possible that a simple linear approximation of ΔG^{vdw}_{bind} from <ΔV^{el}_{X-s}>might be able to account for the non-polar binding contribution. For instance, if σ is considered some appropriate measure of the size of the solute and if the solute-solvent van der Waals interaction energies and the corresponding non-polar free energy contributions (both in water and protein) depend linearly on σ, such that

<Vₚ^{vdw}>=αₚσ, <V_{w}^{vdw}>=α_{w}σ, ΔGₚ^{vdw}=βₚσ and ΔG_{w}^{vdw}=β_{w}σ

then

$$Δ {G}_{bind}^{vdw} = \frac{{β}_{p} - {β}_{w}}{{α}_{p} - {α}_{w}} < Δ {V}_{X - s}^{vdw} >$$

is obtained. Since it seems difficult to derive a factor relating the two quantities in a reliable way from purely theoretical considerations, the approach is taken to empirically try to determine such a relationship which is capable of reproducing experimental binding data. Thus, the free energy of binding is in one embodiment of the invention approximated by

ΔG_{bind}=½<ΔV^{el}_{X-s}>+α<ΔV^{vdw}_{X-s}> (1)

the parameter a being determined by empirical calibration.

Although, as discussed above, a theoretical prediction of the coefficient for <ΔV^{el}_{X-s}>is ½, it may be practically useful to also treat this coefficient as an empirical parameter. This would lead to the free energy of binding being approximated by

ΔG_{bind}−β<ΔV^{el}_{X-s}>+α<ΔV^{vdw}_{X-s}> (1b)

where both parameters, αand β, are determined by empirical calibration.

Finally, in some cases, it seems suitable to add an additional constant term to Equation 1, so that the equation becomes

ΔG_{bind}=½<ΔV^{el}_{X-s}>+α<ΔV^{vdw}_{X-s}>+c (2)

where c is a constant reflecting extrapolation to zero size of the chemical bis-aromatic compound, that is, where the regression line is distinctly offset from origin when moving towards zero size of the chemical bis-aromatic compound. The parameter c may also be used to correct for possible systematic errors due to e.g. the neglect of induced polarisation, possible force field deficiencies etc. In these cases, c will normally assume a value between −10 and 10 kcal/mol, typically between −3 and 3 kcal/mol, such as between −2 and 2 kcal/mol, e.g. between −1 and 1 kcal/mol. However, it is anticipated that in many cases, c can suitably be set to zero, as the extent of deviation will be of minor importance for the usefulness of the predicted values.

If also the electrostatic coefficient i treated as an empirical parameter, the approximation of the binding free energy assumes its most general form, namely

ΔG_{bind}=β<ΔV^{el}_{X-s}>+α<ΔV^{vdw}_{X-s}>+c (2b )

where now both α, β and c are to be determined by empirical calibration.

While the solvent used in the above method is suitably and most often an aqueous solvent like water, it is within the scope of the invention to take any other suitable solvent as a starting point, including, e.g., methanol, ethanol, acetone, acetonitrile, chloroform, hexane, etc., or mixtures thereof or combinations of such solvents or mixtures thereof with water. The selection of the solvent will be of little importance to the predicted values as long as the solvent is one which is able to dissolve or solvate the receptor molecule and the bis-aromatic compound (in the present context this means that a sufficient amount of fumarate reductase or analogue thereof can be homogeneously mixed with the solvent without precipitation so as to allow the determination of binding energies by some suitable method), but there may be cases where it is advantageous to modify the solvent environment (e.g. by modulating the ionic strength) in which the interaction of the bis-aromatic compound and the receptor molecule is to take place. If the environment in which the interaction between the chemical bis-aromatic compound, such as a drug, and a fumarate reductase or an analogue thereof is to take place in the actual use of the drug is the human body, it might be particularly suitable to imitate e.g. human plasma as the solvent.

A thorough discussion of the above-referenced method for determining the binding free energy between two molecules can be found in WO 95/06293.

Thus, the inventors of the present invention will by the use of X-ray crystallography elucidate the mechanism of binding between fumarate reductase and its natural or synthetic ligands thereby identifying an essential part of a defined active site responsible for the binding between ligands and their fumarate reductases, and thus providing a method to enable drug design of fumarate reductase inhibiting anti-parasitic and bacterial compounds.

Having determined the location of a promising active site for inhibitory ligands as described above the computer programs “PLIM” and “PLIM_DBS” (developed by Symbicom AB) could e.g. be used to find templates for families of compounds capable of binding to the active site.

PLIM is a Protein Ligand Interaction Modeller that constructs putative ligands for a protein using thermodynamic criteria. It calculates the energy of interaction between the protein and sample probes that are successively placed at different points on a regular grid around the molecule. For each position and orientation the interaction energy between the probe and the atoms of the protein is calculated. The energies are stored, and the best positions for a particular probe are written out (the basic calculations are described by Goodford (1985) and Boobbyer (1989) and implemented in the commercially available program GRID; the PLIM implementation is somewhat different in that the energy values are converted to discrete points that are associated with the chemical probe, enabling easy output to e.g. data base searching programs). The program then builds up the ligand by incorporating selected probe atoms at positions of energy minima on the grid. The user selects which atoms and groups should be used as probes, and which criteria should be used to determine those that will be incorporated into the ligand.

The energy is calculated as the sum of electrostatic, Van der Waals and hydrogen-bonding contributions as described herein.

The PLIM runs result in a number of suggested positions and orientations of favourable chemical groups in the region near the active site. These groups which have physical properties like charge, hydrogen bonding directionality and extended atom radia, will hereafter be denoted “site points”.

A search for potential ligands is then made by searching a database for known molecular structures that match the positions of these groups of site points, using PLIM_DBS.

The core of PLIM_DBS is an algorithm for subgraph isomorphism (cf. Ullman (1976) and Brint (1987)), where three sitepoints are represented as a distance matrix (“the pattern matrix”). The program looks for this distance pattern in the distance matrix formed from every entry in the database. If the pattern is found, the entry is superimposed on to the sitepoints and if the corresponding atom types match the entry and its orientation is saved in a hit-list. Added to this basic scheme a number of options regarding surface complementarity can be used, i.e. only entries which are matching the protein surface with respect to hydrophobic and steric properties are saved.

PLIM_DBS is thus a database searcher which hunts through a collection of 3-dimensional molecule coordinate sets, looking for entries that contain a certain pattern of atoms. This pattern is specified in terms of atom type, and of spatial position and orientation; for instance a search may be made for compounds containing an sp3 carbon atom that is 4.2 Å from a sp2 oxygen and 5.1 Å from a hydroxyl group that in turn is 5.6 Å from the oxygen. The strictness of the search can be adjusted by the user by varying the tolerance on the distance criteria and the atom-type matching, determining, for instance, whether a sp2 carbon that is a little more than 4.2 Å from an oxygen should be considered as a hit. Those hits that are found are then ranked according to a score that reflects how well the target atoms superimpose on the real molecule, and also on how complementary the molecular surface of the compound is to that of the binding pocket of the protein.

The result from a PLIM_DBS search is a list of molecular structures and their atomic coordinates, superpositioned on to the sitepoints, and given a score (“goodness of fit”). The procedure does not try to optimize the positioning of the structures, nor does it perform any molecular mechanics or dynamics calculations. Both protein and the extracted structures are treated as rigid bodies.

The structures from the database search are displayed in the context of the protein and its surface on a graphics system using a commonly available molecular modelling package. Usually the structures show some unfavourable interactions with the protein, or lack groups to fill out e.g. hydrophobic pockets. Hence, the structures form the database search are regarded as templates, to be modified and improved by an organic chemist. This process also involves choosing compounds which are easy to synthesize, which is of particular interest if the synthesis capacity is limited.

The best of these database hits are thus examined visually using a computer-graphic modelling system, and the most promising of these are selected according to a wealth of physico-chemical reasoning.

The templates may then be modified using a commercially available olecule 3D builder (e.g. MacMimic). Each template gives rise to a compound class. Each modification of the class is assigned a specific number and the coordinates and a description are stored in a tree structure, e.g. using the program ARVS_JAKT developed by Symbicom AB in Sweden. The design should be performed in a collaboration between protein structure experts and organic chemists, in order to provide the best tools possible for the chemists who will actually synthesize the compounds.

The efficacy of these modifications is finally assessed using molecular-dynamics free energy calculations as described herein to study the stability of the protein-ligand complex (cf WO 95/06293).

In order to maximize the efficiency of the above-mentioned methods for identifying/designing bis-aromatic compounds which are capable of interacting with a fumarate reductase, it is preferred that the bis-aromatic compound A is likely to be a bis-aromatic compound which is capable of binding to the selected active site.

In view of the above-described modus operandi for selecting bis-aromatic compounds which should interact with fumarate reductases this can, according to the invention, be accomplished when the bis-aromatic compound A is selected by performing the following steps:

co-crystallizing fumarate reductase or the analogue thereof with a ligand capable of interacting with a site in fumarate reductase or the analogue thereof and establishing the three-dimensional conformation of fumarate reductase or the analogue thereof and the ligand when interacting by means of X-ray crystallography,

using the above-established conformation of fumarate reductase or the analogue thereof to establish a 3-dimensional representation of the site in fumarate reductase or the analogue thereof interacting with the ligand during binding,

selecting a number of distinct chemical groups, X1, and determining the possible spatial distributions of the X1 chemical groups which maximizes the binding free energy between the chemical groups and the site in the fumarate reductase or the analogue interacting with the ligand,

extracting, from a database comprising three-dimensional representations of molecules, a molecule which has the X1 chemical groups in the possible spatial distributions determined above,

optionally modifying the 3-dimensional representation of the molecule extracted from the database, and

identifying the optionally modified molecule as the bis-aromatic compound A.

According to the invention the above-indicated steps are especially preferred when the ligand is a substrate known ligand such as a natural substrate of the fumarate reductase or a part thereof.

### Experimental

### General Methods

Lymphocyte Proliferation Assay

Human blood mononuclear cells (BMNC) from heparinized blood were isolated by metrizoate sodium-Ficoll density gradient centrifugation, washed 3 times in RPMI 1640 medium supplemented with 5% FCS and with 400 IU of penicillin plus 400 μg/ml streptomycin. BMNC were resuspended in the medium and cultured in triplicate, 0.63×10⁵/ml and 160 μl per vial, in round-bottom microtiter plates with 20 μl of various concentrations of the test compound. Immediately prior to incubation, optimum concentrations of the mitogen phytohaemagglutinin (PHA) and the antigen purified protein derivative of tuberculin (PPD) were added to the cultures in a volume of 20 μl. Unstimulated control cultures were always included. Cultures were incubated for 3 or 7 days. The degree of lymphocyte proliferation was estimated by ³H-thymidine (1 μCi per well) addition 24 h before the cells were harvested on glass fiber filters by means of a harvesting machine), and ³H-thymidine incorporation was measured in a liquid scintillation counter. For each set of triplicate values, the median was recorded. Unstimulated cultures were always included as controls.

Cytokine Inhibition Assay

IC₅₀ values are determined as described in the examples under the heading Cytokine production by human peripheral blood mononuclear cells in vitro.

Anticancer Assay

In vitro studies: The selected compounds were tested for inhibition of the growth of two human cell lines CEM and HL60. The cells were grown in conventional tissue culture media in the presence of different concentrations of each compound for 48 hrs. After incubation, the cell number was determined in a Coulter counter, and compared with those in the culture medium alone. Based on these results the IC₅₀ values were determined.

Anti-virus Assay

IC₅₀ values are determined by the virus plaque formation and/or the virus cytopathic assay using standard methods well-known in the art.

Helicobacter Species Assay

20 strains of Helicobacter pylori were used in an agar plate assay. The compounds were incorporated in the agar plates at different concentrations as described in the examples and were then inoculated with different strains of Helicobacter pylori followed by incubation in a microaerophilic atmosphere at 37° C. for 72 hours. The number of colony-forming units (CFU) were counted after the incubation period. The MIC values were determined as the minimal concentration required to inhibit growth of the bacteria.

Leishmania promástigotes Assay

A WHO reference vaccine strain of L. major originally isolated from a patient in Iran and a Kenyan strain of L. donovani (MHOM(/KE/85/NLB 274) were cultured in medium 199 containing 0.02 mg/ml gentamycin, 25 mM Hepes, 4 mM L-glutamine, and 20% heat inactivated fetal calf serum (FCS). Incubation was carried out at 26° C. Promastigotes were harvested on day 3 and 6 of the culture and used for the parasite growth inhibition.

The effect of the test compounds on promastigotes was assessed by a method similar to the one described by Pearson et al., by incubating promastigotes (3×10⁶/ml) at 26° C. for 2 hrs in the presence of a given compound or the medium alone in 96 wells flat bottom microtiter plates. Following incubation, 100 μCi of 3H-thymidine was added to each well and further incubated for 18 hrs. Promastigotes were then harvested on filter paper by means of a cell harvester, extensively washed with distilled water and counted in a scintillation counter. The promastigotes were also counted microscopically and their flagellar motility was assessed.

Plasmodium falciparum Assay

The experiments in which compounds were tested for their ability to inhibit parasite growth were performed by a modification of the method originally described by Jensen et al. (1982). 50 μl of parasitized erythrocytes (parasitemia approximately 1%) in a concentration of 5×10⁸/ml and 50 μl of RPMI medium containing different concentrations of the test compound was added to each well of a 96 well flat-bottomed microtiter plate. The cultures were then incubated for 48 hours, 24 hours before termination of the culture adding 20 μl of 3-H-hypoxanthine (40 uCi/ml) was added to each well. The cultures were then harvested onto glass fiber filters using a Skatron cell harvester, and the incorporation of 3-H-hypoxanthine into the DNA of dividing parasites was determined by liquid scintillation spectrometry. Control cultures with uninfected erythrocytes and infected erythrocytes in RPMI medium without test compounds were always performed in parallel to the test cultures. In some experiments thin smears of parasite cultures were stained by Giemsa and examined under microscope (×1000). The test compounds described above, were diluted in RPMI medium immediately before use. In the experiments, chloroquine phosphate was used as a positive control as a drug known to inhibit parasite growth.

Leishmania major in vivo Assay

BALB/c female mice aged eight weeks old were used throughout the experiment. The WHO reference vaccine strain of L. major originally isolated from a patient in Iran was cultured as described in the Leishmania promastigotes Assay above. For animal infection, mice received s.c. injections (in 0.05 ml of PBS) in the left hind footpad with 1×10⁷ stationary phase promastigotes.

Footpad lesions were measured and expressed as footpad thickness increase (in mm). The footpad thickness of mice was measured before infection and every 3 days after 7 days of infection. From 7 days of infection, mice received injections of the test compound (dissolved in 20 μl of 99% (v/v) ethanol, and then 980 μl of medium 199 was added and the resulting mixture was stored at −20° C. before use) i.p. once a day. After 42 days of injections, some of the mice were killed and the footpads, spleens and livers removed. The parasite loads in the footpads and livers were estimated by a modification of the method described by Liew et al. using 3-H-thymidine uptake. The results were expressed as cpm. The footpads, spleens and livers impression was also estimated.

Leishmania donovani in vivo Assay

Male Syrian golden hamsters (Mesocricetus auratus), 50-70 g body weight, were used throughout the experiment. L. donovani (MHOM/KE/85/NLB 439) promastigotes were used. Animals were intracardially inoculated with 2×10⁷ L. donovani promastigotes in 0.1 ml medium 199 (Day 0). One hour later, one of the animals was killed. The liver and the spleen were weighed. The liver and the spleen impression smears were made. After air-drying, the impression smears were fixed with water-free methanol and stained with Giemsa. Five of the animals were treated (i.p.) with the test compound (dissolved in 20 μl of 99% (v/v) ethanol, and the 980 μl of medium 199 was added, and the resulting mixture was stored at −20° C.) (10 mg/kg body weight two times per day) from Day+1 to Day+7. Another five animals were treated with 0.85% NaCl. The animals were killed on Day+8. The liver and spleen were weighed, and the liver and the spleen impression smears were made. The number of the parasite in the liver and the spleen were counted under microscope. The spleen of the animals were cut into very small pieces, cultured in 15 ml of the culture medium at 26° C. overnight and the parasite load was determined by ³H-thymidine uptake as described above.

In vivo Malaria Parasites Assay

BALB/c or CF1female mice aged eight weeks were used. The Plasmodium sp. causing malaria in humans can only infect certain primates. Therefore it has not been possible to determine whether a given test compound inhibits parasite multiplication of human malaria parasites in vivo. However, there are several Plasmodium sp. that infect rodents. These systems have earlier been used to test the ability of drugs to inhibit malaria infections in vivo. In the experiments described below mice were infected with either P. yoelii YM strain or P. berghei K173 and were compared to the outcome of infection in untreated control animals and in animals treated with the test compound. The parasites were maintained by passage through BALB/c or CF1mice, and the animals were infected by injection of infected erythrocytes obtained from mice with a parasitemia of approximately 40%. The animals were injected intraperitoneally with either 1×10⁶ parasitized erythrocytes diluted in 0.9% NaCl and in a final volume of 0.2 ml. The day of infection was termed day 0. The outcome of infection was assessed microscopically by examination of Giemsa stained blood films. The load of infection (the parasitemia) was calculated as the percentage of infected erythrocytes of the total number of erythrocytes.

Legionella Assay

20 Legionella strains: Five clinical isolates from bronchial secretions and a lung abscess: 2 Legionella pneumophila serogroup 1 and 3 Legionella micdadei (L. detroit, L. bari, L. F 1433). Eight Legionella pneumophila serogroups 1-7 and one strain of each of L. bozemanii, L. dumoffii, L. gormanii, L. micdadei, L. feelei, L. wadswort-hii, L. longbeacheae. Staphylococcus aureus ATCC 25923 was the control strain. The Legionella strains were subcultured on buffered charcoal yeast extract with alfa-ketoglutarate (BCYE-α), and the rest of the strains were subcultured on 10% horse blood agar for 48 hours and 24 hours, respectively. Macrodilution rows were made with buffered yeast extract with alfa-ketoglutarate (BYE-α) with 2 ml aliquots in vials, containing various concentrations of the test compound. Suspensions of Legionella species and the other pathogens and commensals were made in BYE-60 . All the dilution rows were inoculated to give a final concentration of 10⁵ CFU/ml. After incubation at 37° C. for 2 and 24 hours, respectively, aliquots of 10 μl were taken from all dilution steps and plated onto BCYE-x agar plates (all Legionella species) and to 10% horse blood agar (all non-Legionella strains). All the BCYE-x plates were incubated for 48 hours in a humid atmosphere at 37° C. and read. The inoculated 10% horse blood agar plates were incubated in a normal atmosphere at 37° C. for 24 hours and read.

Mycobacteria Assay

63 strains of mycobacteria were used. The bacteria were grown in Dubos broth media before susceptibility testing. The test compound was dissolved in dimethyl sulfoxide (DMSO) and diluted in distilled water to the desired concentration.

Susceptibility testing was performed radiometrically by using a BECTEC 460-TB apparatus in a confined atmosphere (5% CO₂). Bacterial growth was measured as a function-of the ability of the bacteria to catabolize ¹⁴C-labelled palmitic acid in the BECTEC 7H12B TB medium during growth, which resulted in the release of ¹⁴C-labelled CO₂. The growth was expressed as a numerical value called the growth index (GI) which ranged from 1 to 999. The 7H12 vials were inoculated with 0.1 ml of an appropriately diluted Dubos broth culture to give a final inoculum of about 5×10⁴ colony-forming units (CFU) per ml together with 0.1 ml of different concentrations of the test compound. The final concentrations of the test compound ranged from 1.25 μg/ml to 80 μg/ml. A vial without the test compound, but with an inoculum diluted 1:100, was included as a control. The final inoculum was determinated by culturing 0.1 ml from the control vial onto one Lowenstein-Jensen slant. The vials were incubated under stationary conditions at 35° C. and growth was monitored by daily GI determination for 7 days. At day 7, 0.1 ml from each vial with a GI reading <30 was cultured onto one Lowenstein-Jensen slant. Colony counts were enumerated after incubation at 35° C. for 3 weeks.

Minimal inhibitory concentration (MIC) was defined as the lowest concentration of the test compund which could inhibit 99% or more of the mycobacteria population. Minimal bactericidal concentration (MBC) was defined as the lowest concentration of the test compound which killed 99% or more of the mycobacteria population.

Anticoccidial Assay

The experiment was carried out in collaboration with Korn og Foderstof Kompagniet (KFK) at KFK's Experimental Station (Forsøgsgård, Sdr. Forumvej 18, DK-6715 Esbjerg, Denmark). The test compound was mixed manually with chicken feed one week before use. 2.6 g of the test compound was mixed with 1 kg rye flour. The mixture was then mixed with ten kg chicken feed. The prepared feed as well as a standard feed used, containing 70 ppm salinomycin which is a known coccidiostatic agent, were stored at a temperature between 10° C. and 15° C. prior to use. Eimeria tenella sporulated oocysts were obtained from the Agricultural and Food Council Institute for Animal Health, Compton Laboratory, Berkshire, England. The oocysts were washed and resuspended in 30 ml saline to give a concentration of 15×10⁶/30 ml. A volume of 0.1 ml (50,000 oocysts) was given to each chicken. The experimental set-up consisted of 4 groups of 14-days old chickens. During the first 14 days of life, all the chickens received chicken feed containing no coccidiostatic agents. The first 3 groups were given 50,000 E. tenella oocysts per chicken by oral administration on day 14. Feeding the chickens with the feed preparations described above started one day before infection with the parasite (day 13). The treatment continued for 14 days according to the set-up shown in the table below.

Experimental set-up for testing anticoccidial activity of a test compound.

| | | Infection with | |
|---|---|---|---|
| Groups | Number | E. tenella | Treatment |
| 1 | 35 | Yes | Standard KFK feed containing |
| | | | salinomycin |
| 2 | 20 | Yes | Test compound (10 mg/kg |
| | | | chicken body weight/day) |
| 3 | 35 | Yes | None |
| 4 | 35 | Yes | None |

The following parameters were examined and the samples were obtained:

1. All the chickens were weighed once a week as a standard procedure.

2. Mortality of the chickens was observed and recorded on a daily basis.

3. At the end of the experiment (14 days treatment, 28 days old chickens), the chickens were slaughtered and a necropsy was performed for identification of gross pathology. Histopatholology was registered in standard HE sections of 10-15 mm of one cecal sac, one transverse and one longitudinal. The sections from each chicken were examined. The pathology was registered according to J. Johnson and W. M. Reid, Anticoccidial drugs: Lesion scoring techniques in battery and floor pen experiments with chickens. Experimental Parasitology 28 (1970), 30-36.

4. Parasite load in the intestine or number per smear may be determined at the end of the experiment. The number of oocysts in 10 viewfields may be counted at 100×enlargement and the average of 10 fields were used. Oocysts index may calculated as:

$$\frac{Oocysts in infected animal / field \times 100}{Oocysts in control animal / field}$$

and may be recorded as follows:

0=no oocysts

+=1 oocysts/field

++=1-10 oocysts/field

+++=>10 oocysts/field

The QSAR Model

The QSAR Model is based on a predetermined number of compounds selected by a statistical design and their corresponding biological activities (e.g. the IC₅₀ values from an in vitro assay).

Calculations

The low energy conformations of the compounds are predicted by using MacroModel 4.5 (F. Mohamadi, N. G. J. Richards, W. C. Guida, R. Liskamp, M. Lipton, C. Caufield, G. Chang, T. Hendrickson, W. C. Still., MacroModel—An Integrated Software System for Modeling Organic and Bioorganic Molecules Using Molecular Mechanics. J. Comput. Chem. 1990, 11, 440-476. The MacroModel software is available from W.C. Still, Department of Chemistry, Columbia University, New York, N.Y. 10027, U.S.A). The interaction energies are calculated by GRID [version 15 (P. Goodford, GRID, molecular Discovery Ltd, Oxford England 1966)] using a grid with a spacing of 1 Å and the following dimensions (A): Xₘᵢₙ/Xₘₐₓ: −17.5/13.5; Yₘᵢₙ/Yₘₐₓ: −13.5/15.0 and Zₘᵢₙ/Zₘₐₓ: −8.5/10.5, using interactions with three probes (water, methyl and ammonium). The biological activities are used to generate the partial least square projections to latent structures by GOLPE 3.02 (S. Clementi, GOLPE 3.02, Multivariate Infometric Analyses (MIA), Perugia Italy, 1996).

Variables Preselection

GOLPE rejects variables having a total sum of squares (SS) lower than 10⁻⁷. The number of variables were further reduces by D-optimal preselection or region selection before applying variable selection.

D-optimal preselection. Variables were selected according to their position in the weight space, using a D-optimal design criterion. The variables containing most information and having least correlation were selected. The number of variable were reduced by no more than 50% each time, until the R² value starts to decrease.

Region selection (Grouping). A number of seeds (1920) were selected using a D-optimal design criterion in the weight space. Structural difference between different molecules in the series will be reflected in groups of variables, and therefore groups were generated around each seed in the 3D-space. Variables with a distance of no more than 1A to the seeds are included in the groups. If two neighbour groups (distance smaller than 2A) contained the same information, the groups were collapsed. The groups were used in the variable selection procedure replacing the original variables. The effect of the groups on the predictivity were evaluated and groups instead of individual variables were removed from the data file.

Variable Selection

The effect of the variables on the predictivity was evaluated using a Fractional Factor Design (FFD) procedure. A number of reduced models (twice the number of variables) were build removing some of the variables according to the FFD design. The effect of dummy variables (20%) on the predictivity were calculated and only if a variable had a positive effect on the predictivity larger than the effect on the average dummy variable, the variable was included in the final model.

Cross Validation

The models were validated using random groups. Molecules were assigned in a random way to 5 groups of equal size. Reduced models were build keeping out on group at a time. The formation of the groups was repeated 10 times.

Analyses

The NMR spectra were recorded on a Bruker AC-200F spectrometer. Splitting pattern are described as singlet (s), doublet (d), triplet (t), quartet (q) and broad (b). * indicates that signals with similar shifts might be interchanged. Mass spectra were recorded on a JEOL AX505W mass spectrometer. Melting points were determined on an Electrothermal melting point apparatus, and were not corrected. All moisture sensitive reactions were performed under nitrogen using oven-dried glassware. Solvents were dried before use: Tetrahydrofurane was freshly distilled from sodium/benzophenone, toluene was distilled and stored over sodium. HPLC grade dimethylformamide was dried and stored over 4 Å molecular sieves. lodopropane was distilled from CaH₂ before use. Cesium fluoride was dried at 150° C. under oil pump vacuum for 2 h and stored in a desiccator.

Samples of E-1-(4-(2-propenyloxy)phenyl)-3-(2,4-dimethoxyphenyl)-hex-2-enone-1, Z-1-(4-(2-propenyloxy)phenyl)-3-(2,4-dimethoxyphenyl)-hex-2-enone-1 and 3-(2,4-dimethoxyphenyl)-2-propyl-1-(4-(2-propenyloxy)phenyl)prop-2-enone-1 for biological testing were purified by HPLC using a Waters 6000 A pump, a prepacked Knauer column (16×250 mm, LiChrosorp RP18, eluent: acetonitrile-water) and a Shimadzu SPD 6A UV detector at 254 nm. Column chromatography was performed on silica gel (Merck, 0.040-0.063 mm) using mixtures of toluene and ethyl acetate as eluents.

### EXAMPLES

### Example 1

### Preparation of Compounds

### 1) Preparation of 2-(2,4-dimethoxyphenyl)-4-(4-(2-propenyloxy)phenyl)-4-oxobutanenitrile

A stirred solution of 2,4-dimethoxy-4′-(2-propenyloxy)chalcone (19.44 g, 60.0 mmol), sodium cyanide (29.4 g, 600 mmol) and ammonium chloride (3.24 g, 60 mmol) in dimethylformamide (240 ml) was heated to 100° C. for 20 min. The reaction was allowed to cool to room temperature, filtered, 4M HCl slowly added (240 ml), and extracted with ethyl acetate (3×200 ml). The combined organic phase were concentrated in vacuo and the residue freeze-dried to give 17.8 g (84.4%) of 2-(2,4-dimethoxyphenyl)-4-(4-(2-propenyloxy)phenyl)-4-oxobutanenitrile as colourless crystals, mp: 112.2-112.7° C. ¹H NMR (CDCl₃) δ7.90 (AA′ part of an AA′MM′ system, H2′, H6′), 7.37 (d, J 8.4 Hz, H6), 6.91 (MM′ part of an AA′MM′ system, H3′, H5′), 6.50 (dd, J 8.4, 2.0 Hz, H5), 6.47 (bs, H3), 6.03 (ddq, J 17.3, 10.5, 5.2 Hz, H2″), 5.42 (dd, J 17.3, 1.3 Hz, H3″trans), 5.32 (dd, J 10.5, 1.3 Hz, H3″cis), 4.67 (dd, J 8.4, 5.0 Hz, Hβ), 4.58 (bd, J 5.2 Hz, H1″), 3.82 (s, OCH₃), 3.79 (s, OCH₃), 3.58 (dd, J 17.4, 8.4Hz, Hα), 3.40 (dd, J 17.4, 5.0 Hz, Hα′). ¹³C NMR (CDCl₃) δ193.6 (C═O), 162.4* (C4′), 160.6* (C2), 156.8* (C4), 132.0 (C2″), 129.9 (C2′, C6′), 129.0 (C6), 128.6 (C1′), 120.6 (C≡N), 117.7 (C3″), 115.0 (C1), 114.1 (C3′, C5′), 104.2 (C5), 98.5 (C3), 68.4 (C1″), 55.2 (OCH₃), 55.0 (OCH₃), 41.4 (Cα), 26.5 (Cβ).

### 2) Preparation of 2-(2,4-dimethoxyphenyl)-2-methyl-4-(4-(2-propenyloxy)phenyl)-4-oxobutanenitrile

To a cold (−78° C.) solution of 2-(2,4-dimethoxyphenyl)-4-(4-(2-propenyloxy)phenyl)-4-oxobutanenitrile (3.52 g, 10 mmol) in anhydrous tetrahydrofuran (40 ml) was slowly added lithium diisopropylamide (11 ml of a 2 M soln. in diethyl ether, 22 mmol). After 5 min iodomethane (4.0 ml, 50 mmol) was added and the reaction was stirred for additional 10 min. The reaction was heated to room temperature. (30 min) and water (50 ml) was added. The mixture was extracted with ethyl acetate (3×50 ml) and the combined organic phases were concentrated in vacuo and the residue purified by column chromatography to give 2.85 g (78.0%) of 2-(2,4-dimethoxyphenyl)-2-methyl-4-(4-(2-propenyloxy)phenyl)-4-oxobutanenitrile as yellow crystals, mp: 77.8-78.50° C. ¹H NMR (CDCl₃) δ7.88 (AA′ part of an AA′MM′ system, H2′, H6′), 7.47 (d, J 8.5 Hz, H6), 6.91 (MM′ part of an AA′MM′ system, H3′, H5′), 6.50 (dd, J 8.5, 2.3 Hz, H5), 6.46 (d, J 2.3 Hz, H3), 6.04 (ddq, J 17.3, 10.5, 5.2 Hz, H2″), 5.42 (dd, J 17.3, 1.3 Hz, H3″trans), 5.32 (dd, J 10.5, 1.3 Hz, H3″cis), 4.60 (bd, J 5.2 Hz, H1″), 3.96 (d, J 17.1 Hz, Hα), 3.84 (s, OCH₃), 3.80 (s, OCH₃), 3.56 (d, J 17.1 Hz, H-α′), 1.95 (s, CH₃). ¹³C NMR (CDCl₃) δ194.2 (C═O), 162.6* (C4′), 160.6* (C4), 157.6* (C2), 132.4 (C2″), 130.3 (C2′, C6′), 129.8 (C1′), 128.6 (C6), 120.4 (C≡N), 119.3 (C1), 118.2 (C3″), 114.4 (C3′, C5′), 104.3 (C5), 99.8 (C3), 68.8 (C1″), 55.5(OCH₃), 55.3 (OCH₃), 45.2 (Cα), 37.1 (Cβ), 25.2 (β-CH₃).

In an analogous manner, but substituting iodomethane with iodopropane, 3.21 g (81.6%) of 2-(2,4-dimethoxyphenyl)-2-propyl-4-(4-(2-propenyloxy)phenyl)-4-oxobutanenitrile was obtained as colourless crystals, mp: 86.1-87.1° C. ¹H NMR (CDCl₃) δ7.86 (AA′ part of an AA′MM′ system, H2′, H6′), 7.61 (d, J 8.6 Hz, H6), 6.90 (MM′ part of an AA′MM′ system, H3′, H5′), 6.50 (dd, J 8.6, 1.5 Hz, H5), 6.40 (d, J 1.5 Hz, H3), 6.03 (ddq, J 17.3, 10.5, 5.2 Hz, H2″), 5.41 (dd, J 17.3, 1.3 Hz, H3″trans), 5.32 (dd, J 10.5, 1.3 Hz, H3″cis), 4.59 (bd, J 5.2 Hz, H1″), 4.10 (d, J 17.1 Hz, Hα) 3.78 (s, OCH₃), 3.76 (s, OCH₃), 3.56 (d, J 17.1 Hz, Hα′), 2.32 (td, J 12.6, 4.5 Hz, Hβ1), 2.09 (td, J 12.6, 4.5 Hz, Hβ1′), 1.53 (m, Hβ2), 1.21 (m, Hβ2′), 0.91 (t, J 7.3 Hz, HβS). ¹³C NMR (CDCl₃) δ194.4 (C═O), 162.4* (C4′), 160.1* (C4), 157.1* (C2), 132.3 (C2″), 130.6 (C6), 130.1 (C2′, C6′), 129.8 (C1′), 122.7 (C1), 118.1 (C3″), 117.1 (C≡N), 114.2 (C3′, C5′), 104.0 (C5), 99.6 (C3), 68.7 (C1″), 55.2 (OCH₃), 44.5 (Cα), 43.8 (Cβ), 39.4 (Cβ1), 18.7 (Cβ2), 13.9 (Cβ3).

### 3) Preparation of 1-(4-(2-propenyloxy)phenyl)-3-(2,4-dimethoxyphenyl)-but-2-en-1-one

To a solution of 2-(2,4-dimethoxyphenyl)-2-methyl-4-(4-(2-propenyloxy)phenyl)-4-oxobutane-nitrile (1.38 g, 3.8 mmol) in anhydrous toluene (60 ml) was added sodium hydride (1.53 g 60% dispersion, 38.0 mmol) and refluxed for 20 min. Water was added and the mixture was extracted with ethyl acetate (2×90 ml). The combined organic phases were concentrated in vacuo and the residue purified by column chromatography to give 1.18 g (91.4%) of 1-(4-(2-propenyloxy)phenyl)-3-(2,4-dimethoxyphenyl)-but-2-enone-1 as yellow crystals, mp: 79.7-80.1° C. ¹H NMR (CDCl₃) δ7.97 (AA′ part of an AA′MM′ system, H2′, H6′), 7.20 (d, J 9.0 Hz, H6), 6.96 (s, Hα), 6.91 (MM′ part of an AA′MM′ system, H3′, H5′), 6.50 (m, H3, H5), 6.06 (ddq, J 17.3, 10.5, 5.2 Hz, H2″), 5.41 (dd, J 17.3, 1.3 Hz, H3″trans), 5.31 (dd, J 10.5, 1.3 Hz, H3″cis), 4.59 (bd, J 5.2 Hz, H1″), 3.83 (s, OCH₃), 2.50 (s, β-CH₃). In a NOESY spectrum correlations between Hα and H2′, H6′ and H6 were found. ¹³C NMR (CDCl₃) δ190.4 (C═O), 161.8* (C4′), 160.9* (C4), 157.7* (C2), 154.3 (Cβ), 132.4 (C2″), 130.4 (C2′, C6′), 129.6 (C6), 126.1 (C1′), 123.3 (Cα), 118.6 (C1), 118.6* (C3″), 114.1 (C3′, C5′), 104.1 (C5), 98.8 (C3), 68.7 (C1″), 55.4 (OCH₃), 55.3 (OCH₃), 20.5 (β-CH₃).

### 4) Preparation of 1-(4-(2-propenyloxy)phenyl)-3-(2,4-dimethoxyphenyl)-hex-2-en-1-one

To a solution of 2-(2,4-dimethoxyphenyl)-2-propyl-4-(4-(2-propenyloxy)phenyl)-4-oxobutanenitrile (0.79 g, 2.0 mmol) in anhydrous toluene (40 ml) was added sodium hydride (0.15 g of 60% NaH in oil, 3.8 mmol) and refluxed for 90 min. Water was added and the mixture was extracted with ethyl acetate (2×50 ml). The combined organic phases were concentrated in uacuo and the residue purified by column chromatography to give 0.13 g (18.1%) of E-1-(4-(2-propenyloxy)-phenyl)-3-(2,4-dimethoxyphenyl)-hex-2-enone-1, and 0.37 g (51.0%) of Z-1-(4-(2-propenyloxy)-phenyl)-3-(2,4-dimethoxyphenyl)-hex-2-enone-1 as yellow oils.

E-1-(4-(2-propenyloxy)phenyl)-3-(2,4-dimethoxyphenyl)-hex-2-enone-1: ¹H NMR (CDCl₃) δ7.97 (AA′ part of an AA′MM′ system, H2′, H6′), 7.14 (d, J 9.0 Hz, H6), 6.93 (MM′ part of an AA′MM′ system, H3′, H5′), 6.83 (s, Hα), 6.50 (d, J 2.2 Hz, H3), 6.47 (dd, J 9.0, 2.2 Hz, H5), 6.03 (ddq, J 17.3, 10.5, 5.2 Hz, H2″), 5.41 (dd, J 17.3, 1.3 Hz, H3″trans), 5.32 (dd, J 10.5, 1.3 Hz, H3″cis), 4.59 (bd, J 5.2 Hz, H1″), 3.81 (s, OCH₃), 2.97 (t, J 7.5 Hz, Hβ1), 1.40 (6.tet, J 7.5 Hz, Hβ2), 0.88 (t, J 7.5 Hz, Hβ3). In a NOESY spectrum correlations between Hα and H2′, H6′ and H6 were found. ¹³C NMR (CDCl₃) δ190.2 (C═O), 161.8* (C4′), 160.7* (C4), 158.3* (C2), 157.6 (Cβ), 132.4 (C2″), 132.3 (C1′), 130.4 (C2′, C6′), 130.0 (C6), 124.7 (C1), 124.0 (Cα), 117.8 (C3″), 114.1 (C3′, C5′), 104.0 (C5), 98.6 (C3), 68.6 (C1″), 55.3 (OCH₃), 55.1 (OCH₃), 34.4 (Cβ1), 21.8 (Cβ2), 14.0 (Cβ3). HRMS (FAB+): 367.1937 (calc. C₂₃H₂₇O₄: 367.1909, [M+H]).

Z-1-(4-(2-propenyloxy)phenyl)-3-(2,4-dimethoxyphenyl)-hex-2-enone-1: ¹H NMR (CDCl₃) δ7.82 (AA′ part of an AA′MM′ system, H2′, H6′), 6.90 (d, J 7.9 Hz, H6), 6.81 (MM′ part of an AA′MM′ system, H3′, H5′), 6.63 (s, Hα), 6.35 (dd, J 9.0, 2.3 Hz, H5), 6.31 (d, J 2.3 Hz, H3), 6.03 (ddq, J 17.3, 10.5, 5.2 Hz, H2″), 5.41 (dd, J 17.3, 1.3 Hz, H3″trans), 5.32 (dd, J 10.5, 1.3 Hz, H3″cis), 4.59 (bd, J 5.2 Hz, H1″), 3.70 (s, OCH₃), 3.61(s, OCH₃), 2.52 (t, J 7.1 Hz, Hβ1), 1.45 (6.tet, J 7.1 Hz, Hβ2), 0.94 (t, J 7.1 Hz, Hβ3). In a NOESY spectrum correlations between Hα and H2′, H6′ and the allylic protons in the propyl group were found. ¹³C NMR (CDCl₃) δ191.8 (C═O), 161.7* (C4′), 160.3* (C4), 156.9* (C2), 152.7 (Cβ), 132.6 (C2″), 131.4 (C1′), 130.7 (C2′, C6′), 129.7 (C6), 124.5 (Cα), 121.8 (C1), 117.9 (C3″), 113.8 (C3′, C5′), 103.9 (C5), 98.5 (C3), 68.6 (C1″), 55.1 (OCH₃), 55.0 (OCH₃), 41.0 (Cβ1), 21.0 (Cβ2), 13.7 (Cβ3). HRMS (FAB+): 367.1938 (calc. C₂₃H₂₇O₄: 367.1909, [M+H]).

### 5) Preparation of 2-(2,4-dimethoxyphenyl)-4-(4-(2-propenyloxy)phenyl-4-tertbutyldimethylsilyloxy-but-3-enenitrile

To a solution of 2-(2,4-dimethoxyphenyl)-4-(4-(2-propenyloxy)phenyl)-4-oxobutanenitrile (3.5 g, 10 mmol) and tertbutyldimethylsilyl chloride (2.0 g, 13 mmol) in anhydrous tetrahydrofuran (50 ml) was added sodium hydride (1.6 g, 60% dispersion, 40 mmol). After violent stirring for 60 min, water (30 ml) was slowly added. The mixture was extracted with ethyl acetate (2×50 ml) and the combined organic phases were concentrated in vacuo and the residue purified by column chromatography to give 3.4 g (74.0%) of 2-(2,4-dimethoxyphenyl)-4-(4-(2-propenyloxy)phenyl)-4-tertbutyldimethylsilyloxy-but-3-enenitrile as a yellow oil. ¹H NMR (CD₃CN) δ7.41(AA′ part of an AA′MM′ system, H2′, H6′), 7.31 (d, J 8.3 Hz, H6), 6.90 (MM′ part of an AA′MM′ system, H3′, H5′), 6.58 (d, J 2.3 Hz, H3), 6.53 (dd, J 2.3, 8.3 Hz, H5), 5.99 (ddq, J 17.3, 10.5, 5.2 Hz, H2′), 5.40 (dd, J 17.3, 1.5 Hz, H3″trans), 5.36* (d, J 9.7 Hz, Hα), 5.27 (dd, J 10.5, 1.5 Hz, H3″cis), 5.20* (d, J 9.7 Hz, Hβ, 4.54 (bd, J 5.2 Hz, H1″), 3.87 (s, OCH₃), 3.79 (s, OCH₃), 1.00(s, C(CH₃)₃), 0.04 (s, Si—CH₃), −0.13 (s, Si—CH₃). ¹³C NMR (CD₃CN) δ160.4* (C2), 158.5* (C4), 156.9* (C4′), 151.9 (C—OSi), 132.9 (C2″), 129.8 (C1′), 128.2 (C6), 127.3 (C2′, C6′), 120.1 (C1), 116.4 (C3″), 115.8 (C≡N), 113.6 (C3′, C5′), 104.4 (C5), 102.5 (Cα), 98.2 (C3), 67.9 (C1″), 54.9 (OCH₃), 54.5 (OCH₃), 26.1 (Cβ), 24.5 ((CH₃)₃—C—Si), 17.3 (C—Si), −5.3 (CH₃—Si), −5.4 (CH₃—C—Si).

### 6) Preparation of 2-(2,4-dimethoxyphenyl)-3-methyl-4-oxo-4-(4-(2-propenyloxy)phenyl)-butanenitrile

To a solution of 2-(2,4-dimethoxyphenyl)-4-(4-(2-propenyloxy)phenyl)-4-tertbutyldimethylsilyloxy-but-3-enenitrile (1.16 g, 2.5 mmol) and iodomethane (313 μl, 5.0 mmol) in anhydrous dimethylformamide (12.5 ml) was added caesium fluoride (0.76 g, 5.0 mmol). The mixture was stirred for 6 h, water (10 ml) was added and the mixture was extracted with ethylacetate (4×20 ml). The combined organic phases were concentrated in vacuo to give a mixture of the two epimeric racemic pairs of 2-(2,4-dimethoxyphenyl)-3-methyl-4-oxo-4-(4-(2-propenyloxy)phenyl)-butanenitrile (2.2 mmol, 89.0%) as colourless crystals, mp: 123.6-124.7° C. ¹H NMR (CDCl₃) δ7.95/7.86 (AA′ part of an AA′MM′ system, H2′, H6′), 7.29/7.23 (d, J 9.1/9.1 Hz, H6), 6.94/6.90 (MM′ part of an AA′MM′ system, H3′, H5′), 6.42 (m, H5, H3), 6.03 (ddq, J 17.3, 10.5, 5.2 Hz, H2″), 5.42 (dd, J 17.3, 1.3 Hz, H3″trans), 5.32 (dd, J 10.5, 1.3 Hz, H3″cis), 4.58 (bd, J 5.2 Hz, H1″), 4.44/4.50 (d, J 8.9/7.1 Hz, Hβ), 4.03 (bp, J≈8 Hz, Hα), 3.82/3.85 (s, OCH₃), 3.80/3.76 (s, OCH₃), 1.10/1.39(d, J 7.1/7.1 Hz, CH₃). ¹³C NMR (CDCl₃) δ198.8/198.6 (C═O), 162.6* (C4′), 160.8* (C2), 157.6/157.2* (C4), 132.2 (C2″), 130.4(C2′, C6′), 130.3(C6), 128.4 (C1′) 119.6 (C N), 118.1 (C3″), 114.7(C1), 114.4(C3′, C5′), 104.6/104.2 (C5), 98.6/98.9(C3), 68,7 (C1″), 55.4 (OCH₃), 55.2 (OCH₃), 41.7/41.9(Cα), 33.4/34.9 (Cβ), 16.3/15.6 (CH₃).

In an analogous manner, but substituting iodomethane with iodopropane, a mixture of the two epimeric racemic pairs of 2-(2,4-dimethoxyphenyl)-3-propyl-4-oxo-4-(4-(2-propenyloxy)phenyl)butanenitrile (1.2 mmol, 62.1%) was obtained as a yellow oil. ¹H NMR (CDCl₃) δ7.87/7.80(AA′ part of an AA′MM′ system, H2′, H6′), 7.28/7.12 (d, J 9.0/9.0 Hz, H6), 6.91/6.87 (MM′ part of an AA′MM′ system, H3′, H5′), 6.43/6.36 (m, H5, H3), 6.03 (ddq, J 17.3, 10.5, 5.2 Hz, H2″), 5.42 (dd, J 17.3, 1.3 Hz, H3″trans), 5.32 (dd, J 10.5, 1.3 Hz, H3″cis), 4.58 (bd, J 5.2 Hz, H1″), 4.44/4.34 (d, J 7.9/8.6 Hz, Hβ), 4.10 (m, Hα), 3.84/3.85 (s, OCH₃), 3.76/3.71 (s, OCH₃), 1.8/1.9 (m, Hα1), 1.2 (m, Hα2), 0.79/0.85 (t, J 7.2 Hz, Hα3). ¹³C NMR (CDCl₃) δ199.0/198.9 (C═O), 162.5* (C4′), 160.8* (C2), 157.4* (C4), 132.2 (C2″), 130.4/130.5 (C1′), 130.3/130.2 (C2′, C6′), 130.0/130.1 (C6), 120.0/119.7 (C≡N), 118.0 (C3″), 114.1/114.5 (C1), 114.3/114.2 (C3′, C5′), 104.4/104.3 (C5), 98.6/98.9 (C3), 68.7 (C1″), 55.4 (OCH₃), 55.1 (OCH₃), 47.0/46.3 (Cα), 35.2 (Cβ), 32.9/33.2 (Cα1), 19.8/20.1 (Cα2), 13.7/13.8 (Cα3).

### 7) Preparation of 3-(2,4-dimethoxyphenyl)-2-methyl-1-(4-(2-propenyloxy)phenyl)prop-2-enone-1

To a solution of 2-(2,4-dimethoxyphenyl)-3-methyl-4-oxo-4-(4-(2-propenyloxy)phenyl)-butanenitrile (50 mg, 0.14 mmol) in anhydrous toluene (10 ml) was added sodium hydride (50 mg 60% dispersion, 12.5 mmol) and refluxed for 2 h. Water (5 ml) was added, the mixture was extracted with ethyl acetate (2×10 ml) and the combined organic phases were concentrated in vacuo. The residue was purified by column chromatography to give 43.1 mg (91.0%) of 3-(2,4-dimethoxyphenyl)-2-methyl-1-(4-(2-propenyloxy)phenyl)prop-2-enone-1 as colourless crystals, mp: 60.3-61.7° C. ¹H NMR (CDCl₃) δ7.79 (AA′ part of an AA′MM′ system, H2′, H6′), 7.38 (d, J 8.5 Hz, H6), 7.31 (bs, Hβ), 6.95 (MM′ part of an AA′MM′ system, H3′, H5′), 6.54 (dd, J 8.5. 2.3 Hz, H5), 6.46 (d, J 2.3 Hz, H3), 6.03 (ddq, J 17.3, 10.5, 5.2 Hz, H2″), 5.42 (dd, J 17.3, 1.3 Hz, H3″trans), 5.32 (dd, J 10.5, 1.3 Hz, H3″cis), 4.58 (bd, J 5.2 Hz, H1″), 3.84 (s, OCH₃), 3.77 (s, OCH₃), 2.18 (d, J 1.4 Hz, α-CH₃). NOE: α-CH₃—H2′H6′; α-CH₃—H6. ¹³C NMR (CDCl₃) δ198.1 (C═O), 161.5* (C4′), 161.4* (C4), 158.9* (C2), 136.7 (C2″), 134.7 (Cα), 132.7 (Cα), 132.7 (C2′, C6′), 132.0 (Cβ), 131.4 (C1′), 130.9 (C6), 118.1 (C3″), 118.0 (C1), 114.0 (C3′, C5′), 104.1 (C5), 98.2 (C3), 68.8 (C1″), 55.4 (OCH₃), 14.8 (α-CH₃).

### 8) Preparation of 3-(2,4-dimethoxyphenyl)-2-propyl-1-(4-(2-propenyloxy)phenyl)prop-2-en-1-one

To a solution of 2(2,4-dimethoxyphenyl)-3-propyl-4-oxo-4-(4-(2-propenyloxy)phenyl)butanenitrile (197 mg, 0.5 mmol) in anhydrous toluene (20 ml) was added sodium hydride (200 mg, 60% dispersion, 15 mmol) and refluxed for 6h. Water (10 ml) was added, the mixture was extracted with ethyl acetate (2×20 ml) and the combined organic phases was concentrated in vacuo. The residue was purified by column chromatography to give 135.8 mg (74.1%) of 3-(2,4-dimethoxyphenyl)-2-propyl-1-(4-(2-propenyloxy)phenyl)prop-2-enone-1 as a yellow oil. ¹H NMR (CDCl₃) δ7.88 (AA′ part of an AA′MM′ system, H2′, H6′), 7.33 (d, J 8.4 Hz, H6), 7.16 (bs, Hβ), 6.94 (MM′ part of an AA′MM′ system, H3′, H5′), 6.56 (dd, J 8.4. 2.4 Hz, H5), 6.47 (d, J 2.4 Hz, H3), 6.03 (ddq, J 17.3, 10.5, 5.2 Hz, H2″), 5.42 (dd, J 17.3, 1.3 Hz, H3″trans), 5.32 (dd, J 10.5, 1.3 Hz, H3″cis), 4.58 (bd, J 5.2 Hz, H1″), 3.85 (s, OCH₃), 3.72 (s, OCH₃), 2.65 (bt, J≈8 Hz, Hα1), 1.55 (6.tet., J≈8 Hz, Hα2), 0.94 (t, J≈8 Hz, Hα3). In the NOESY spectrum correlations between the protons in the propyl group and H6 were found. ¹³C NMR (CDCl₃) δ198.1 (C═O), 161.5* (C440 ), 161.1* (C4), 158.5* (C2), 140.1 (Cα), 135.0 (C2″), 132.5 (Cβ), 132.0 (C2′, C6′), 131.4 (C1′), 130.0 (C6), 117.9 (C3″), 117.6 (C1), 113.8 (C3″, C5″), 104.0 (C5), 98.1 (C3), 68.7 (C1″), 55.3 (OCH₃), 55.2 (OCH₃), 30.1 (Cα1), 21.9 (Cα2), 14,2 (Cα3). HRMS (FAB+): 367.1949 (calc. C₂₃H₂₇O₄: 367.1909, [M+H]).

### 9) Preparation of 1-(4-hydroxyphenyl)-3-(5-(1,1-dimethylpropenyl)-4-hydroxy-2-methoxyphenyl)propan-1-one

Trifluoroacetic acid (770 μl, 10.0 mmol) was slowly added to a stirred suspension of Licochalcone A (0.507 g, 1.5 mmol) in dichloromethane (5 ml) and triethylsilane (240 μl, 1.5 mmol). Water was added after stirring for 2 h. The aqueous phase was extracted with dichloromethane (2×10 ml) and the combined organic phases were concentrated in vacuo and the residue purified by column chromatography to give 0.359 g (70.4%) of 1-(4-hydroxyphenyl)-3-(5-(1,1-dimethylpropenyl)-4-hydroxy-2-methoxyphenyl)propan-1-one as colourless crystals, mp: 153.5-154.4° C. ¹H NMR (CDCl₃) δ7.91 (AA′ part of an AA′MM′ system, H2′, H6′), 7.00 (s, H6), 6.90 (MM′ part of an AA′MM′ system, H3′, H5′), 6.40 (s, H3), 6.24 (dd J 10.5, 17.7 Hz, H2″), 5.33 (dd, J 17.7, 1.0 Hz, H3″trans), 5.28 (dd, J 10.5, 1.0 Hz, H3″cis), 3.76 (s, OCH₃), 3.18 (bt, J 7.0 Hz, Hα), 2.96 (bt, J 7.0 Hz, Hβ), 1.38 (s, CH₃). ¹³C NMR (CDCl₃) δ200.6 (C═O), 160.8* (C4′), 157.2* (C2), 154.0* (C4), 148.3 (C2″), 130.9 (C2′, C6′), 129.8 (C1′), 127.7 (C6), 123.4 (C5), 121.0 (C1), 115.4 (C3′, C5′), 113.3 (C3″), 100.7 (C3), 55.3 (OCH₃), 39.7 (C1″), 39.2 (Cα), 27.0 (CH₃), 26.1 (Cβ). Anal. (C₂₁H₂₄O₄) C, H.

### 10) Preparation of 2-hydroxy-4-(2-tetrahydropyranyl)oxybenzaldehyde (procedure A)

A solution of 2,4-dihydroxybenzaldehyde (2.76 g, 20 mmol), pyridinium-toluenesulfonate (100 mg, 0.4 mmol) and 3,4-dihydro-2H-pyran (2.7 ml, 30 mmol) in methylene chloride (30 ml) was stirred for 4 h at room temperature. The solution was washed with 1M sodium carbonate (20 ml) and concentrated in vacuo to give 4.3 g of an brown oil, which according to the ¹H NMR spectrum consisted of almost pure 2-hydroxy-4-(2-tetrahydropyranyl)oxybenzaldehyde. ¹H NMR (CDCl₃) δ 11.4 (s, OH), 9.72 (s, CHO), 7.43 (d, J 8.4 Hz, H6), 6.66 (dd, J 8.4, 2.5 Hz, H5), 6.62 (d, J 2.5 Hz, H3), 5.50 (t, J 3 Hz, H2′), 3.83 (bt, J 9.2 Hz, H6a′), 3.65 (bt, J 5.4 Hz, H6e′), 2.0-1.5 (m, H340 -540 ).

### 11) Preparation of 2-methoxy-4-(2-tetrahydropyranyl)oxybenzaldehyde (procedure B)

The crude product obtained from procedure A (4.3 g), sodium hydroxide (3.2 g) and iodomethane (2.5 ml, 40 mmol) were suspended in dimethyl sulfoxide (15 ml), stirred for 60 min at room temperature and added water (150 ml). The mixture was extracted with methylene chloride (4×150 ml) and the combined organic phase were washed with water (3×50 ml) and concentrated in vacuo to give 4.5 g of a brown oil, which according to the ¹H NMR spectrum consisted of almost pure 2-methoxy-4-(2-tetrahydropyranyl)oxybenzaldehyde. ¹H NMR (CDCl₃) δ10.3 (s, CHO), 7.79 (d, J 8.6 Hz, H6), 6.71 (dd, J 8.6, 2.2 Hz, H5), 6.63 (d, J 2.2 Hz, H3), 5.54 (t, J 3 Hz, H2′), 3.90 (s, OCH₃), 3.83 (bt, J 9.2, H6a′), 3.67 (bt, J 5.4 Hz, H6e′), 2.0-1.5 (m, H3′-5′).

### 12) Preparation of 2-methoxy-4,4′-dihydroxychalcone/Echinatin (procedure C)

A solution of the crude product obtained from procedure B (1.18 g), 4-(2-tetrahydropyranyl)oxyacetophenone (S. Sogawa et al. J. Med. Chem. 1993, 36, 3904-3909) (1.10 g, 5 mmol) and sodium hydroxide (50 mg) in anhydrous ethanol (10 ml) was stirred for 18h at room temperature. The solution was added 4M hydrochloric acid (2 ml), stirred for additional 15 min and added water (40 ml). The mixture was extracted with ethyl acetate (4×50 ml) and the combined organic phases were concentrated in vacuo to give 2-methoxy-4,4′-dihydroxychalcone/Echinatin (1.2 g, 84% overall yield based on 2,4-dihydroxybenzaldehyde) as yellow crystals, mp: 209-212° C. The ¹H NMR and ¹³C NMR of 2-methoxy-4,4′-dihydroxychalcone were consistent with those of Echinatin (K. Kajiyama et al. Glycyrrhiza Inflata. Phytochemistry, 1992, 31, 3229-3232).

### 13) Preparation of 2,4-dihydroxy-5-propyl-benzaldehyde (procedure D)

Phosphorus oxychloride (0.800 ml, 8.6 mmol) was added dropwise with stirring to dimethylformamide (2.6 ml, 33.6 mmol), the temperature being kept at 10-20° C. This reagent was slowly added to a solution of 2,4-dihydroxy-5-propyltoluene (C. M. Suter et al. J. Am. Chem. Soc. 1939, 61, 232-236) (0.59 g, 3.9 mmol) in dimethylformamide (2.6 ml), the temperature being kept at 20-30° C. After 30 min. the mixture was poured in 2M NaOH (20 ml). The organic phase was extracted with 2M NaOH (2×10 ml). The combined aqueous phases were neutralised with 4M hydrochloric acid and extracted with ethyl acetate (3×20 ml). The combined organic phases were concentrated in vacuo and the residue purified by column chromatography to give 0.44 g (63%) of 2,4-dihydroxy-5-propyl-benzaldehyde as grey crystals, mp: 92.8-93.6° C. ¹H NMR (CD₃OD) δ 9.65 (s, —CHO), 7.28 (s, H6), 6.28 (s, H3), 4.94 (b, OH), 2.48 (t, J 7.3 Hz, H1′), 1.56 (six., J 7.3 Hz, H2′), 0.91 (t, J 7.3 Hz, H3′). ¹³C NMR (CD₃OD) δ 193.5 (C═O), 163.4* (C4), 161.8* (C2), 134.0 (C6), 121.8 (C5), 113.7 (C1), 100.7 (C3), 30.4 (C1′), 21.9 (C2′), 12.3 (C3′).

### 14) Preparation of 2-hydroxy-4-(2-tetrahydropranyl)oxy-5-propylbenzaldehyde

2-hydroxy-4-(2-tetrahydropyranyl)oxy-5-propylbenzaldehyde (1.25 g) as an almost pure yellow oil, was synthesised according to procedure A using 2,4-dihydroxy-5-propyl-benzaldehyde (0.45 g, 2.5 mmol) as starting material. ¹H NMR (CDCl₃) δ 11.30 (s, —OH), 9.68 (s, —CHO), 7.21 (s, H6), 6.68 (s, H3), 5.49 (bt, H2′), 3.85(b, H6a′), 3.60 (b, H6e′), 2.55 (t, J 7 Hz, H1″), 1.58 (six, J 7 Hz, H2″), 2.1-1,4 (m, H3′-5′), 0.91 (t, J 7 Hz, H3″).

### 15) Preparation of 2-methoxy-4-(2-tetrahydropyranyl)oxy-5-propylbenzaldehyde

2-methoxy-4-(2-tetrahydropyranyl)oxy-5-propylbenzaldehyde (1.26 g) as a relatively pure brown oil, was synthesised according to procedure B using crude 2-hydroxy-4-(2-tetrahydropyranyl)oxy-5-propylbenzaldehyde (1.25 g) as starting material. ¹H NMR (CDCl₃) δ 10.28 (s, CHO), 7.58 (s, H6), 6.73 (s, H3), 5.55 (b, H2′), 3.81 (s, OCH₃), 3.8 (b, H6a′), 3.6 (b, H6e′), 2.55 (t, J 7 Hs, H1″), 1.61 (m, H2″), 2.1-1,4 (m, H3′-5′), 0.91 (t, J 7 Hz, H3″).

### 16) Preparation of 2-methoxy-4,4′-dihydroxy-5-propylchalcone

2-methoxy-4,4′-dihydroxy-5-propylchalcone was synthesised according to procedure C using crude 2-methoxy-4-(2-tetrahydropyranyl)oxy-5-propylbenzaldehyde (1.26 g) and 4-(2-tetrahydropyranyl)oxyacetophenone (S. Sogawa et al. J. Med. Chem. 1993, 36, 3904-3909) (0.48 g, 2.2 mmol) as starting materials: Orange-brown crystals (0.32 g, 41% overall yield based on 2,4-dihydroxy-5-propyl-benzaldehyde), mp: 126.5° C.-128.0° C. ¹H NMR ((CD₃)₂CO) δ8.11 (d, J 15.5 Hz, Hβ), 8.06 (AA′ part of an AA′MM′ system, H2′, H6′), 7.73 (d, J 15.5 Hz, Hα), 7.63 (s, H6), 6.97 (MM′ part of an AA′MM′ system, H3′, H5′), 6.59 (s, H3), 3.85 (s, OCH₃), 2.58 (t, J 7.3 Hz, H1″), 1.64 (six., J 7.3 Hz, H2″), 0.95 (t, J 7.3 Hz, H3″). ¹³C NMR ((CD₃)₂CO) δ 188.4 (CHO), 162.3*(C4′), 159.7*(C2), 159.5*(C4), 139.5 (Cβ), 131.7 (C1′), 131.6 (C2′, C6′), 131.3 (C6), 122.4 (C5), 119.1 (Cα), 116.3 (C1), 116.0 (C3′, C5′), 99.7 (C3), 55.9 (OCH₃), 32.2 (C1″), 23.9 (C2″), 14.3 (C3″). Anal. Calcd. (C₁₉H₂₀O₄, ½H₂O): C, 71.01; H, 6.59. Found: C, 71.18; H, 6.66.

### 17) Preparation of 2,4-dihydroxy-6-hexylbenzaldehyde

2,4-dihydroxy-5-hexylbenzaldehyde was synthesised according to procedure D using 2,4-dihydroxy-5-hexyltoluene (1.39 g, 7.2 mmol) as starting material: Yellow-white crystals (0.74 g, 46%), mp: 108.2° C.-109.5° C. ¹H NMR (CD₃OD) δ 9.65 (s, OH, CHO), 7.30 (s, H6), 6.29 (s, H3), 4.94 (b, OH), 2.50 (t, J 7.3 Hz, H1′), 1.56 (m, H2′), 1.30 (m, H3′-H5′), 0.91 (t, J 7.3, H6′). ¹³C NMR (CD₃OD) δ 193.5 (C═O), 163.3*(C4), 161.8*(C2), 133.8 (C6), 122.0 (C5), 113.7 (C1), 100.7 (C3), 30.9 (C1′), 28.8 (C2′), 28.2 (C3′, C4′), 21.7 (C5′), 12.5 (C6′).

### 18) Preparation of 2-hydroxy-4-(2-tetrahydropyranyl)oxy-5-hexylbenzaldehyde

2-hydroxy-4-(2-tetrahydropyranyl)oxy-5-hexylbenzaldehyde (0.62 g) as a relatively pure yellow oil, was synthesised according to procedure A using 2,4-dihydroxy-5-hexylbenzaldehyde (0.23 g, 1.0 mmol) as starting material. ¹H NMR (CDCl₃) δ 11.28 (s, —OH), 9.69 (s, —CHO), 7.23 (s, H6), 6.66 (s, H3), 5.50 (bt, H2′), 3.9(b, H6a′), 3.6 (b, H6e′), 2.55 (t, J7 Hz, H1″), 2.1-1,4 (m, H3′-5′, H2″-H5″), 0.90 (t, J 7 Hz, H6″).

### 19) Preparation of 2-methoxy-4-(2-tetrahydropyranyl)oxy-5-hexylbenzaldehyde

2-methoxy-4-(2-tetrahydropyranyl)oxy-5-hexylbenzaldehyde (0.61 g) as a relatively pure brown oil, was synthesised according to procedure B using crude 2-hydroxy-4(2-tetrahydropyranyl)oxy-5-hexylbenzaldehyde (0.62 g) as starting material. ¹H NMR (CDCl₃) δ 10.28 (s, CHO), 7.58 (s, H6), 6.75 (s, H3), 5.58 (b, H2′), 3.88 (s, OCH₃), 3.80 (b, H6a′), 3.61 (b, H6e′), 2.60 (m, H1″), 2.1-1,4 (m, H3′-H5′, H2″-H5″), 0.90 (bt, J 7.1 Hz, H6″).

### 20) Preparation of 2-methoxy-4,4′-dihydroxy-5-hexylchalcone

2-methoxy-4,4′-dihydroxy-5-hexylchalcone was synthesised according to procedure C using crude 2-methoxy-4-(2-tetrahydropyranyl)oxy-5-hexylbenzaldehyde (0.61 g) and 4-(2-tetrahydropyranyl)oxyacetophenone (S. Sogawa et al. J. Med. Chem. 1993, 36, 3904-3909) (0.10 g, 0.45 mmol) as starting materials: An orange amorphous powder (0.19 g, 51% overall yield based on 2,4-dihydroxy-5-hexylbenzaldehyde). ¹H NMR ((CD₃)₂CO) δ 8.10 (d, J 15.6 Hz, Hβ), 8.05 (AA′ part of an AA′MM′ system, H2′, H6′), 7.73 (d, J 15.6 Hz, Hα), 7.64 (s, H6), 6.96 (MM′ part of an AA′MM′ system, H3′, H5′), 6.58 (s, H3), 3.85 (s, OCH₃), 2.60 (t, J 7 Hz, H1″), 1.33 (m, H2″), 1.1 (m, H3″-H5″), 0.88 (bt, J 7 Hz, H6″). ¹³C NMR ((CD₃)₂CO) δ 188.5 (CHO), 162.3*(C4′), 159.6*(C2), 159.4*(C4), 139.6 (Cβ), 131.7 (C1′), 131.6 (C2′, C6′), 131.3 (C6), 122.6 (C5), 119.0 (Cα), 116.2 (C1), 116.0 (C3′, C5′), 99.7 (C3), 55.9 (OCH₃), 32.5 (C1″), 30.9 (C2″), 30.1 (C3″), 29.9 (C4″), 23.3 (C5″), 14.4 (C6″). Anal. Calcd. (C₂₂H₂₆O₄): C, 74.55; H, 7.39. Found: C, 74.63; H, 7.53.

### 21) Preparation of 3-methyl-2,4-dihydroxybenzaldehyde

3-methyl-2,4-dihydroxybenzaldehyde was synthesised according to procedure D using 3-methyl-2,4-dihydroxytoluene (1.48 g, 11.9 mmol) as starting material: White crystals (1.46 g, 80%), mp: 149.6° C.-150.3° C. ¹H NMR (CD₃OD) δ9.61 (s, OH, CHO), 7.30 (d J 8,6 Hz, H6), 6.47 (d J 8,6 Hz, H5), 4.94 (b, OH), 2.04 (s, CH₃) ¹³C NMR (CD₃OD) δ 196.1 (C═O), 164.9*(C4), 163.2*(C2), 134.2 (C6), 115.7 (C1), 112.1 (C3), 108.9 (C5), 7.3 (CH₃).

### 22) Preparation of 2-hydroxy -3-methyl-4-(2-tetrahydropyranyl)oxybenzaldehyde

2-hydroxy-3-methyl-4-(2-tetrahydropyranyl)oxybenzaldehyde (0.60 g) as a relatively pure yellow oil, was synthesised according to procedure A using 3-methyl-2,4-dihydroxybenzaldehyde (0.38 g, 2.5 mmol) as starting material. ¹H NMR (CDCl₃) δ 9.68 (s, OH, CHO), 7,31 (d, J 9.1 Hz, H6), 6,78 (d, J 9.1 Hz, H5), 5.58 (bt, H2′), 3.9(b, H6a′), 3.6 (b, H6e′), 2.18 (s, CH₃), 2,1-1,4 (m, H3′-5′).

### 23) Preparation of 2-methoxy-3-methyl-4-(2-tetrahydropyranyl)oxybenzaldehyde

2-methoxy-3-methyl-4-(2-tetrahydropyrany)oxybenzaldehyde (0.69 g) as a relatively pure brown oil, was synthesised according to procedure B using crude 2-hydroxy-3-methyl-4-(2-tetrahydropyranyl)oxybenzaldehyde (0.60 g) as starting material. ¹H NMR (CDCl₃) δ 10.2 (s, CHO), 7.70 (d, J 8.9 Hz, H6), 6.98 (d, J 8.9 Hz, H5), 5.55 (bt, H2′), 3.85 (s, OCH₃), 3.82(b, H6a′), 3.64 (b, H6e′), 2.20 (s, CH₃), 2.1-1,4 (m, H3′-5′).

### 24) Preparation of 2-methoxy-3-methyl-4,4′-dihydroxychalcone

2-methoxy-3-methyl-4,4′-dihydroxychalcone was synthesised according to procedure C using crude 2-methoxy-3-methyl-4-(2-tetrahydropyranyl)oxybenzaldehyde (0.69 g) and 4-(2-tetrahydropyranyl)oxyacetophenone (S. Sogawa et al. J. Med. Chem. 1993, 36, 3904-3909) (0.38 g, 1.7 mmol) as starting materials: Yellow crystals (0.38 g, 54% overall yield based on 3-methyl-2,4-dihydroxybenzaldehyde), mp: 185.4° C.-186.7° C. ¹H NMR ((CD₃)₂CO) δ 8.07 (AA′ part of an AA′MM′ system, H2′, H6′), 8.03 (d, J 15.6 Hz, Hβ), 7.71 (d, J 15.6 Hz, Hα), 7.67 (d, J 8.5 Hz, H6), 6.98 (MM′ part of an AA′MM′ system, H3′, H5′), 6.76 (d, J 8.5 Hz, H5), 3.77 (s, OCH₃), 2.18 (s, —CH₃). ¹³C NMR ((CD₃)₂CO) δ 188.6 (C═O), 162.3*(C4), 160.6*(C4′), 159.4*(C2), 139.3 (Cβ), 131.7 (C2′, C6′), 131.6 (C1′), 127.0 (C6), 120.9 (C1), 120.3 (Cα), 118.9 (C3), 116.1 (C3′, C5′), 112.4 (C5), 61.8 (OCH₃), 9.1 (CH₃). Anal. Calcd. (C₁₇H₁₆O₄): C, 71.82; H, 5.67. Found: C, 71.65; H, 5.70.

### 25) Preparation of 2,4-dihydroxy-6-methylbenzaldehyde

2,4-dihydroxy-6-methylbenzaldehyde was synthesised according to procedure D using 2,4-dihydroxy-6-methyltoluene (2.9 g, 20.8 mmol) as starting material: Orange crystals (65%), mp: 173.7° C.-177.3° C. ¹H NMR (CD₃OD) δ 10.2 (s, OH, CHO), 6.18 (d J 2 Hz, H5), 6.08 (d, J 2 Hz, H3), 4.95 (s, OH) 2.47 (s, CH₃). ¹³C NMR (CD₃OD) δ 192.3 (C═O), 165.8*(C4), 165.2*(C2), 144.3 (C6), 112.0 (C1), 109.9 (C5), 99.5 (C3), 16.4 (CH₃).

### 26) Preparation of 2-hydroxy-4-(2-tetrahydropyranyl)oxy-6-methylbenzaldehyde

2-hydroxy-4-(2-tetrahydropyranyl)oxy-6-methylbenzaldehyde (1.80 g) as a relatively pure yellow oil, was synthesised according to procedure A using 2,4-dihydroxy-6-methylbenzaldehyde (0.76 g, 5.0 mmol) as starting material. ¹H NMR (CDCl₃) δ 10.09 (s, OH, CHO), 6.45 (d, J 2 Hz, H5), 6.37 (d, J2 Hz, H3), 5.49 (bt, H2′), 3.9(b, H6a′), 3.6 (b, H6e′), 2.52 (s, CH₃), 2.1-1,4 (m, H3′-5′).

### 27) Preparation of 2-methoxy-4-(2-tetrahydrofuranyl)oxy-6-methylbenzaldehyde

2-methoxy-4-(2-tetrahydropyranyl)oxy-6-methylbenzaldehyde (1.74 g) as a relatively pure brown oil, was synthesised according to procedure B using crude 2-hydroxy-4-(2-tetrahydropyranyl)oxy-6-methylbenzaldehyde (1.80 g) as starting material.

¹H NMR (CDCl₃) δ 10.5 (s, CHO), 6.48 (b, H3, H5), 5.55 (bt, H2′), 3.85 (s, OCH₃), 3.8(b, H6a′), 3.6 (b, H6e′), 2.55 (s, CH₃), 2.1-1,4 (m, H3′-5′).

### 28) Preparation of 2-methoxy-4,4′-dihydroxy-6-methylchalcone

2-methoxy-4,4′-dihydroxy-6-methylchalcone was synthesised according to procedure C using crude 2-methoxy-4-(2-tetrahydropyranyl)oxy-6-methylbenzaldehyde (1.74 g) and 4-(2-tetrahydropyranyl)oxyacetophenone (S. Sogawa et al. J. Med. Chem. 1993, 36, 3904-3909) (0.80 g, 3.6 mmol) as starting materials: Yellow crystals (0.73 g, 51% overall yield based on 2,4-dihydroxy-6-methylbenzaldehyde), mp: 197.0° C.-197.9° C. ¹H NMR ((CD₃)₂CO) δ 8.02 (d J 15.7 Hz, Hβ), 8.00 (AA′ part of an AA′MM′ system, H2′, H6′), 7.83 (d, J 15.7 Hz, Hα), 6.97 (MM′ part of an AA′MM′ system, H3′, H5′), 6.46 (d, J 2 Hz, H5), 6.40 (d, J 2 Hz, H3), 3.93 (s, OCH₃), 2.43 (s, CH₃). ¹³C NMR ((CD₃)₂CO) δ188.8 (CHO), 162.7*(C4), 162.1*(C4′), 160.5*(C2), 142.8 (C6), 138.0 (Cβ), 131.8 (C1′), 131.5 (C2′, C6′), 122.9 (Cα), 116.1 (C3′, C5′), 115.5 (C1), 111.2 (C5), 98.1 (C3), 55.9 (OCH₃), 21.5 (—CH₃). Anal. Calcd. (C₁₇H₁₆O₄, ½H₂O): C, 69.61; H, 5.84. Found: C, 69.74; H, 6.04.

### 29) Preparation of 2-hydroxy-3-(3-methyl-2-butenyl)-4-(2-tetrahydropyranyl)oxybenzaldehyde

2-hydroxy-3-(3-methyl-2-butenyl)-4-(2-tetrahydropyranyl)oxybenzaldehyde (0.32 g) as a almost pure brown oil, was synthesised according to procedure A using 2,4-dihydroxy-3-(3-methyl-2-butenyl)benzaldehyde (K.-H. Glussenkamp et al. J. Org. Chem. 1986, 51, 4481-4483) (0.19 g, 0.94 mmol) as starting material. ¹H NMR (CDCl₃) δ 11.2 (s, OH), 9.71 (s, CHO), 7.34 (d, J 8.7 Hz, H6), 6.80 (d, J 8.7 Hz, H5), 5.59 (bt, J 3 Hz, H2′), 5.25 (t.sept., J 7.5 1.3, H2″), 3.83 (td, J 10.1 3.2, H6a′), 3.66 (m, H6e′), 3.41 (bt, J 3 Hz, H1″), 2.1-1.6 (m, H3′-5′), 1.80 (s, H3″), 1.69 (s, H4″).

### 30) Preparation of 2-methoxy-3-(3-methyl-2-butenyl)-4-(2-tetrahydropyranyl)oxybenzaldehyde

2-methoxy-3-(3-methyl-2-butenyl)-4-(2-tetrahydropyranyl)oxybenzaldehyde (0.42 g) as a relatively pure brown oil, was synthesised according to procedure B using crude 2-hydroxy-3-(3-methyl-2-butenyl)-4-(2-tetrahydropyranyl)oxybenzaldehyde (0.32 g) as starting material. ¹H NMR (CDCl₃) δ 10.2 (s, CHO), 7.70 (d, J 8.6 Hz, H6), 7.00 (d, J 8.6 Hz, H5), 5.56 (t, J 3 Hz, H2′), 5.21 (m, H2″), 3.82 (s, OCH₃), 3.7 (m, H6′), 3.41 (bt, J 3 Hz, H1″), 2.0-1.5 (m, H3′-5′), 1.80 (s, H3″), 1,69 (s, H4″).

### 31) Preparation of 2-methoxy-3-(3-methyl-2-butenyl)-4,4′-dihydroxychalcone/Licochalcone C

2-methoxy-3-(3-methyl-2-butenyl)-4,4′-dihydroxychalcone as an yellow amorphous powder (0.16 g, 50% overall yield based on 2,4-dihydroxy-3-(3-methyl-2-butenyl)benzaldehyde), was synthesised according to procedure C using crude 2-methoxy-3-(3-methyl-2-butenyl)-4-(2-tetrahydropyranyl)oxybenzaldehyde (0.32 g) and 4-(2-tetrahydropyranyl)-oxyacetophenone (S. Sogawa et al. J. Med. Chem. 1993, 36, 3904-3909) (0.12 g, 0.54 mmol) as starting materials. The ¹H NMR and ¹³C NMR of 2-methoxy-3-(3-methyl-2-butenyl)-4,4′-dihydroxychalcone were consistent with those of LicC (K. Kajiyama et al. Glycyrrhiza Inflata. Phytochemistry, 1992, 31, 3229-3232).

### 32) Preparation of 2-methyl-3,5-dimethoxy-N,N-diethylbenzamide

To a cold solution (−78° C.) of 3,5-dimethoxy-N,N-diethylbenzamide (K.-H. Glussenkamp et al. J. Org. Chem. 1986, 51, 4481-4483) (0.9 g, 3.9 mmol) in anhydrous tetrahydrofuran (22.5 ml) was slowly (3 min) added secbutyllithium (5.8 ml of 1.3 M solution, 7.5 mmol). After stirring for 15 min, iodomethane (2.04 ml, 30 mmol) was added and the mixture was stirred for 10 min at −78° C. followed by slowly heating to room temperature. Water (30 ml) was added and the mixture was extracted with ethyl acetate (2×30 ml). The combined organic phases were concentrated in vacuo and the residue purified by column chromatography to give 2-methyl-3,5-dimethoxy-N,N-diethylbenzamide (0.71 g, 75%) as a yellow oil. ¹H NMR (CDCl₃) δ 6.50 (d, J 2.4 Hz, H6), 6.30 (d, J 2.4 Hz, H4), 3.79 (s, OCH₃), 3.75 (s, OCH₃), 3.64 (pent., J 7.1 Hz, H1″a), 3.35 (pent., J 7.1 Hz, H1″b), 3.10 (q, J 7.1 Hz, H1′a), 3.07 (q, J 7.1 Hz, H1′b), 1.97 (s, CH₃), 1.19 (t, J 7.1 Hz, H2″), 0.99 (t, J 7.1 Hz, H2′). ¹³C NMR (CDCl₃) δ 169.4 (C═O), 158.7* (C3), 158.6* (C5), 139.0 (C1), 113.9 (C2), 101.1 (C6), 97.6 (C4), 55.0 (OCH₃), 54.8 (OCH₃), 42.1* (N—CH₂—), 38.1* (N—CH₂—), 13.1 (—CH₂CH₃), 8.9 (—CH₂CH₃), 10.8 (—CH₃).

### 33) Preparation of 2-methyl-3,5-dimethoxybenzaldehyde

An ice cooled solution of diisobutylaluminium hydride (42 ml of 1M solution, 42 mmol) was slowly added n-butylithium (17 ml of 2.5 M solution, 42.5 mmol). The solution was stirred for 30 min and added to a solution of 2-methyl-3,5-dimethoxy-N,N-diethylbenzamide (1.0 g, 4 mmol) in anhydrous tetrahydrofuran (42 ml). After 22 h water (50 ml) was added and the mixture was extracted with ethyl acetate (2×50 ml). The combined organic phases were concentrated in vacuo and the residue purified by column chromatography to give 2-methyl-3,5-dimethoxybenzaldehyde (0.59 g, 82%) as brown crystals, mp: 63.4-65.1° C. ¹H NMR(CDCl₃) δ 10.4 (s, CHO), 6.95 (d, J 2.5 Hz, H6), 6.68 (d, J 2.5 Hz, H4), 3.84 (s, OCH₃), 2.36 (s, CH₃).

### 34) Preparation of 2-methyl-3,5-dimethoxy-4y-(2-propenyloxy)chalcone (procedure E)

To a solution of 4-(2-propenyloxy)acetophenone (R. T. Arnold et al. J. Am. Chem. Soc. 1942, 64, 1315-1317) (0.20 g, 1.1 mmol) and 2-methyl-3,5-dimethoxybenzaldehyde (0.20 g, 1.1 mmol) in anhydrous ethanol (1 ml) was added sodium hydroxide (6 mg). The solution was stirred for 16 h, neutralised with 4M hydrochloric acid, added water (2 ml) and extracted with ethyl acetate (3×2 ml). The combined organic phases were concentrated in vacuo and the residue purified by column chromatography to give 2-methyl-3,5-dimethoxy-4′-(2-propenyloxy)chalcone (0.14 g, 81%) as yellow crystals mp: 123.1-124.5° C. ¹H NMR (CDCl₃) δ 8.10 (d, J 15.5 Hz, Hβ), 8.04 (AA′ part of an AA′MM′ system, H2′, H6′), 7.40 (d, J 15.5 Hz, Hα), 7.00 (MM′ part of a AA′MM′ system, H3′, H5′), 6.78 (d, J 2.3 Hz, H6), 6.51 (d, J 2.3 Hz, H4), 6.03 (ddq, J 17.3, 10.5, 5.2 Hz, H2″), 5.42 (dd, J 17.3, 1.3 Hz, H3″trans), 5.32 (dd, J 10.5, 1.3 Hz, H3″cis), 4.58 (bd, J 5.2 Hz, H1″), 3.86 (s OCH₃), 3.83 (s, OCH₃), 2.26 (s, CH₃). ¹³C NMR (CDCl₃) δ186.4 (C═O), 162.4 (C4′), 157.3 (C3, C5), 143.1 (Cβ), 135.2 (C1), 132.5 (C2″), 131.1 (C1′), 129.8 (C2′, C6′), 122.3 (Cα), 118.0 (C3″), 114.3 (C3′, C5′), 114.0 (C2), 104.1 (C6), 100.2 (C4), 69.1 (C1″), 55.7 (OCH₃), 10.2 (CH₃). Anal. Calcd. (C₂₁H₂₂O₄): C, 74.54; H, 6.55. Found: C, 74.61; H, 6.34.

### 35) Preparation of 4-methyl-3,5-dimethoxy-4′-(2-propenyloxy)chalcone

4-methyl-3,5-dimethoxy-4′-(2-propenyloxy)chalcone was synthesised according to procedure E using 4-methyl-3,5-dimethoxybenzaldehyde (U. Azzena et al. Synthesis, 1990, 313-314) (0.52 g, 2.3 mmol) and 4-(2-propenyloxy)acetophenone R. T. Arnold et al. J. Am. Chem. Soc. 1942, 64, 1315-1317) (0.40 g, 2.3 mmol) as starting materials: Yellow crystals (0.44 g, 57%), mp: 143.1-144.2° C. ¹H NMR (CDCl₃): δ 8.05 (AA′ part of an AA′MM′ system, H2′, H6′), 7.78 (d, J 14.4 Hz, Hβ), 7.48 (d, J 14.4 Hz, Hα), 7.00 (MM′ part of a AA′MM′ system, H3′, H5′), 6.80 (s, H2, H6), 6.03 (ddq, J 17.3, 10.5, 5.2 Hz, H2″), 5.42 (dd, J 17.3, 1.3 Hz, H3″trans), 5.32 (dd, J 10.5, 1.3 Hz, H3″cis), 4.58 (bd, J 5.2 Hz, H1″), 3.88 (s, OCH₃), 2.13 (s, CH₃).

¹³C NMR (CDCl₃): δ 188.9 (C═O), 162.2 (C4′), 158.5 (C3, C5), 144.8(Cβ), 133.3 (C1), 132.5 (C2″), 131.3 (C1′), 130.8 (C2′, C6′), 121.0(Cα), 118.2 (C3″), 117.9 (C4), 114.5 (C3′, C5′), 103.6 (C2, C6), 68.9 (C1″), 55.8 (OCH₃), 8.6 (—CH₃). Anal. Calcd. (C₂₁H₂₂O₄): C, 74.54; H, 6.55. Found: C, 74.35; H, 6.64.

### 36) Preparation of 4-butyl-3,5-dimethoxy-4′-(2-propenyloxy)chalcone

4-butyl-3,5-dimethoxy-4′-(2-propenyloxy)chalcone was synthesised according to procedure E using 4-butyl-3,5-dimethoxybenzaldehyde (U. Azzena et al. Synthesis, 1990, 313-314) (40 mg, 0.18 mmol) and 4-(2-propenyloxy)acetophenone (R. T. Arnold et al. J. Am. Chem. Soc. 1942, 64, 1315-1317) (32 mg, 0.18 mmol) as starting materials: Yellow crystals (44 mg, 64%), mp: 74-75° C.

¹H NMR (CDCl₃): δ 8.03 (AA′ part of an AA′MM′ system, H2′, H6′), 7.75 (d, J 15.9 Hz, Hβ), 7.45 (d, J 15.9 Hz, Hα), 6.99 (MM′ part of a AA′MM′ system, H3′, H5′), 6.80 (s, H2, H6), 6.03 (ddq, J 17.3, 10.5, 5.2 Hz, H2″), 5.42 (dd, J 17.3, 1.3 Hz, H3″trans), 5.32 (dd, J 10.5, 1.3 Hz, H3″cis), 4.58 (bd, J 5.2, H1″), 3.87 (s, OCH₃), 2.65 (t, J 7.4 Hz, H_{bu}1), 1.40 (m, H_{bu}2, H_{bu}3), 0.92 (t, J 7.4 Hz, H_{bu}4). ¹³C NMR (CDCl₃): δ 188.9 (C═O), 162.3 (C4″), 158.4 (C3, C5), 144.9 (Cβ), 133.4 (C1) 132.5 (C2″), 131.4 (C1′), 130.8 (C2′, C6′), 123.0 (C4), 121.0(Cα), 118.2 (C3″), 114.5 (C3′, C5′), 103.8 (C2, C6), 68.9 (C1″), 55.8 (OCH₃), 31.3 (C_{bu}1), 23.0 (C_{bu}2), 22.9 (C_{bu}3), 14.1 (C_{bu}4). Anal. Calcd. (C₂₄H₂₈O₄): C, 75.76; H, 7.42. Found: C, 75.53; H, 7.41.

### 37) Preparation of 4-hexyl-3,5-dimethoxy-4′-(2-propenyloxy)chalcone

4-hexyl-3,5-dimethoxy-4′-(2-propenyloxy)chalcone was synthesised according to procedure E using 4-hexyl-3,5-dimethoxybenzaldehyde (U. Azzena et al. Synthesis, 1990, 313-314) (0.10 g, 0.4 mmol) and 4-(2-propenyloxy)acetophenone (R. T. Arnold et al. J. Am. Chem. Soc. 1942, 64, 1315-1317) (0.07 g, 0.4 mmol) as starting materials: White crystals (0.16 g, 90%), mp: 77.9-78.2° C. ¹H NMR (CDCl₃): δ 8.03 (AA′ part of an AA′MM′ system, H2′, H6′), 7.77 (d, J 15.4 Hz, Hβ), 7.47 (d, J 15.4 Hz, Hα), 7.00 (MM′ part of a AA′MM′ system, H3′, H5′), 6.80 (s, H2, H6), 6.03 (ddq, J 17.3, 10.5, 5.2 Hz, H2″), 5.42 (dd, J 17.3, 1.3 Hz, H3″trans), 5.32 (dd, J 10.5, 1.3 Hz, H3″cis), 4.58 (bd, J 5.2, H1″), 3.87 (s, OCH₃), 2.65 (t, J 7.4 Hz, Hₕₑₓ1), 1.40 (m, Hₕₑₓ2-Hₕₑₓ5), 0.89 (t, J 7 Hz, Hₕₑₓ6). ¹³C NMR (CDCl₃): δ 188.9 (C═O), 162.3 (C4), 158.4 (C3, C5), 144.9 (Cβ), 133.4 (C1), 132.5 (C2″), 131.3 (C1′), 130.8 (C2′, C6′), 121.0(Cα), 118.2 (C3″), 117.3 (C4), 114.5 (C3′, C5′), 103.8 (C2, C6), 68.9 (C1″), 55.8 (OCH₃), 31.8 (Cₕₑₓ1), 29.5 (Cₕₑₓ2), 29.0 (Cₕₑₓ3), 23.2 (Cₕₑₓ4), 22.7 (Cₕₑₓ5), 14.2 (Cₕₑₓ6). Anal. Calcd. (C₂₆H₃₂O₄): C, 76.44; H, 7.90. Found: C: 76.07, H, 7.84.

### 38) Preparation of 4-phenoxy-4′-cyclohexylchalcone

4-phenoxy-4′-cyclohexylchalcone was synthesised according to procedure E using 4-phenoxybenzaldebyde (0.86 ml, 4.9 mmol) and 4-cyclohexylacetophenone (1.0 g, 4.9 mmol) as starting material: White crystals (1.47 g, 79%), mp: 160.9-161.1° C. ¹H NMR (CDCl₃) δ 8.11 (AA′ part of an AA′MM′ system, H2′, H6′), 7.96 (d, J 15.5 Hz, Hβ), 7.9 (m, H2, H3, H3′, H5′), 7.79 (MM′ part of an AA′MM′ system, H3′, H5′), 7.70 (d, J 15.5 Hz, Hα), 7.41 (m, H3″, H5″), 7.26 (m, H2″, H4″, H6″), 2.61 (b, H1^{c}), 1.9 (m, H2^{c}, H6^{c}), 1.5 (H3^{c}-H5^{c}) (Due to low solubility only ¹H NMR was performed). Anal. Calcd. (C₂₇H₂₆O₂): C, 84.78; H, 6.85. Found: C, 84.74; H, 6.85.

### 39) Preparation of 4-dimethylamino-4′-nitrochalcone (procedure F)

A solution of 4-nitroacetophenone (0.50 g, 3.0 mmol) and 4-dimethylaminobelzaldehyde (0.45 g, 3.0 mmol) in ethanol (3 ml), was saturated with hydrochloric acid. The crystals were filtered off after 24 h to give 4-dimethylamino-4′-nitrochalcone (0.28 g, 32%) as brown crystals, mp: 214.0-214.7° C. ¹H NMR (CDCl₃) δ 8.34 (AA′ part of an AA′MM′ system, H3′, H5′), 8.12 (MM′ part of an AA′MM′ system, H2′, H6′), 7.82 (d, J 15.3 Hz, Hβ), 7.60 (AA′ part of an AA′MM′ system, H2, H6), 7.28 (d, J 15.3 Hz, Hα), 6.71 (MM′ part of an A′AMM′ system, H3, H5), 3.09 (s, NCH₃) (Due to low solubility only ¹H NMR was performed). Anal. Calcd. (C₁₇H₁₆N₂O₃): C, 68.91; H, 5.44; N, 9.45. Found: C, 68.61; H, 5.44; N, 9.13.

### 40) Preparation of 4,4′-dimethylaminochalcone

4,4′-dimethylaminochalcone was synthesised according to procedure E using 4-dimethylaminobenzaldehyde (0.12 g, 0.8 mmol) and 4-dimethylaminoacetophenone (H. Gilman et al. J. Am. Chem. Soc. 1933, 55, 1265-1270) (0.13 g, 0.8 mmol) as starting materials: Orange crystals (0.18 g, 76%), mp: 149.8-150.7° C.¹H NMR (CDCl₃) δ8.00 (AA′ part of an AA′MM′ system, H2′, H6′), 7.77 (d, J 15.4 Hz, Hβ), 7.54 (AA′ part of an AA′MM′ system, H2, H6), 7.40 (d, J 15.4 Hz, Hα), 6.70 (MM′ part of an AA′MM′ system, H3, H5, H3′, H5′), 3.06 (s, NCH₃), 3.02 (s, NCH₃). ¹³C NMR (CDCl₃) δ 188.9 (C═O), 152.3*(C4), 148.4*(C4), 143.4 (Cβ), 130.5*(C3′, C5′), 130.0*(C3, C5), 127.1 (C1′), 123.0 (C1), 117.0 (Cα), 111.9*(C2, C6), 110.8*(C2′, C6′), 40.5 (NCH₃), 40.1 (NCH₃). Anal. Calcd. (C₁₉H₂₂N₂O): C, 77.52; H, 7.53; N, 9.52. Found: C: 77.09, H, 7.64; N, 9.39.

### 41) Preparation of 4-phenoxy-4′-fluorochalcone

4-phenoxy-4′-fluorochalcone was synthesised according to procedure E using 4-phenoxybenzaldehyde (0.29 ml, 1.5 mmol) and 4-fluoroacetophenone (0.18 ml, 1.5 mmol) as starting materials: White crystals (0.42 g, 90%), mp: 115.7-116.0° C. ¹H NMR (CDCl₃) δ 8.26 (m, H2′, H6′), 7.90 (AA′ part of an AA′MM′ system, H2, H6), 7.84 (d, J 15.4 Hz, Hβ), 7.3-7.0 (H_{left}). Anal. Calcd. (C₂₁H₁₅O₂F): C, 79.23; H, 4.75. Found: C: 79.11, H: 4.86. (Due to low solubility only ¹H NMR was performed.)

### 42) Preparation of 4-fluoro-4′-cyclohexylchalcone

4-fluoro-4′-cyclohexylchalcone was synthesised according to procedure E using 4-fluorobenzaldehyde (0.53 ml, 4.9 mmol) and 4-cyclohexylacetophenone (1.0 g, 4.9 mmol) as starting materials: White crystals (1.3 g, 86%), mp: 166.8-167-9° C. ¹H NMR (CDCl₃) δ 7.95 (AA′ part of an AA′MM′ system, H2′, H6′), 7.78 (d, J 15.7 Hz, Hβ), 7.60 (MM′ part of an AA′MM′ system, H3′, H5′), 7.45 (d, J 15.7 Hz, Hβ), 7.38 (dd, J 8.3, 6.5 Hz, H2, H6), 7.05 (dd, J 9.9, 8.3 Hz), 2.55 (b, H1^{c}), 1.87 (m, H2^{c}, H6^{c}), 1.41 (m, H3^{c}-H5^{c}). ¹³C NMR (CDCl₃) δ 189.9 (C═O), 160.4 (C4), 153.4 (C4′), 143.7 (Cβ), 136.1 (C1′), 129.8 (C1), 128.7 (C2′, C6′), 127.0 (C2, C6), 124.1 (Cα), 119.7 (C3′, C5′), 118.4 (C3, C5), 44.7 (C1^{c}), 34.1 (C2^{c}, C6^{c}), 26.7 (C3^{c}, C5^{c}), 26.0 (C4^{c}). Anal. Calcd. (C₂₁H₂₁OF): C, 81.79; H, 6.86. Found: C, 81.61; H, 6.94.

### 43) Preparation of 4,4′-dinitro-chalcone

4,4′-dinitro-chalcone was synthesised according to procedure F using 4-nitrobenzaldehyde (0.46 g, 3.0 mmol) and 4-nitroacetophenone (0.50 g, 3.0 mmol) as starting materials: Yellow crystals (0.35 g, 40%), mp: decompose. ¹H NMR# (CDCl₃) δ 8.39 (AA′ part of an AA′MM′ system, H3′, H5′), 8.31 (AA′ part of an AA′MM′ system, H3, H5), 8.16 (MM′ part of an AA′MM′ system, H2′, H6′), 7.84 (d, J 15.8 Hz, Hβ), 7.80 (MM′ part of an AA′MM′ system, H2, H6), 7.62 (d, J 15.8 Hz, Hα). Anal. Calcd. (C₁₅H₁₀N₂O₅): C, 60.41; H, 3.38; N, 9.39. Found: C, 60.28; H, 3.48; N, 9.38.

### 44) Preparation of 4-nitro-4′-dimethylaminochalcone

4-nitro-4′-dimethylaminochalcone was synthesised according to procedure E using 4-nitrobenzaldehyde (0.17 g, 1.1 mmol) and 4-dimethylaminoacetophenone (H. Gilman et al. J. Am. Chem. Soc. 1933, 55, 1265-1270) (0.20 g, 1.1 mmol) as starting materials: Orange crystals (0.29 g, 80%), mp: 198.5-199.5° C. ¹H NMR# (CDCl₃) δ 8.40 (d, J 15.5 Hz, Hβ), 8.30 (AA′ part of an AA′MM′ system, H3, H5), 8.1 (m, H2, H6, H2′, H6′), 7.74 (d, J 15.5 Hz, Hα), 6.81 (MM′ part of an AA′MM′ system, H3′, H5′), 3.11 (s, NCH₃). Anal. Calcd. (C₁₇H₁₆N₂O₃): C, 68.91; H, 5.44; N, 9.45. Found: C, 68.76; H, 5.53; N, 9.52.

### 45) Preparation of 4,4′-difluorochalcone

4,4′-difluorochalcone was synthesised according to procedure E using 4-fluorobenzaldehyde (0.78 ml, 7.2 mmol) and 4-fluoroacetophenone (0.89 ml, 7.2 mmol) as starting materials: White crystals (1.2 g, 68%), mp: 112.0-112.9° C. ¹H NMR# (CDCl₃) δ 8.05 (dd, J 8.7, 5.6 Hz, H2′, H6′), 7.78 (d, J 15.7 Hz, Hβ), 7.63 (dd, J 8.7, 5.6 Hz, H2, H6), 7.43 (d, J 15.7 Hz, Hα), 7.1 (m, H3, H5, H3′, H5′). Anal. Calcd. (C₁₅H₁₀OF₂): C, 73.77; H, 4.13. Found: C, 73.45; H, 4.21.

### 46) Preparation of 3,5-dimethoxy-2′-butoxychalcone

3,5-dimethoxy-2′-butoxychalcone was synthesised according to procedure E using 3,5-dimethoxybenzaldehyde (2.0 g, 12.0 mmol) and 2-butoxyacetophenone (E. R. Bockstacler et al. J. Am. Chem. Soc. 1949, 71, 3760-3766) (2.3 g, 12.0 mmol) as starting materials: Yellow crystals (3.8 g, 92%), mp: 69.5-70.1° C. ¹H NMR (CDCl₃) δ 7.66 (dd, J 7.6, 1.8 Hz, H6′), 7.57 (d, J 15.9 Hz, Hβ), 7.44 (d, J 15.9 Hz, Hα), 7.43 (t, J 7.7 Hz, H4′), 7.00 (t, J 7.7 Hz, H5′), 6.95 (bd, J 7.7 Hz, H3′), 6.73 (d, J 2.2 Hz, H2, H6), 6.49 (t, J 2.2 Hz, H4), 4.03 (bt, J 6.3 Hz, H1″), 3.79 (s, OCH₃), 1.77 (5., J 7.3 Hz, H2″), 1.45 (6., J 7.3 Hz, H3″), 0.87 (t, J 7.3 Hz, H4″). ¹³C NMR (CDCl₃) δ 192.4 (C═O), 160.7 (C3, C5), 157.7 (C2′), 142.1 (Cβ), 136.9 (C1), 132.9 (C4′), 130.4 (C6′), 128.9 (C1′), 127.4 (Cα), 120.4 (C5′), 112.1 (C3′), 105.9 (C2, C6), 102.2 (C4), 68.0 (C1″), 55.1 (OCH₃), 31.1 (C2″), 19.1 (C3″), 13.5 (C4″). Anal. Calcd. (C₂₁H₂₄O₄): C, 74.09; H, 7.11. Found: C, 74.41; H, 6.92.

### 47) Preparation of 3,5-dimethoxy-2′-dimethylaminochalcone

3,5-dimethoxy-2′-dimethylaminochalcone was synthesised according to procedure E using 3,5-dimethoxybenzaldehyde (1.5 g, 8.9 mmol) and 2′-dimethylaminoacetophenone (H. Rupe et al. Berichte, 1901, 34, 3523-3524) (1.45 g, 8.9 mmol) as starting materials: Yellow crystals (1.9 g, 66%), mp: 159.8-160.5° C. ¹H NMR (CDCl₃) δ 7.63 (d, J 15.9 Hz, Hβ), 7.53 (dd, J 7.6, 1.6 Hz, H6′), 7.37 (dt, J 7.6, 1.7 Hz, H4′), 7.33 (d, J 15.9 Hz, Hα), 6.98 (bt, J 7.6 Hz, H5′), 6.75 (bd, J 7.6 Hz, H3′), 6.72 (d, J 2.2 Hz, H2, H6), 6.48 (t, J 2.2 Hz, H4), 3.78 (s, OCH₃), 2.80 (s, NCH₃). ¹³C NMR (CDCl₃) δ 194.8 (C═O), 160.9 (C3, C5), 152.2 (C2′), 142.4 (Cβ), 137.1 (C1), 132.0 (C4′), 131.3 (C1″), 130.4 (C6″), 126.6 (Cα), 120.0 (C5′), 116.9 (C3′), 106.1 (C2, C6), 102.1 (C4), 55.3 (OCH₃), 44.3 (NCH₃). Anal. Calcd. (C₁₉H₂₁NO₃): C, 73.29; H, 6.80; N, 4.50. Found: C, 73.17, H, 6.96; N, 4.12.

### 48) Preparation of 3,5-dimethoxy-2′-nitrochalcone

3,5-dimethoxy-2′-nitrochalcone was synthesised according to procedure F using 3,5-dimethoxybenzaldehyde (1.0 g, 6.1 mmol) and 2′-nitroacetophenone (1.0 g, 6.1 mmol) as starting materials: Yellow crystals (0.45 g, 23%), mp: 98.4-98.9° C. ¹H NMR (CDCl₃) δ 8.18 (dd, J 8.0, 1.2 Hz, H3′), 7.78 (td, J 7.4, 1.2 Hz, H5′), 7.66 (td, J 8.0, 1.5 Hz, H4′), 7.50 (dd, J 7.4, 1.5 Hz, H6′), 7.11 (d, J 15.9 Hz, Hβ), 6.92 (d, J 15.9 Hz, Hα), 6.61 (d, J 2.2 Hz, H2, H6), 6.49 (t, J 2.2 Hz, H4), 3.79 (s, OCH₃). ¹³C NMR (CDCl₃) δ 192.9 (C═O), 160.9 (C3, C5), 146.4 (C2′), 146.2 (Cβ), 136.1 (C1), 135.6 (C1′), 134.0 (C5′), 130.5 (C4′), 128.7 (C6′), 126.6 (Cα), 124.4 (C3′), 106.2 (C2, C6), 103.2 (C4), 55.3 (OCH₃). Anal. Calcd (C₁₇H₁₅NO₅): C, 65.17; H, 4.83; N, 4.47. Found: C, 65.23; H, 4.86; N, 4.24.

### 49) Preparation of 3,5-dimethoxy-2′-fluorochalcone

3,5-dimethoxy-2′-fluorochalcone was synthesised according to procedure E using 3,5-dimethoxybenzaldehyde (2.4 g, 14.4 mmol) and 2′-fluoroacetophenone (2.0 g, 14.4 mmol) as starting materials: Yellow crystals (2.9 g, 71%), mp: 71.6-72.5° C. ¹H NMR (CDCl₃) δ 7.81 (dt, J 7.6, 1.8 Hz, H6′), 7.64 (d, J 15.7 Hz, Hβ), 7.57 (m, H4′), 7.34 (d, J 15.7 Hz, Hα), 7.3-7.1 (m, H3′, H5′), 6.76 (d, J 2.2 Hz, H2, H6), 6.53 (t, J 2.2 Hz, H4), 3.83 (s, OCH₃). ¹³C NMR (CDCl₃) δ 189.0 (C═O), 161.0 (C3, C5), 158.6 (C2′), 144.8 (Cβ), 136.5 (C1), 134.1 (C4′), 131.0 (C6′), 126.9 (C1′), 125.9 (Cα), 124.5 (C5′), 116.3 (C3′), 106.4 (C2, C6), 102.9 (C4), 55.4 (OCH₃). Anal. Calcd (C₁₇H₁₅O₃F): C, 71.32; H, 5.28; F, 6.64. Found: C, 71.15; H, 5.31; F, 6.78.

### 50) Preparation of 2,4-dimethoxy-3′-butoxychalcone

2,4-dimethoxy-3′-butoxychalcone was synthesised according to procedure E using 2,4-dimethoxybenzaldehyde (1.62 g, 9.7 mmol) and 3′-butoxyacetophenone (E. R. Bockstacler et al. J. Am. Chem. Soc. 1949, 71, 3760-3766) (1.87 g, 9.7 mmol) as starting materials: Yellow crystals (3.0 g, 92%), mp: 80.4-81.3° C. ¹H NMR (CDCl₃) δ 8.07 (d, J 15.8 Hz, Hβ), 7.56 (m, H2′, H6′, H6), 7.51 (d, J 15.8 Hz, Hα), 7.36 (t, J 7.8 Hz, H5′), 7.07 (ddd, J 8.2, 2.6, 0.9 Hz, H4′), 6.50 (dd, J 2.2 Hz, H5), 6.43 (d, J 2.2 Hz, H3), 4.00 (t, J 6.4 Hz), 3.85 (s), 1.78 (5., J 7.2, H3″), 1.47 (6., J 7.2, H2″), 0.97 (t, J 7.2, H1″). ¹³C NMR (CDCl₃) δ 190.5 (C═O), 162.8*(C4), 160.1*(C2), 159.1 (C3′), 140.1 (Cβ), 139.9 (C1′), 130.5 (C5′), 129.1 (C6), 120.5 (Cα), 119.9 (C6′), 118.9 (C4′), 116.7 (C1″), 113.2 (C2′), 105.2 (C5), 98.1 (C3), 67.6 (C1), 55.2 (OCH₃), 55.1 (OCH₃), 31.0 (C241 ), 19.0 (C3″), 13.6 (C4′). Anal Calcd. (C₂₁H₂₄O₄): C: 74.09; H, 7.11. found: C, 73.80; H, 7.15.

### 51) Preparation of 3,5-dimethoxy-3′-dimnethylaminochalcone

3,5-dimethoxy-3′-dimethylaminochalcone was synthesised according to procedure E using 3,5-dimethoxybenzaldehyde (2.0 g, 12.3 mmol) and 3′-dimethylaminoacetophenone (H. Rupe et al. Berichte, 1901, 34, 3523-3524) (2.0 g, 12.3 mmol) as starting materials: Yellow crystals (2.6 g, 49 %), mp: 81.8-82.6° C. ¹H NMR (CDCl₃) δ 7.70 (d, J 15.7 Hz, Hβ), 7.45 (d, J 15.7 Hz, Hα), 7.33 (m, H2′, H4′, H5′), 6.76 (d, J 2.2 Hz, H2, H6), 6.51 (t, J 2.2 Hz, H4), 6.93 (m, H6′), 3.82 (s, OCH₃), 3.00 (s, —N(CH3)₂). ¹³C NMR (CDCl₃) δ 191.1 (C═O), 160.8 (C3, C5), 150.5 (C3′), 144.1 (Cβ), 138.7 (C1′), 136.8 (C1), 128.9 (C5′), 123.0 (Cα), 116.7 (C6′, C4′), 111.5 (C2′), 106.1 (C2, C6), 102.4 (C4), 55.3 (OCH₃), 40.4 (—N(CH₃)₂). Anal Calcd (C₁₉H₂₁NO₃): C, 73.29; H, 6.80; N, 4.50. Found: C: 73.51; H, 6.85; N, 4.60.

### 52) Preparation of 2,4-dimethoxy-3′-nitrochalcone

2,4-dimethoxy-3′-nitrochalcone was synthesised according to procedure E using 2,4-dimethoxybenzaldehyde (0.5 g, 3.0 mmol) and 3′-nitroacetophenone (0.5 g, 3.0 mmol) as starting materials: Grey crystals (0.61 g, 64%), mp: 88.1-88.7° C. ¹H NMR (CDCl₃) δ 8.82 (t, J 2.0 Hz, H2′), 8.52 (dt, J 8.0, 1.4 Hz, H4 ′), 8.46 (ddd, J 8.0, 2.0, 1.4Hz, H6′), 8.17 (d, J 15.6 Hz, Hβ), 7.87 (d, J 8.0 Hz, H6), 7.86 (t, J 8.0 Hz, H5), 7.80 (d, J 15.6 Hz, Hα), 6.64 (bs, H3), 6.62 (dd, J 8.0, 2.1 Hz, H5), 3.97 (s, OCH₃), 3.89 (s, OCH₃). ¹³C NMR (CDCl₃) δ 186.0 (C═O), 162.9*(C4), 159.6*(C2), 143.3 (C3′), 139.9 (Cβ), 139.2 (C1′), 133.2 (C6′), 129.6 (C5′), 129.3 (C6), 125.7 (C4′), 121.8 (Cα), 117.3 (C2′), 115.3 (C1), 105.3 (C5), 97.2 (C3), 54.4 (OCH₃), 54.2 (OCH₃). Anal Calcd. (C₁₇H₁₅NO₅): C: 65.17, H: 4.83, N: 4.47; found: C: 65.00; H: 4.96, N: 5.25.

### 53) Preparation of 3,5-dimethoxy-3′-fluorochalcone

3,5-dimethoxy-3′-fluorochalcone was synthesised according to procedure E using 3,5-dimethoxybenzaldehyde (2.4 g, 14.4 mmol) and 3′-fluoroacetophenone (1.78 ml, 14.4 mmol) as starting materials: White crystals (2.3 g, 56%), mp: 89.5-90.3° C. ¹H NMR (CDCl₃) δ 7.79 (bd, J˜8 Hz, H6′), 7.73 (d, J 15.7 Hz, Hβ), 7.69 (dt, J˜9, 29 Hz, H2′), 7.49 (m, H4′), 7.48 (d, J 15.7 Hz, Hα), 7.28 (td, J˜8, ˜2 Hz, H540 ), 6.78 (d, J 2.2 Hz, H2, H6), 6.51 (t, J 2.2 Hz, H4), 3.84 (s, OCH₃). ¹³C NMR (CDCl₃) δ 189.0 (C═O), 165.1 (C3′), 160.9 (C3, C5), 145.5 (Cβ), 139.9 (C1′), 136.4 (C1), 130.3 (C5′), 124.1 (Cα), 121.8 (C6′), 119.9 (C4′), 115.4 (C2′), 106.3 (C2, C6), 102.9 (C4), 55.4 (OCH₃). Anal. Calcd. 30(C₁₇H₁₅O₃F): C: 71.32 H: 5.28, F: 6.64. Found: C: 71.20, H: 5.52, F: 6.50.

### 54) Preparation of 3,5-dimethoxy-4′-cyclohexylchalcone

3,5-dimethoxy-4′-cyclohexylchalcone was synthesised according to procedure E using 3,5-dimethoxybenzaldehyde (0.82 g, 4.9 mmol) and 4-cyclohexylacetophenone (1.0 g, 4.9 mmol) as starting materials: Brown crystals (1.6 g, 92%), mp: 75.5-76.2° C. ¹H NMR (CDCl₃) δ 7.95 (AA′ part of an AA′MM′ system, H2′, H6′), 7.64 (d, J 15.9 Hz, Hβ), 7.48 (d, J 15.9 Hz, Hα), 7.33 (MM′ part of AA′MM′ system, H3′, H5′), 6.78 (d, J 2.2 Hz, H2, H6), 6.52 (t, J 2.2 Hz, H4), 3.84 (s, OCH₃), 2.58 (b, H1′), 1.8 (m, H2″, H6″), 1.4 (m, H3″-H5″). ¹³C NMR (CDCl₃) δ 189.9 (C═O), 161.0 (C3, C5), 153.7 (C4′), 144.3 (Cβ), 137.0 (C1), 136.0 (C1′), 128.8 (C2′, C6′), 127.1 (C3′, C5′), 122.6 (Cα), 106.3 (C2, C6), 102.6 (C4), 55.5 (OCH₃), 44.7 (C1″), 34.1 (C2″, C6″), 26.7 (C3″, C5), 26.0 (C4″). Anal. Calcd. (C₂₃H₂₆O₃): C:78.83; H, 7.48. Found: C: 78.54, H: 7.62.

### 55) Preparation of 3,5-dimethoxy-4′-dimethylaminochalcone

3,5-dimethoxy-4′-dimethylaminochalcone was synthesised according to procedure E using 3,5-dimethoxybenzaldehyde (0.67 g, 4.0 mmol) and 4-dimethylaminoacetophenone (H. Gilman et al. J. Am. Chem. Soc. 1933, 55, 1265-1270) (0.66 g, 4.0 mmol) as starting materials: Orange crystals (0.91 g, 73%), mp: 155.1-156.2° C. ¹H NMR ((CD₃)₂CO) δ 8.10 (AA′ part of an AA′MM′ system, H2′, H6′), 7.92 (d, J 15.6 Hz, Hβ), 7.63 (d, J 15.6 Hz, Hα), 7.15 (d, J 2.1 Hz, H2, H6), 6.79 (MM′ part of an AA′MM′ system, H3′, H5′), 6.56 (t, J 2.1 Hz, H4), 3.85 (s, OCH₃), 3.10 (s, —N(CH₃)₂). ¹³C NMR ((CD₃)₂CO) δ 186.8 (C═O), 161.8 (C3, C5), 154.2 (C4′), 142.4 (Cβ), 138.1 (C1), 131.4 (C2′, C6′), 126.4 (C1′), 123.5 (Cα), 111.5 (C3′, C5′), 106.9 (C2, C6), 102.8 (C4), 55.7 (OCH₃), 39.9 (—N(CH₃)₂). Anal. Calcd. (C₁₉H₂₁NO₃): C: 73.29, H: 6.80, N: 4.50; found: C: 73.20, H, 6.86; N: 4.59.

### 56) Preparation of 3,5-dimethoxy-4′-nitrochalcone

3,5-dimethoxy-4′-nitrochalcone was synthesised according to procedure E using 3,5-dimethoxybenzaldehyde (1.0 g, 6.1 mmol) and 4-nitroacetophenone (1.0 g, 6.1 mmol) as starting materials: Yellow crystals (1.5 g, 77%), mp: 165.5-166.3° C. ¹H NMR (CDCl₃) δ 8.36 (AA′ part of an AA′MM′ system, H3′, H5′), 8.14 (MM′ part of an AA′MM′ system, H2′, H6′), 7.75 (d, J 15.3 Hz, Hβ), 7.42 (d, J 15.3 Hz, Hα), 6.78 (d, J2.2 Hz, H2, H6), 6.56 (t, J2.2 Hz, H4), 3.84 (s, OCH₃). ¹³C NMR (CDCl₃) δ 189.4 (C═O), 161.4 (C3, C5), 150.4 (C4′), 143.2 (C1′), 140.7 (Cβ), 136.4 (C1), 132.5 (C2′, C6′), 129.7 (Cα), 127.9 (C3′, C5′), 106.8 (C2, C6), 103.7 (C4), 55.6 (OCH₃). Anal. Calcd. (C₁₇H₁₅NO₅): C: 65.17, H: 4.83; N, 4.47. found: C, 65.32; H, 5.03; N: 4.33.

### 57) Preparation of 4-fluorochalcone

4-fluorochalcone was synthesised according to procedure E using benzaldehyde (0.10 ml, 1.0 mmol) and 4-fluoroacetophenone (0.14 g, 1.0 mmol) as starting materials: Yellow crystals (0.20 g, 90%), mp: 102.1-103.2° C. ¹H NMR (CDCl₃) δ 8.04 (m, H2′, H6′), 7.80 (d, J 15.5 Hz, Hβ), 7.62 (m, H3′, H5′), 7.49 (d, J 15.5 Hz, Hα), 7.40 (m, H2, H4, H6), 7.15 (m, H3, H5).¹³C NMR δ 188.1 (C═O), 168.1 (C4′), 145.0 (Cβ), 134.7 (C1), 134.4 (C1′), 131.0 (C2′, C6′), 130.6 (C3, C5), 129.0 (C2, C6), 128.5 (C4), 121.4 (Cα), 115.9 (C3′, C5′). Anal. Calcd. (C₁₅H₁₁OF): C, 79.63; H, 4.90. Found: C, 80.01; H, 4.97.

### 58) Preparation of 2-butoxy-2′,3′,4-trimethoxychalcone

2-butoxy-2′,3′,4′-trimethoxychalcone was synthesised according to procedure E using 2-butoxybenzaldehyde (1.7 g, 9.5 mmol) and 2,3,4-trimethoxyacetophenone (E. R. Bockstacler et al. J. Am. Chem. Soc. 1949, 71, 3760-3766) (1.7 ml, 9.5 mmol) as starting materials: Yellow crystals (3.0 g, 86%), mp: 129.4-130.2° C. ¹H NMR (CDCl₃) δ 8.04 (d, J 16.0Hz, Hβ), 7.62 (dd, J 7.7, 1.5 Hz, H6), 7.55 (d, J 16.0 Hz, Hα), 7.46 (d, J 8.8 Hz, H6′), 7.31 (td, J 7.7, 1.5 Hz, H4), 6.94 (bt, J 7.7 Hz, H5), 6.89 (bd, J 7.7 Hz, H3), 6.74 (d, J 8.8 Hz, H5′), 4.00 (t, J 6.4 Hz, H1″), 3.97 (s, OCH₃), 1.80 (5.kt. J 7.4 Hz, H2″), 1.48 (6.kt, J 7.4 Hz, H3″), 0.96 (t, J 7.3 Hz, H4″). ¹³C NMR (CDC₃) δ 191.4 (C═O), 157.8 (C2), 156.3 (C4′), 153.1 (C2′), 141.6 (C3′), 138.7 (Cβ), 131.2 (C4), 128.4 (C6), 126.6 (C1), 126.5 (Cα), 125.2 (C6′), 123.6 (C1′), 120.1 (C5), 111.7 (C3), 106.8 (C5′), 67.7 (C1″), 61.7 (OCH₃), 60.6 (OCH₃), 55.7 (OCH₃), 30.8 (C2″), 19.0 (C3″), 13.5 (C4″). Anal. Calcd. (C₂₂H₂₆O₅): C, 71.33; H: 7.07. Found: C: 71.45, H: 7.17.

### 59) Preparation of 2-nitro-2′,3′,4′-trimethoxychalcone

2-nitro-2′,3′,4′-trimethoxychalcone was synthesised according to procedure F using 2-nitrobenzaldehyde (0.72 g, 4.8 mmol) and 2,3,4-trimethoxyacetophenone (0.8 ml, 4.8 mmol) as starting materials: Red-brown crystals (0.22 g, 13%), mp: 107.1-108.0° C. ¹H NMR (CDCl₃) δ 8.04 (d, J 15.7 Hz, Hβ), 8.03 (dd, J 8.0, 1.2 Hz, H3), 7.76 (dd, J 7.8, 1.7 Hz, H6), 7.67 (td, J 7.8, 1.2 Hz, H5), 7.55 (td, J8.0, 1.8 Hz, H4), 7.53 (d, J8.8Hz, H6′), 7.37 (d, J 15.7Hz, Hα), 6.79 (d, J 8.8 Hz, H5′), 3.94 (s, OCH₃), 3.92 (s, OCH₃), 3.91 (s, OCH₃). ¹³C NMR (CDCl₃) δ 190.0 (C═O), 157.3 (C4′), 153.9 (C2′), 142.1 (C2), 137.7 (Cβ), 136.0 (C3′), 133.3 (C5), 131.3 (C1), 131.2 (C4), 129.9 (C6), 129.0 (Cα), 126.0 (C3), 125.8 (C1′), 124.8 (C6′), 107.3 (C5′), 62.1 (OCH₃), 61.0 (OCH₃), 56.0 (OCH₃). Anal. Calcd. (C₁₈H₁₇NO₆): C: 62.97, H: 4.99, N: 4.08. Found: C: 63.09; H: 5.06, N: 4.22.

### 60) Preparation of 2-fluoro-2′,3′,4′-trimethoxychalcone

2-fluoro-2′,3′,4′-trimethoxychalcone was synthesised according to procedure E using 2-fluorobenzaldehyde (1.7 ml, 16.1 mmol) and 2,3,4-trimethoxyacetophenone (2.9 ml, 16.1 mmol) as starting materials: White crystals (3.6 g, 81%), mp: 75.0-75.8° C. ¹H NMR (CDCl₃) δ 7.82 (d, J 16.0 Hz, Hβ), 7.64 (td, J 7.6, 1.4 Hz, H4), 7.62 (d, J 16.0 Hz, Hα), 7.52 (d, J 8.8 Hz, H6′), 7.35 (m, H6), 7.17 (bt, J˜8 Hz, H5), 7.10 (bt, J˜8 Hz, H3), 6.77 (d, J 8.8 Hz, H5′), 3.96 (s, OCH₃), 3.94 (s, OCH₃), 3.91 (s, OCH₃). ¹³C NMR (CDCl₃) δ 190.4 (C═O), 164.8 (C4′), 157.0 (C2), 154.1 (C2′), 136.0 (C3′), 135.1 (Cβ), 131.4 (C4), 129.1 (C6), 128.5 (Cα), 126.2 (C1), 125.8 (C6′), 124.3 (C5), 123.8 (C1′), 116.2 (C3), 107.1 (C5′), 61.8 (OCH₃), 60.8 (OCH₃), 55.9 (OCH₃). Anal Calcd. (C₁₈H₁₇O₄F): C: 68.35, H: 5.42, F: 6.01. Found: C: 68.57, H: 5.48, F: 6.14.

### 61) Preparation of 3-phenoxy-2′,3′,4′-trimethoxychalcone

3-phenoxy-2′,3′,4-trimethoxychalcone was synthesised according to procedure E using 3-phenoxybenzaldehyde (0.82 ml, 4.8 mmol) and 2,3,4-trimethoxyacetophenone (0.87 ml, 4.8 mmol) as starting materials: Yellow crystals (1.1 g, 58%), mp: 63.3-64.0° C. ¹H NMR (CDCl₃) δ 7.63 (d, J 15.8 Hz, Hβ), 7.48 (d, J 8.8 Hz, H6′), 7.45 (d, J 15.8 Hz, Hα), 7.4-7.0 (m, H2, H3, H6, H5′, H3″-H5″), 7.12 (t, J 7.8 Hz, H5), 7.02 (MM′ part of an AA′MM′ system, H2′, H6″), 3.91 (s, OCH₃), 3.87 (s, OCH₃).

¹³C NMR (CDCl₃) δ 190.2 (C═O), 157.6 (C3, C1″), 157.0 (C4′), 156.4 (C2′), 141.8 (Cβ), 137.6 (C3″), 136.7 (C1), 130.0 (C5), 129.6 (C3″), 126.9 (Cα), 126.2 (C1′), 125.7 (C6′), 123.4 (C4″), 123.1 (C6), 120.1 (C4), 118.9 (C2″), 117.6 (C2), 107.1 (C5′), 61.7 (OCH₃), 60.8 (OCH₃), 55.9 (OCH₃). Anal. Calcd. (C₂₄H₂₂O₅): C, 73.83;,H: 5.68, Found: C: 73.94, H: 5.67.

### 62) Preparation of 3-dimethylamino-2′,3′,4-trimethoxychalcone

3-dimethylamino-2′,3′,4′-trimethoxychalcone was synthesised according to procedure E using 3-dimethylaminobenzaldehyde (W. Cocker et al. J. Chem. Soc. 1938, 751-753) (1.0 g, 6.9 mmol) and 2,3,4-trimethoxyacetophenone (1.3 ml, 6.9 mmol) as starting materials: Orange crystals (2.1 g, 90%), mp: 87.2-87.9° C.

¹H NMR (CDCl₃) δ 7.66 (d, J 15,8 Hz, Hβ), 7.48 (d, J 8.8 Hz, H6′), 7.46 (d, J 15.8 Hz, Hα), 7.27 (t, J 7.9 Hz, H5), 7.02 (bd, J 7.9 Hz, H6), 6.92 (b, H2), 6.78 (bd, J 7.9 Hz, H4), 6.75 (d, J 8.8 Hz, H5′), 3.93 (s, OCH₃), 3.92 (s, OCH₃), 2.99 (s, —(N(CH₃)₂). ¹³C NMR (CDCl₃) δ 191.1 (C═O), 156.8 (C4′), 153.6 (C2′), 144.4 (Cβ), 141.9 (C3), 135.7 (C1), 136.8 (C3′) 129.4 (C5), 126.8 (C1′), 126.6 (C6′), 126.1 (Cα), 116.2 (C6), 114.4 (C4), 112.3 (C2), 107.1 (C5′), 62.0 (OCH₃), 61.0 (OCH₃), 56.0 (OCH₃), 40.5 (—N(CH₃)₂). Anal. Calcd. (C₂₀H₂₃NO₄): C, 70.36; H, 6.79; N, 4.10. Found: C: 70.42; H, 6.85; N, 4.21.

### 63) Preparation of 3-nitro-2′, 3′,4′-trimethoxychalcone

3-nitro-2′,3′,4′-trimethoxychalcone was synthesised according to procedure E using 3-nitrobenzaldehyde (0.72 g, 4.8 mmol) and 2,3,4-trimethoxyacetophenone (0.87 ml, 4.8 mmol) as starting materials: Yellow crystals (1.0 g, 64%) , mp: 130.1-130.7° C. ¹H NMR (CDCl₃) δ 8.46 (bt, J 1.7 Hz, H2), 8.22 (dt, J 8.1, 1.2 Hz, H6), 7.92 (bd, J 7.8 Hz, H4), 7.72 (d, J 15.8 Hz, Hβ), 7.66 (d, J 15.8 Hz, Hα), 7.61 (t, J 8.0 Hz, H5), 7.55 (d, J 8.8 Hz, H6′), 6.80 (d, J 8.8 Hz, H5′), 3.96 (s, OCH₃), 3.95 (s, OCH₃). ¹³C NMR (CDCl₃) δ 189.6 (C═O), 157.4 (C4′), 153.8 (C2′), 141.9 (C3), 139.2 (Cβ), 136.8 (C1), 135.1 (C3′) 133.8 (C6), 129.8 (C5), 129.0 (Cα), 125.9 (C6), 125.8 (C4), 124.1 (C1′), 122.3 (C2), 107.3 (C5′), 61.9 (OCH₃), 60.9 (OCH₃), 56.0 (OCH₃). Anal. Calcd. (C₁₈H₁₇NO₆): C, 62.97; H, 4.99; N, 40.8. found: C, 62.65; H, 5.03; N, 4.06.

### 64) Preparation of 3-fluoro-2′,3′,4′-trimethoxychalcone

3-fluoro-2′,3′,4-trimethoxychalcone was synthesised according to procedure E using 3-fluorobenzaldehyde (1.7 ml, 16.1 mmol) and 2,3,4-trimethoxyacetophenone (2.9 ml, 16.1 mmol) as starting materials: Yellow crystals (1.7 g, 34%), mp: 79.3-80.2° C. ¹H NMR (CDCl₃) δ 7.65 (d, J 15.8 Hz, Hβ), 7.52 (d, J8.8 Hz, H6′), 7.50 (d, J 15.8 Hz, Hα), 7.42-7.28 (m, H4H6), 7.1 (m, H2), 6.77 (d, J 8.8 Hz, H5′), 3.94 (s, OCH₃), 3.93 (s, OCH₃). ¹³C NMR (CDCl₃) δ 190.5 (C═O), 165.3 (C4′), 157.9 (C3), 153.8 (C2′), 141.3 (Cβ), 138.0 (C1), 136.1 (C3′) 130.5 (C5), 127.7 (Cα), 126.3 (C1′), 126.0 (C6′), 124.5 (C6), 117.2 (C4), 114.6 (C2), 107.4(C5′), 62.1 (OCH₃), 61.1 (OCH₃), 56.2 (OCH₃). Anal. Calcd. (C₁₈H₁₇O₄F): C, 68.35; H, 5.42; F, 6.01. Found: C, 68.21; H, 5.30; F, 5.91.

### 65) Preparation of 4-dimethylamino-2′,3′,4-trimethoxychalcone

4-dimethylamino-2′,3′,4-trimethoxychalcone was synthesised according to procedure E using 4-dimethylaminobenzaldehyde (0.71 g, 4.8 mmol) and 2,3,4-trimethoxyacetophenone (0.87 ml, 4.8 mmol) as starting materials: Yellow crystals (0.86 g, 53%), mp: 168.1-169.3° C. ¹H NMR (CDCl₃) δ 7.64 (d, J 15.9 Hz, Hβ), 7.51 (AA′ part of an AA′MM′ system, H2, H6), 7.43 (d, J 8.8 Hz, H6′), 7.28 (d, 15.9 Hz, Hα), 6.75 (d, J 8.8 Hz, H5′), 6.68 (MM′ part of an AA′MM′ system, H3, H5), 3.92 (s, OCH₃), 3.90 (s, OCH₃), 3.03 (s, —N(CH₃)₂). ¹³C NMR (CDCl₃) δ 190.9 (C═O), 156.2* (C4′), 152.1* (C2′), 144.7 (Cβ), 143.8 (C4), 130.3 (C2, C6), 130.2 (C3′), 127.1 (C1), 126.1 (C1′), 125.5 (Cα), 121.6 (C6″), 111.8 (C3, C5), 107.1 (C5′), 62.1* (OCH₃), 61.1* (OCH₃), 56.1* (OCH₃), 40.2 (NCH₃). Anal Calcd. (C₂₀H₂₃NO₄): C, 70.36; H, 6.79; N, 4.10. found: C, 70.23; H, 6.86; N, 4.16.

### 66) Preparation of 4-cyano-2′,3′,4′-trimethoxychalcone

4-cyano-2′,3′,4′-trimethoxychalcone was synthesised according to procedure E using 4-cyanobenzaldehyde (1.3 g, 9.5 mmol) and 2,3,4-trimethoxyacetophenone (1.7 ml, 9.5 mmol) as starting materials: Yellow crystals (2.4 g, 81%), mp: 131.7-133.0° C. ¹H NMR (CDCl₃) δ 7.7-7.6 (m, Hα, Hβ, H2, H3, H5, H6), 7.55 (d, J 8.8 Hz, H6′), 6.78 (d, J 8.8 Hz, H5′), 3.95 (OCH₃), 3.92 (s, OCH₃).

¹³C NMR (CDCl₃) δ 189.6 (C═O), 157.3 (C4′), 154.2 (C2′), 139.6 (Cβ), 139.5 (C1), 136.7 (C3′), 132.4 (C2, C6), 129.4 (Cα), 128.4 (C3, C5), 126.0 (C6′), 125.9 (C1′), 118.9 (CN), 112.8 (C4), 107.3 (C5′), 62.0* (OCH₃), 60.9* (OCH₃), 56.0* (OCH₃). Anal. Calcd. (C₁₉H₁₇NO₄): C, 70.58; H, 5.30; N, 4.33. Found: C, 70.97; H, 5.45; N, 4.45.

### 67) Preparation of 3,5-dimethoxy-2′-hydroxychalcone

3,5-dimethoxy-2′-hydroxychalcone was synthesised according to procedure E using 3,5-dimethoxybenzaldehyde (0.47 g, 2.8 mmol) and 2-hydroxyacetophenone (0.34 ml, 2.8 mmol) as starting materials: Orange crystals (0.27 g, 34%), mp: 110.2-110.9° C. ¹H NMR (CDCl₃) δ 7.91 (dd, J 8.4, 1.0 Hz, H6′), 7.82 (d, J 15.6 Hz, Hβ), 7.59 (d, J 15.6 Hz, Hα), 7.50 (td, J 8.4, 1.0 Hz, H4′), 7.03 (d, J 8.4 Hz, H3′), 6.95 (t, J 8.4 Hz, H5′), 6.79 (d, J 2.2 Hz, H2, H6), 6.55 (t, J 2.2 Hz, H4), 3.85 (s, OCH₃). ¹³C NMR (CDCl₃) δ 193.4 (C═O), 163.4 (C2′), 161.1 (C3, C5), 145.5 (Cβ), 136.4 (C1), 136.5 (C4′), 129.7 (C6′), 120.5 (Cα), 120.0 (C1′), 118.9 (C5′), 118.6 (C3′), 106.5 (C2, C6), 103.0 (C4), 55.5 (OCH₃). Anal. Calcd. (C₁₇H₁₆O₄): C, 71.82; H, 5.67; found: C, 71.59; H, 5.72.

### 68) Preparation of 3,5-dimethoxy-3′-hydroxychalcone

3,5-dimethoxy-3′-hydroxychalcone was synthesised according to procedure E using 3,5-dimethoxybenzaldehyde (0.47 g, 2.8 mmol) and 3-hydroxyacetophenone (0.38 g, 2.8 mmol) as starting materials: Yellow crystals (0.25 g, 32%), mp: 126.2-126.8° C. ¹H NMR (CDCl₃) δ 9.66 (s, OH), 7.74 (d, J 16.0Hz, βH), 7.66 (d, J 16.0 Hz, αH), 7.59 (bd, J˜8 Hz, H4′), 7.52 (bt, J˜2 Hz, H2′), 7.36 (t, J 7.8 Hz, H5′), 7.10 (dd, J 7.8, 2.4 Hz, H6′), 6.95 (d, J 2.2 Hz, H2, H6), 6.58 (t, 2.2 Hz, H4), 3.82 (s, OCH₃). ¹³C NMR (CDCl₃) δ 188.8 (C═O), 160.3 (C3, C5), 157.3 (C3′), 143.2 (Cβ), 138.6 (C1′), 136.1 (C1), 129.1 (C5′), 122.0 (Cα), 119.5 (C6′), 118.9 (C4′), 114.1 (C2′), 105.8 (C2, C6), 101.9 (C4), 54.5 (OCH₃). Anal. Calcd. (C₁₇H₁₆O₄): C, 71.82; H, 5.67. Found: C, 72.11; H, 5.79.

### 69) Preparation of 2-hydroxy-2′,3′,4′-trimethoxychalcone

2-hydroxy-2′,3′,4′-trimethoxychalcone was synthesised according to procedure E using 2-hydroxybenzaldehyde (0.25 ml, 2.4 mmol) and 2,3,4-trimethoxyacetophenone (0.43 ml, 2.4 mmol) as starting materials: Brown crystals (0.29 g, 39%), mp: 122.6-123.0° C. ¹H NMR (CD₃CN/DMSO) δ 7.84 (d, J 16.0 Hz, Hβ), 7.61 (bd, J 7.7 Hz, H6), 7.47 (d, J 16.0 Hz, Hα), 7.35 (d, J 8.7 Hz, H6′), 7.25 (bt, J˜8, 7.7 Hz, H4), 6.94 (bd, J 8.2 Hz, H3), 6.85 (m, H5′, H5), 3.88 (OCH₃), 3.87 (OCH₃), 3.82 (OCH₃). ¹³C NMR (CD₃CN/DMSO) δ 190.6 (C═O), 156.6 (C2), 156.5 (C4′), 156.0 (C2′), 141.6 (Cβ), 138.1 (C4), 136.4 (C3′), 131.0 (C6), 128.1 (Cα), 126.2 (C1), 125.6 (C6′), 124.4 (C5), 118.9 (C1′), 115.7 (C3), 106.9 (C5′), 60.9 (OCH₃), 59.7 (OCH₃), 55.2 (OCH₃). Anal. Calcd. (C₁₈H₁₈O₅): C, 68.78; H, 5.77. Found: C, 68.98; H, 5.99.

### 70) Preparation of 3,5,2′,3′,4′-pentamethoxychalcone

3,5,2′,3′,4′-pentamethoxychalcone was synthesised according to procedure E using 3,5-dimethoxybenzaldehyde (0.79 g, 4.8 mmol) and 2,3,4-trimethoxyacetophenone (0.87 ml, 4.8 mmol) as starting materials: Yellow crystals (1.4 g, 83%), mp: 111.6-112.5° C. 1H NMR (CDCl₃) δ 7.60 (d, J 15.8 Hz, Hβ), 7.49 (d, J 8.8 Hz, H6′), 7.46 (d, J 15.8 Hz, Hα), 6.76 (d, J 8.8 Hz, H5′), 6.75 (d, J 2.2 Hz, H2, H6), 6.50 (t, J 2.2 Hz, H4), 3.92 (s, OCH₃), 3.79 (s, OCH₃). ¹³C NMR (CDCl₃) δ 190.5 (C═O), 160.7 (C3, C6), 156.8 (C4′), 153.5 (C2′), 142.7 (Cβ), 141.8 (C1), 136.8 (C3′), 126.8 (Cα), 126.4 (C1), 125.6 (C6′), 107.1 (C5′), 106.0 (C6), 102.1 (C4), 61.8*(OCH₃), 60.8*(OCH₃), 55.8*(OCH₃), 55.1 (C3/5-OCH₃). Anal Calcd. (C₂₀H₂₂O₆): C, 67.03; H, 6.19. Found: C, 67.43; H, 6.25.

### 71) Preparation of 2,4,3′-trimethoxychalcone

2,4,3′-trimethoxychalcone was synthesised according to procedure E using 2,4-dimethoxybenzaldehyde (0.84 g, 5.0 mmol) and 3-methoxyacetophenone (0.67 ml, 5.0 ml) as starting materials: Yellow crystals (1.6 g, 91%), mp: 75.6-78.9° C. ¹H NMR (CDCl₃) δ 8.06 (d, J 15.8 Hz, Hβ), 7.7-7.6(m, H6, H6′, H2′), 7.55 (d, J 15.8 Hz, Hα), 7.43 (t, J 8.0 Hz, H5′), 7.07 (dt, J 8.0, 1.0 Hz, H4′), 6.55 (dd, J 8.4, 2.4 Hz, H5), 6.49 (d, J 2.4 Hz, H3), 3.90 (s, OCH₃), 3.88 (s, OCH₃), 3.86 (s, OCH₃). ¹³C NMR (CDCl₃) δ 191.1 (C═O), 163.2*(C3′), 160.4*(C4), 159.8*(C2), 140.6 (Cβ), 140.3 (C1′), 131.0 (C5′), 129.4 (C6), 121.1 (Cα), 120.4 (C6′), 118.8 (C4′), 112.8 (C2′), 112.2 (C1), 105.4 (C5), 98.4 (C3), 55.5 (OCH₃), 55.4 (OCH₃). Anal. Calcd. (C₁₈H₁₈O₄): C, 72.47; H, 6.08. Found: C, 72.27; H, 6.16.

### 72) Preparation of 2,4-dimethoxy-2′-bromochalcone

2,4dimethoxy-2′-bromochalcone was synthesised according to procedure E using 2,4-dimethoxybenzaldehyde (0.42 g, 2.5 mmol) and 2-bromoacetophenone (0.33 ml, 2.5 mmol) as starting materials: Yellow crystals (0.65 g, 71%), mp: 91.3-91.8° C. ¹H NMR (CDCl₃) δ 7.67 (d, J 16.2 Hz, Hβ), 7.61 (dd, J 7.0, 1.0 Hz, H6′), 7.48 (d, J 8.6 Hz, H6), 7.5-7.25 (m, H3′-H5′), 7.10 (d, J 16.2 Hz, Hα), 6.51 (dd, J 8.6, 2.3 Hz, H5), 6.43 (d, J 2.3 Hz, H3), 3.83 (s, OCH₃), 3.82 (s, OCH₃). ¹³C NMR (CDCl₃) δ 195.2 (C═O), 163.4*(C4), 160.3*(C2), 142.6 (Cβ), 141.6 (C1′), 133.3 (C4′), 131.0 (C3′), 130.9 (C6′), 129.1 (C6), 127.2 (Cα), 124.3 (C5′), 119.5 (C2′), 116.5 (C1), 105.5 (C5), 98.3 (C3), 55.5 (OCH₃). Anal. Calcd. (C₁₇H₁₅O₃Br): C, 58.81; H, 4.35; Br, 23.01. Found: C, 58.62; H, 4.34; Br, 23.18.

### 73) Preparation of 1,3-dimethoxy-4-(2,2-dibromoethenyl)benzene

To a solution of 2,4-dimethoxybenzaldehyde (0.88 g, 5 mmol) and triphenyl phosphine (2.62 g, 10 mmol) in anhydrous dichloromethane (10 ml) was added a solution of tetrabromomethane (1.91 g, 5.8 mmol) in dichloromethane (3 ml), keeping the temperature below 5° C. The reaction mixture was stirred for additional 30 min, filtered and concentrated in vacuo and the residue purified by column chromatography to give 1.27 g (72.1% ) of 1,3-dimethoxy-4-(2,2-dibromoethenyl)benzene as colourless crystals. mp: 147.0-147.8° C. ¹H NMR (CDCl₃) δ 7.67 (d, J 8.4Hz, H5), 7.51 (s, H1′), 6.46 (dd, J 8.4, 2.5 Hz, H6), 6.38 (d, J 2.5 Hz, H2), 3.79 (s, OCH₃), 3.75 (s, OCH₃). ¹³C NMR (CDCl₃) δ 161.2* (C1), 157.8* (C3), 132.2 (C1′), 129.6 (C5), 117.0 (C4), 104.0 (C6), 98.0 (C2), 87.5 (C2′), 55.4 (OCH₃), 55.3 (OCH₃).

### 74) Preparation of 1,3-dimethoxy-4-ethynylbenzene

A solution of 1,3-dimethoxy-4-(2,2-dibromoethenyl)benzene (1.10 g, 3.0 mmol) in tetrahydrofuran (20 ml) was cooled to −78° C., and slowly added n-butyllithium (2.52 ml of a 2.5 M soln., 6.3 mmol). The mixture was stirred for 15 min, added water (10 ml) and extracted with ethyl acetate (20 ml). The organic phase was concentrated in vacuo and the residue purified by column chromatography to give 0.296 g (65.6%) of 1,3-dimethoxy-4-ethynylbenzene as a clear oil. ¹H NMR (CDCl₃) δ 7.37 (d, J 9.0 Hz, H5), 6.44 (dd, J 9.0, 2.3 Hz, H6), 6.42 (d, J 2.3 Hz, H2), 3.86 (s, OCH₃), 3.80 (s, OCH₃), 3.25 (s, H2′). ¹³C NMR (CDCN) δ 166.9* (C3), 166.7* (C1), 139.6 (C5), 110.1 (C6), 108.2 (C4), 103.2 (C2), 85.2 (C2′), 85.1 (C1′), 60.3 (OCH₃), 60.1 (OCH₃).

### 75) Preparation of 1-(4-(2-propenyloxy)phenyl)-3-(2,4-dimethoxyphenyl)-2-propyn-1-one

A solution of 1,3-dimethoxy-4-ethynylbenzene (1.3 ml, 6.15 mmol), 4-allyloxybenzoyl chloride (R. Laliberté et al. Can. J. Pharm. Sci. 1967, 37-43) (2.42 g, 12.3 mmol), copper(I)iodide (0.24 g, mmol) and bis[triphenylphosphine]dichloropalladium(II) (0.24 g) in toluene (9 ml) and triethylamine (19 ml) was stirred for 16 h at room temperature. The mixture was washed with water and the organic phase was concentrated in vacuo. The residue was purified by column chromatography to give 0.56 g (28.3%) of 1-(4-(2-propenyloxy)phenyl)-3-(2,4-dimethoxyphenyl)-2-propynone-1 as yellow crystals, mp: 109.8-110.6° C. ¹H NMR (CDCl₃) δ 8.26 (AA′ part of an AA′MM′ system, H2′, H6′), 7.54 (d, J 8.4 Hz, H6), 6.98 (MM′ part of an AA′MM′ system, H3′, H5′), 6.51 (dd, J 8.4, 2.3 Hz, H5), 6.46 (d, J 2.3 Hz, H3), 6.05 (ddq, J 17.3, 10.5, 5.2 Hz, H2″), 5.44 (dd, J 17.3, 1.5 Hz, H3″trans), 5.33 (dd, J 10.5, 1.5 Hz, H3″cis), 4.62 (bd, J 5.2 Hz, H1″), 3.9 (s, OCH₃), 3.85 (s, OCH₃). ¹³C NMR (CDCl₃) δ 176.8 (C═O), 163.5* (C2), 163.3* (C4′), 163.1* (C4), 136.3 (C2″), 132.4 (C6), 132.0 (C2′, C6′), 130.8 (C1′), 118.2 (C3″), 114.4 (C3′, C5′), 105.5 (C5), 102.1 (C1), 98.3 (C3), 91.2* (Cβ), 91.1*(Cα), 68.9 (C1″), 55.9 (OCH₃), 55.6 (OCH₃).

### Example 2

Effect of Chalcones on in vitro Growth of Helicobacter pylori

Experiment A

The following compounds were tested:

1. SBC-24m4′bc: 2,4-dimethoxy-4′-butoxychalcone

2. SBC-24m4′ac: 2,4-dimethoxy-4′-allyloxychalcone

3. SBC-26m4′bc: 2,6-dimethoxy-4′-butoxychalcone

4. SBC-35m4′dac: 3,5-dimethoxy-4′-diaminochalcone

5. SBC-35m4′nic: 3,5-dimethoxy-4′-nitrochalcone

6. SBC-24m3′bc: 2,4-dimethoxy-3′-butoxychalcone

Stock solutions were made at a concentration of 1 mg/ml by dissolving 1 mg of each compound in 100 μl DMSO (dimethylsulfoxide) followed by addition of 900 μl of heat-inactivated fetal claf serum (HFCS). These stock solutions were diluted with HFCS to make solutions having the following concentrations: 320 μg/ml, 160 μg/ml, 80 μg/ml, 40 μg/ml and 20 μg/ml. Dilution rows were made with 3 ml aliquots in vials containing the compounds in the following final test concentrations: 16 μg/ml, 8 μg/ml, 4 μg/ml, 2 μg/ml and 1 μg/ml. As control was used DMSO-HFCS containing an amount of DMSO equal to the amount of test compound in the solution with 320 μg/ml compound.

Bacteria: 20 strains of Helicobacter pylori were used in an agar plate assay. The compounds were incorporated in the agar plates at different concentrations and then they were inoculated with different strains of Helicobacter pylori followed by incubation at microaerophilic atmosphere at 37° C. in 72 hours. The number of colony-forming units (CFU) were counted after the incubation period. The MIC values were determined as the minimal concentration required to inhibit growth of the bacteria.

Results

SBC-35m4′nic at a concentration of 16 μg/ml inhibited 12 out of 20 strains of Helicobacter pylori.

Experiment B

The following compounds were tested:

1. Licochalcone A

2. SBC-2ni2′3′4′mc: 2-nitro-2′,3′,4′-trimethoxychalcone

3. SBC-35n4′nic: 3,5-dimethoxy-4′-nitrochalcone

4. SBC-35m3′bc: 3,5-dimethocy-3′-butoxychalcone

5. SBC-24m4′bc: 2,4-dimethoxy-4′-butoxychalcone

6. SBC-24m4′ac: 2,4-dimethoxy-4′-allyloxychalcone

7. SBC-35m4′dac: 3,5-dimethoxy-4′-diaminochalcone

8. SBC-24m3′bc: 2,4-dimethoxy-3′-butoxychalcone

9. PH104: 3,5-dimethoxy-4′-cyclohexylchalcone

10. PH135-2A: 3,5-dimethoxy-2′fluorochalcone

11. PH136-3B: 3,5-dimethoxy-3′fluorochalcone

12. PH74: 2,4-difluoro-2′,4-dimethoxychalcone

13. PH98: 2-nitro-2′,3′,4′-trimethoxychalcone

14. PH105: 3,5-dimethoxy-4′nitrochalcone

Stock solutions were made at a concentration of 1 mg/ml by dissolving 1 mg of each compound in 100 μl DMSO (dimethylsulfoxide) followed by addition of 900 μl of heat-inactivated fetal claf serum (HFCS). These stock solutions were diluted with HFCS to make solutions having the following concentrations: 2560 μg/ml, 1280 μg/ml, 640 μg/ml, 320 μg/ml, 160 82 g/ml and 80 μg/ml. Dilution rows were made with 3 ml aliquots in vials containing the compounds in the following final test concentrations: 128 μg/ml, 64 μg/ml, 32 μg/ml, 12 μg/ml, 8 μg/ml and 4 μg/ml. As control was used DMSO-HFCS containing an amount of DMSO equal to the amount of test compound in the solution with 320 μg/ml compound.

Bacteria: 20 strains of Helicobacter pylori were used in an agar plate assay. The same method as described above under Experiment A was employed and MIC values were determined.

Results

1. Licochalcone A at a concentration of 16 μg/ml inhibited all 20 strains of Helicobacter pylori.

2. SBC-35m4′nic at a concentration of 16 μg/ml inhibited 12 out of 20 strains of Helicobacter pylori.

3. SBC-2ni2′,3′,4′mc at a concentration of 16 μg/ml inhibited 4 out of 20 strains of Helicobacter pylori.

Further studies were made to investigate the minimum inhibiting concentration (MIC) and the following results were obtained:

Licochalcone A: 20 out of 20 strains gave a MIC value equal to or less than 16 μg/ml

SBC-24A: 4 out of 20 strains gave a MIC value equal to or less than 16 μg/ml

SBC-24H: 14 out of 20 strains gave a MIC value equal to or less than 16 μg/ml

SBC-34: 4 out of 20 strains gave a MIC value equal to or less than 16 μg/ml

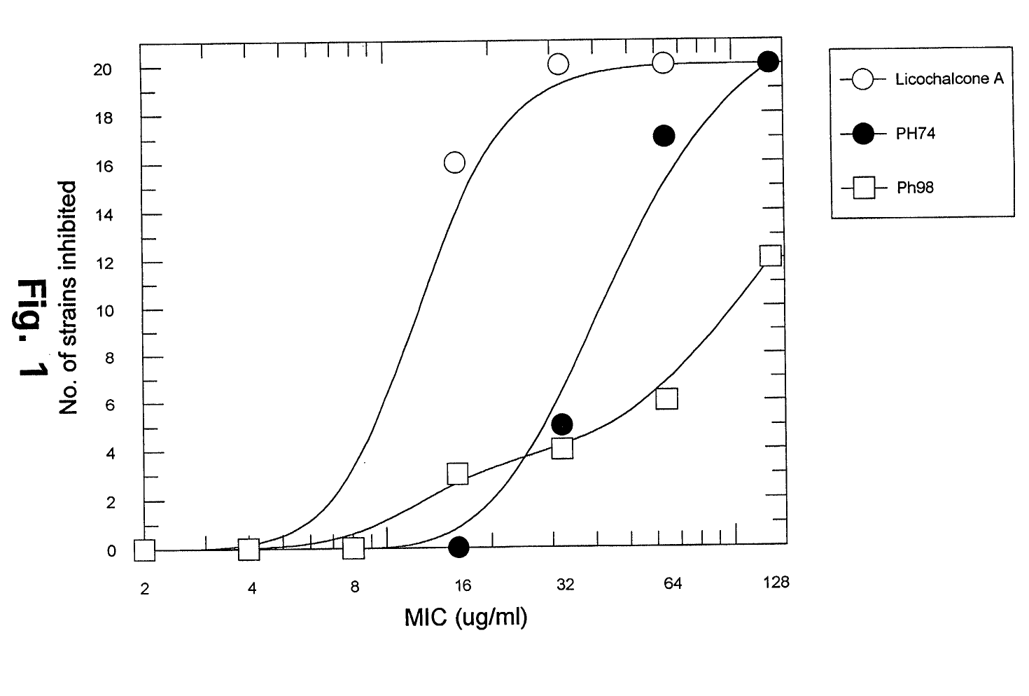

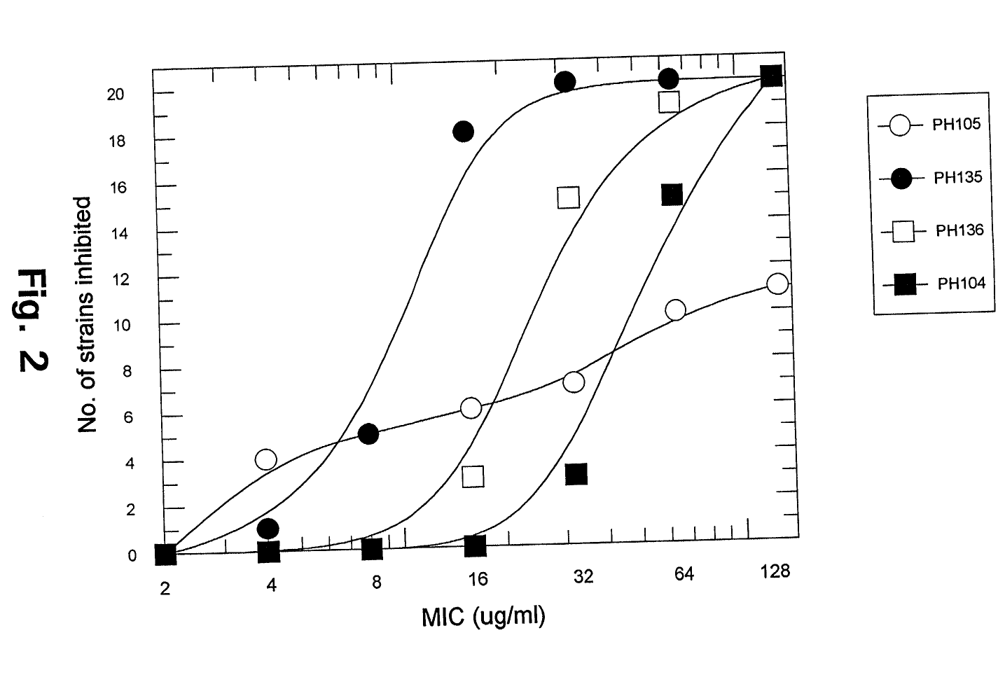

The results are shown in the following tables and FIGS. 1 and 2, wherein FIG. 1 shows the minimal inhibitory concentrations (MIC) as given in μg/ml of licochalcone A, PH74 and PH98 against 20 different strains of Helicobacter pylori and FIG. 2 shows the minimal inhibitory concentrations (MIC) as given in μg/ml of PH104, PH105, PH135 and PH136 against 20 different strains of Helicobacter pylori.

These results are presented as minimal inhibitory concentrations (MIC) in μg/ml vs. the number of H. pylori strains inhibited.

| | | No. strains inhibited/ |
|---|---|---|
| Compounds | MIC (μg/ml) | No. strains tested |
| Licochalcone A | 16 | 20/20 |
| PH32 | >128 | 0/20 |
| PH74 | 64 | 17/20 |
| PH81 | 16 | 5/20 |
| PH98 | 16 | 6/20 |
| PH104 | 64 | 15/20 |
| PH105 | 16 | 12/20 |
| PH135 | 32 | 20/20 |
| PH136 | 32 | 15/20 |
| PH142 | 128 | 2/20 |

Conclusions

As shown in FIGS. 1 and 2, several of the tested chalcones such as licochalcone A, PH74, PH104, PH135 and PH136 inhibited the growth of all the strains of H. pylori. The most effective compounds appear to be PH135 and licochalcone A PH74 and PH136 also show very good activities.

It should be noticed that according to other studies we have carried out the target molecule for the chalcones appears to be fumarate reductase. H. pylori uses fumarate reductase to generate ATP when grown under anaerobic conditions. These conditions are somewhat similar to those in the in vivo environment of the host. Under the in vitro screening conditions it is not possible to grow H. pylori under complete anaerobic conditions. The growth conditions used in the present screening studies have been with 5% oxygen. Therefore it is possible that the concentrations required to kill the bacteria in the in vivo environment might be lower than those in vitro.

### Example 3

Effect of Chalcones on Anaerobic Bacteria

The growth of three species of anaerobic bacteria Clostridium perfringes, Bacteroides fragilis and Peptostreptococcus were completely inhibted by licochalcone A at a concentration of 5 μg/ml.

Significance. This finding provides evidence for fumarate reductase as a target for chalcones, hence anaerobic bacteria utilise the pathway involving fumarate reductase for energy generation.

### Example 4

Effect of Oxygenated Chalcones on the Activity of Helicobacter pylori Fumarate Reductase

Bacteria: Helicobacter pylori (007 strain) were supplied by Dr. Lief P. Andersen, Legionella pneumophilia, Taflachia, micdadei, E. coli, Seerratia and Vibrio cholena were supplied by Dr. Alice Friis-Moller.

Extraction of (FRD)Helicobacter pylori Fumarate Reductase:

1) Extraction of soluble FRD: Bacteria were harvested by centrifugation at 18,000 g for 10 min at 4° C., and were washed 2 times in an isotonic phosphate saline buffer (50 mM sodium phosphate, pH 7.2, 90 mM NaCl, 5 mM Kcl). Bacteria were lysed in 5 mM Tris-HCl, pH 7.4, for 15 minutes, and centrifuged 18,000 g for 10 minutes. Membrane associated proteins were solubilized from the pellet in 150 mM KCl, pH 7.0, in ice bath for 60 min, and then the supernatant was collected after centrifugation (18,000 g for 10 minutes) for the measurement of FRD activity.

2) Extraction of FRD by sonication: Bacteria were harvested by centrifugation at 18,000 g for 10 min at 4° C., were sonicated, and then the supernatant was collected after centrifugation (18,000 g for 10 minutes) for the measurement of FRD activity.

NADH-fumarate Reductase Activity:

NADH-FRD activity was determined as the rate of NADH-oxidation upon addition of 1 mM fumarate in the KCl-solubilized fraction or sonicated fraction. The reaction was monitored spectrophotometrically at 340 nm (∈=6.2 mM⁻¹ cm⁻¹) using 100 μM NADH and usually 3 mg protein in both reference and sample cuvettes. The measurement was carried out in a Shimadzu UV-190 double-beam spectrophotometer. Protein concentrations were determined using a Bio-Rad protein assay (Bio-Rad Laboratories, 200 Alfred Nobel Dr. Hercules, Calif. 94547, USA).

Effect of Oxygenated Chalcones on the FRD Activity of H. pylori:

KCl solubilised fraction of bacteria was incubated with different concentrations of various oxygenated chalcones at 28° C. and the remaining FRD activity was measured. The IC₅₀ values were determined as the concentration at which 50% of the activity was inhibited.

References:

1. A. Denicola-Seoane et al. Mol-Biochem-Parasitol. 1992, 54(1):43-50.

2. H. Rubbo et al. Arch-Biochem-Biophy. 1994, 308(1):96-102.

3. G. L. Mendez et al. Arch-Biochem-Biophy. 1995, 321(1):153-159.

Results

Effect of licochalcone A and PH-105 on FRD of Helicobacter pylori. Data are given as mean±SD from three experiments.

| | FRD activity (mu/mg of protein) |
|---|---|
| Control | 19.2 ± 2.5 |
| Licochalcone A | |
| 50 | 0.0 |
| 25 | 0.9 ± 0.6 |
| 10 | 2.5 ± 1.1 |
| 5 | 6.8 ± 1.7 |
| 1 | 14.5 ± 2.9 |
| PH105 | |
| 50 | 0.0 |
| 25 | 1.7 ± 0.7 |
| 10 | 4.1 ± 1.5 |
| 5 | 8.5 ± 1.8 |
| 1 | 15.8 ± 2.0 |

Conclusion

The acitivities of NADH-fumarate reductase, succinate dehydrogenase, NADH dehydrogenase of Helicobacter pylori were investigated. The presence of NADH-fumarate reductase was demonstrated in the sonicate of H. pylori and fumarate could reduce H₂O₂ production from NADH indicating fumarate to be an endogenous substrate for accepting electrons from NADH. 2-thenoyltrifluoroacetone at concentration of 0.2 mM almost completely inhibited the activity of NADH-fumarate reductase, whereas succinate, malonate and antimycin could not inhibit it. Fumarate and malonate exhibited a concentration-dependent inhibitory effect on the activity of succinate dehydrogenase, while antimycin could no inhibit it. These date indicate that the oxidation of succinate to fumarate could be reversed by increasing the concentration of fumarate, whereas the reduction of fumarate to succinate could not be reversed by increasing the concentration of succinate. These results indicate that the fumarate to succinate pathway is predominant in H. pylori and this bacterium might use fumarate as a terminal electron acceptor.

### Example 5

Determination of Leishmania Fumarate Reductase

Parasites: A WHO reference vaccine strain of L. major originally isolated from a patient in Iran kindly provided by R. Behin, WHO Immunology Research and Training Centre, Lausanne, Switzerland, and two Kenyan strains of L. donovani (MHOM/KE/85/NLB 274 and MHOM/KE/85/NLB 439) kindly provided by Kenya Medical Research Institute, Nairobi, Kenya, were used. Promastigotes were cultured in completed medium (RPMI 199 containing with 0.02 mg/ml gentamycin, 25 mM Hepes, 4 mM L-glutamine, and 10% heat inactivated fetal calf serum (56° C., 30 min) at 26° C. Parasites for fumarate reductas (RD) study was harvested after 4 days.

Extraction of Soluble FRD

Parasites were harvested by centrifugation at 1000 g for 10 min, and were washed 2 times in an isotonic phosphate saline buffer (50 mM sodium phosphate, pH 7.2, 90 mM NaCl, 5 mM KCl). 1-8×1010/ml parasites were lysed in 5 mM Tris-HCl, pH 7.4, for 15 minutes, and centrifuged 4,550 g for 10 minutes. Membrane associated proteins were solubilized from the pellet in 150 mM KCl, pH 7.0, in ice bath for 30 min, and then the supernatant was collected after centrifugation (4,550 g for 10 minutes) for the measurement of FRD activity.

NADH-fumarate Reductase Activity

NADH-FRD activity was determined as the rate of NADH-oxidation upon addition of 1 mM fumarate in the KCl-solubilized fraction. The reaction was monitored spectrophotometrically at 340 nm ((=6.2 mM⁻¹ cm⁻¹) using 100 μM NADH and usually 0.1 mg protein in both reference and sample cuvettes. The measurement was carried out in a Shimadzu UV-190 double-beam spectrophotometer. Protein concentrations were determined using a Bio-Rad protein assay (Bio-Rad Laboratories, 200 Alfred Nobel Dr. Hercules, Calif. 94547, USA).

Effect of Oxygenated Chalcones on the FRD Activity of Leishmania

KCl solubilized fraction of Leishmania major promastigotes was incubated with different concentrations of various oxygenated chalcones at 28° C. and the remaining FRD activity was measured.

Purification of FRD:

All chromatography was performed on a FPLC system. Crude extract was concentrated on a MonoQ anion-exchange chromatography column and eluted in 1.5 M NaCl, 25 mM Tris-HCl pH 8.0. Elution was submitted to size exclusion chromatography on a Hi-Load superdex-200 column and eluted in 75 mM NaCl, 25 mM Tris-HCl pH 8.0. Fractions were tested for FR activity, and submitted for anion-exchange chromatography on a MonoQ column. The enzyme was eluted in a 0.075 M NaCl gradient, peaks were tested for FR activity, desalted on a PD-10 column and purity was assessed by SDS-PAGE on a 15% gel.

Procedure of Purification of FR from L. donovani:

Lysis of parasites by 5 mM Tris-HCl
↓
Concentration of sample
MonoQ column
↓
Gel filtration
Hiload superdex 200 column
separates proteins in range 104-6x105 Da
↓
Ion exchange chromatography
MonoQ column
↓
SDS-PAGE

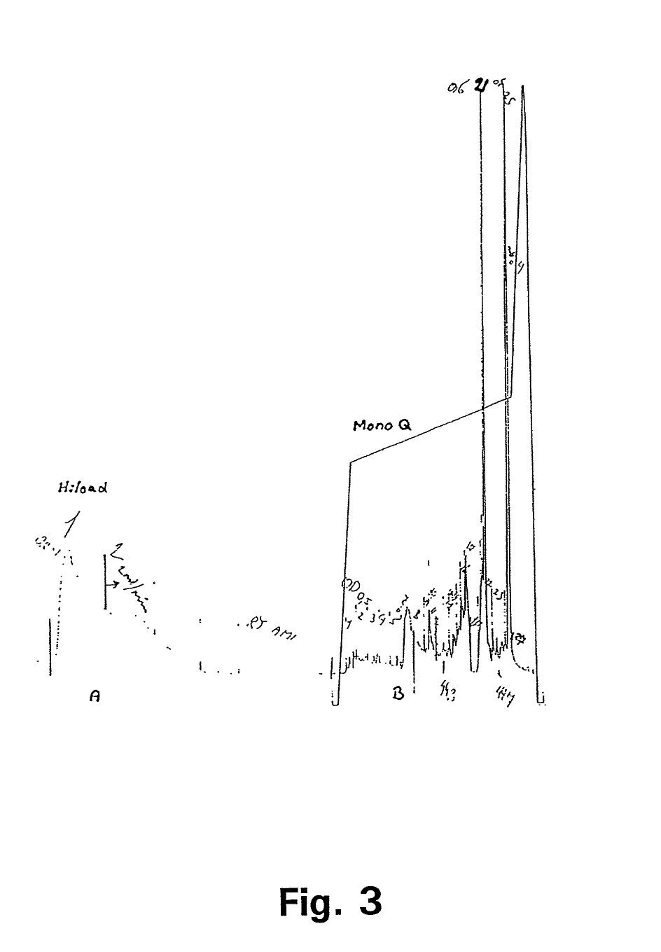

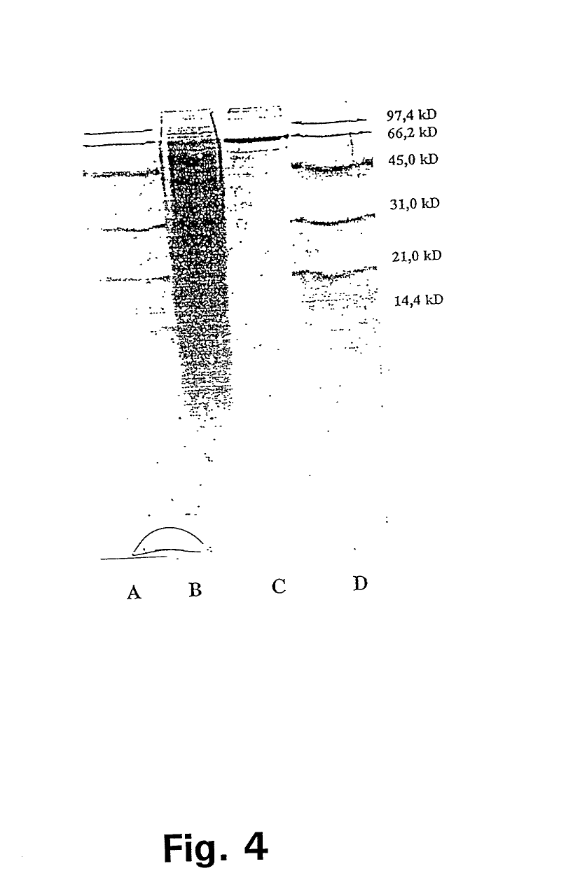

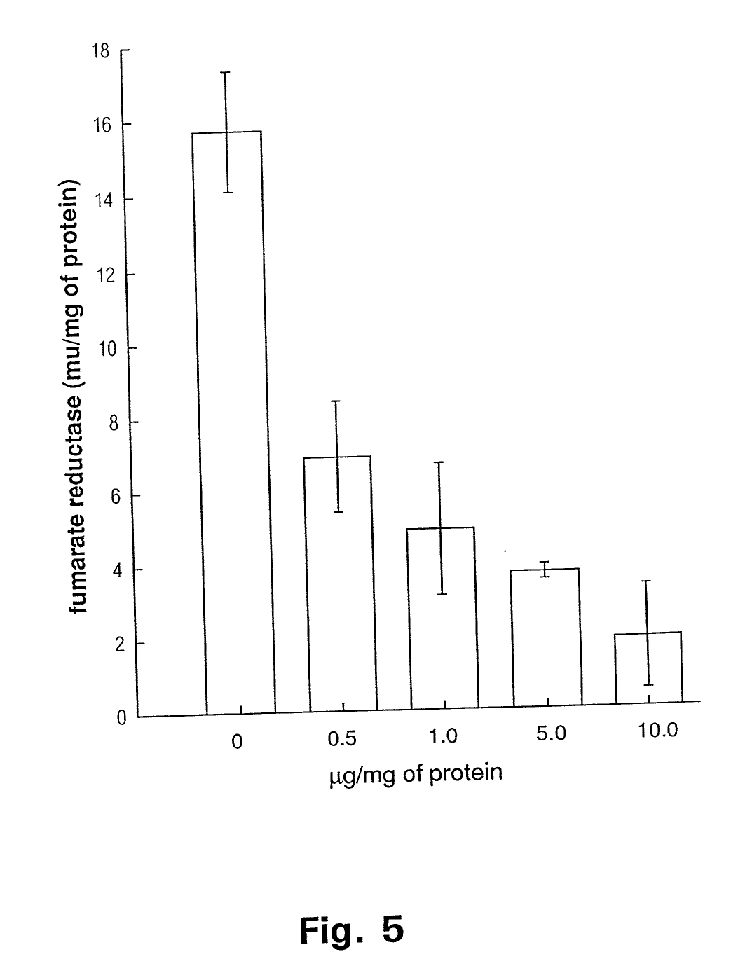

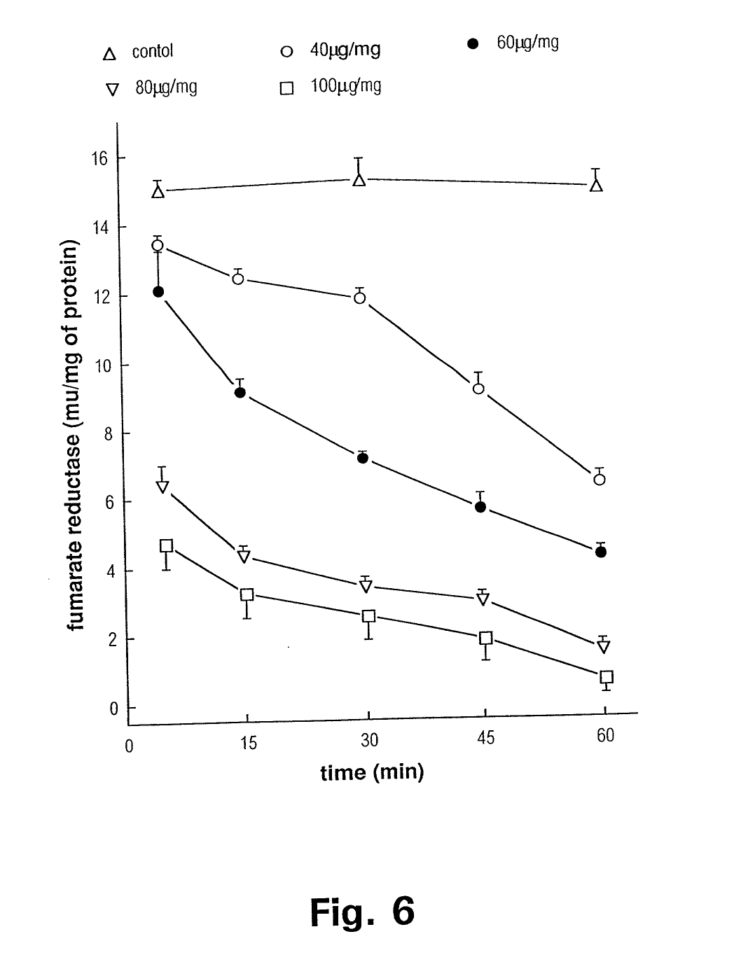

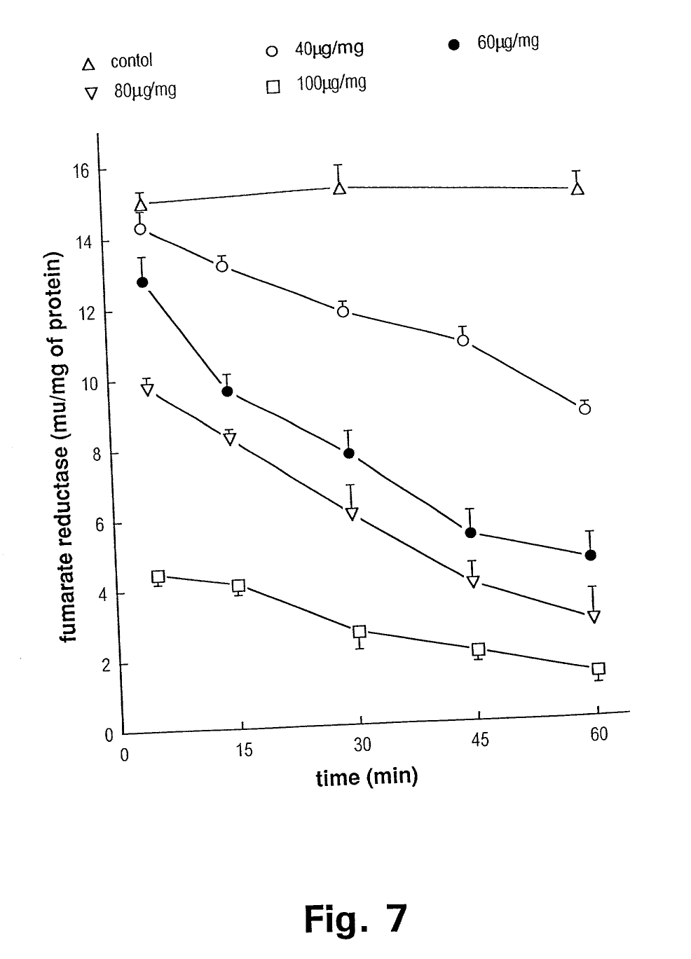

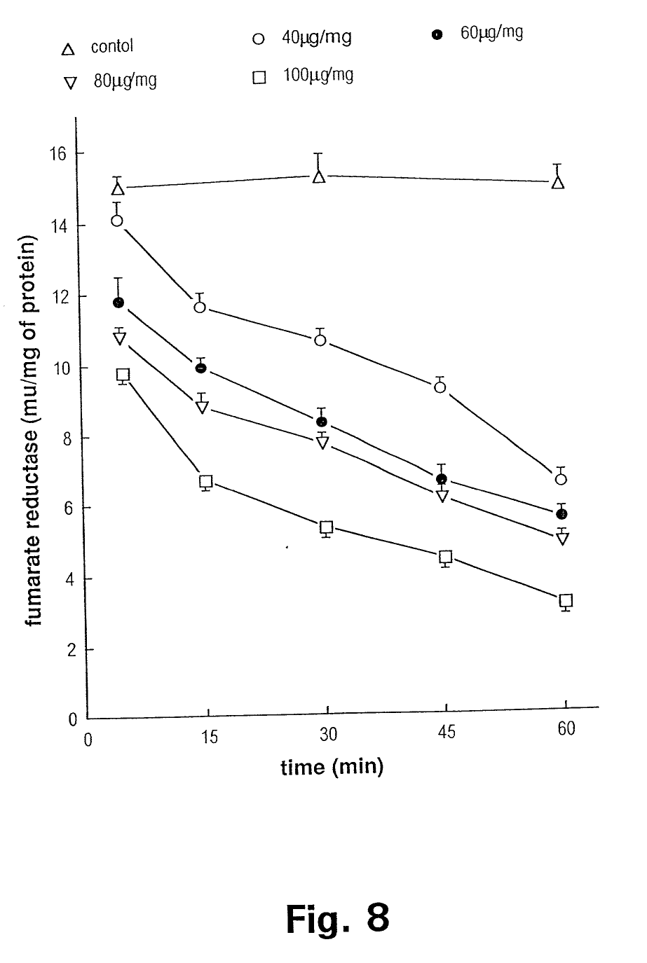

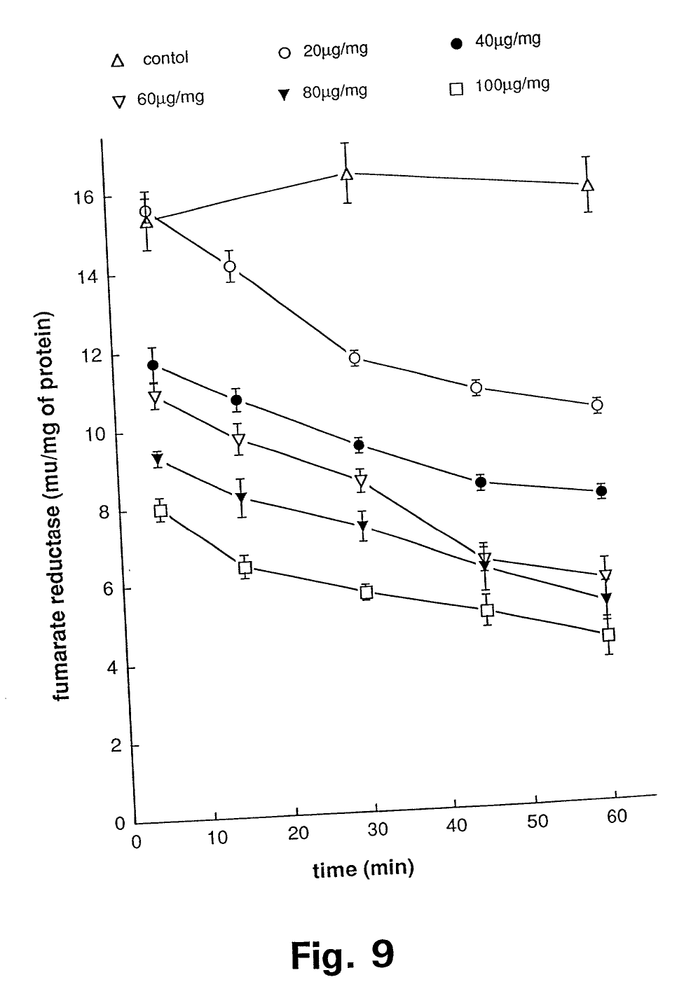

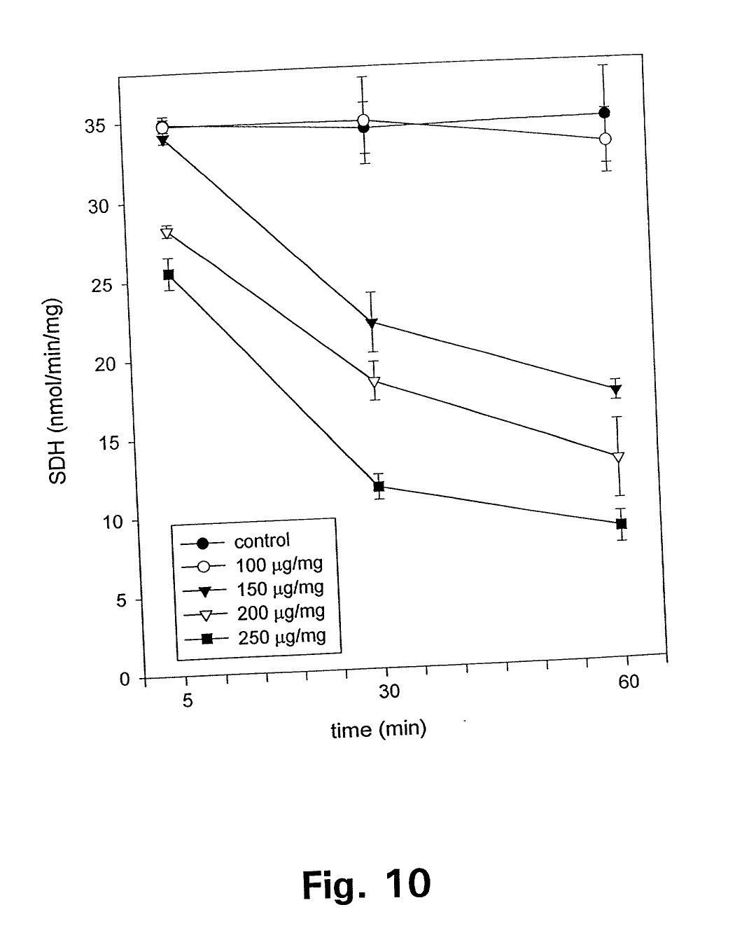

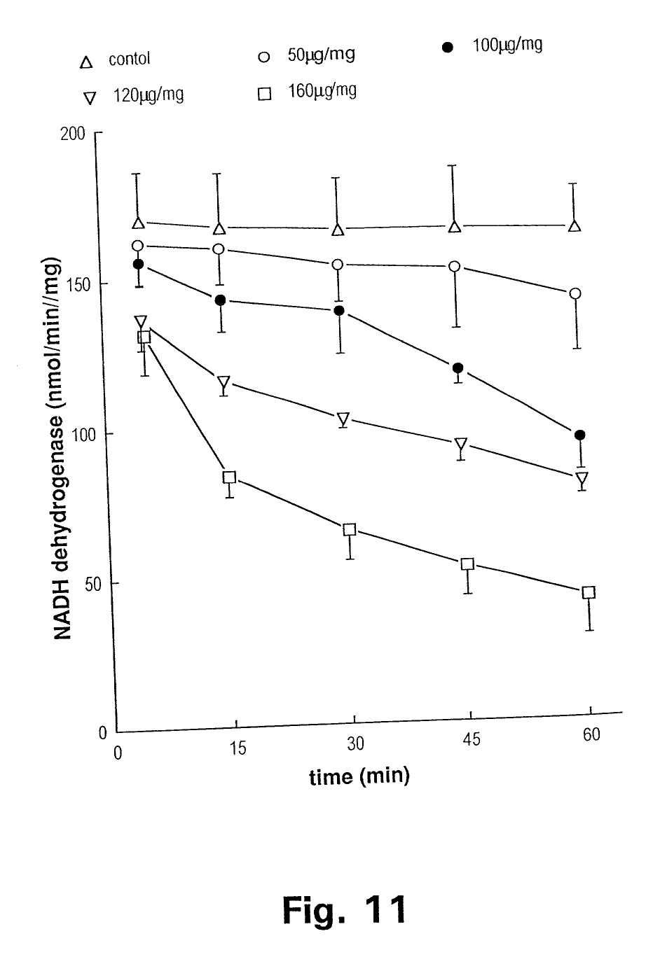

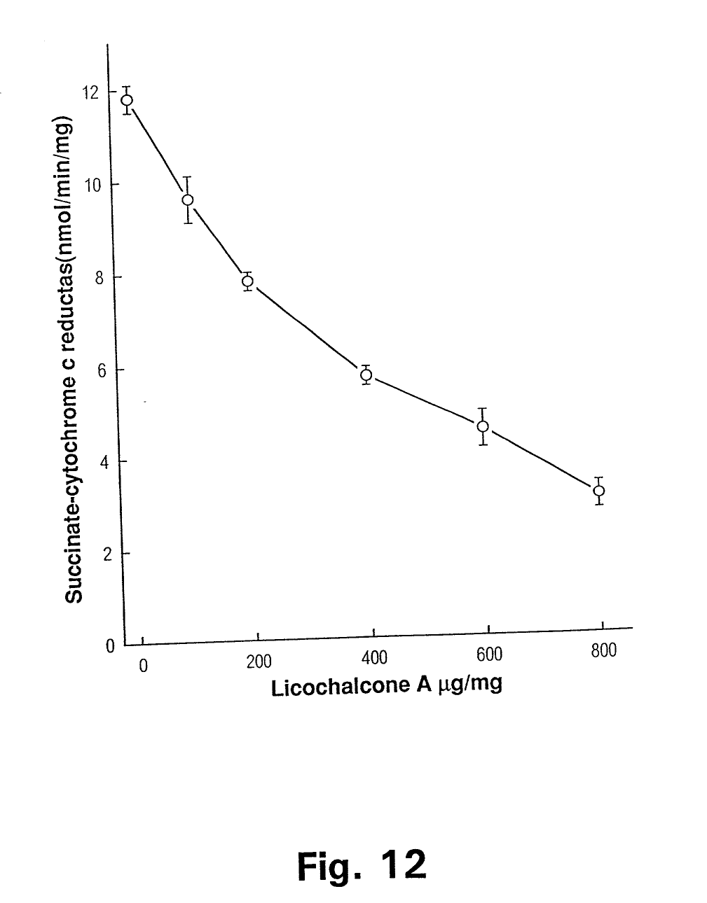

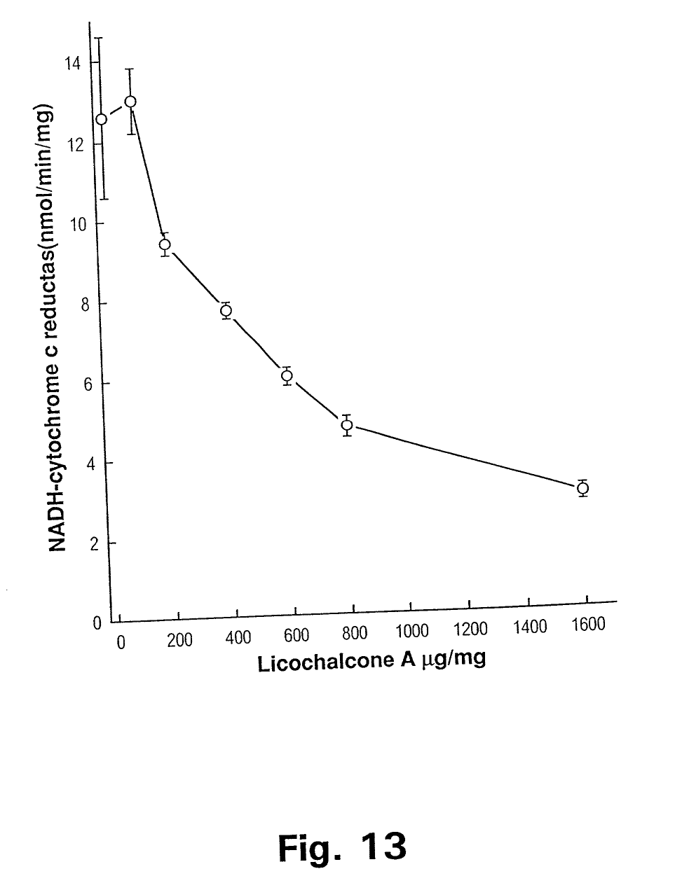

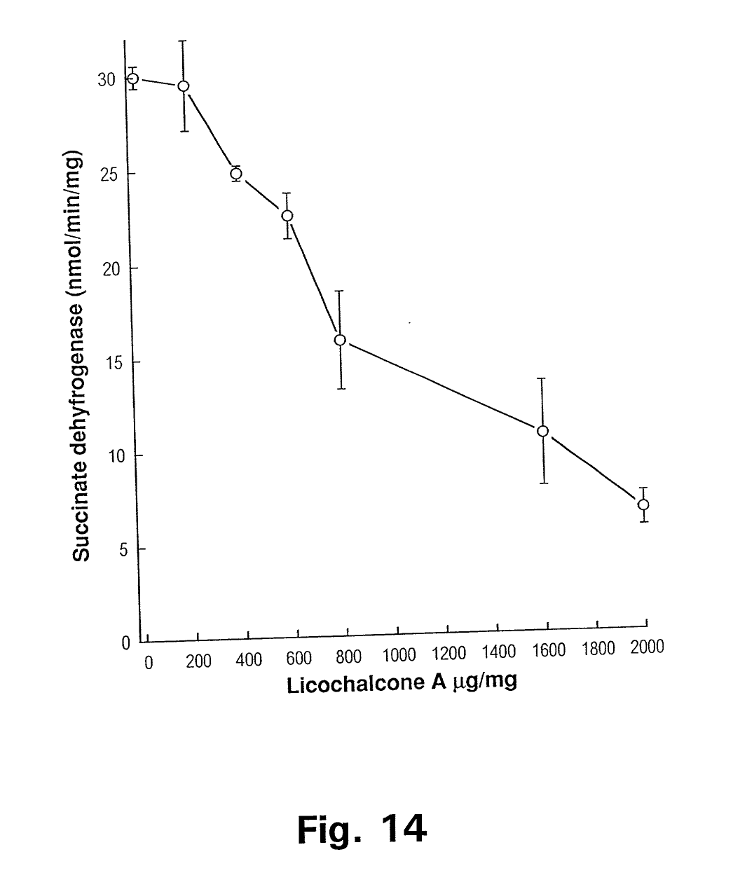

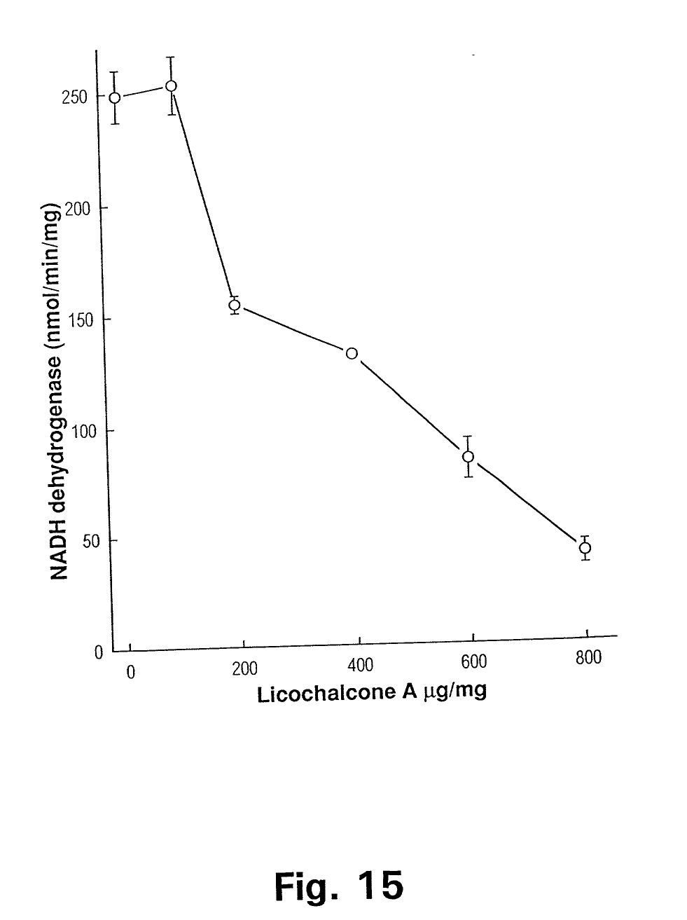

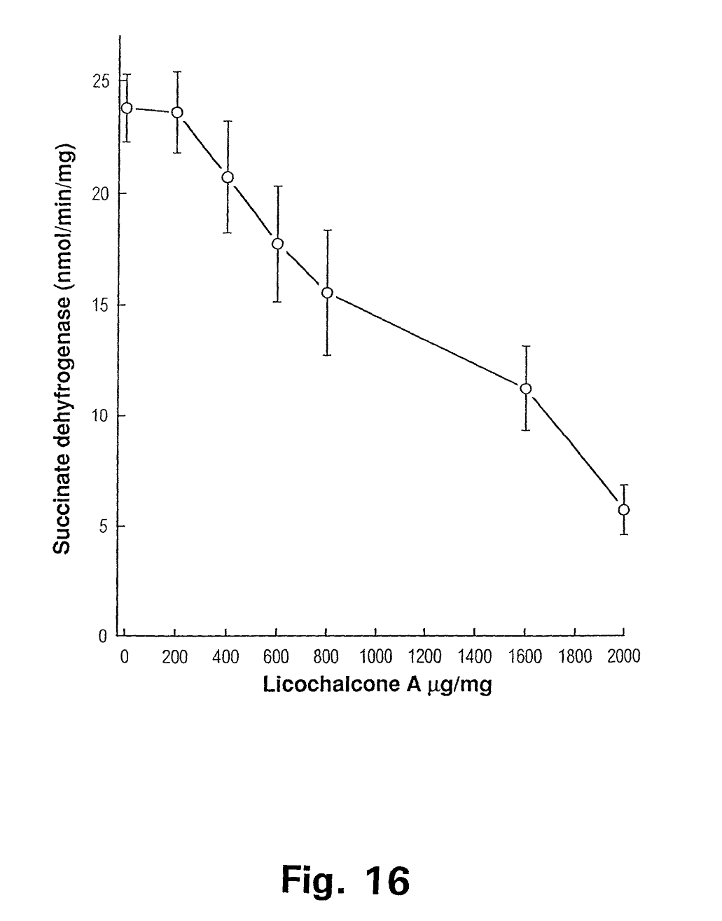

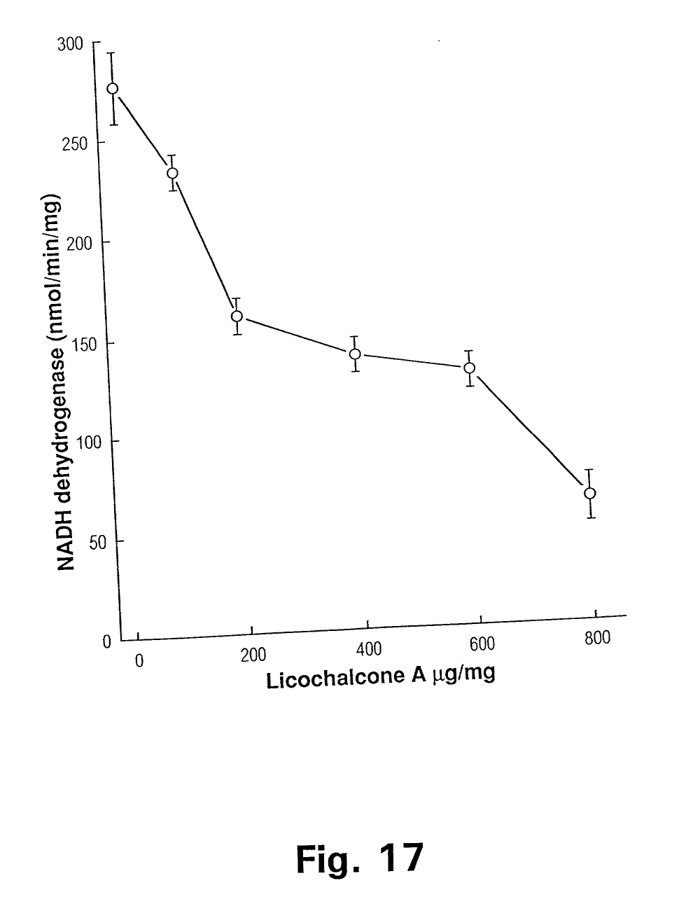

The results are given in FIGS. 3-17 and in the following tables.

TABLE 1
Purification of fumarate reductase
from L. donovani promastigotes

| | | | Specific activity | |
|---|---|---|---|---|
| Purification | Total protein | Total activity | (units/mg of | |
| step | (mg) | (units) | protein) | % Yield |
| Crude extract | 16.9 | 207.87 | 12.3 | 100 |
| Run through | 14.5 | 58.44 | 4.0 | 28.1 |
| Mono Q 7 | 1.9 | 19.95 | 10.5 | 9.6 |
| Mono Q 8 | 0.108 | 8.10 | 75.0 | 3.9 |
| Mono Q 9 | 0.02 | 1.38 | 69.0 | 0.7 |
| Mono Q 10 | 0.012 | 0.73 | 60.6 | 0.4 |
| Mono Q 16 | 0.018 | 2.13 | 118.3 | 1.0 |
| Mono Q 17 | 0.021 | 2.94 | 139.8 | 1.4 |
| Mono Q 19 | 0.018 | 1.16 | 64.5 | 0.6 |
| Mono Q 20 | 0.007 | 0.98 | 139.8 | 0.5 |
| Mono Q 21 | 0.0086 | 6.33 | 735.6 | 3.0 |
| Mono Q 25 | 0.0038 | 8.32 | 2189.2 | 4.0 |
| Mono Q 26 | 0.003 | 0.42 | 140.6 | 0.2 |

TABLE 2
Effect of licochalcone A on FRD, SDH, NDH, SCC and NCC
of L. major Promastigotes and SDH and NDH of human
MNC and J774 cells (incubation at 28° C. for 5 min)

| Enzyme | IC₅₀ (μg/mg protein) |
|---|---|
| FRD (L. major) | 54.7 |
| SDH (L. major) | >250* |
| SDH (human MNC) | 799.7 |
| SDH (J774) | 1122.2 |
| NDH (L. major) | 415.1 |
| NDH (human MNC) | 454.2 |
| NDH (J774) | 547.7 |
| SCC (L. major) | 647.6 |
| NCC (L. major) | 387.5 |

*29% inhibition at 250 μg/mg protein
FRD: Fumarate reductase
SDH: Succinate dehydrogenase
NDH: NADH-dehydrogenase
SCC: Succinate-cytochrome c reductase
NCC: NADH-cytochrome c reductase

Conclusions

We have demonstrated FRD activity in L. donovani and L. major parasites. A fast and convenient method for purification of μg of fumarate reductase from Leishmania parasite have been established.

An active enzyme has been purified, and the measured molecular weight of about 65 kD corresponds approximately with the molecular weight of the flavoprotein subunit which is the active subunit of other known fumarate reductase (The flavoprotein cDNA of Plasmodium falciparum codes for 620 amino acid residues=approx. 68 kD-K. Kita, NCBI international database. 1996).

FRD is a key enzyme in the energy metabolism in a number of microorganisms, including Leishmania and malaria parasites, but absent in human cells. For this reason, FRD is a potentially important target for antileishmanial drugs.

We have already cloned the gene for this enzyme from Plasmodium falciparum and are in the process of cloning the gene/s for this enzyme from Leishmania and Helicobacter pylori and express the genes in appropriate expression vectors. The recombinant enzyme will be crystallised and the crystal structure of the enzyme will be used for molecular modelling to identify compounds with inhibitory activity on this enzyme.

Main References:

A. Denicola-Seoane et al. Mol-Biochem-Parasitol. 1992 54(1):43-50.

H. Rubbo et al. Arch-Biochem-Biophy. 1994 308(1):96-102.

### Example 6

Toxicology

The Danish Toxicology Center has been in charge of all the toxicological tests initiated by the Statens Serum Institut. The testing has primarily been carried out at Inveresk Toxicology, United Kingdom, or Scantox, Denmark.

A 14 day preliminary oral toxicity study and an Ames test were carried out on 6 compounds. All of them were well tolerated and negative in the Ames test. Further mutagenicity testing in vitro of one of these compounds gave positive results in the chromosomal aberration assay in CHO cells and in the mouse lymphona mutation assay. These results initiated screening of several other compounds; both positive and negative results were found.

Single Dose Toxicity

Single dose toxicity of the lead structure, Licochalcone A, in mice showed an LD_{low} po>1000 mg/kg. Also in rats, LD_{low} po>1000 mg/kg was found. Studies carried out later in the project using the dosing schedule, also used to measure effects against the malaria parasites, confirmed the very good oral tolerance to all compounds tested.

Repeated Dose Toxicity and Toxicokinetics

A preliminary 14 days oral toxicity study in rats was carried out with 6 compounds. Observations included clinical signs, body weights, food intake, haematology, clinical chemistry and macroscopic pathology.

No toxic effects were found with

Licochalcone A;

24m4′ac: 2,4-dimethoxy-4′allyloxychalcone,

35m4′ac: 3,5-dimethoxy-4-allyloxychalcone,

24m4′hc: 2,4-dimethoxy-4′hydroxychalcone, or

34m4′ac: 3,4-dimethoxy-4′-allyloxychalcone,

even at the highest dose level used, i.e. 1000 mg/kg/day. An inconclusive effect was seen at this dose level with 4′-hydroxychalcone.

Blood samples were taken 2-4 hours after dosing in the last week and analysed for the compound administered (parent compound) and metabolite(s). Only low levels (parent compound concentration<1 μg/ml) or non-detectable concentrations were found. In a subsequent study, rats were administered 500 mg/kg/day peroral for 3 days. Urine was collected daily, and serum was taken 24 hours after the last dosing. The studies indicate that the peroral bioavailability is low and/or the clearance is high. In mice, the ratio between the effective dose against the malaria parasites following i.p. and p.o. administration points to an acceptable absorption.

Mutagenicity

Ames test has been carried out with 9 compounds. All tests were negative.

Following selection of SBC-35ma (3,5-dimethoxy-4′-allyloxychalcone) as a development candidate, this compound was studied in vitro using the chromosomal aberration assay with CHO cells and the mouse lymphoma tk assay. SBC-35ma showed a clear clastogenic response in the CHO cells both with and without metabolic activation (S9 mix). The mouse lymphoma tk assay was positive with S9 mix and negative/inconclusive without S9 mix. Due to these positive responses, a number of other compounds were studied for their clastogenic potential in a screening design of the CHO cell chromosomal aberration assay. The main overall conclusions drawn from this screening were as follows.

The compounds which were less toxic to the cells also showed to be non- or less clastogenic. Metabolic activation results in higher toxicity and clastogenicity. Four compounds with a more lipophilic side chain (i.e. butoxy or hexoxy in the 4′position) were all negative.

Comparison of the results found in the chromosomal aberration assay with CHO cells and the mouse lymphoma tk assay suggests that the mouse lymphoma tk assay with S9 mix is the most sensitive to show mutagenic effect in this series of compounds. Therefore, a screening design of this assay was used in the search for non-mutagenic compounds. In total, 29compounds have been studied either in the mouse lymphoma tk assay or in the screening design of the assay, with the following results: 7 compounds were negative, 6 were questionable negative, 6 were questionable positive, and 10 were positive. Thus it is expected that non-mutagenic compounds do occur in this series. In the screening design, three known drugs were included, namely chloroquine, pyrimethamine and artesunate. Chloroquine was questionable negative, pyrimethamine was negative, and artesunate was positive.

This series of compounds may be characterised as follows:

All six compounds tested for oral toxicity in a 14 day preliminary study were well tolerated.

Two of the above compounds and another five compounds tested for oral toxicity in a single high-dose study were also well tolerated.

All 14 compounds examined in the Ames test were negative and one was positive.

The in vitro chromosomal aberration assay in CHO cells of 21 compounds showed 6 negative and 15 positive. In total, 29 compounds have been studied either in the mouse lymphoma tk assay or in the screening design of the assay, with the following results: 7 compounds were negative, 6 were questionable negative, 6 were questionable positive, and 10 were positive. Thus it is expected that non-mutagenic compounds do occur in this series.

Thus, overall, it can be concluded that

none exept one of the compounds studied were positive in Ames test

some compounds were negative in clastogen assay

some compounds were negative in mouse lymphoma mutagen assay

a lipophilic side chain decreased the toxicity and the mutagenic response

Non-mutagenic compounds exist amongst the chalcone class of compounds

### Example 7

Anticancer Studies of Bis-aromatic Ketones Like Chalcones and Related Substances

The anti-cancer studies were carried out at Rigshospitalet, Finsen Center, Afdeling L, Copenhagen, Denmark.

Compounds

Compounds which have shown strong activity against human lymphocyte proliferation in the anti-parasite screening studies were chosen for anticancer studies. Based on these data the following compounds were selected for in vitro anticancer studies:

Licochalcone A

Butein

Homobutein

SBC-2,4m4′ac: 2,4-dimethoxy-4′ allyloxychalcone

SBC-4′ac: 4′-allyloxychalcone

SBC-2,3m4′hc: 2,3-dimethoxy-4′-hydroxychalcone

SBC-2,5m4′hc: 2,5-dimethoxy-4′hydroxychalcone

In vitro Studies

The selected compounds were tested for inhibition of the growth of two human cell lines CEM and HL60. The cells were grown in the presence of different concentrations of each compound for 48 hrs, after which cell number was determined in a Coulter counter. Based on these results the IC₅₀ values are determined.

Results

Almost all of the selected compounds at concentrations of 5 μg/ml and higher exhibited potent inhibitory activity against both cell lines.

Two compounds 2,3-dimethoxy-4′-hydroxychalcone and 2,5-dimethoxy-4′hydroxychalcone were selected for in vivo studies. The selection was based on high potency in vitro.

### Example 8

Anti-viral Activity of Bis-aromatic Ketones

In the following is reported the results from a Vero cell culture infected with different viruses. The cell culture used as well as the methods applied are well known to a person skilled in the art.

Experiment 1
Cytotoxicity and antiviral activity of compounds in Vera cell cultures

| | Minimum | Minimum inhibitory concentration^{b} | | | | |
|---|---|---|---|---|---|---|
| | Cytotoxic | Parainfluenza-3 | | Sindbis | Coxsackie | Simliki |
| Compound | Concentration^{a} | Virus | Reovirus-1 | virus | virus B4 | forest virus |
| (1) LicochalconeA | <1/100 | <1/100 | <1/100 | 1/600 | <1/100 | <1/100 |
| (3) 2m4P4′ec | <1/100 | <1/100 | <1/100 | 1/1000 | <1/100 | <1/100 |
| (4) 2,4m4′dc | <1/100 | <1/100 | <1/100 | <1/100 | <1/100 | <1/100 |
| (5) 3,4m4′hc | <1/100 | <1/100 | <1/100 | <1/100 | <1/100 | <1/100 |
| (6) 3,4m4′ac | <1/100 | 1/6000 | <1/100 | 1/60000 | 1/60000 | 1/2000 |
| (7) 3,5m4′ac | <1/100 | <1/100 | <1/100 | <1/100 | <1/100 | <1/100 |
| (8) 2,4m2′hc | <1/100 | <1/100 | <1/100 | <1/100 | <1/100 | <1/100 |
| (9) 2,5m4′ac | <1/100 | <1/100 | <1/100 | <1/100 | <1/100 | <1/100 |
| (10) 2,4mc | <1/100 | <1/100 | <1/100 | <1/100 | <1/100 | <1/100 |
| (11) 2,3m4′ac | <1/100 | <1/100 | <1/100 | <1/100 | <1/100 | <1/100 |
| (15) 2,6m4′hc | <1/100 | 1/600 | <1/100 | 1/2000 | 1/600 | 1/600 |
| (16) 2,4m4′hhc | <1/100 | 1/600 | <1/100 | 1/6000 | 1/2000 | 1/600 |
| (17) 2,4m2′pc | <1/100 | 1/600 | <1/100 | 1/6000 | 1/600 | 1/600 |
| (18) 2,4m4′dpc | <1/100 | 1/600 | <1/100 | 1/2000 | 1/600 | 1/600 |
| (19) 2,4mfc | <1/100 | 1/2000 | 1/200 | 1/2000 | 1/600 | 1/600 |
| (20) 2,4mac | <1/100 | 1/2000 | <1/100 | 1/6000 | 1/2000 | 1/600 |
| (21) 3,4,5m4′ac | <1/100 | 1/2000 | <1/100 | 1/2000 | 1/2000 | 1/600 |
| (22) 2,4,6m4′ac | <1/100 | 1/2000 | <1/100 | 1/2000 | 1/600 | 1/600 |
| (23) dim Lica | <1/100 | 1/2000 | <1/100 | 1/2000 | 1/2000 | 1/600 |
| (24) 2,4,5m4′ac | <1/100 | 1/2000 | <1/100 | 1/2000 | 1/600 | 1/600 |
| CM-1 | <1/3000 | <1/10000 | <1/10000 | <1/10000 | <1/10000 | <1/10000 |

(control virus inhibitor)

Activity of compounds against
cytomegaloviurs in human embryonic lung (HEL) cells

| | Antiviral activity | | |
|---|---|---|---|
| | IC₅₀^{a} | | |
| | AD-169 strain | Davis strain | Cytotoxicity |
| Compound | Assay 1 | Assay 1 | CC₅₀^{b} |
| 1. Licochalcone A | 1/15000 | 1/50000 | 1/600 |
| 3. 2m4p4′ec | <1/300 | 1/500 | 1/300 |
| 4. 2,4m4′dc | 1/200 | 1/500 | <1/60 |
| 5. 3,4m4′hc | 1/45 | 1/55 | <1/30 |
| 6. 3,4m4′ac | <1/30 | 1/100 | <1/30 |
| 7. 3,5m4′ac | <1/60 | <1/60 | <1/60 |
| 8. 2,4m2′hc | <1/60 | <1/60 | <1/60 |
| 9. 2,5m4′ac | 1/35 | 1/55 | <1/30 |
| 10. 2,4mc | <1/30 | 1/35 | <1/30 |
| 11. 2,3m4′ac | <1/30 | <1/30 | <1/30 |
| 15. 2,6m4′hc | 1/150 | 1/300 | 1/80 |
| 16. 2,4m4′hhc | 1/100 | 1/200 | <1/60 |
| 17. 2,4m2′pc | 1/120 | 1/200 | <1/30 |
| 18. 2,4m4′dpc | 1/130 | 1/160 | 1/70 |
| 19. 2,4mfc | 1/200 | 1/200 | 1/40 |
| 20. 2,4mpoc | <1/60 | 1/60 | <1/60 |
| 21. 3,4,5m4′ac | 1/150 | 1/85 | <1/60 |
| 22. 2,4,6m4′ac | 1/200 | 1/120 | <1/30 |
| 23. dim Lica | 1/100 | 1/140 | <1/30 |
| 24. 2,4,5m4′ac | 1/60 | <1/30 | <1/30 |
| CM-1 | 1/2000 | 1/2000 | <1/500 |
| DS 5000 | 0.85 μg/ml | 0.5 μg/ml | >200 μg/ml |

^{a}50% Inhibitory concentration, or concentration required to reduce virus plaque formation by 50%. Virus input was 100 plaque forming units (PFU).
^{b}50% Cytotoxic concentration, or concentration required to reduce cell growth by 50%.

### Example 9

Chalcones as Inhibitors of Inflammatory Cytokines

The objective of this study was to investigate the effect of certain oxygenated chalcones on the induction, production and secretion of inflammatory cytokines in in vitro and in vivo models and on protection of mice from LPS-induced septic shock.

The following test systems have been employed:

1a. Production of TNF-α by human peripheral blood mononuclear cells stimulated in vitro by LPS or SPAG. Detection in culture supernatant (ELISA).

1b. Production of TNF-α by human peripheral blood mononuclear cells stimulated in vitro by LPS or SPAG. Detection of intracellular TNF-α (Flow cytometry).

2a. Production of TNF-α in a mouse model of LPS-induced septic shock. Detection in plasma (ELISA).

2b. Protection of mice from a lethal effect of LPS-induced septic shock. Efficacy (survival of treated animals).

3. Protection of mice from a lethal effect of cerebral malaria. Efficacy (survival of treated animals).

1a. Production of TNF-α by Human Peripheral Blood Mononuclear Cells Stimulated in vitro by LPS or SPAG. Detection in Culture Supernatant (ELISA).

Materials and Methods

Cells: Peripheral blood mononuclear cells obtained from healthy individuals. The cells were separated by a conventional method lymphoprep) and suspended in RMPI 1640 containing 5% human serum.

Compounds: Licochalcone A, SBC-35ma (3,5-dimethoxy-4′-allyloxychalcone), SBC-24ma (2,4-dimethoxy-4′-allyloxychalcone) and SBC-24mbc (2,4-dimethoxy-4′-butoxychalcone) and several other compounds as shown in the results section were synthesised either by Clauson Kaas AS, Farum, Denmark according to GMP rules or by one of the inventors.

Malaria exoantigens and bacterial LPS: Exoantigens were affinity purified from culture medium of P. falciparum essentially as described previously (Jepsen & Andersen, 1981; Jakobsen et al., 1988) using as ligand a pool of IgG from clinically immune African adults. Before chromatography the culture medium was centrifuged at 7000 g for 10 min, filtered through a 0.22 μm membrane and dialysed overnight at 4° C. against column buffer. LPS was purchased from Sigma Chem. Co.

Stimulation of cytokine-release from human mononuclear cells: Human peripheral blood mononuclear cells from Danish donors were suspended in 1-10% (v/v) human serum in RPMI 1640 and adjusted to 2×10⁶ cells per ml. 0.1 ml volumes were then dispensed into wells of 96-well microtiter plates. Drugs in two-fold dilutions (0.25-5 μg/ml) and stimulation antigens (LPS or malaria parasite exoantigens) at optimal dilutions diluted in RPMI 1640 were then added to a total volume of 0.2 ml per well. In some experiments cells were preincubated with drugs for 2 hrs before addition of stimulation antigens. The cultures were incubated overnight for TNF-α and IL-6 assays.

Supernatants were collected and assayed for TNF-α, IL-1α and IL-6 by ELISA procedures (Jakobsen et al., 1993, Fomsgaard et al., 1988, Hansen et al., 1991) with the following modifications. ELISA Maxisorp plates (Nunc, Roskilde, Denmark) were coated for 24 hrs at 4 C with 100 μl per well of 2.5 μg/ml rabbit polyclonal IgG to human recombinant TNF, IL-1α or IL-6 in 100 mM sodiumhydrogencarbonate. Non-attached sites were blocked for 1 h by 200 μl per well of 2% human serum slbumin in PBS (1 h at 37° C.), and the wells were washed 4 times in 2.5% NaCl, 0.1% Tween 20 (Merck, Darmstadt, Germany). This washing procedure was done after each of the following incubating steps:

1) 100 μl culture supernatants made to 50% (v/v) in incubation buffer (4% (v/v) normal rabbit serum, DAKO code X902), 1% polyethylene glycol (MW 6,000), 2.5% NaCl, 0.1% Tween 20 in phosphate buffered saline (PBS) were incubated 2 hrs at 37° C., dilutions of native as well as recombinant cytokine standards in the same incubation buffer were asssayed in parallel with the tested supernatants,

2) 100 μl per well of biotinilated rabbit antibodies to recombinant TNF-α, IL-1α or IL-6 (1.5 μg/ml in 0.5% human serum albumin, 0.1% Tween 20 in PBS) were added at 37° for 2 hrs,

3) 100 μg per well of a 1:1000 dilution of streptavidin-peroxidase (Amersham, UK), at room temperature for 30 min.

Enzyme activities were quantitated after addition of 100 μl per well of 0.67 mg/ml 1,2-phenyldiamine hydrochloride (DAKO) dissolved in 100 mM citric acid-phosphate buffer, pH 5.0 containing 0.015% (v/v) H₂O₂. The reactions were stopped by adding 50 μl per well of 2.5 M H₂SO₄, and the optical densities are measured in an ELISA scanner at 490 nm against a test reference at 620 nm.

Results

Table 1. Effect of Licochalcone A and ABC-35ma on TNF-α production by human peripheral blook mononuclear cells stimulated either with a malarial exoantigen SPAG (diluted 1:12.5) or bacterial LPS (diluted 1:1000). The cells were preincubated with different concentrations of each of the compounds for 2 hrs and then incubated in the presence of antigens at 27° C. overnigth. The supernatants were then harvested and used in the ELISA assay. Data are presented as percentage inhibition of cell response as compared to stimulated cells in the absence of the compounds.

| | | |
|---|---|---|
| Drug Conc. | Licochalcone A | SBV-35ma |

| μg/ml | SPAG | LPS | SPAG | LPS |
|---|---|---|---|---|
| 20 | 97 | 81 | 100 | 73 |
| 10 | 44 | 40 | 55 | 56 |
| 5 | 13 | 0 | 0 | 1 |
| 1 | 0 | 0 | 0 | 0 |

Table 2. Effect of Licochalcone A and SBC-35ma on TNF-α production by human peripheral blood mononuclear cells stimulated either with a malarial exoantigen SPAG (diluted 1:12.5) or bacterial LPS (diluted 1:1000). The cells were preincubated with different concentrations of each of the compounds for 2 hours and then incubated in the presence of antigens at 37° C. overnight. The supernatants were then harvested and used in the ELISA assay. Data are presented as percentage inhibition of cell response as compared to stimulated cells in the absence of the compounds.

| | | |
|---|---|---|
| Drug Conc. | Licochalcone A | SBC-35ma |

| μg/ml | SPAG | LPS | SPAG | LPS |
|---|---|---|---|---|
| 20 | 100 | 54 | 100 | 33 |
| 12.5 | 94 | 38 | 100 | 28 |
| 6.25 | 82 | 3 | 99 | 23 |
| 1.25 | 54 | 4 | 87 | 2 |

Based on the results presented in the above tables 1 and 2 it was questioned whether the lack of TNF-α in the supernatant of cells preincubated with these compounds was due to inhibition of the production of TNF-α or secretion of the produced product from the cells. Therefore, experiments were performed to examine the effect of Licochalcone A and SBC-35ma on secretion of TNF-α from the stimulated cells. These experiments included detection of the cytokines in the cell lysate by ELISA and staining of the intracellular cytokines by using monoclonal antibodies against these cytokines and cytokine receptors.

By using ELISA method it was shown that there was no intracellular TNF-α present in the lysate of cells preincubated with either Licochalcone A or SBC-35ma and stimulated with SPAG or LPS.

The results of intracelular staining of the cytokines are shown in Table 3

Table 3. Effect of Licochalcone A and SBC-35ma on the presence of intracellur cytokines in the human peripheral blood mononuclear cells stimulated either with a malarial exoantigen SPAG (diluted 1:12.5) or bacterial LPS (diluted 1:1000). The cells were preincubated with 25 μg/ml of each of the compounds for 2 hours and then incubated in the presence of antigens at 37° C. overnight. The cells were permeabilized and then fixed in paraformaldehyde Cytokine specific monoclonal antibodies were used for the detection of positive cells for each cytokine. Data are presented as percentages of positive cells for each cytokine.

| | IL-1α | IL-1β | IL-1ra | IL-2 | IL-4 | IL-6 | TNF-α |
|---|---|---|---|---|---|---|---|
| SPAG | 11 | 13 | 6.6 | 3.1 | 0 | 5.6 | 0.96 |
| SPAG + | 9.1 | 13 | 2.9 | 5.2 | 0.69 | 6.2 | 1.0 |
| Licochalcone A | | | | | | | |
| SPAG + | 0 | 0 | 0.14 | 0 | 0 | 0 | 0 |
| SBC-35ma | | | | | | | |
| LPS | 13 | 9.1 | 5.1 | 2.0 | 0 | 4.3 | 0.30 |
| LPS + | 15 | 12 | 7.6 | 3.6 | 0 | 6.7 | 0.70 |
| Licochalcone A | | | | | | | |
| LPS + | 0 | 0 | 0.78 | 0 | 0 | 0 | 0 |
| SBC-35ma | | | | | | | |

Further tests were made where the experimental set-up was the same as described above.

Compounds:

PH32: 4′-dimethylamino-3,5-dimethoxychalcone

PH81: 3′-butoxy-2,4-dimethoxychalcone

PH104: 4′-cyclohexyl-3,4-dimethoxychalcone

PH105: 4′-nitro-3,5-dimethoxychalcone

PH135: 2′-fluoro-3,5-dimethoxychalcone

PH136: 3′-fluoro-3,5-dimethoxychalcone

PH142: 2′-butoxy-2,4-dimethoxychalcone

Results

PH81, PH135, PH136 and PH142 at concentrations of 12.5 μg/ml and higher inhibited the production of TNF-α by greater than 95%

PH104 at a concentration of 12.5 μg/ml inhibited the production of TNF-α by approximately 75% and at higher concentrations greater than 75%,

PH32 and PH105 had no or very little inhibitory effect on TNF-α production

Determination of IC₅₀ values of TNF-α production by human peripheral blood mononuclear cells in vitro

A dilution row was prepared and the inhibition of the TNF-α activity was determined as described above. IC₅₀ values were then calculates as the concentration at which 50% of the TNF-α production was inhibited by the compound investigated.

Still further experiments were performed in accordance with the experimental set-up described above.

Compounds tested: PH32, PH104, PH105, PH135, PH136 and PH142.

Cells: Peripheral blood mononuclear cells were isolated from healthy individuals by Lymphoprep separation.

Assay: The compounds were prepared as stock solution at a concentration of 1 mg/ml in medium containing 10% DMSO. The dilutions were made in medium. The cells were stimulated with either SPAG (a malaria antigen TNF-inducer) or bacterial LPS. The supernatant of cell cultures were assayed for TNF-α using a commercial ELISA kit.

Two series of experiments were conducted in order to obtain information on a dose-response inhibitory activity of chalcones on TNF-α production in vitro.

Results

Experiment 1. Effect of some selected chalcones on TNF-α production by human peripheral blood mononuclear cells stimulated in vitro. The data are shown as OD values.

| Compounds (μg/ml) | SPAG | LPS |
|---|---|---|
| PH32: | | |
| 100 μg/ml | 0.75 | 0.94 |
| 50 | 1.74 | 1.28 |
| 25 | 2.26 | 1.55 |
| 12.5 | 3.90 | 1.23 |
| PH104: | | |
| 100 μg/ml | 0.01 | 0.31 |
| 50 | 0.03 | 0.41 |
| 25 | 0.13 | 0.59 |
| 12.5 | 0.93 | 1.15 |
| PH105: | | |
| 100 μg/ml | 2.3 | 0.99 |
| 50 | 4.00 | 1.69 |
| 25 | 2.00 | 1.13 |
| 12.5 | 1.94 | 1.07 |
| PH135: | | |
| 100 μg/ml | 0.01 | 0.02 |
| 50 | 0.01 | 0.01 |
| 25 | 0.01 | 0.01 |
| 12.5 | 0.02 | 0.01 |
| PH136: | | |
| 100 μg/ml | 0.00 | 0.01 |
| 50 | 0.01 | 0.00 |
| 25 | 0.00 | 0.00 |
| 12.5 | 0.01 | 0.00 |
| PH142: | | |
| 100 μg/ml | 0.02 | 0.10 |
| 50 | 0.02 | 0.18 |
| 25 | 0.02 | 0.24 |
| 12.5 | 0.02 | 0.26 |
| Control | 3.73 | 1.17 |

Experiment 2. Effect of some selected chalcones on TNF-α production by human peripheral blood mononuclear cells stimulated in vitro. The data are shown as OD values.

| Compounds (μg/ml) | SPAG | LPS |
|---|---|---|
| PH104: | | |
| 12.5 μg/ml | 0.43 | 0.91 |
| 6.25 | 1.50 | 1.08 |
| 3.125 | 2.44 | 1.41 |
| 1.562 | 2.47 | 1.54 |
| PH135: | | |
| 12.5 μg/ml | 0.01 | 0.04 |
| 6.25 | 0.03 | 0.09 |
| 3.125 | 0.04 | 0.49 |
| 1.562 | 0.93 | 0.89 |
| PH136: | | |
| 12.5 μg/ml | 0.02 | 0.08 |
| 6.25 | 1.39 | 2.18 |
| 3.125 | 4.00 | 1.81 |
| 1.562 | 3.29 | 1.70 |
| PH142: | | |
| 12.5 μg/ml | 0.02 | 0.16 |
| 6.25 | 0.04 | 0.65 |
| 3.125 | 0.55 | 1.00 |
| 1.562 | 1.86 | 1.58 |
| Control | 2.28 | 2.06 |

1b. Production of TNF-α by Human Peripheral Blood Mononuclear Cells Stimulated in vitro by LPS or SPAG. Detection of Intracellular TNF-α (Flow cytometry)

PBMC (2 × 10⁶/ml) + compound + LPS (1 μg/ml) + monensin
↓
Incubate at 37° C. in CO₂ incubator overnight
↓
Wash in PBS
↓
Label with PE-conjugated anti-CD14
↓
Wash and fix with 2% formaldehyde
↓
Wash twice in 1% saponin buffer
↓
Incubate with FITC conjugated anti-TNF-α antibody (30 min)
↓
Wash twice in 1% saponin buffer and once in PBS
↓
Cell analysis by flow cytometry (forward and side scatter gatings)

Experiment 1—Effect of PH104 and PH135 on Intracellular TNF-α in Human Mononuclear Cells

Compounds: PH104 and PH135. Final concentrations of 25, 12.5 and 6.25 μg/ml.

Cells: Peripheral blood mononuclear cells (MNC) 2×10⁶/ml.

Mitogens: LPS at a final conc. of 0.5 μg/ml.

Incubation condition: Cells and mitogen added at the same time and the compound solution added immediately after in polypropylene tubes and incubated at 37° C. in CO2 incubator overnight. The following day the cells and the supernatant were harvested for TNF-α determination.

Results:

| μg/ml PH135 | % TNF-α producing cells | % inhibition |
|---|---|---|
| 0 (DMSO) | 13.8 | |
| 6.25 | 4.1 | 70 |
| 12.5 | 2.3 | 83 |
| 25.0 | 2.2 | 84 |
| μg/ml PH104 | % TNF-α producing cells | |
| 0 (DMSO) | 13.8 | |
| 6.25 | ND (not done because | |
| | of shortage of cells) | |
| 12.5 | 2.4 | 83 |
| 25.0 | 1.4 | 90 |

Experiment 2—Effect of PH104 and PH135 on Intracellular and Extracellular TNF-α in Human Peripheral Blood Mononuclear Cells

Compounds: PH104 and PH135. Final concentrations of 12.5, 6.25 and 3.15 μg/ml.

Cells: Peripheral blood mononuclear cells (MNC) 2×10⁶/ml.

Mitogens: LPS at a final conc. of 1 μg/ml for TNF-α and PMA+inomycin for IFN-γ.

Incubation condition: Cells and mitogen added at the same time and the compound solution added immediately after in polypropylene tubes and incubated at 37° C. in CO2 incubator overnight. The following day the cells and the supernatant were harvested for TNF-α determination.

Results:

| | Intracellular | Extracellular | |
|---|---|---|---|
| μg/ml PH135 | % TNF-α producing cells | % inhibition | % inhibition |
| 0 (DMSO) | 4.0 | 0 | 0 |
| 3.125 | 4.3 | 0 | 96 |
| 6.25 | 1.7 | 58 | 94 |
| 12.5 | 2.0 | 50 | 94 |
| μg/ml PH104 | % TNF-α producing cells | | |
| 0 (DMSO) | 4.0 | 0 | 0 |
| 3.125 | 2.4 | 40 | 82 |
| 6.25 | 1.0 | 75 | 90 |
| 12.5 | 1.7 | 58 | 93 |

| | % IFN-γ producing cells | % inhibition |
|---|---|---|
| DMSO Control | 16.4 | |
| PH135 12.5 μg/ml | 14.2 | 14 |
| PH104 12.5 μg/ml | 17.0 | 0 |

Experiment 3—Effect of PH104 and PH135 on Intracellular and Extracellular TNF-α in Human Peripheral Blood Mononuclear Cells

Compounds: PH104 and PH135. Final concentrations of 12.5, 6.25 and 3.15 μg/ml.

Cells: Peripheral blood mononuclear cells (MNC) 2×10⁶/ml.

Mitogens: LPS at a final conc. of 1 μg/ml for TNF-α and PMA+inomycin for IFN-γ.

Incubation condition: Cells and mitogen added at the same time and the compound solution added immediately after in polypropylene tubes and incubated at 37° C. in CO2 incubator overnight. The following day the cells and the supernatant were harvested for TNF-α determination.

Results:

| | Intracellular | Extracellular | |
|---|---|---|---|
| μg/ml PH135 | % TNFα- producing cells | % inhibition | % inhibition |
| 0 (DMSO) | 10.1 | 0 | 0 |
| 3.125 | 1.7 | 83 | 93 |
| 6.25 | 1.2 | 88 | 92 |
| 12.5 | 1.0 | 90 | 94 |
| μg/ml PH104 | % TNF-α producing cells | | |
| 0 (DMSO) | 10.1 | 0 | 0 |
| 3.125 | 2.9 | 71 | 54 |
| 6.25 | 1.1 | 89 | 70 |
| 12.5 | 1.2 | 88 | 76 |

Lots of IFN-γ production and no inhibition by PH135 and PH104 at concentration of 12.5 μg/ml was observed.

Experiment 4—Effect of PH104 and PH135 on Intracellular and Extracellular TNF-α in Human Peripheral Blood Mononuclear Cells

Compounds: PH104 and PH135. Final concentrations of 12.5, 6.25 and 3.15 μg/ml.

Cells: Peripheral blood mononuclear cells (MNC) 2×10⁶/ml.

Mitogens: LPS at a final conc. of 1 μg/ml for TNF-α and PMA+inomycin for IFN-gamma.

Incubation condition: Cells and mitogen added at the same time and the compound solution added immediately after in polypropylene tubes and incubated at 37° C. in CO2 incubator overnight. The following day the cells and the supernatant were harvested for TNF-α determination.

Results:

| | Intracellular | Extracellular | |
|---|---|---|---|
| μg/ml PH135 | % TNF-α producing cells | % inhibition | % inhibition |
| 0 (DMSO) | 14.6 | 0 | 0 |
| 3.125 | 3.5 | 76 | 99 |
| 6.25 | 2.1 | 86 | 99 |
| 12.5 | 1.8 | 88 | 98 |
| μg/ml PH104 | % TNF-α producing cells | | |
| 0 (DMSO) | 14.6 | 0 | 0 |
| 3.125 | 3.0 | 79 | 0 |
| 6.25 | 0.8 | 95 | 78 |
| 12.5 | 1.0 | 93 | 86 |

Lots of IFN-γ production and no inhibition by PH135 and PH104 at concentration of 12.5 μg/ml was observed.

2a. Production of TNF-α in a Mouse Model of LPS-induced Septic Shock. Detection in Plasma (ELISA)

Effect of PH135 and PH104 by Oral Route on LPS-induced TNF-α Production in BALB/c Mice

Mice: BALB/c, female, 20 g. Each group consists of 8 mice. Animals were maintained under standard laboratory conditions with food and water ad libitum. Mice have been immunized with BCG (standard BCG vaccine from SSI) 0.2 ml/mouse for 12 days before LPS administration.

LPS: Lipopolysaccharide B, Escherichia coli J5, Sigma, 250 μg/kg (200 μl of 25 μg LPS/ml in sterile pyrogen-free saline, equal to 5 μg/mouse), i.p.

PH135 (A) and PH104 (B) will be micronized in 0.2% Na-CMC and 0.01% tween 80 and given til mice by oral route in total dose of 150 mg/kg.

Treatment Schedule:

Group I. (compound A). 300 μl of the 3.3 mg/ml suspension per mouse by oral route at: 1 hour before LPS injection, same time as LPS, an 1 hour after LPS injection (3 treatments, total 150 mg/kg).

Group II. (compound B). 300 μl of the 3.3 mg/ml suspension per mouse by oral route at: 1 hour before LPS injection, same time as LPS, an 1 hour after LPS injection (3 treatments, total 150 mg/kg).

Group III (compound A). 300 μl of the 10 mg/ml suspension per mouse by oral route at: same time as LPS injection (one treatment, total 150 mg/kg).

Group IV (compound B). 300 μl of the 10 mg/ml suspension per mouse by oral route at: same time as LPS injection (one treatment, total 150 mg/kg).

Group V. control (no treatment, only LPS injection).

Blood (10 mM EDTA-plasma) will be taken from each mouse for TNF-α and LPS measurements at: 1) before LPS injection, 2) 1 hour after LPS injection, 3) 2 hours after LPS injection, and 4) 4 hours after LPS injection.

| group | 7:30 | 8:00 | 8:30 | 9:30 | 10:30 | 12:30 |
|---|---|---|---|---|---|---|
| I | 1^{st} adm. | TAP-0 | LPS + | 3^{rd} adm | TAP-2 | TAP-3 |
| | | | 2^{nd} adm | TAP-1 | | |
| II | 1^{st} adm. | TAP-0 | LPS + | 3^{rd} adm. | TAP-2 | TAP-3 |
| | | | 2^{nd} adm. | TAP-1 | | |
| III | | TAP-0 | LPS + | TAP-1 | TAP-2 | TAP-3 |
| | | | oral adm. | | | |
| IV | | TAP-0 | LPS + | TAP-1 | TAP-2 | TAP-3 |
| | | | oral adm. | | | |
| V | | TAP-0 | LPS | TAP-1 | TAP-2 | TAP-3 |

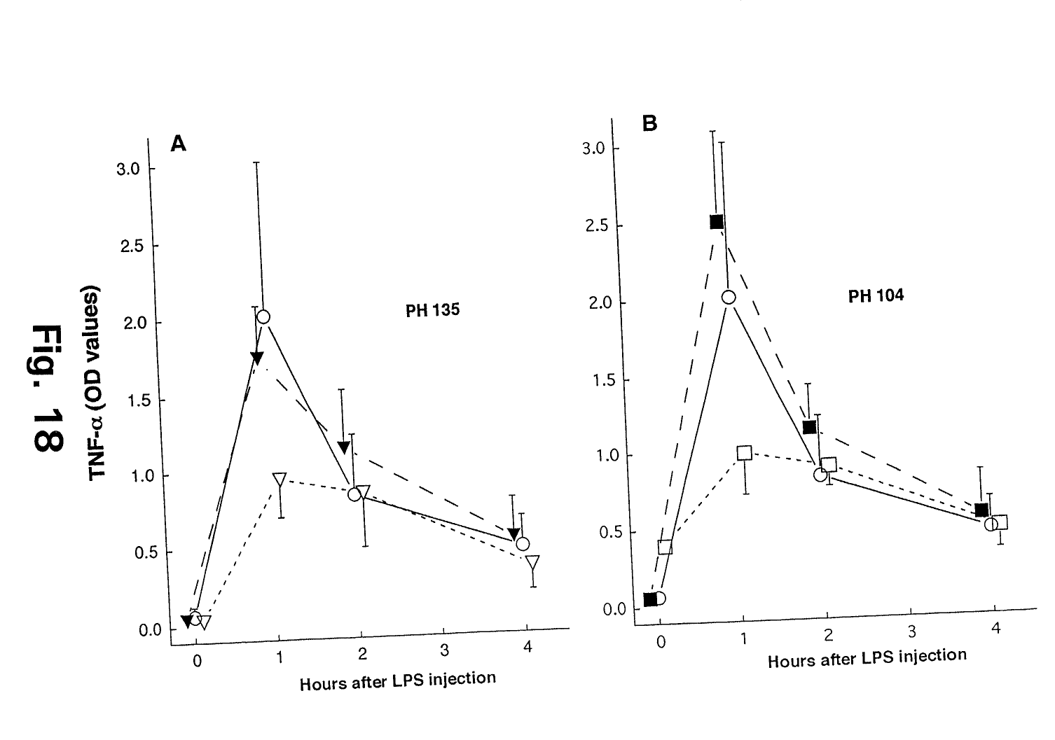

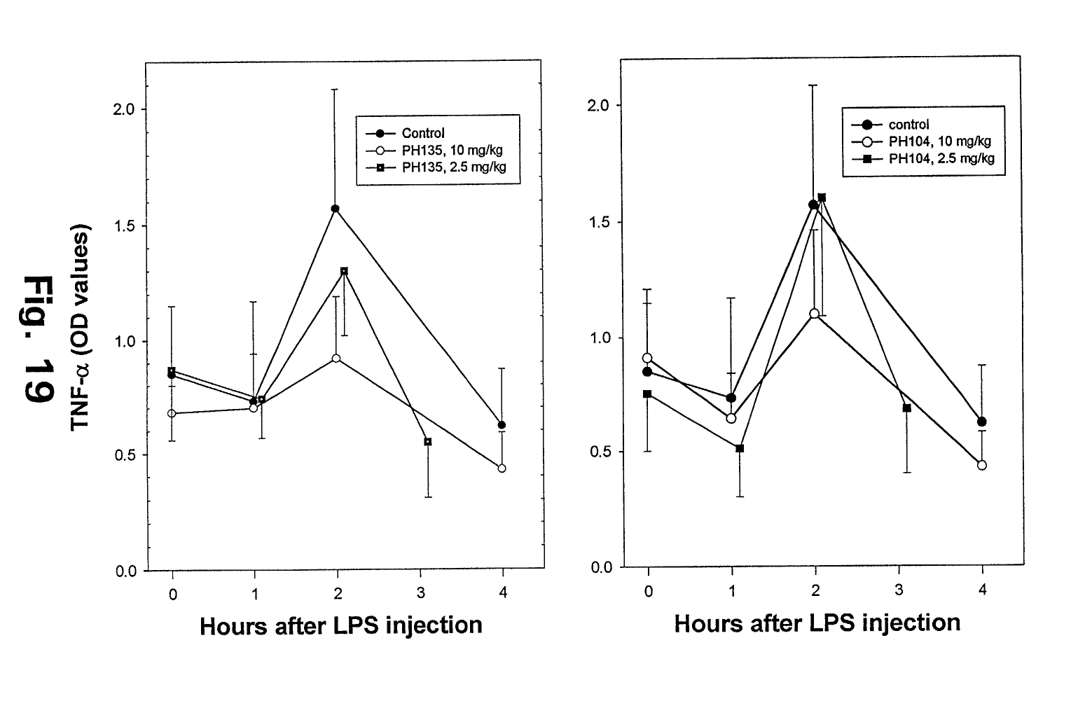

The results are shown in FIGS. 18 and 19.

2b. Production of Mice from a Lethal Effect of LPS-induces Septic Shock. Efficacy (Survival of Treated Animals)

Series of experiments were conducted in order to obtain information on a dose-response inhibitory activity of chalcones on TNF-α production iv vivo.

Experiment 1—Effect of Oral Administration of SBC-24mbc on LPS-induced Septic Shock in BABL/C Mice

Mice: BABL/c, female 20 g. Each group consisted of three mice. Animals were allowed free access to water, but not to food for 24 hrs before the experiment.

LPS: Lipopolysaccharide B, Escherichia coli 055:B5, Sigma, 50 mg/kg (1 mg/mouse), i.p.

SBC-24mbc was suspended in 0.2% Na-CMC and 0.01% Tween 80 and was given to mice by oral route in a dose of 50 mg/kg (3.3 mg/ml)

Treatment Schedule:

1 h before LPS injection

2 hrs after LPS injection

8 hrs after LPS injection

24 hrs after LPS injection

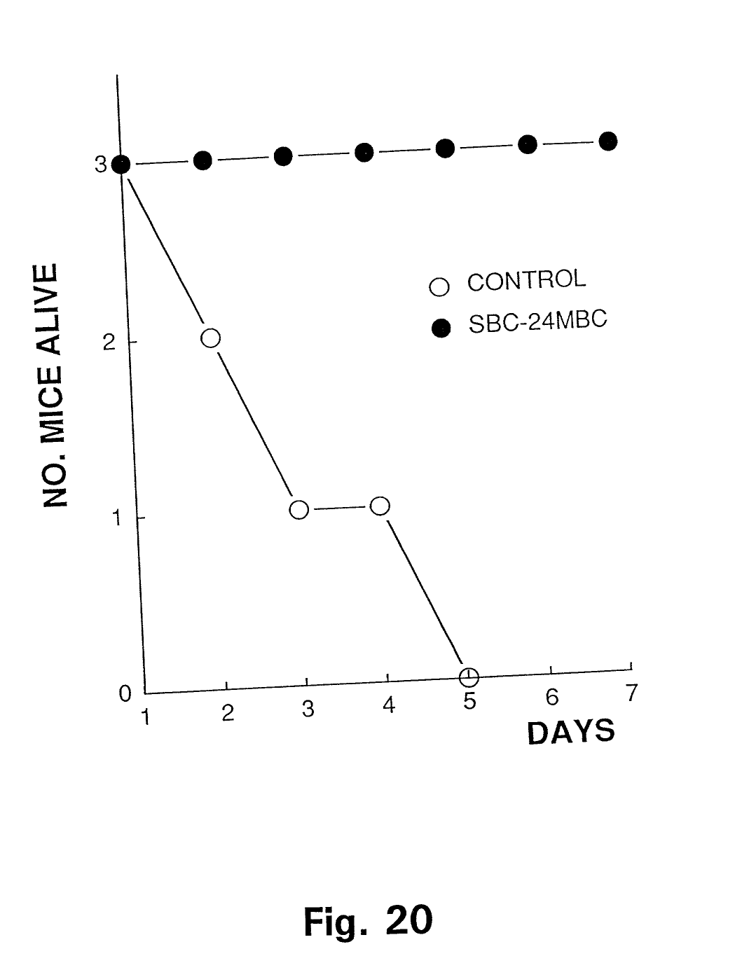

The results are given in FIG. 20.

Experiment 2—Effect of Oral Administration of SBC-24mbc on LPS-induced Septic Shock in BABL/c Mice Using Different Treatment Schedules

Mice: BABL/C, female 20 g. Each group consisted of three mice. Animals were allowed free access to water, but no food was allowed for 24 hrs before the experiment.

LPS: : Lipopolysaccharide B, Escherichia coli 055:B5, Sigma, 50 mg/kg (200 μl of 5 mg LPS/ml in pyrogen-free saline equal to 1 mg/mouse), administered i.p.

SBC-24mbc was suspended in 0.2% Na-CMC and 0.01% Tweeb 80 and was given to mice by oral route in a dose of 50 mg/kg (3.3 mg/ml)

Treatment Schedule:

Group I: 2 hrs before LPS injection, same time as LPS, and 2 hrs after LPS injection (a total of 150 mg/kg)

Group II: 1 h before LPS injection, 2 hrs after LPS injection, and 4 hrs after LPS injection (a total of 150 mg/kg)

Group III: 1 h before LPS injection and 2 hrs after LPS injection (a total of 100 mg/kg)

Group IV: same time as LPS and 2 hrs after LPS injection (a total of 100 mg/kg)

Group V: 1 h after LPS injection and 2 hrs after LPS injection (a total of 100 mg(kg)

Group VI (control): no treatment,

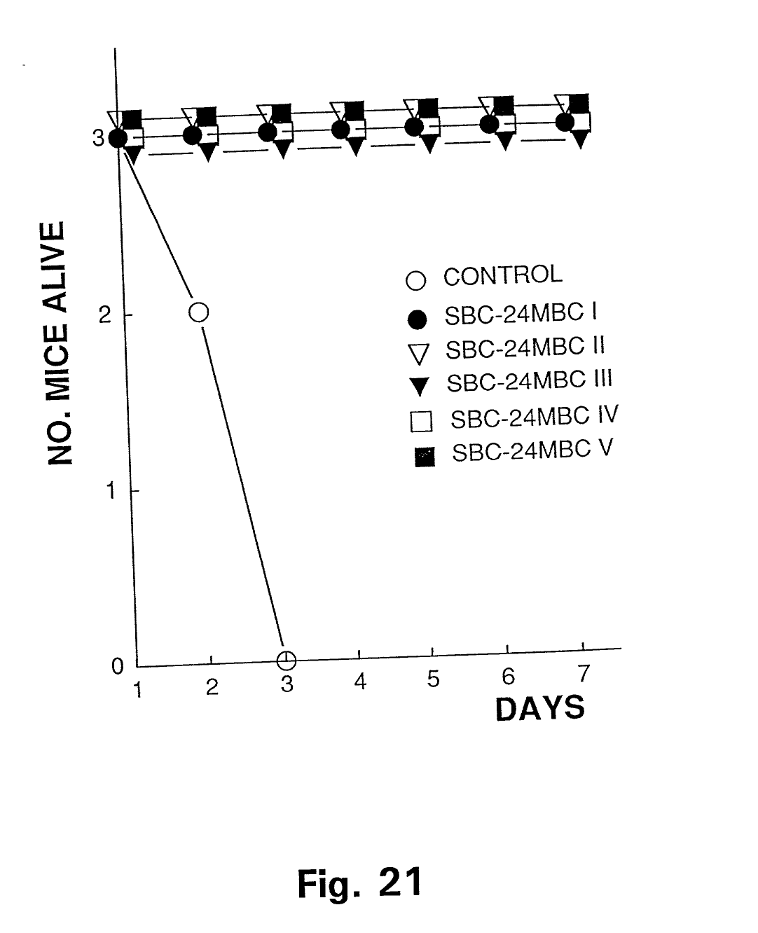

The results are given in FIG. 21.

Experiment 3—Effect of i.p. Administration of SBC-24mbc on LPS-induced Septic Shock in BABL/c Mice

Mice: BABL/c, female, 20 g. Each group consited of 4 mice (group D only two mice) and the mice were maintained under standard laboratory conditions with food and water ad libitum.

LPS: Lipopolysaccharide B, Escherichia coli 055:B5, Sigma, 50 mg/kg (200 μl of 5 mg LPS/ml in sterile pyrogen-free saline, equal to 1 mg/mouse), i.p. The LPS injection started at 12.30

SBC-24mbc was dissolved in 100 μl DMSO and then suspended in sterile pyrogen-free saline and was given to mice by i.p. route at doses of 30 mg and 10 mg/kg (equal to 200 μl of 3 mg/ml and 1 mg/ml), at 1 h before LPS injection, the same time as LPS injection and 2 hrs after LPS injection.

Treatment Schedule

Group A: 30 mg/kg (a total of 90 mg/kg)

Group B: 10 mg/kg (a total of 30 mg/kg)

Group C: Control 1 (200 μl of 10% DMSO in sterile pyrogen-free saline/mouse)

Group D: Control 2 (200 μl in sterile pyrogen-free saline/mouse). Two mice.

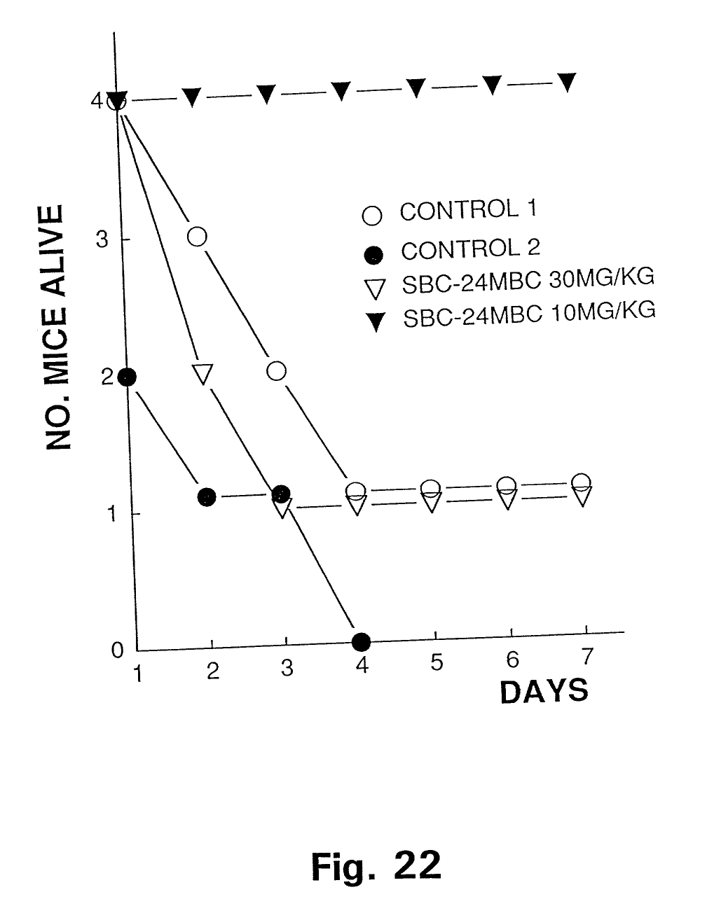

The results are given in FIG. 22.

Experiment 4—Dose Response Study of SBC-24mbc and Chlorpromazine (CPZ) by i.p. Route on LPS-induced Septic Shock in BABL/c Mice

Mice: BABL/C, female, 20 g. Each group consisted of 4 mice. Animals were maintained under standard laboratory conditions with food and water ad libitum.

LPS: Lipopolysaccharide B, Escherichia coli 055:B5, Sigma, 50 mg/kg (200 μl of 5 mg LPS/ml in sterile pyrogen-free saline, equal to 1 mg/mouse), i.p. the LPS injection started at 12:30.

SBC-24mbc was dissolved in 100 μl DMSO and then suspended in sterile pyrogen-free saline and was given to mice by i.p. route in doses of 10 mg, 5 mg, and 2.5 mg/kg (equal to 200 μl of 1 mg/ml, 0.5 mg/ml and 0.25 mg/ml), at 1 h before LPS injection, the same time as LPS injection and 1 h after LPS injection.

CPZ DAK Lab., Copenhagen, Denmark (batch No. 70490511) was dissolved in sterile pyrogen-free saline and was given to mice by i.p. route at a dose of 4 mg/kg (equal to 200 μl of 0.4 mg/ml)

Treament Schedule

Group A: 10 mg/kg (a total of 30 mg/kg)

Group B: 5 mg/kg (a total of 15 mg/kg)

Group C: 2.5 mg/kg (a total of 7.5 mg/kg)

Group D: control (200 μl of 10% DMSO in sterile pyrogen-free saline/mouse)

Group E: CPZ 4 mg/kg, the same time as the LPS injection.

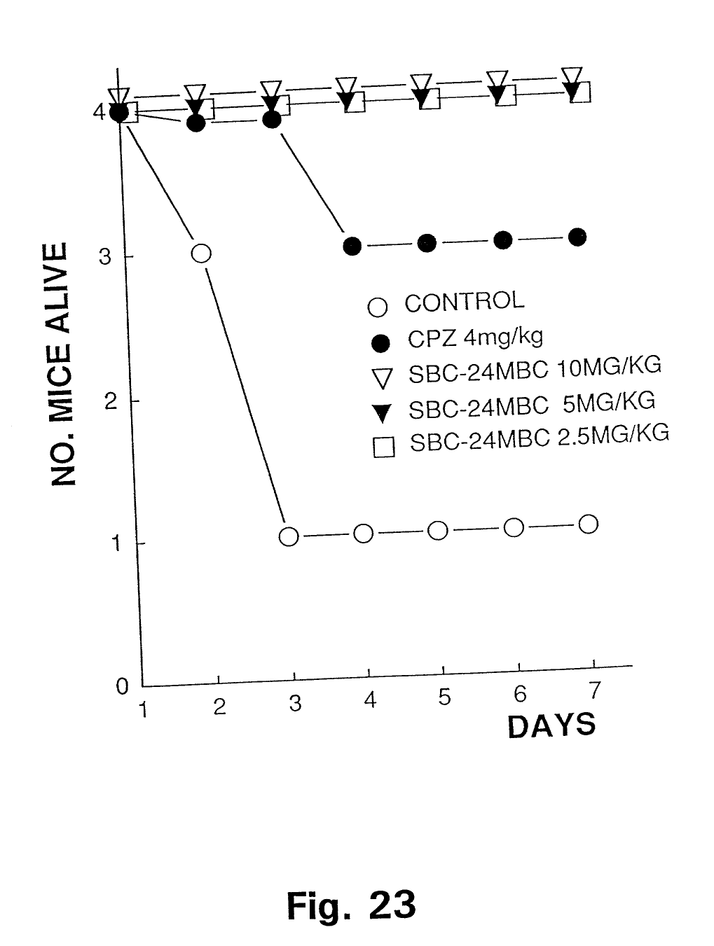

The results are given in FIG. 23.

Experiment 5—Effect of PH135 and PH104 by Oral Route on LPS-induced Septic Shock in BALB/c Mice

Mice: BALB/c, female, 20 g. Each group consists of 7 mice. Animals were maintained under standard laboratory conditions with food and water ad libitum.

LPS: Lipopolysaccaride B, Escherichia coli 055:B5, Sigma, 50 mg/kg (200 μl g 5 mg LPS/ml in sterile pyrogen-free saline, equal to 1 mg/mouse), i.p. injection.

PH135 (A) and PH104 (B) were micronized in 0.2% Na-CMC and 0.01% tween 80 and given to mice by oral route in total dose of 150 mg/kg. PH135 (A) and PH104 (B) were given to mice by oral route in 300 μl of 3.3 mg/ml, at 1 hour before LPS injection, the same time as LPS injection and 1 hour after LPS injection (3 treatments of 50 mg/kg each). Group C. control (300 μl of 0.2% Na-CMC and 0.01% tween 80 buffer)

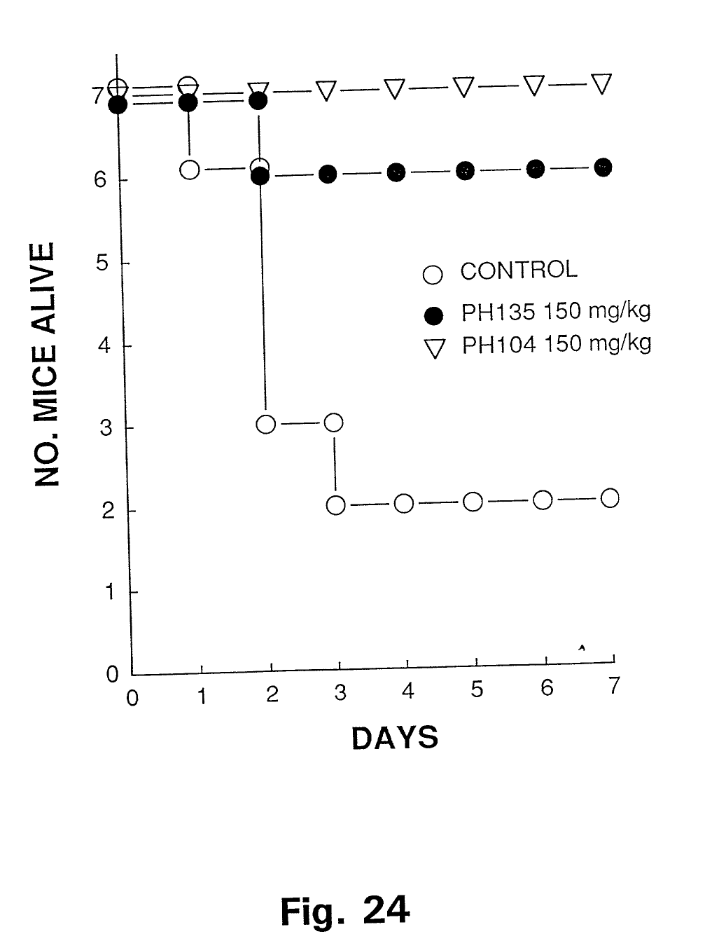

The results are given in FIG. 24

Experiment 6—Effect of PH 135 and PH104 by Oral Route on LPS-induced Septic Shock in BALB/c Mice

Mice: BALB/c, female, 20 g. Each group consists of 7 mice. Animals were maintained under standard laboratory conditions with food and water ad libitumn.

LPS: Lipopolysaccaride B, Escherichia coli 055:B5, Sigma, 50 mg/kg (200 μl g 5 mg LPS/ml in sterile pyrogen-free saline, equal to 1 mg/mouse), i.p. injection.

PH135 (A) and PH104 (B) were micronized in 0.2% Na-CMC and 0.01% tween 80 and given to mice by oral route in total dose of 50 mg/kg. PH135 (A) and PH104 (B) were given to mice by oral route in 100 μl of 3.3 mg/ml, at 1 hour before LPS injection, the same time as LPS injection and 1 hour after LPS injection (3 treatments of 16.6 mg/kg each). Group C. control (100 μl of 0.2% Na-CMC and 0.01% tween 80 buffer)

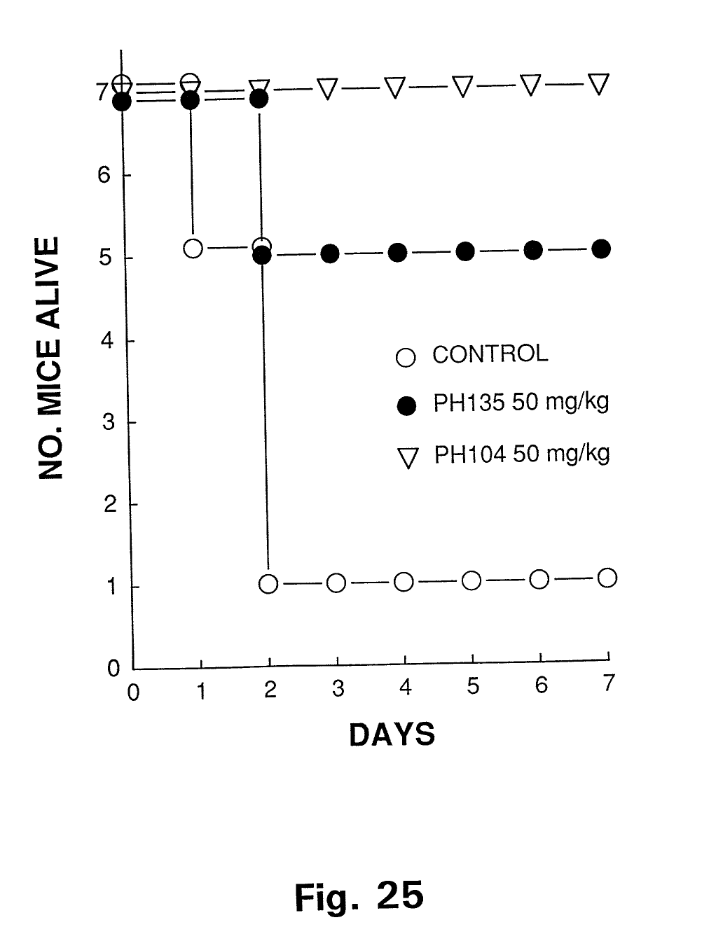

The results are given in FIG. 25

3. Protection of Mice from a Lethal Effect of Cerebral Malaria. Efficacy (Survival of Treated Animals)

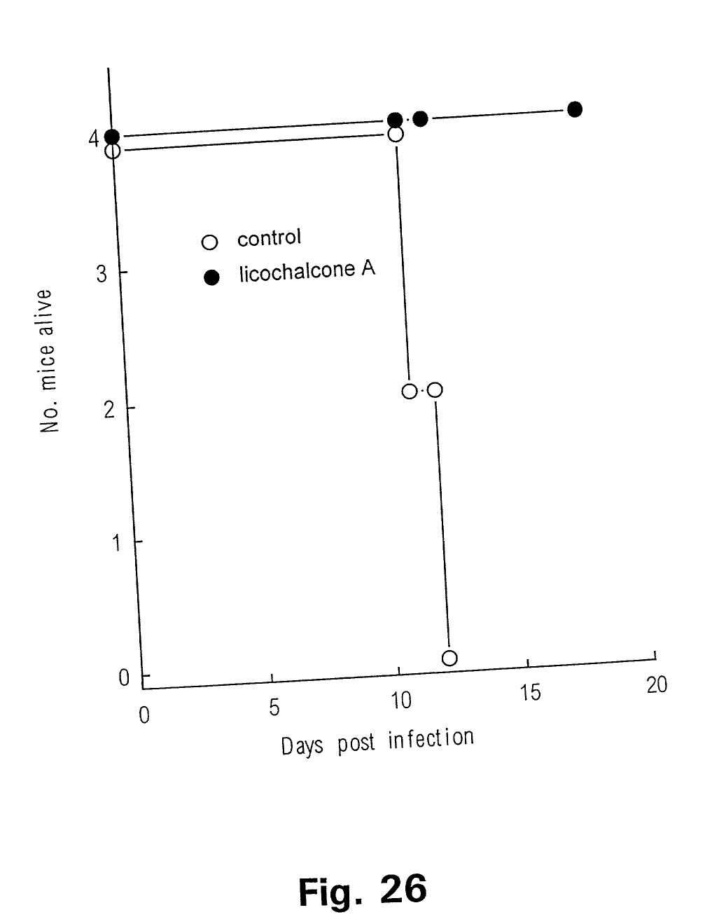

The effect of oral administration of licochalocen A on protection of C57 BL/6 mice from cerebral malaria infected with P. berghei K173 strain is shown in FIG. 26. Mice received 50 mg/kg of licochalcone A once daily over 5 days.

Overall Conclusions

The experiments performed so far clearly demonstrate that:

1) Some oxygenated chalcones inhibit production of TNF-α by human blood mononuclear cells stimulated in vitro.

This is shown by measurement of TNF-α in cell culture supernant and by detection of intracellular TNF-α.

The preliminary studies indicate that the inhibitory effect appears to be at the level of synthesis or processing of TNF-α and not at the TNF target level.

The inhibition of TNF-α production was not due to the toxic effect on cells as shown by trypan blue dye exclusion, lymphocyte proliferation and monocyte chemiluminescence assays.

2) Some oxygenated chalcones administered orally at a dose of 50 mg/kg and intraperitoneally at a dose of 10 mg/kg reduced the serum level of TNF-α by about 50% in an LPS-induced septic shock model in BALB/c mice.

3) Some oxygenated chalcones administered orally at a dose of 50 mg/kg protected mice from an LPS-induced septic shock death.

4) Some oxygenated chalcones administered intraperitoneally at a dose of 2.5 mg/kg protected mice from an LPS-induced septic shock death.

5) The protection was achieved by administration of the compound before, at the same time and after LPS injection with some compounds.

6) Some oxygenated chalcones administered orally protected mice from mortality due to TNF-α associated cerebral malaria.

Implications

Inhibition of inflammatory cytokines particularly TNF-α can have major clinical implications.

These results indicate that oxygenated chalcones can provide the basis for a potential drug as TNF inhibitor.

### References:

Michie et al. N. Eng. J. Med 318:1481, 1988

Waage et al J. Exp. Med. 169:333, 1989

Beutler et al Science 229:869, 1985

Tracey et al Nature 330:662, 1987

Grau et al Science 237:210, 1987

Saukkonen et al J. Exp. Med. 171:439, 1990

Piquet et al J. Exp. Med. 170:655, 1989

Colletti et al J. Clin. Invest. 85:1936, 1990

Shalaby et al Transplantation 46:1057, 1989

Bone et al N. Eng. J. Med. 317:635, 1987

Batt et al Med. Chem. 36:1434-1442, 1993

Jepsen S. and Andersen B. J. Acta Pathol. Microbiol. Scand. Sect. C. 89:99, 1981

Jakobsen P. H. et al Parasite Immunol. 10:593, 1988

Jakobsen P. H. et al. Parasite Immunol. 15:229, 1993

Fomsgaard A. et al Scan. J. Immunol. 27:143, 1988

Hansen M. B. et al. Scan. J. Immunol. 33:777, 1991

### Example 10

Preparation of a QSAR Model for the Prediction of Biological Activities QSAR Analysis

A search for chalcones with optimum activity against Plasmodium or Leishmania parasites and minimum inhibition of the immune system as screened by inhibition of phyto-hemmoglutinin A provoked proliferation of lymphocytes was initiated. An experimental design based on a 2D and a 3D QSAR (Quantitative Structure Activity Relationship) analyses was chosen for a rational selection of chalcones to be synthesised.

The importance of the substitution pattern at the two aromatic nuclei was investigated by variation according to the principles of experimental design. The importance of the substituents was elucidated by syntheses of chalcones covering the different properties such as volume, lipophilicity (π value), charge, Taft constants, σ value, Hammet value, and combinations hereof as completely as possible. Based on the principles of experimental design, 150 compounds were synthesised and a 2D and a 3D QSAR analysis performed to reveal the optimum substitution pattern for antileishmanial and antimalarial activity and minimum inhibition of lymphocyte proliferation. The QSAR analyses were based on the QSAR Model described herein. The value of

The results from the QSAR analysis are given below.

The values for the four variables is known for 100 aromatic substituents (see Skagerberg, B. et al. Quant. Struct.-Act. Relat. 1989, 8, 32,38.). 62 substituents were selected based on considerations with respect to availability and ease of synthesis. A principal component analysis was performed for the 62 substituents based on the values for lipophilicity (π), molecular refractivity (MR), and electron distribution in the two aromatic rings (σₘ and σₚ). The variables are scaled so that each of the variables becomes equally important (auto-scaling to the variance 1). The principal component analysis revealed that the number of variables could be reduced to two principal components, the first explaining 56.5% and the second 32.5% of the variation. The four variables contributed with approximately the same loading to both of the two principal components, but the two electronic variables (σ) both had negative signs in the first principal component and positive signs in the second principal component, cf. the table below.

| | Variable | Autoscaling | | |
|---|---|---|---|---|
| Variable | average | factor | p1 | p2 |
| π | 0.53 | 0.95 | 0.47 | 0.49 |
| MR | 15.4 | 0.13 | 0.42 | 0.59 |
| σₘ | 0.13 | 4.46 | −0.57 | 0.38 |
| σₚ | 0.01 | 2.84 | −0.52 | 0.51 |

p1 and p2 are the loadings for each of the variables in the principal components PC1 and PC2.

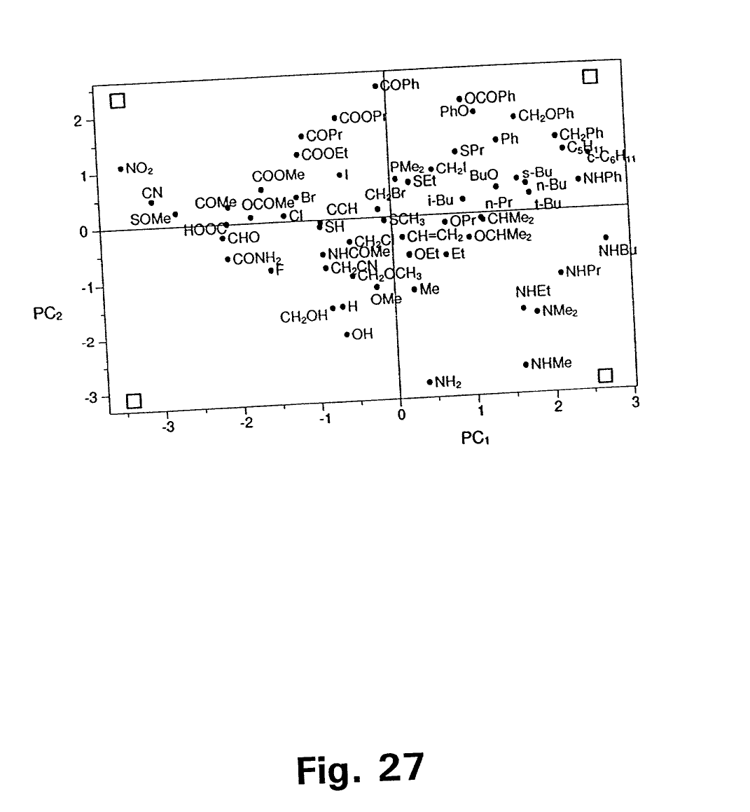

The positions of the individual substituents in the plane constituted by the two principal components are shown in FIG. 27.

As the two principal components PC₁ and PC₂ are the new variables, the figure can be used in the design of new compounds. The figure may also be used for the comparison of substituent properties, for grouping of substituents which have similar properties, and for identification of substituents which diverge from other substituents, i.e. so-called “outliners”.

The design was constructed so that the variability of properties was covered in an effective way and so that the following criteria were considered: (a) the compound should be relatively active so that no solubility problems would arise, (b) ease of synthesis, and (c) information from any previously synthesised compounds should be included in the design.

Based on the result from the principal analysis and the consideration taken with respect to (a)-(c), six 2² designs were made, one for each of the three possibilities for substituting each the two aromatic rings in the chalcone skeleton. The selected 24 compounds were:

| PC1 | PC2 | Compound |
|---|---|---|
| + | + | 2′-butoxy-3,5-dimethoxychalcone |
| + | − | 2′-dimethylamino-3,5-dimethoxychalcone |
| − | + | 2′-nitro-3,5-dimethoxychalcone |
| − | − | 2′-fluoro-3,5-dimethoxychalcone |
| + | + | 3′-butoxy-2,4-dimethoxychalcone |
| + | − | 3′-dimethylamino-3,5-dimethoxychalcone |
| − | + | 3′-nitro-2,4-dimethoxychalcone |
| − | − | 3′-fluoro-3,5-dimethoxychalcone |
| + | + | 4′-cyclohexyl-3,5-dimethoxychalcone |
| + | − | 4′-dimethylamino-3,5-dimethoxychalcone |
| − | + | 4′-nitro-3,5-dimethoxychalcone |
| − | − | 4′-fluorochalcone |
| + | + | 2-butoxy-2′,3′,4′-trimethoxychalcone |
| − | + | 2-nitro-2′,3′,4′-trimethoxychalcone |
| − | − | 2-flouro-2′,3′,4′-trimethoxychalcone |
| + | + | 3-butoxy-2′,3′,4′-trimethoxychalcone |
| + | − | 3-dimethylamino-2′,3′,4′-trimethoxycalcone |
| − | + | 3-nitro-2′,3′,4′-trimethoxychalcone |
| − | − | 3-flouro-2′,3′,4′-trimethoxychalcone |
| + | + | 4′-fluoro-4-phenoxychalcone |
| + | − | 4-dimethylamino-2′,3′,4′-trimethoxychalcone |
| − | + | 4-cyano-2′,3′,4′-trimethoxychalcone |
| − | − | 4′-cyclohexyl,4-fluorochalcone |
| − | − | 4,4′-difluorochalcone |

2D-QSAR analysis was performed based on the compound above. The variables lipophilicity (π), molecular refractivity (MR), and electron distribution in the two aromatic rings (σₘ and σₚ) were selected.

Antileishmanial Activities

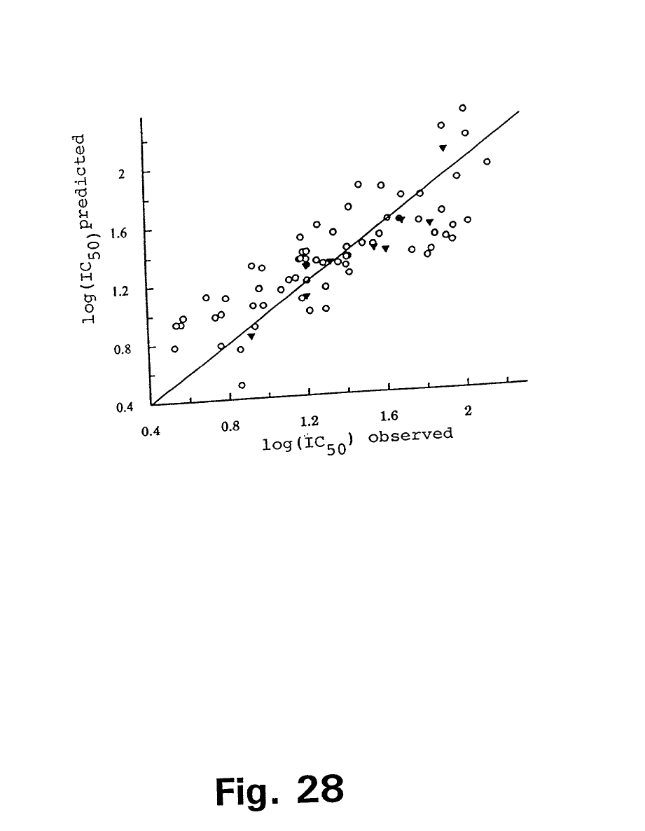

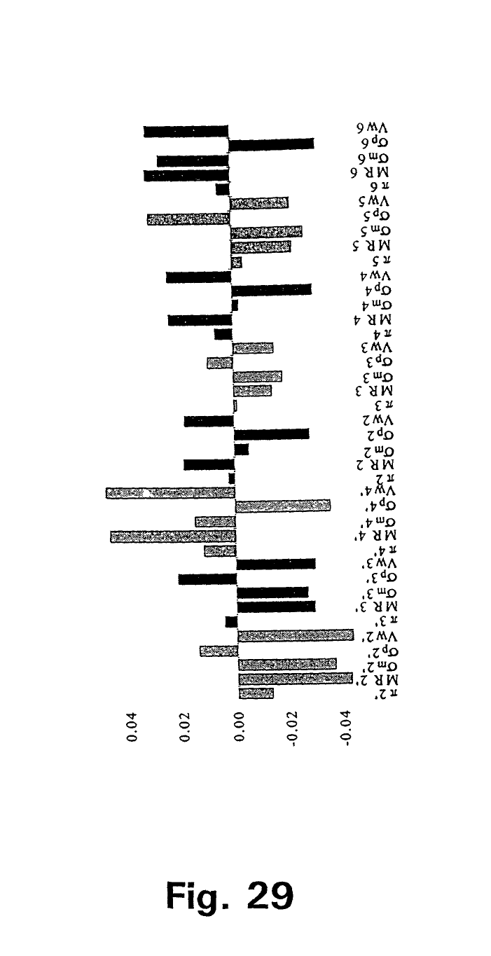

Beside the 24 chalcones, selected as described in the paragraph above, an additional, separate, distinct 52 chalcones were tested for in vitro antileishmanial activity. A 2D QSAR model was calculated using the programme SIMCA P 2.1. A number of the chalcones elucidate the same variables and were selected for validation of the model. The selected compounds describe the entire range of biological activities and chemical variability. The calculated QSAR model has one significant dimension and the results of the internal validation (R²=0.64, Q²=0.56) reveal good predicitivity as depicted in FIG. 28. The expected activities of new chalcones may be predicted on the basis of FIG. 29.

Lymphocyte Suppressing Activities

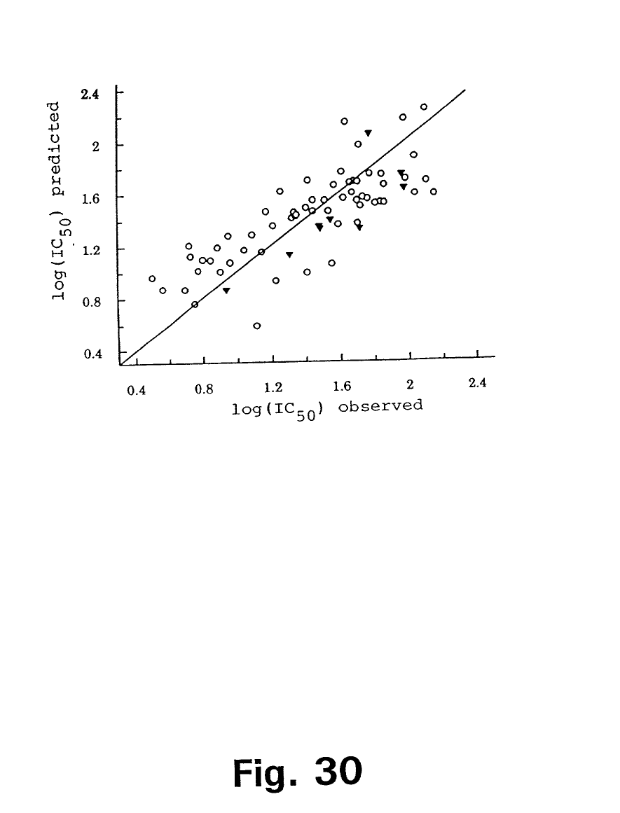

Beside the 24 chalcones selected by experimental design, an additional 46 chalcones of the 52 groups were tested for their ability to inhibit the phytohaemagglutinin A provoked proliferation of human lymphocytes. The same nine compounds were selected for validation of the model. The calculated model has one significant dimension and good predictivity (R²0.62, Q²=0.50), see FIG. 30.

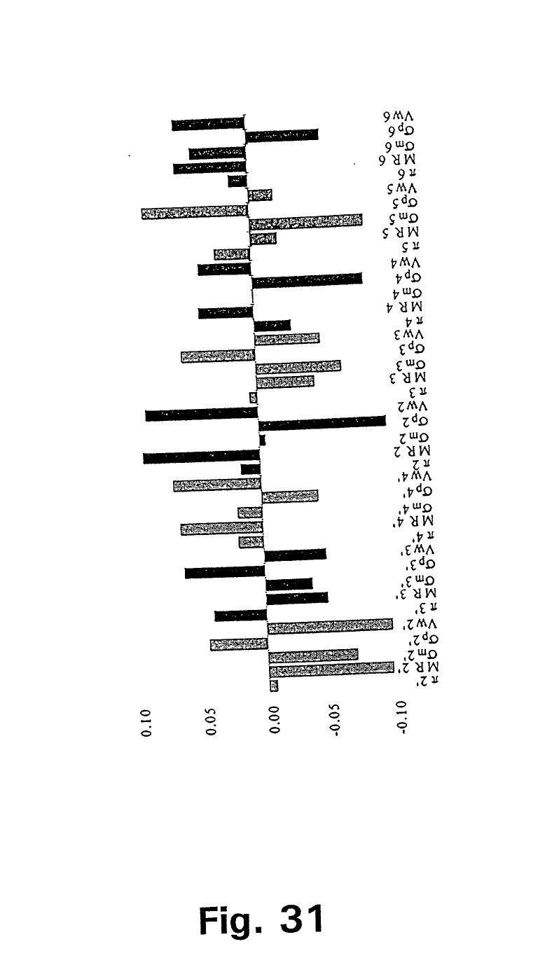

As illustrated in FIG. 31, substitution in all positions except positions 5′ and 6′ has a major influence on the activity, as was seen for the antileishmanial activities.

Comparison of the influence of the substituents on antileishmanial and lymphocyte suppressing activities.

A comparison of FIGS. 29 and 31 reveals that large electronegative substituents like butoxy in position 2′, 3′, or 5 should increase activity. The models only encompass monosubstituted chalcones, but it is tempting to guess that a combination of the above substitutions might give compounds with higher potencies. Notice that the models predict different effect on antileishmanial and lymphocytes suppressing activities by substitution in the positions 2 and 5. Large substituents in position 5 increase the antileishmanial activities more that the lymphocytes suppressing activities. Large substituents in position 2 decreases the lymphocyte suppressing activities to a greater extent than the antileishmanial activities.

3D QSAR

A 3D QSAR based on the same data as used for the 2D QSAR using a GRID-GOLPE combination was performed. A three dimensional grid with a distance of 1 Å between the points covering the molecule was constructed yielding 57200 variables for each molecule. The number of variables was reduced to 1365 by grouping and selection of the variables by D optimal design. The obtained model has a high quality as shown in the figures (R²=0.73, Q²=0.63). Notice that the 2D and 3D models suggest the same substitution pattern for highly active chalcones, except for a minor difference of the effect of a substituent in position 5.

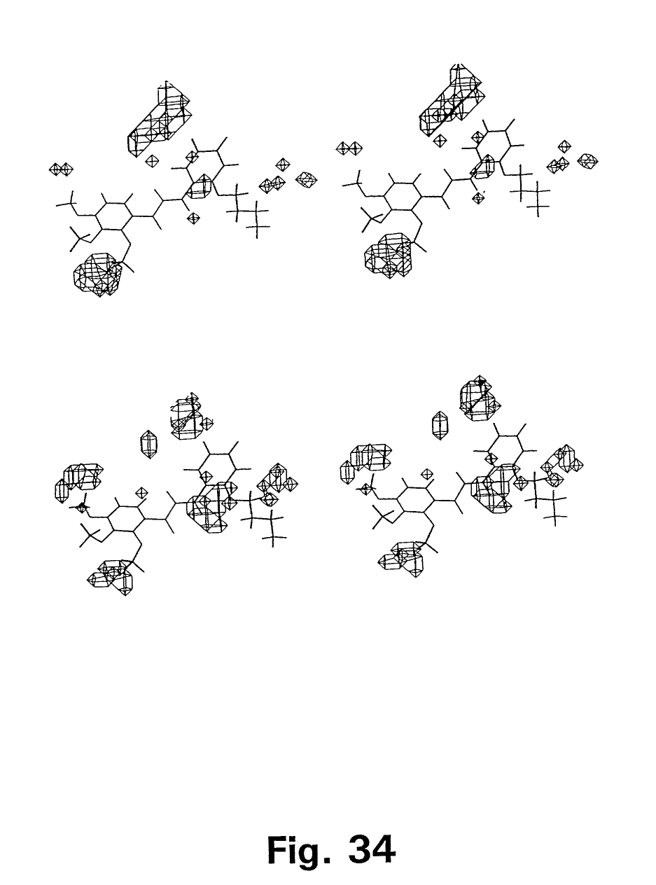

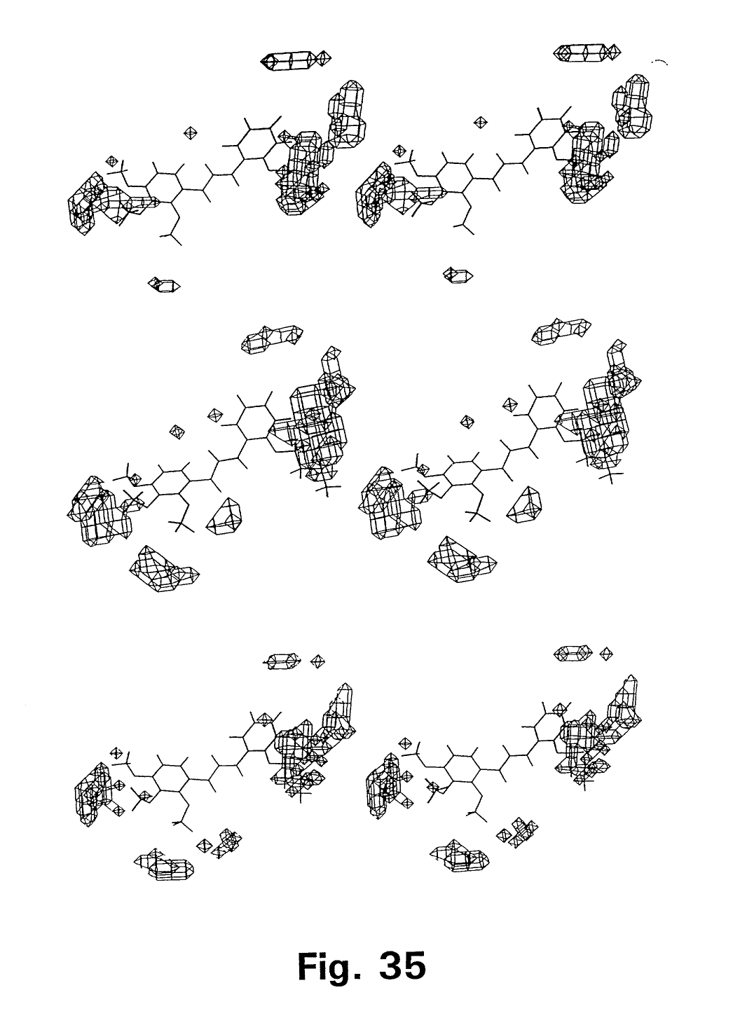

A similar 3D QSAR was performed for the lymphocyte suppressing activity of the chalcones to give a high quality model as depicted in FIGS. 34 and 35 (R²=0.90, Q²=0.80).

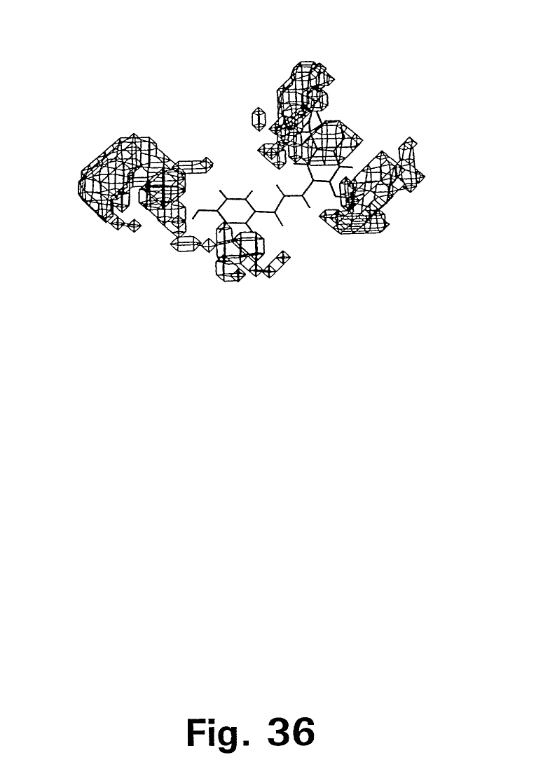

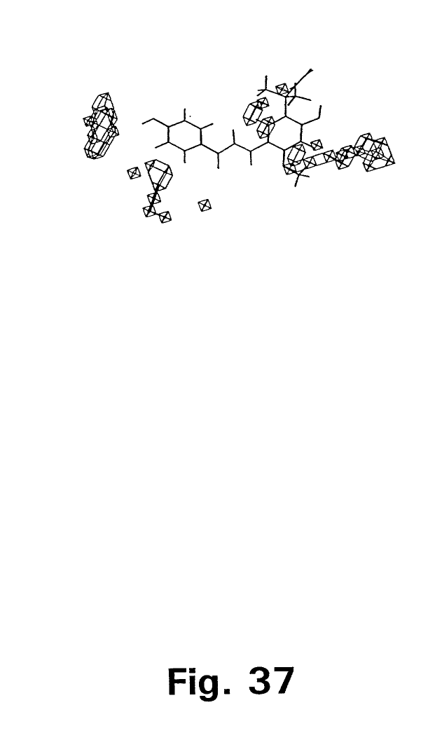

A similar 3D QSAR was performed for the antiplasmodium activity of the chalcones to give a model as depicted in FIGS. 36 and 37 (R²=0.75, Q²=0.57). It should be noticed, that this model is of a lower quality than the leishmania and the lymphocyte models. This model is interesting by indicating that a large substituent in the 2 and 5 positions would increase the antiplasmodium activity.

Conclusion

Examination of FIGS. 36 and 37 reveals that bulky substituents in the positions 2, 3, 2′, 4, 5′, and 6′ all increases the antiplasmodium activity. Substituents in the 3′ position might decrease the antiplasmodium activity. Comparison of FIG. 35 and FIG. 36 reveals the possibility of preparing chalcones selectively inhibiting malaria parasites by introducing bulky substituents in position 2 and 3.

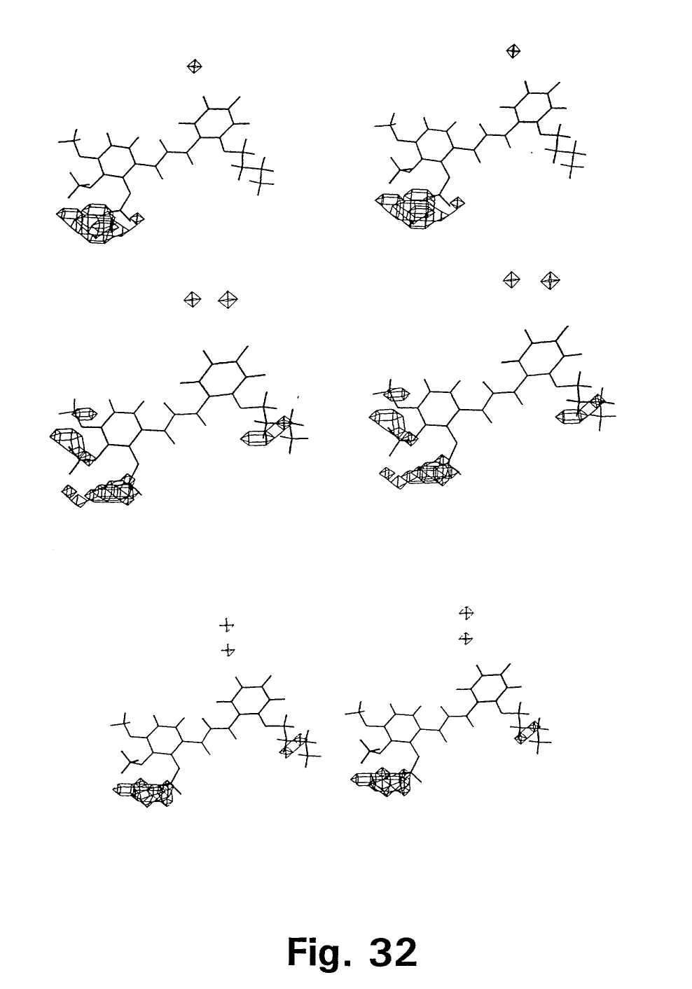

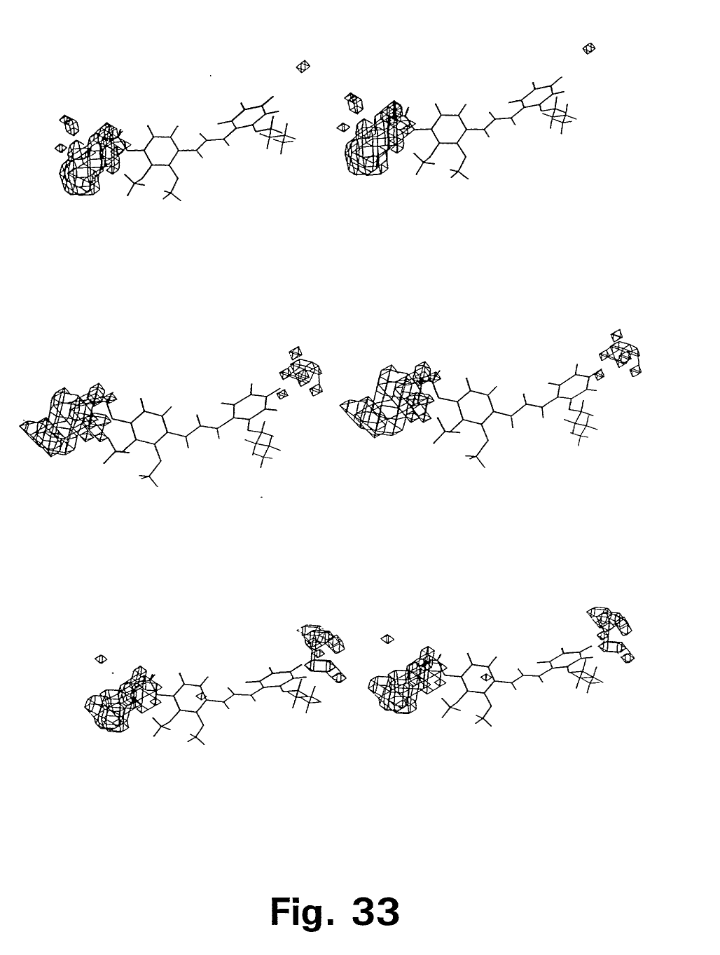

Comparison of FIGS. 32 and 33 with FIGS. 34 and 35 reveals that the antileishmanial activity and the lymphocyte suppressing activity both are improved by bulky substituents in the 2′ position. In the case of lymphocyte suppressing activity a bulky substituent in the 2, 4 and 3′ position decreases the activity, whereas bulky substituents in the 3′ and 4′ positions, especially the 4′position decreases the antileishmanial activity. In conclusion bulky substituents in the 2′ position and to a minor extent a bulky substituent in the 2 position is expected to increase the antileishmanial activity and a bulky substituent in the 3′ position increases the antileishmanial activity and decreases the lymphocyte suppressing activity.

Biological Results for Bis-aromatic Compound

A number of compound have been tested in the Leishmania Promastogotes Assay, in the Plasmodium falciparum Assay, and in the Lymphocytes proliferation Assay. The results from these biological test are shown below. Some of these results have been used in the QSAR analyses described above.

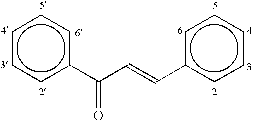

The specification of substituents relates to the above structure.

| | | Lympho- | |
|---|---|---|---|
| | Leishmania | cytes | Plasmodium |
| | IC₅₀ ± SD | IC₅₀ ± SD | IC₅₀ ± SD |
| Compound | μM | μM | μM |
| Licochalcone A | 13 ± 1 | 48 ± 5 | 5.6 |
| 2-OCH₃-4,4′-OH | 68 ± 4 | 94 ± 4 | 19 |
| 2-OCH₃-4,4′-OH-5-propyl | 26 ± 2 | 41 ± 3 | 10 |
| 2-OCH₃-4,4′-OH-5-hexyl | 9.7 ± 0.9 | 46 ± 3 | 5.1 |
| 2-OCH₃-3-methyl-4,4′-OH | 22 ± 3 | 27 ± 4 | 33 |
| Licochalcone C | 55 ± 3 | 53 ± 2 | 16 |
| 2-OCH₃-4,4′-OH-6-methyl | 39 ± 5 | 53 ± 6 | 28 |
| 3,5-OCH₃-4′-allyloxy | 27 ± 2 | 25 ± 2 | — |
| 4-C₄H₉-3,5-OCH₃-4′-allyloxy | 90 ± 14 | — | — |
| 4-C₆H₁₃-3,5-OCH₃-4′-allyloxy | 19 ± 4 | 15 ± 1 | 11 |
| 4′-cC₆H₁₁-4-OC₆H₅ | — | — | 200 |
| 4′-NO₂-4-N(CH₃)₂ | — | — | — |
| 4′,4-N(CH₃)₂ | 30 ± 4 | — | 25 |
| 4′-F-4-OC₆H₅ | 42 ± 6 | 33 ± 2 | — |
| 4′-cC₆H₁₁-4-F | 27 ± 2 | 74 ± 2 | — |
| 4′,4-NO₂ | 26 ± 4 | 51 ± 3 | — |
| 4′-N(CH₃)₂-4-NO₂ | — | — | 93 |
| 4′,4-F | 15 ± 2 | 22 ± 1 | 43 |
| 2′-OC₄H₉-3,5-OCH₃ | 7.2 ± 1.2 | 13 ± 1 | 25 |
| 2′-N(CH₃)₂-3,5-OCH₃ | 7.3 ± 1.0 | 5.7 ± 0.3 | 13 |
| 2′-NO₂-3,5-OCH₃ | 3.4 ± 0.5 | 3.7 ± 0.2 | 7.4 |
| 2′-F-3,5-OCH₃ | 3.5 ± 0.8 | 6.0 ± 0.4 | 8.7 |
| 3′-OC₄H₉-2,4-OCH₃ | 20 ± 3 | 65 ± 4 | 16 |
| 3′-N(CH₃)₂-3,5-OCH₃ | 5.8 ± 0.7 | 5.0 ± 0.2 | 29 |
| 3′-NO₂-2,4-OCH₃ | 8.6 + 1.1 | 18 ± 2 | — |
| 3′-F-3,5-OCH₃ | 5.5 ± 0.3 | 9.2 ± 0.5 | 8.1 |
| 4′-cC₆H₁₁-3,5-OCH₃ | 31 ± 2 | 39 ± 2 | 15 |
| 4′-N(CH₃)₂-3,5-OCH₃ | — | — | 17 |
| 4′-NO₂-3,5-OCH₃ | 16 ± 1 | 20 ± 1 | — |
| 4′-F | 15 ± 1 | 23 ± 2 | 51 |
| 2-OC₄H₉-2′,3′,4′-OCH₃ | 26 ± 1 | 52 ± 3 | 7.8 |
| 2-NO₂-2′,3′,4′-OCH₃ | 5.9 ± 0.4 | 7.0 ± 0.6 | 9.3 |
| 2-F-2′,3′,4′-OCH₃ | 15 ± 1 | 11 ± 1 | — |
| 3-OC₆H₅-2′,3′,4′-OCH₃ | 8.7 ± 0.5 | 17 ± 1 | 8.9 |
| 3-N(CH₃)₂-2′,3′,4′-OCH₃ | 20 ± 3 | 26 ± 1 | 12 |
| 3-NO₂-2′,3′,4′-OCH₃ | 17 ± 2 | 36 ± 2 | 5.5 |
| 3-F-2′,3′,4′-OCH₃ | 9.7 ± 0.9 | 6.4 ± 0.4 | 29 |
| 4-N(CH₃)₂-2′,3′,4′-OCH₃ | 21 ± 2 | 35 ± 2 | 22 |
| 4-CN-2′,3′,4′-OCH₃ | 8.6 ± 0.9 | 5.4 ± 0.3 | 7.6 |
| 2′-OH-3,5-OCH₃ | 3.8 ± 0.9 | 8.1 ± 0.8 | 20 |
| 3′-OH-3,5-OCH₃ | 3.7 ± 0.6 | 3.2 ± 0.4 | 28 |
| 2-OH-2′,3′,4′,-OCH₃ | 9.3 ± 1.0 | 9.0 ± 0.7 | 10 |
| 2′,3′,4′,3,5-OCH₃ | 8.3 ± 0.9 | 8.7 ± 0.5 | — |
| 3′,2,4-OCH₃ | 18 ± 3 | 42 ± 2 | — |
| 2′-Br-2,4-OCH₃ | 14 ± 2 | 35 ± 3 | 26 |
| 3′-Br-2,4-OCH₃ | 16 ± 1 | 37 ± 2 | 17 |
| 2′,4′-F-2,4-OCH₃ | 26 ± 2 | 74 ± 4 | 49 |
| 2′,4′-OCH₃-2,4-F | 27 ± 4 | 12 ± 0 | 9 |
| 2′,3′,4′-OCH₃-2,4-Cl | 6.3 ± 0.6 | 7.8 ± 0.9 | 7.9 |
| 2′,4′-OCH₃-3,4-F | 16 ± 1 | 5.3 ± 0.6 | 10 |
| 2′,5′-OCH₃-2,4-Cl | 7.8 ± 0.7 | 5.7 ± 0.5 | — |
| 4′-4-OH | 83 ± 5 | 59 ± 3 | 17 |
| 2′,4′,3,4-OH | 66 ± 2 | 16 ± 1 | 49 |
| 4′,4-OCH₃ | 106 ± 9 | 112 ± 8 | 51 |
| 2,6-OCH₃-4′-OH | 82 ± 8 | 60 ± 2 | — |
| 2,3-OCH₃-4′-OH | 89 ± 9 | 70 ± 4 | 25 |
| 3,5-OCH₃-4′-OH | 12 ± 1 | 14 ± 1 | 25 |
| 2′,4′,4-OH-3-OCH₃ | 16 ± 2 | 31 ± 2 | 43 |
| 3,4,5-OCH₃-4′-allyloxy | 41 ± 4 | 30 ± 1 | — |
| 2,4,6-OCH₃-4′-OH | 106 ± 8 | 44 ± 2 | — |
| Chalcon | 20 ± 1 | 21 ± 2 | 44 |
| 4′-OH | 16 ± 1 | 28 ± 1 | 21 |
| 2,5-OCH₃-4′-allyloxy | 73 ± 4 | 55 ± 2 | 23 |
| 3,4-OCH₃-4′-allyloxy | 60 ± 5 | 51 ± 6 | 32 |
| 2,4,2′-OCH₃ | 16 ± 1 | 26 ± 2 | — |
| 2,4-OCH₃-4′-OC₆H₁₃ | 136 ± 15 | — | 14 |
| 2,4-OCH₃-4′-OC₄H₉ | 95 ± 10 | — | — |
| 4′-OC₆H₁₃ | 62 ± 5 | 99 ± 7 | 17 |
| 2,5-OCH₃-4′-OH | 23 ± 2 | 22 ± 1 | — |
| 2,4-OCH₃ | 15 ± 0 | 48 ± 2 | 34 |
| 2,6-OCH₃-4′-OC₄H₉ | 104 ± 9 | 127 ± 10 | 14 |
| 2,6-OCH₃-4′-allyloxy | 81 ± 7 | 96 ± 8 | — |
| 2,3-OCH₃-4′-allyloxy | 49 ± 3 | 96 ± 6 | 26 |
| 3,5-OCH₃-4′-OC₄H₉ | 35 ± 3 | 52 ± 3 | 17 |
| 2,4,5,-OCH₃-4′-OH | 16 ± 2 | 28 ± 2 | 13 |
| 2,4-OCH₃-2′-OH | 35 ± 2 | 144 ± 9 | 28 |
| 2,4-OCH₃-2′-allyloxy | 5.0 ± 0.7 | — | — |
| 2,4-OCH₃-4′-OH | 48 ± 3 | 71 ± 5 | 11 |
| 2,5,4′-allyloxy | 38 ± 2 | 61 ± 3 | — |
| 2-OCH₃-4-OH-4′-N(CH₃)₂ | 49 ± 4 | 111 ± 7 | 23 |
| 4-N(CH₃)₂-4′-OH | 79 ± 6 | 130 ± 13 | — |

— : Not tested

Compounds marked with bold have been used to validate the QSAR Model.

1. The use of a compound selected from 1,3-bis-aromatic-prop-2-en-1-one, 1,3-bis-aromatic-propan-1-one, and 1,3-bis-aromatic-prop-2-yn-1-one for the preparation of a pharmaceutical composition for the treatment or prophylaxis of conditions relating to harmful effects of inflammatory cytokines, wherein the bis-aromatic compound has an IC₅₀ value in the Cytokine inhibition Assay defined herein of at the most 100 μg/ml, such at the most, e.g., 75 μg/ml, 60 μg/ml, 50 μg/ml, 40 μg/ml, 30 μg/ml, 20 μg/ml, or 10 μg/ml.

2. The use according to claim 1, wherein the compound, at the IC₅₀ concentration in the Cytokine inhibition Assay, shows a reduction of the thymidine uptake by human lymphocytes in the Lymphocyte Proliferation Assay, as defined herein, using phytomemagglutinin (PHA), of less than 50%, preferably less than 40%, especially less than 30%, in particular less than 20%.

3. The use according to any of the claims 1-2, wherein the ratio between the IC₅₀ value in the Cytokine inhibition Assay and the IC₅₀ value in the Lymphocyte Proliferation Assay for the compound in question is equal to or less than 1, such as less than, e.g., 0.9, 0.8, 0.7, 0.6, 0.5, 0.4, 0.3, or 0.2.

4. The use of a compound selected from 1,3-bis-aromatic-prop-2-en-1-one, 1,3-bis-aromatic-propan-1-one, and 1,3-bis-aromatic-prop-2-yn-1-one for the preparation of a pharmaceutical composition for the treatment or prophylaxis of conditions involving infection by Helicobacter species, wherein the bis-aromatic compound has an MIC value in the Helicobacter pylori Assay of at the most 200 μg/ml, such as at the most, e.g., 100 μg/ml, 75 μg/ml, 50 μg/ml, 40 μg/ml, 30 μg/ml, 20 μg/ml, or 10 μg/ml.

5. The use according to claim 4, wherein the compound, at the MIC concentration in the Helicobacter species Assay, shows a reduction of the thymidine uptake by human lymphocytes in the Lymphocyte Proliferation Assay, as defined herein, using phytomemagglutinin (PHA), of less than 50%, preferably less than 40%, especially less than 30%, in particular less than 20%.

6. The use according to any of the claims 4-5, wherein the ratio between the MIC value in the Helicobacter species Assay and the IC₅₀ value in the Lymphocyte Proliferation Assay for the compound in question is less than 2, such as less than, e.g., 1.5, 1.2., 1.0, 0.9, 0.8, 0.7, 0.6, 0.5, 0.4, 0.3, or 0.2.

7. The use of a compound selected from 1,3-bis-aromatic-prop-2-en-1-one, 1,3-bis-aromatic-propan-1-one, and 1,3-bis-aromatic-prop-2-yn-1-one for the preparation of a pharmaceutical composition for the treatment or prophylaxis of conditions involving infection by viruses, wherein the bis-aromatic compound has an IC₅₀ value in the Virus plaque formation and/or Virus cytopathic Assay of at the most 50 μg/ml, such as at the most, e.g. 40 μg/ml, 30 μg/ml, 20 μg/ml, 10 μg/ml, or 5 μg/ml.

8. The use according to claim 7, wherein the compound, at the IC₅₀ concentration in the Virus plaque formation and/or Virus cytopathic Assay, shows a reduction of the thymidine uptake by human lymphocytes in the Lymphocyte Proliferation Assay, as defined herein, using phytomemagglutinin (PHA), of less than 50%, preferably less than 40%, especially less than 30%, in particular less than 20%.

9. The use according to any of the claims 7-8, wherein the ratio between the IC₅₀ value in the Virus plaque formation and/or Virus cytopathic Assay and the IC₅₀ value in the Lymphocyte Proliferation Assay for the compound in question is equal to or less than 1, such as less than, e.g., 0.9, 0.8, 0.7, 0.6, 0.5, 0.4, 0.3, or 0.2.

10. The use of a compound selected from 1,3-bis-aromatic-prop-2-en-1-one, 1,3-bis-aromatic-propan-1-one, and 1,3-bis-aromatic-prop-2-yn-1-one for the preparation of a pharmaceutical composition for the treatment or prophylaxis of neoplastic disorders, wherein the bis-aromatic ketone has an IC₅₀ value in the Anti-cancer Assay of at the most 100 μg/ml, such at the most, e.g., 75 μg/ml, 60 μg/ml, 50 μg/ml, 40 μg/ml, 30 μg/ml, 20 μg/ml, or 10 μg/ml.

11. The use according to any of the preceding claims, wherein the compound is of the general formula I
(WA)ₘ—Ar¹—CO—V—Ar²—(AW)ₙ I
wherein
Ar¹ and Ar² each designate an aromate selected from phenyl and 5- or 6-membered unsaturated heterocyclic rings containing one, two or three heteroatoms selected from oxygen, sulphur, and nitrogen, such as furanyl, thiophenyl, pyrrolyl, imidazolyl, isoxazolyl, oxazolyl, thiazolyl, pyrazolyl, pyridinyl, or pyrimidinyl, which aromate may be substituted with one or more substituents selected from halogen; cyano; nitro; nitroso; and C₁₋₁₂, preferably C₁₋₆, straight, branched or cyclic aliphatic hydrocarbyl which may be saturated or may contain one or more unsaturated bonds selected from double bonds and triple bonds, which hydrocarbyl may be substituted with one or more substituents selected from hydroxy, halogen, amino, and amino which is optionally alkylated with one or two C₁₋₆ alkyl groups;
each of the further substituents AW independently designates a group AR_{H}, AH, or a group AZ, wherein each A independently is selected from —O—, —S—, —NH—, or —NR_{H}—, R_{H} designates C₁₋₆ straight, branched or cyclic aliphatic hydrocarbyl which may be saturated or may contain one or more unsaturated bonds selected from double bonds and triple bonds, which hydrocarbyl may be substituted with one or more substituents selected from hydroxy, halogen, amino, and amino which is optionally alkylated with one or two C₁₋₆ alkyl groups, and Z designates (when the compound is a prodrug) a masking group which is readily decomposed under conditions prevailing in the animal body to liberate a group AH, in which A is as defined above; m designates the number of further substituents AW on the aromate Ar¹ and is 0, 1, 2, 3 or 4, and n designates the number of further substituents on the aromate Ar² and is 0, 1, 2 or 3, with the proviso that n and m are not both 0,
V is either —CRR—CRR—, —CR═CR— or —C≡C—, wherein each R independently designates hydrogen, cyano, nitro, nitroso, amino, and halogen, R_{H}, and AW.

12. The use according to claim 11, wherein each R independently is selected from hydrogen, C₁₋₃ alkyl, cyano, and, halogen.

13. The use according to claim 11 or 12, wherein V is selected from —CHR—CHR—, —CR═CH—, —CH═CR—, —CH₂—CHR—, and —CHR—CH₂—.

14. The use according to any of claims 11-13, wherein Z is selected from the below groups (A)-(F)
—CO—R″ (A)
—CON(CH₃)₂ (B)
—CR*R**—O—R″ (C)
—CR*R**—O—CO—R″ (D) 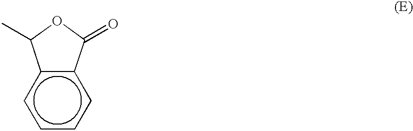
—CO—R* (F)
wherein R* and R** each independently designate hydrogen or C₁₋₃ alkyl, R″ designates C₁₋₆ alkyl or an optionally substituted aromate Ar¹ or Ar² as defined in claim 15.

15. The use according to claim 14, in which Z is selected from pivaloyl, pivaloyloxymethyl, N,N-dimethylcarbamoyl, and C₁₋₆ acyl.

16. The use according to any of claims 11-15, wherein at least one of Ar¹ and Ar² is optionally substituted phenyl, preferably both of Ar¹ and Ar² are optionally substituted phenyl.

17. The use according to claim 16, wherein, if m is 1, 2, 3 or 4, at least one of the groups AW is in a position in Ar¹ most remote relative to and/or next to the position through which Ar¹ is bound to the carbonyl group.

18. The use according to claim 16, wherein, if n is 1, 2 or 3, at least one of the groups AW is in a position in Ar² most remote relative to and/or next to the position through which Ar² is bound to V.

19. The use according to any of claims 11-18, wherein m is 1, 2, 3, or 4, preferably 2, 3, or 4.

20. The use according to any of claims 11-19, wherein n is 1, 2, or 3, preferably 2 or 3.

21. The use according to any of the claims 11-20, wherein the sum n+m is at least 2, preferably at least 3, in particular at least 4.

22. The use according to any of the claims 11-21, in which each A independently is selected from —O— and —NR_{H}—.

23. The use according to claim 22, wherein at least one A is —O—, preferably each A is —O—.

24. The use according to any of the claims 11-23, wherein R_{H} designates C₁₋₆ straight, branched, or cyclic aliphatic hydrocarbyl which may be saturated or may contain a double bond, which hydrocarbyl may be substituted with one or more substituents selected from hydroxy, halogen, amino, and amino which is optionally alkylated with one or two C₁₋₆ alkyl groups, preferably R_{H} designates C₁₋₆ straight, branched, or cyclic aliphatic hydrocarbyl which may be saturated or may contain a double bond.

25. The use according to claim 24, wherein R_{H} is selected from methyl, ethyl, propyl, isopropyl, butyl, isobutyl, secbutyl, tertbutyl, pentyl, 1-methylbutyl, 1,1-dimethylpropyl, hexyl, 1-methyl-pentyl, 1-ethylbutyl, cyclopentyl, cyclohexyl, prop-2-enyl, 1,1-dimethyl-prop-2-enyl, 3-methylbutyl, and 3-methylbut-2-enyl.

26. The use according to any of the claims 11-25, wherein any optional substituents on the aromates Ar¹ and Ar² are selected from halogen; cyano; nitro; and C₁₋₆, straight, branched or cyclic aliphatic hydrocarbyl which may be saturated or may contain a double bond, which hydrocarbyl may be substituted with one or more substituents selected from hydroxy, halogen, amino, and amino which is optionally alkylated with one or two C-₁₋₆ alkyl groups.

27. The use according to claim 26, wherein any substituents on the aromates Ar¹ and Ar² are selected from methyl, ethyl, propyl, isopropyl, butyl, isobutyl, secbutyl, tertbutyl, pentyl, 1-methylbutyl, 1,1-dimethylpropyl, hexyl, 1-methylpentyl, 1-ethylbutyl, cyclopentyl, cyclohexyl, prop-2-enyl, 1,1-dimethyl-prop-2-enyl, 3-methylbutyl, and 3-methylbut-2-enyl.

28. The use according to any of claims 11-27, in which the compound of formula I is a compound of one of the formulae II or IIa
(WA)ₘ—Ph—C(O)—CR═CR—Ph—(AW)ₙ II
(WA)ₘ—Ph—C(O)—CRR—CRR—Ph—(AW)ₙ IIa
wherein Ph designates optionally substituted phenyl, and W, A, R, n, and m are as defined in any of the claims 11-27.

29. The use according to claim 28, in which each AW is selected from OH, a group OR_{H}, in which R_{H} is as defined in claim 11, 24 or 25; and OZ, in which Z is a masking group which is readily decomposed under conditions prevailing in the animal body to liberate the group OH, in particular one of the groups (A)-(F) as defined in claim 14, preferably pivaloyl, pivaloyloxymethyl, N,N-dimethylcarbamoyl, or C₁₋₆ acyl.

30. The use according to claim 28 or claim 29, in which the compound is a bis-aromatic α,β-unsaturated ketone of the formula II.

31. The use according to claim 28 or claim 29, in which the compound is a bis-aromatic ketone of the formula IIa.

32. The use according to claim 30, wherein the compound is selected from the compounds defined in any of claims 57-253.

33. The use according to claim 31, wherein the compound is selected from the compounds defined in any of claims 254-274.

34. The use according to claim 30, wherein the bis-aromatic α,β-unsaturated ketone has the general formula 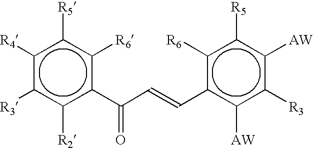
wherein R₃, R₅, and R₆, each independently is selected from hydrogen, cyano, nitro, nitroso, halogen, R_{H}, and AW, preferably hydrogen and R_{H}; R′₂, R′₃, R₄′, R′₅, and R′₆ each independently is selected from hydrogen, cyano, nitro, nitroso, halogen, R_{H}, and AW, preferably hydrogen and AW, wherein R_{H} and AW are as defined in any of claims 11-25.

35. The use according to claim 34, wherein the compound is selected from the compounds defined in any of claims 210-253.

36. The use according to claim 30, wherein the bis-aromatic α,β-unsaturated ketone has the general formula 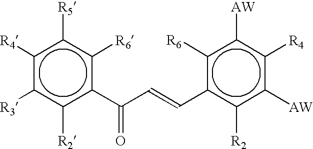
wherein R₂, R₄, and R₆ each independently is selected from hydrogen, cyano, nitro, nitroso, halogen, R_{H}, and AW, preferably hydrogen and R_{H}; R′₂, R′₃, R₄′, R′₅, and R′₆ each independently is selected from hydrogen, cyano, nitro, nitroso, halogen, R_{H}, and AW, preferably hydrogen and AW, wherein R_{H} and AW are as defined in any of claims 11-25.

37. The use according to claim 30, wherein the bis-aromatic α,β-unsaturated ketone has the general formula 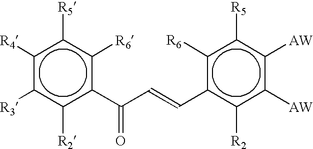
wherein R₂, R₅, and R₆, each independently is selected from hydrogen, cyano, nitro, nitroso, halogen, R_{H}, and AW, preferably hydrogen and R_{H}; R′₂, R′₃, R₄′, R′₅, and R′₆ each independently is selected from hydrogen, cyano, nitro, nitroso, halogen, R_{H}, and AW, preferably hydrogen and AW, wherein R_{H} and AW are as defined in any of claims 11-25.

38. The use according to claim 30, wherein the bis-aromatic α,β-unsaturated ketone has the general formula 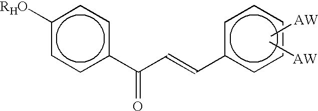
wherein AW is as defined in any of claims 11-25, preferably hydroxy or lower alkoxy such as methoxy or ethoxy, and R_{H} is as defined in any of claims 11-25, preferably prop-2-enyl.

39. The use according to claim 30, wherein the bis-aromatic α,β-unsaturated ketone has the general formula 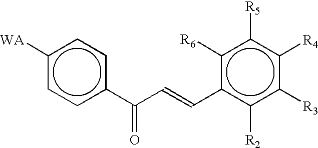
wherein R₂, R₃, R₄, R₅, and R₆ each independently is selected from hydrogen, cyano, nitro, nitroso, halogen, R_{H}, and AW, preferably hydrogen and R_{H}, wherein R_{H} and AW are as defined in any of claims 11-25.

40. The use according to claim 39, wherein the bis-aromatic α,β-unsaturated ketone has the general formula 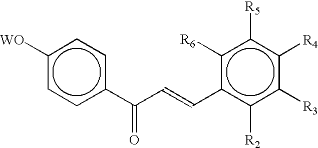
wherein R₂, R₃, R₅, and R₆ each independently is selected from hydrogen, cyano, nitro, nitroso, halogen, R_{H}, and AW, preferably hydrogen and R_{H}, wherein R_{H}, AW, and W are as defined in any of claims 11-25.

41. The use according to claim 34, wherein the bis-aromatic α,β-unsaturated ketone has the general formula 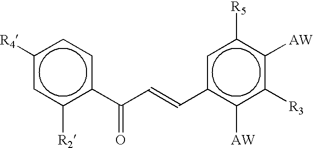
wherein R₃ and R₅ each independently is selected from hydrogen, cyano, nitro, nitroso, halogen, R_{H}, and AW, preferably hydrogen and R_{H}; R₂′ and R₄′ each independently is selected from hydrogen, cyano, nitro, nitroso, halogen, R_{H}, and AW, preferably hydrogen and AW, wherein R_{H} and AW are as defined in any of claims 11-25.

42. The use according to claim 36, wherein the bis-aromatic α,β-unsaturated ketone has the general formula 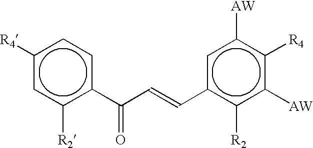
wherein R₂ and R₄ each independently is selected from hydrogen, cyano, nitro, nitroso, halogen, R_{H}, and AW, preferably hydrogen and R_{H}; R₂′ and R₄′ each independently is selected from hydrogen, cyano, nitro, nitroso, halogen, R_{H}, and AW, preferably hydrogen and AW, wherein R_{H} and AW are as defined in any of claims 11-25.

43. The use according to claim 37, wherein the bis-aromatic α,β-unsaturated ketone has the general formula 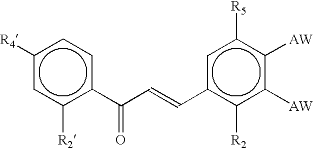
wherein R₂ and R₅ each independently is selected from hydrogen, cyano, nitro, nitroso, halogen, R_{H}, and AW, preferably hydrogen and R_{H}; R₂′ and R₄′ each independently is selected from hydrogen, cyano, nitro, nitroso, halogen, R_{H}, and AW, preferably hydrogen and AW, wherein R_{H} and AW are as defined in any of claims 11-25.

44. The use according to claim 41, wherein the bis-aromatic α,β-unsaturated ketone has the general formula 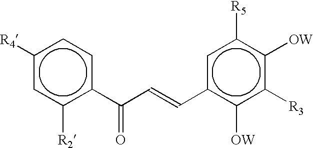
wherein R₃ and R₅ each independently is selected from hydrogen, cyano, nitro, nitroso, halogen, R_{H}, and AW, preferably hydrogen and R_{H}; R₂′ and R₄′ each independently is selected from hydrogen, cyano, nitro, nitroso, halogen, R_{H}, and AW, preferably hydrogen and AW, wherein R_{H}, AW and W are as defined in any of claims 11-25.

45. The use according to claim 44, wherein the bis-aromatic α,β-unsaturated ketone has the general formula 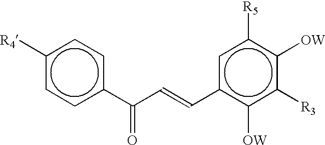
wherein R₃ and R₅ each independently is selected from hydrogen, cyano, nitro, nitroso, halogen, R_{H}, and AW, preferably hydrogen and R_{H}; R₄′ is selected from hydrogen, cyano, nitro, nitroso, halogen, R_{H}, and AW, preferably hydrogen and AW, wherein R_{H}, AW and W are as defined in any of claims 11-25.

46. The use according to claim 45, in which the bis-aromatic α,β-unsaturated ketone has the general formula 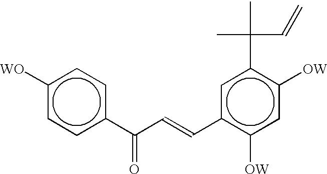
wherein W is as defined in any of claims 11-25.

47. The use according to claim 46, in which the bis-aromatic α,β-unsaturated ketone has the general formula 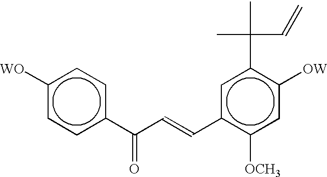
wherein W is as defined in any of claims 11-25.

48. The use according to claim 47, in which the bis-aromatic α,βunsaturated ketone has the general formula 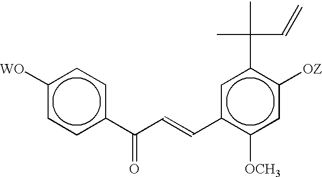
wherein Z is as defined in claim 14.

49. The use according to claim 48, wherein Z designated pivaloyl, pivaloyloxymethyl or N,N-dimethylcarbamoyl.

50. The use according to claim 42, wherein the bis-aromatic α,β-unsaturated ketone has the general formula 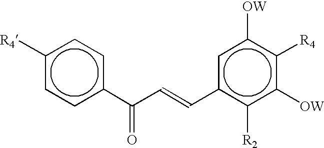
wherein R₂ and R₄ each independently is selected from hydrogen, cyano, nitro, nitroso, halogen, R_{H}, and AW, preferably hydrogen and R_{H}; R₄′ is selected from hydrogen, cyano, nitro, nitroso, halogen, R_{H}, and AW, preferably hydrogen and AW, wherein R_{H}, AW and W are as defined in any of claims 11-25.

51. The use according to claim 43, wherein the bis-aromatic α,β-unsaturated ketone has the general formula 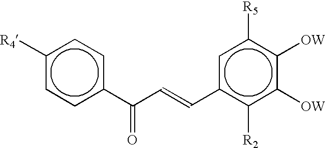
wherein R₂ and R₅ each independently is selected from hydrogen, cyano, nitro, nitroso, halogen, R_{H}, and AW, preferably hydrogen and R_{H}; R₄′ is selected from hydrogen, cyano, nitro, nitroso, halogen, R_{H}, and AW, preferably hydrogen and AW, wherein R_{H}, AW and W are as defined in any of claims 11-25.

52. The use according to claim 45, wherein the bis-aromatic α,β-unsaturated ketone has the general formula 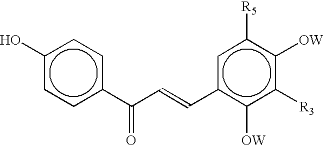
wherein R₃ and R₅ each independently is selected from hydrogen, cyano, nitro, nitroso, halogen, R_{H}, and AW, preferably hydrogen and R_{H}, wherein R_{H}, AW and W are as defined in any of claims 11-25.

53. The use according to claim 50, wherein the bis-aromatic α,β-unsaturated ketone has the general formula 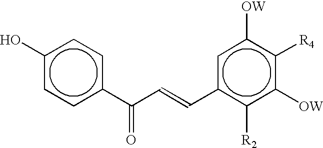
wherein R₂ and R₄ each independently is selected from hydrogen, cyano, nitro, nitroso, halogen, R_{H}, and AW, preferably hydrogen and R_{H}, wherein R_{H}, AW and W are as defined in any of claim 11-25.

54. The use according to claim 51, wherein the bis-aromatic α,β-unsaturated ketone has the general formula 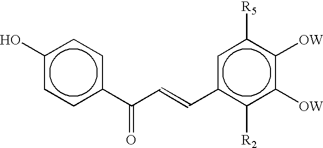
wherein R₂ and R₅ each independently is selected from hydrogen, cyano, nitro, nitroso, halogen, R_{H}, and AW, preferably hydrogen and R_{H}, wherein R_{H}, AW and W are as defined in any of claims 11-25.

55. The use according to claim 30, wherein the bis-aromatic α,β-unsaturated ketone has the general formula 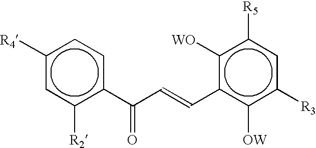
wherein R₃ and R₅ each independently is selected from hydrogen, cyano, nitro, nitroso, halogen, R_{H}, and AW, preferably hydrogen and R_{H}; R₂′ and R₄′ each independently is selected from hydrogen, cyano, nitro, nitroso, halogen, R_{H}, and AW, preferably hydrogen and AW, wherein R_{H}, AW and W are as defined in any of claims 11-25.

56. The use according to claim 30 in which the bis-aromatic α,β-unsaturated ketone has the general formula 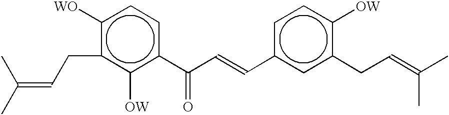
wherein W is as defined in any of claims 11-25.

57. A compound of the general formula 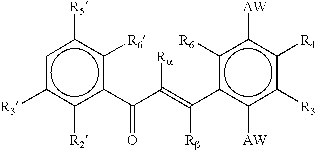
wherein
R₃, R₄ and R₆ each independently is selected from hydrogen, R_{H}, cyano, nitro, nitroso, amino, and halogen; R₆₀ , R₆₂ , R′₂, R′₃, R′₅, and R′₆ each independently is selected from hydrogen, cyano, nitro, nitroso, halogen, R_{H}, and AW, wherein each A independently is selected from —O—, —S—, —NH—, and —NR_{H}—, preferably —O— and —NR_{H}—, and each W independently is selected from hydrogen, R_{H}, and R_{H}CO—, preferably R_{H}, where R_{H} is selected from C₁₋₆ straight, branched and cyclic aliphatic hydrocarbyl which may be saturated or may contain one or more unsaturated bonds selected from double bonds and triple bonds, which hydrocarbyl may be substituted with one or more substituents selected from C₁₋₆ alkoxy, hydroxy, halogen, amino, and amino which is optionally alkylated with one or two C₁₋₆ alkyl groups;
with the first proviso that R_{α} and R_{β} are not cyano, and
with the second proviso that the compound is not one of:
1,1′-(1,3-phenylene)bis[3-(2,5-dimethoxyphenyl)-2-propenone,
1,3-bis(2,5dimethoxyphenyl)-2-propenone,
3-(2,5-dimethoxyphenyl)-1-(2-hydroxy-6-methoxyphenyl)-2-propenone,
3-(2,5-dimethoxyphenyl)-1-(2-hydroxyphenyl)-2-propenone,
1-phenyl-3-(2,3,4,6-tetramethoxyphenyl)-2-propenone,
3-(2-hydroxy-3,4,6-trimethoxyphenyl)-1-phenyl-2-propenone,
2′-chloro-2,5-dimethoxychalcone,
3-(2,5-dimethoxyphenyl)-1-(2-hydroxy-3,5-dimethylphenyl)-2-propen-1-one,
3-(2,5-dimethoxyphenyl)-1-phenyl-2-propenone,
2′-hydroxy-2,5-dimethoxy-5′-nitro-2-propen-1-one,
2-hydroxy-2′,3,4,5,5′,6-hexamethoxychalcone,
3-(2,5-dimethoxyphenyl)-1-(2-hydroxyphenyl)-2-propenone,
2-hydroxy-3,4,5,6-tetramethoxychalcone,
4-benzyloxy-2-hydroxy-3,5,6-trimethoxychalcone,
2′-hydroxy-2,4,5-trimethoxychalcone,
2,2′,5-trihydroxy-4-methoxychalcone,
2,3′-dihydroxy-4,5-dimethoxychalcone,
2-hydroxy-5- methoxychalcone,
2-hydroxy-2′,3,4,5,5′,6′-hexamethoxychalcone,
2-hydroxy-3,4,5,6-tetramethoxychalcone,
4-benzyloxy2-hydroxy-3,5,6-trimethoxychalcone,
2,5-dimethoxy-3′,5′-dimethyl-2′-hydroxychalcone,
4-hydroxy-2,5-dimethoxy-3-nitrochalcone,
2-hydroxy-2,5-dimethoxy4-nitrochalcone,
2-hydroxy-2,5-dimethoxy-5-nitrochalcone,
2-hydroxy-3,4,6-trimethoxychalcone,
2,3,4,6-tetramethoxychalcone,
2,5-dihydroxychalcone,
2′-hydroxy-2,5,6′-trimethoxychalcone,
2-hydroxy-3,4,6-trimethoxychalcone,
2,2′,5,5′-tetramethoxychalcone,
2,5dimethoxy-2′-hydroxy-chalcone,
2,5,2′-trihydroxychalcone,
2,5-dihydroxychalcone,
2,5,2′-trihydroxy-5′-methylchalcone, or
2,2′,5,5′-tetrahydroxychalcone.

58. A compound according to claim 57, wherein each of R_{α} and R_{β} independently is selected from C₁₋₃ alkyl, cyano, and halogen.

59. A compound according to claim 57, wherein R_{α} is hydrogen and R_{β} is as defined in claim 58.

60. A compound according to claim 57, wherein R_{β} is hydrogen and R_{α} is as defined in claim 58.

61. A compound according to claim 57, wherein R_{α} and R_{β} are both hydrogen.

62. A compound according to any of claims 57-61, wherein R_{H} is selected from C₁₋₆straight, branched and cyclic aliphatic hydrocarbyl which may be saturated or may contain one or more unsaturated bonds selected from double bonds and triple bonds.

63. A compound according to claim 62, wherein R_{H} is selected from methyl, ethyl, propyl, isopropyl, butyl, isobutyl, secbutyl, tertbutyl, pentyl, 1-methylbutyl, 1,1-dimethylpropyl, hexyl, 1-methylpentyl, 1-ethylbutyl, cyclopentyl, cyclohexyl, prop-2-enyl, 1,1-dimethyl-prop-2-enyl, and 3-methyl-but-2-enyl.

64. A compound according to any of claims 57-63, wherein R₃, R₄, and R₆ each independently is selected from hydrogen and R_{H}.

65. A compound according to any of claims 57-64, wherein R′₂, R′₃, R′₅, and R′₆ each independently is selected from hydrogen, R_{H}, and AW.

66. A compound according to any of claims 57-64 with the general formula

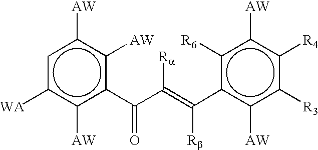

67. A compound according to claim 66 wherein AW is OR_{H}.

68. A compound according to claim 67 wherein R₃, R₄, and R₆ are hydrogen.

69. A compound according to claim 68 selected from
2,5,2′,3′,5′,6′-hexamethoxychalcone,
2,5,2′,3′,5′,6′-hexaethoxychalcone,
2,5,2′,3′,5′,6′-hexapropoxychalcone,
2,5,2′,3′,5′,6′-hexa-isopropoxychalcone,
2,5,2′,3′,5′,6′-hexabutoxychalcone,
2,5,2′,3′,5′,6′-hexa-isobutoxychalcone,
2,5,2′,3′,5′,6′-hexa-tertbutoxychalcone,
2,5,2′,3′,5′,6′-hexapentoxychalcone,
2,5,2′,3′,5′,6′-hexa-(1-methylbutyl)oxychalcone,
2,5,2′,3′,5′,6′-hexahexoxychalcone,
2,5,2′,3′,5′,6′-hexa-(1-methylpentyl)oxychalcone,
2,5,2,3′,5′,6′-hexa-(1-ethylbutyl)oxychalcone, and
2,5,2′,3′,5′,6′-hexacyclohexoxychalcone.

70. A compound according to any of claims 57-65 with the general formula

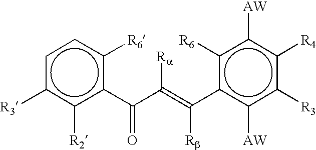

71. A compound according to claim 70 with the general formula

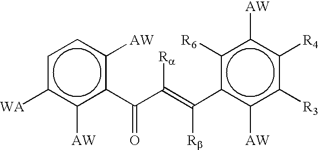

72. A compound according to claim 71 wherein AW is OR_{H}.

73. A compound according to claim 72 wherein R₃, R₄, and R₆ are hydrogen.

74. A compound according to claim 73 selected from
2,5,2′,3′,6′-pentamethoxychalcone,
2,5,2′,3′,6′-pentaethoxychalcone,
2,5,2′,3′,6′-pentapropoxychalcone,
2,5,2′,3′,6′-penta-isopropoxychalcone,
2,5,2′,3′,6′-pentabutoxychalcone,
2,5,2′,3′,6′-penta-isobutoxychalcone,
2,5,2′,3′,6′-penta-tertbutoxychalcone,
2,5,2′,3′,6′-pentapentoxychalcone,
2,5,2′,3′,6′-penta-(1-methylbutyl)oxychalcone,
2,5,2′,3′,6′-pentaethoxychalcone,
2,5,2′,3′,6′-penta-(1-methylpentyl)oxychalcone,
2,5,2′,3′,6′-pental -ethylbutyl)oxychalcone, and
2,5,2′,3′,6′-pentacyclohexoxychalcone.

75. A compound according to claim 70 with the general formula

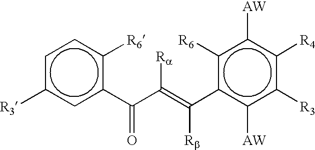

76. A compound according to claim 75 with the general formula

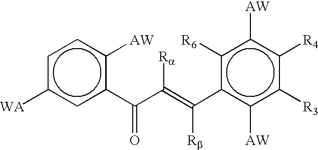

77. A compound according to claim 76 wherein AW is OR_{H}.

78. A compound according to claim 77 wherein R₃, R₄, and R₆ are hydrogen.

79. A compound according to claim 70 with the general formula

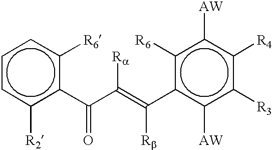

80. A compound according to claim 79 with the general formula

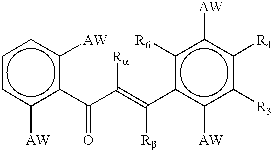

81. A compound according to claim 80 wherein AW is OR_{H}.

82. A compound according to claim 81 wherein R₃, R₄, and R₆ are hydrogen.

83. A compound according to any of claims 57-65 with the general formula

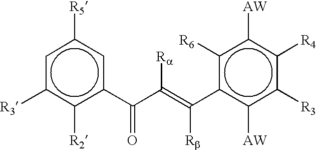

84. A compound according to claim 83 with the general formula

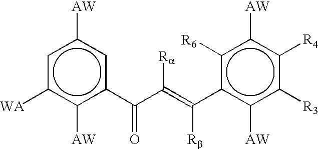

85. A compound according to claim 84 wherein AW is O_{R}_{H}.

86. A compound according to claim 85 wherein R₃, R₄, and R₆ are hydrogen.

87. A compound according to claim 86 selected from
2,5,2′,3′,5′-pentamethoxychalcone,
2,5,2′,3′,5′-pentaethoxychalcone,
2,5,2′,3′,5′-pentapropoxychalcone,
2,5,2′,3′,5′-pentaisopropoxychalcone,
2,5,2′,3′,5′-pentabutoxychalcone,
2,5,2′,3′,5′-pentabutoxychalcone,
2,5,2′,3′,5′-penta-tertbutoxychalcone,
2,5,2′,3′,5′-pentapentoxychalcone,
2,5,2′,3′,5′-penta-(1-methylbutyl)oxychalcone,
2,5,2′,3′,5′-pentaethoxychalcone,
2,5,2′,3′,5′-penta-(1-methylpentyl)oxychalcone,
2,5,2′,3′,5′-penta-(1-ethylbutyl)oxychalcone, and
2,5,2′,3′,5′-pentacyclohexoxychalcone.

88. A compound according to claim 83 with the general formula

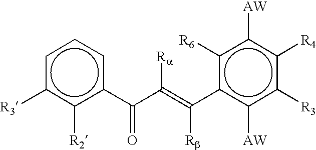

89. A compound according to claim 88 with the general formula

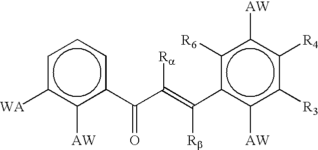

90. A compound according to claim 89 wherein AW is OR_{H}.

91. A compound according to claim 90 wherein R₃, R₄, and R₆ are hydrogen.

92. A compound according to claim 91 selected from
2,5,2′,3′-tetramethoxychalcone,
2,5,2′,3′-tetraethoxychalcone,
2,5,2′,3′-tetrapropoxychalcone,
2,5,2′,3′-tetra-isopropoxychalcone,
2,5,2′,3′-tetrabutoxychalcone,
2,5,2′,3′-tetra-isobutoxychalcone,
2,5,2′,3′-tetra-tertbutoxychalcone,
2,5,2′,3′-tetrapentoxychalcone,
2,5,2′,3′-tetra-(1-methylbutyl)oxychalcone,
2,5,2′,3′-tetrahexoxychalcone,
2,5,2′,3′-tetra-(1-methylpentyl)oxychalcone,
2,5,2′,3′-tetra-(1-ethylbutyl)oxychalcone, and
2,5,2′,3′-tetracyclohexoxychalcone.

93. A compound according to claim 88 with the general formula

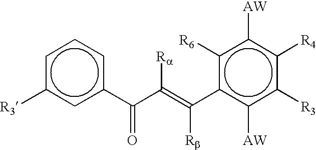

94. A compound according to claim 93 with the general formula

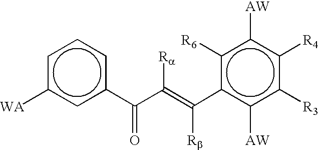

95. A compound according to claim 94 wherein AW is OR_{H}.

96. A compound according to claim 95 wherein R₃, R₄, and R₆ are hydrogen.

97. A compound according to claim 96 selected from
2,5,3′-trimethoxychalcone,
2,5,3′-triethoxychalcone,
2,5,3′-tripropoxychalcone,
2,5,3′-tri-isopropoxychalcone,
2,5,3′-tributoxychalcone,
2,5,3′-tri-isobutoxychalcone,
2,5,3′-tri-tertbutoxychalcone,
2,5,3′-tripentoxychalcone,
2,5,3′-tri-(1-methylbutyl)oxychalcone,
2,5,3′-trihexoxychalcone,
2,5,3′-tri-(1-methylpentyl)oxychalcone,
2,5,3′-tri-(1-ethylbutyl)oxychalcone, and
2,5,3′-tricyclohexoxychalcone.

98. A compound according to claim 83 with the general formula

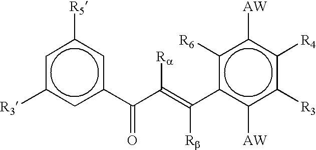

99. A compound according to claim 98 with the general formula

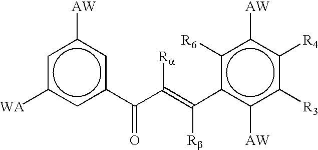

100. A compound according to claim 99 wherein AW is OR_{H}.

101. A compound according to claim 100 wherein R₃, R₄, and R₆ are hydrogen

102. A compound according to claim 88 with the general formula

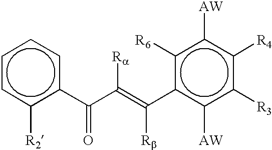

103. A compound according to claim 102 with the general formula

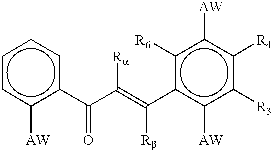

104. A compound according to claim 103 wherein AW is OR_{H}.

105. A compound according to claim 104 wherein R₃, R₄, and R₆ are hydrogen.

106. A compound according to claim 105 selected from
2,5,2′-trimethoxychalcone,
2,5,2′-triethoxychalcone,
2,5,2′-tripropoxychalcone,
2,5,2′-tri-isopropoxychalcone,
2,5,2′-tributoxychalcone,
2,5,2′-tri-isobutoxychalcone,
2,5,2′-tri-tertbutoxychalcone,
2,5,2′-tripentoxychalcone,
2,5,2′-tri-(1-methylbutyl)oxychalcone,
2,5,2′-trihexoxychalcone,
2,5,2′-tri-(1-methylpentyl)oxychalcone,
2,5,2′-tri-(1-ethylbutyl)oxychalcone, and
2,5,2′-tricyclohexoxychalcone.

107. A compound of the general formula 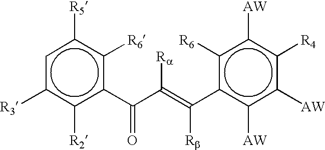
wherein R₄ and R₆ each independently is selected from hydrogen, R_{H}, cyano, nitro, nitroso, amino, and halogen; R_{α}, R_{β}, R′₂, R′₃, R′₅, and R′₆ each independently is selected from hydrogen, cyano, nitro, nitroso, halogen, R_{H}, and AW, wherein each A independently is selected from —O—, —S—, —NH—, and —NR_{H}—, preferably —O— and —NR_{H}, and each W independently is selected from hydrogen, R_{H}, and R_{H}CO—, preferably R_{H}, wherein R_{H} is selected from C₁₋₆ straight, branched and cyclic aliphatic hydrocarbyl which may be saturated or may contain one or more unsaturated bonds selected from double bonds and triple bonds, which hydrocarbyl may be substituted with one or more substituents selected from C₁₋₆ alkoxy, hydroxy, halogen, amino, and amino which is optionally alkylated with one or two C₁₋₆ alkyl groups.

108. A compound according to claim 107, wherein each of R_{α} and R_{β} independently is selected from C₁₋₃ alkyl, cyano, and halogen.

109. A compound according to claim 107, wherein R_{α} is hydrogen and R_{β} is as defined in claim 108.

110. A compound according to claim 107, wherein R_{β} is hydrogen and R_{α} is as defined in claim 108.

111. A compound according to claim 107, wherein R_{α} and R_{β} are both hydrogen.

112. A compound according to any of claims 107-111, wherein R_{H} is selected from C₁₋₆ straight, branched and cyclic aliphatic hydrocarbyl which may be saturated or may contain one or more unsaturated bonds selected from double bonds and triple bonds.

113. A compound according to claim 112, wherein R_{H} is selected from methyl, ethyl, propyl, isopropyl, butyl, isobutyl, secbutyl, tertbutyl, pentyl, 1-methylbutyl, 1,1-dimethylpropyl, hexyl, 1-methylpentyl, 1-ethylbutyl, cyclopentyl, cyclohexyl, prop-2-enyl, 1,1-dimethyl-prop-2-enyl, and 3-methyl-but-2-enyl.

114. A compound according to any of claims 107-113, wherein R₄ and R₆ each independently is selected from hydrogen and R_{H}.

115. A compound according to any of claims 107-114, wherein R′₂, R′₃, R′₅, and R′₆ each independently is selected from hydrogen, R_{H}, and AW.

116. A compound according to any of claims 107-114 with the general formula

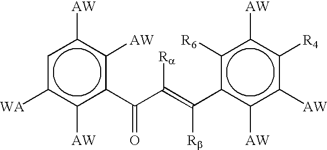

117. A compound according to claim 116 wherein AW is OR_{H}.

118. A compound according to claim 117, wherein R₄ and R₆ are hydrogen.

119. A compound according to claim 118 selected from
3-methoxy-2,5,2′,3′,5′,6′-hexamethoxychalcone,
3-methoxy-2,5,2′,3′,5′,6′-hexaethoxychalcone,
3-methoxy-2,5,2′,3′,5′,6′-hexapropoxychalcone,
3-methoxy-2,5,2′,3′,5′,6′-hexa-isopropoxychalcone,
3-methoxy-2,5,2′,3′,5′,6′-hexabutoxychalcone,
3-methoxy-2,5,2′,3′,5′,6′-hexa-isobutoxychalcone,
3-methoxy-2,5,2′,3′,5′,6′-hexa-tertbutoxychalcone,
3-methoxy-2,5,2′,3′,5′,6′-hexapentoxychalcone,
3-methoxy-2,5,2′,3′,5′,6′-hexa-(1-methylbutyl)oxychalcone,
3-methoxy-2,5,2′,3′,5′,6′-hexahexoxychalcone,
3-methoxy-2,5,2′,3′,5′,6′-hexa-(1-methylpentyl)oxychalcone,
3-methoxy-2,5,2′,3′,5′,6′-hexa-(1-ethylbutyl)oxychalcone, and
3-methoxy-2,5,2′,3′,5′,6′-hexacyclohexoxychalcone.

120. A compound according to any of claims 107-115 with the general formula

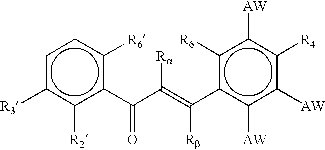

121. A compound according to claim 120 with the general formula

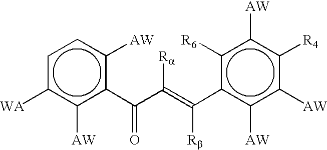

122. A compound according to claim 121 wherein AW is OR_{H}.

123. A compound according to claim 122, wherein R₄ and R₆ are hydrogen.

124. A compound according to claim 123 selected from
3-methoxy-2,5,2′,3′,6′-pentamethoxychalcone,
3-methoxy-2,5,2′,3′,6′-pentaethoxychaleone,
3-methoxy-2,5,2′,3′,6′-pentapropoxychaleone,
3-methoxy-2,5,2′,3′,6′-penta-isopropoxychalcone,
3-methoxy-2,5,2′,3′,6′-pentabutoxychalcone,
3-methoxy-2,5,2′,3′,6′-penta-isobutoxychalcone,
3-methoxy-2,5,2′,3′,6′-penta-tertbutoxychalcone,
3-methoxy-2,5,2′,3′,6′-pentapentoxychalcone,
3-methoxy-2,5,2′,3′,6′-penta-(1-methylbutyl)oxychalcone,
3-methoxy-2,5,2′,3′,6′-pentahexoxychalcone,
3-methoxy-2,5,2′,3′,6′-penta-(1-methylpentyl)oxychalcone,
3-methoxy-2,5,2′,3′,6′-penta-(1-ethylbutyl)oxychalcone, and
3-methoxy-2,5,2′,3′,6′-pentacyclohexoxychalcone.

125. A compound according to claim 120 with the general formula

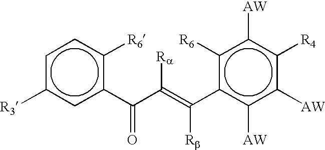

126. A compound according to claim 125 with the general formula

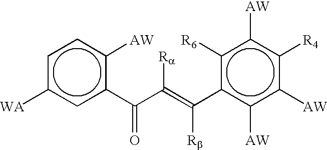

127. A compound according to claim 126 wherein AW is OR_{H}.

128. A compound according to claim 127 wherein R₄ and R₆ are hydrogen.

129. A compound according to claim 120 with the general formula

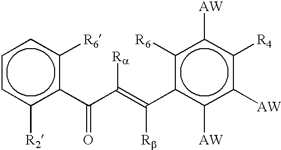

130. A compound according to claim 129 with the general formula

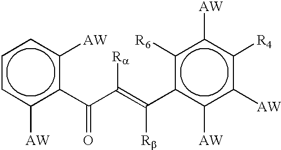

131. A compound according to claim 130 wherein AW is OR_{H}.

132. A compound according to claim 131, wherein R₄ and R₆ are hydrogen.

133. A compound according to claim 129 with the general formula

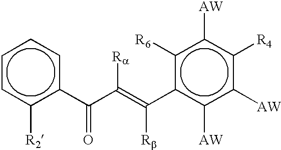

134. A compound according to claim 133 with the general formula

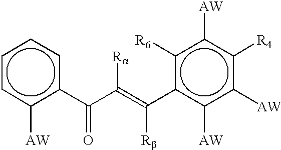

135. A compound according to claim 134 wherein AW is OR_{H}.

136. A compound according to claim 135, wherein R₄ and R₆ are hydrogen.

137. A compound according to claim 136 selected from
3-methoxy-2,5,2′-trimethoxychalcone,
3-methoxy-2,5,2′-triethoxychalcone,
3-methoxy-2,5,2′-tripropoxychalcone,
3-methoxy-2,5,2′-tri-isopropoxychalcone,
3-methoxy-2,5,2′-tributoxychalcone,
3-methoxy-2,5,2′-tri-isobutoxychalcone,
3-methoxy-2,5,2′-tri-tertbutoxychalcone,
3-methoxy-2,5,2′-tripentoxychalcone,
3-methoxy-2,5,2′-tri-(1-methylbutyl)oxychalcone,
3-methoxy-2,5,2′-trihexoxychalcone,
3-methoxy-2,5,2′-tri-(1-methylpentyl)oxychalcone,
3-methoxy-2,5,2′-tri-(1-ethylbutyl)oxychalcone, and
3-methoxy-2,5,2′-tricyclohexoxychalcone.

138. A compound according to any of claims 107-115 with the general formula

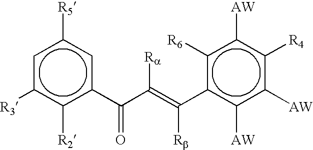

139. A compound according to claim 138 with the general formula

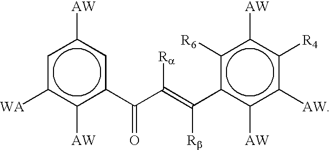

140. A compound according to claim 139 wherein AW is OR_{H}.

141. A compound according to claim 140, wherein R₄ and R₆ are hydrogen.

142. A compound according to claim 141 selected from
3-methoxy-2,5,2′,3′,5′-pentamethoxychalcone,
3-methoxy-2,5,2′,3′,5′-pentaethoxychalcone,
3-methoxy-2,5,2′,3′,5′-pentapropoxychalcone,
3-methoxy-2,5,2′,3′,5′-penta-isopropoxychalcone,
3-methoxy-2,5,2′,3′,5′-pentabutoxychalcone,
3-methoxy-2,5,2′,3′,5′-penta-isobutoxychalcone,
3-methoxy-2,5,2′,3′,5′-penta-tertbutoxychalcone,
3-methoxy-2,5,2′,3′,5′-pentapentoxychalcone,
3-methoxy-2,5,2′,3′,5′-penta-(1-methylbutyl)oxychalcone,
3-methoxy-2,5,2′,3′,5′-pentahexoxychalcone,
3-methoxy-2,5,2′,3′,5′-penta-(1-methylpentyl)oxychalcone,
3-methoxy-2,5,2′,3′,5′-penta-(1-ethylbutyl)oxychalcone, and
3-methoxy-2,5,2′,3′,5′-pentacyclohexoxychalcone.

143. A compound according to claim 138 with the general formula

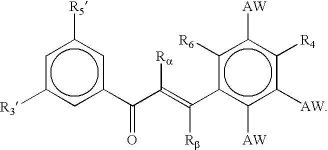

144. A compound according to claim 143 with the general formula

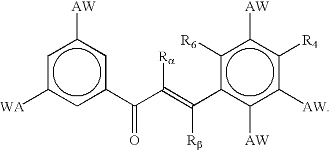

145. A compound according to claim 144 wherein AW is OR_{H}.

146. A compound according to claim 145, wherein R₄ and R₆ are hydrogen.

147. A compound according to claim 138 with the general formula

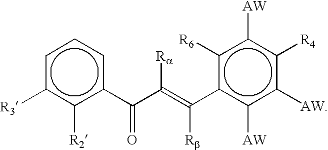

148. A compound according to claim 147 with the general formula

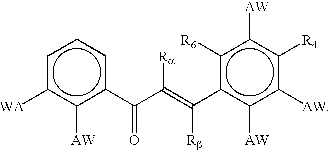

149. A compound according to claim 148 wherein AW is OR_{H}.

150. A compound according to claim 149, wherein R₄ and R₆ are hydrogen.

151. A compound according to claim 150 selected from
3-methoxy-2,5,2′,3′-tetramethoxychalcone,
3-methoxy-2,5,2′,3′-tetraethoxychalcone,
3-methoxy-2,5,2′,3′-tetrapropoxychalcone,
3-methoxy-2,5,2′,3′-tetra-isopropoxychalcone,
3-methoxy-2,5,2′,3′-tetrabutoxychalcone,
3-methoxy-2,5,2′,3′-tetra-isobutoxychalcone,
3-methoxy-2,5,2′,3′-tetra-tertbutoxychalcone,
3-methoxy-2,5,2′,3′-tetrapentoxychalcone,
3-methoxy-2,5,2′,3′-tetra-(1-methylbutyl)oxychalcone,
3-methoxy-2,5,2′,3′-tetrahexoxychalcone,
3-methoxy-2,5,2′,3′-tetra-(1-methylpentyl)oxychalcone,
3-methoxy-2,5,2′,3′-tetra-(1-ethylbutyl)oxychalcone, and
3-methoxy-2,5,2′,3′-tetracyclohexoxychalcone.

152. A compound according to claim 147 with the general formula

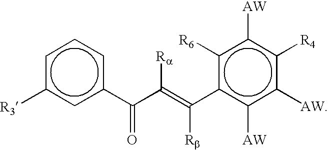

153. A compound according to claim 152 with the general formula

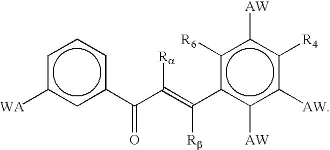

154. A compound according to claim 153 wherein AW is OR_{H}.

155. A compound according to claim 154, wherein R₄ and R₆ are hydrogen.

156. A compound according to claim 155 selected from
3-methoxy-2,5,3′-trimethoxychalcone,
3-methoxy-2,5,3′-triethoxychalcone,
3-methoxy-2,5,3′-tripropoxychalcone,
3-methoxy-2,5,3′-tri-isopropoxychalcone,
3-methoxy-2,5,3′-tributoxychalcone,
3-metboxy-2,5,3′-tri-isobutoxychalcone,
3-methoxy-2,5,3′-tri-tertbutoxychalcone,
3-methoxy-2,5,3′-tripentoxychalcone,
3-methoxy-2,5,3′-tri-(1-methylbutyl)oxychalcone,
3-methoxy-2,5,3′-trihexoxychalcone,
3-methoxy-2,5,3′-tri-(1-methylpentyl)oxychalcone,
3-methoxy-2,5,3′-tri-(1-ethylbutyl)oxychalcone, and
3-methoxy-2,5,3′-tricyclohexoxychalcone.

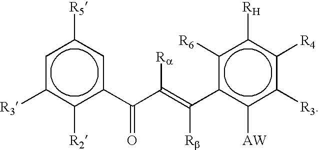

157. A compound of the general formula
wherein R₃, R₄, and R₆ each independently is selected from hydrogen, R_{H}, cyano, nitro, nitroso, amino, and halogen; R_{α}, R_{β}, R′₂, R′₃, R′₅, and R′₆ each independently is selected from hydrogen, cyano, nitro, nitroso, halogen, R_{H}, and AW, wherein each A independently is selected from —O—, —S—, —NH—, and —NR_{H}—, preferably —O— and —NR_{H}, and each W independently is selected from hydrogen, R_{H}, and R_{H}CO—, preferably R_{H}, wherein R_{H} is selected from C₁₋₆ straight, branched and cyclic aliphatic hydrocarbyl which may be saturated or may contain one or more unsaturated bonds selected from double bonds and triple bonds, which hydrocarbyl may be substituted with one or more substituents selected from C₁₋₆ alkoxy, hydroxy, halogen, amino, and amino which is optionally alkylated with one or two C₁₋₆ alkyl groups,
with the proviso that the compound is not one of
3-(2,6-dihydroxy-4-methoxy-3-methylphenyl)-1-phenyl-2-propen-1-one,
3-(3-[4,6-bis(acetyloxy)-2-methoxy-3-methylphenyl]-1-phenyl-2-propenone,
2,6-dihydroxy-4-methoxy-3-methylphenyl)-1-phenyl-2-propen-1-one,
1-phenyl-3-(2,4,6-trimethoxy-3-methylphenyl)-2-propen-1-one,
3-(4,6-dihydroxy-2-methoxy-3-methylphenyl)-1-phenyl-2-propenone,
3-(2-hydrxoy-5-methylphenyl)-1-phenyl-2-propen-1-one,
3-[2-hydroxy-5-(1-methyl-1-phenylethyl)phenyl]-1-phenyl-2-propen-1-one,
3-hydroxy-3-(2-hydroxy-4,6-dimethoxy-3-methylphenyl)-1-phenyl-2-propen-1-one,
1,3-bis[5-(1,1-dimethylethyl)-2-methoxyphenyl]-2-prop-1-one,
1,3-bis[5-(1,1-dimethylethyl)-2-methylphenyl]-2-prop-1-one,
5-isopropyl-4-methoxy-2-methylchalcone,
4-hydroxy-5-isopropyl-2-methylchalcone,
2,2′-dihydroxy-5-methylchalcone,
3-(2,6-dihydroxy-4-methoxy-3-methylphenyl)-1-phenyl-2-propen-1-one,
1-(5-chloro-2-hydroxyphenyl)-3-(2-hydroxy-5-methylphenyl)-2-propen-1-one,
1,3-bis(2-hydroxy-5-methylphenyl)-2-propen-1-one, or
1,3-bis(2,5-dimethylphenyl)-2-buten-1-one.

158. A compound according to claim 157, wherein each of R_{α} and R_{β} independently is selected from C₁₋₃ alkyl, cyano, and halogen.

159. A compound according to claim 157, wherein R_{α} is hydrogen and R_{β} is as defined in claim 158.

160. A compound according to claim 157, wherein R_{β} is hydrogen and R_{α} is as defined in claim 158.

161. A compound according to claim 157, wherein R_{α} and R_{β} are both hydrogen.

162. A compound according to any of claims 157-161, wherein R_{H} is selected from C₁₋₆ straight, branched and cyclic aliphatic hydrocarbyl which may be saturated or may contain one or more unsaturated bonds selected from double bonds and triple bonds.

163. A compound according to claim 162, wherein R_{H} is selected from methyl, ethyl, propyl, isopropyl, butyl, isobutyl, secbutyl, tertbutyl, pentyl, 1-methylbutyl, 1,1-dimethylpropyl, hexyl, 1-methylpentyl, 1-ethylbutyl, cyclopentyl, cyclohexyl, prop-2-enyl, 1,1-dimethyl-prop-2-enyl, and 3-methyl-but-2-enyl.

164. A compound according to any of claims 157-163, wherein R₃, R₄ and R₆ each independently is selected from hydrogen and R_{H}.

165. A compound according to any of claims 157-164, wherein R′₂, R′₃, R′₅, and R′₆ each independently is selected from hydrogen, R_{H}, and AW.

166. A compound according to any of claims 157-165 with the general formula

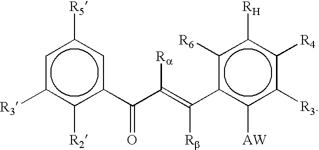

167. A compound according to claim 166 with the general formula

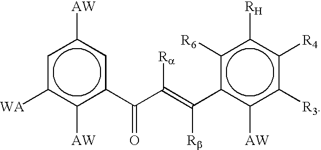

168. A compound according to claim 167 wherein AW is OR_{H}.

169. A compound according to claim 168 wherein R₃, R₄, and R₆ are hydrogen.

170. A compound according to claim 169 selected from
2,2′,3′,5′-tetramethoxy-5-ethylchalcone,
2,2′,3′,5′-tetraethoxy-5-ethylchalcone,
2,2′,3′,5′-tetrapropoxy-5-ethylchalcone,
2,2′,3′,5′-tetrabutoxy-5-ethylchalcone,
2,2′,3′,5′-tetraisobutoxy-5-ethylchalcone,
2,2′,3′,5′-tetrapentoxy-5-ethylchalcone,
2,2′,3′,5′-tetra-(1-methylbutyl)oxy-5-ethylchalcone,
2,2′,3′,5′-tetra-(1-ethylpropyl)oxy-5-ethylchalcone,
2,2′,3′,5′-tetrahexoxy-5-ethylchalcone,
2,2′,3′,5′-tetra-(1-methylpentyl)oxy-5-ethylchalcone,
2,2′,3′,5′-tetra-(1-ethylbutyl)oxy-5-ethylchalcone, and
2,2′,3′,5′-tetra-(1-propylpropyl)oxy-5-ethylchalcone.

171. A compound according to claim 166 with the general formula

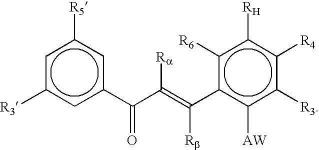

172. A compound according to claim 171 with the general formula

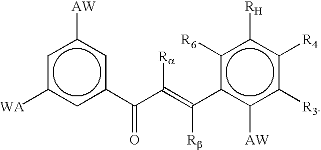

173. A compound according to claim 172 wherein AW is OR_{H}.

174. A compound according to claim 173 wherein R₃, R₄, and R₆ are hydrogen.

175. A compound according to claim 174 selected from
2,3′,5′-trimethoxy-5-ethylchalcone,
2,3′,5′-triethoxy-5-ethylchalcone,
2,3′,5′-tripropoxy-5-ethylchalcone,
2,3′,5′-tributoxy-5-ethylchalcone,
2,3′,5′-triisobutoxy-5-ethylchalcone,
2,3′,5′-tripentoxy-5-ethylchalcone,
2,3′,5′-tri-(1-methylbutyl)oxy-5-ethylchalcone,
2,3′,5′-tri-(1-ethylpropyl)oxy-5-ethylchalcone,
2,3′,5′-trihexoxy-5-ethylchalcone,
2,3′,5′-tri-(1-methylpentyl)oxy-5-ethylchalcone,
2,3′,5′-tri-(1-ethylbutyl)oxy-5-ethylchalcone, and
2,3′,5′-tri-(1-propylpropyl)oxy-5-ethylchalcone.

176. A compound according to any of claims 157-165 with the general formula

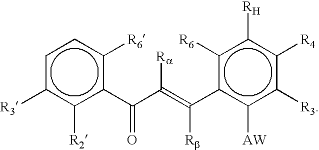

177. A compound according to claim 176 with the general formula

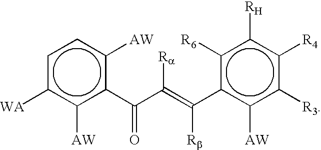

178. A compound according to claim 177 wherein AW is OR_{H}.

179. A compound according to claim 178 wherein R₃, R₄, and R₆ are hydrogen.

180. A compound according to claim 179 selected from
2,2′,3′,6′-tetramethoxy-5-ethylchalcone,
2,2′,3′,6′-tetraethoxy-5-ethylchalcone,
2,2′,3′,6′-tetrapropoxy-5-ethylchalcone,
2,2′,3′,6′-tetrabutoxy-5-ethychalcone,
2,2′,3′,6′-tetraisobutoxy-5-ethylchalcone,
2,2′,3′,6′-tetrapentoxy-5-ethylchalcone,
2,2′,3′,6′-tetra-(1-methylbutyl)oxy-5-ethylchalcone,
2,2′,3′,6′-tetra-(1-ethylpropyl)oxy-5-ethylchalcone,
2,2′,3′,6′-tetrahexoxy-5-ethylchalcone,
2,2′,3′,6′-tetra-(1-methylpentyl)oxy-5-ethylchalcone,
2,2′,3′,6′-tetra-(1-ethylbutyl)oxy-5-ethylchalcone, and
2,2′,3′,6′-tetra-(1-propylpropyl)oxy-5-ethylchalcone.

181. A compound according to claim 176 with the general formula

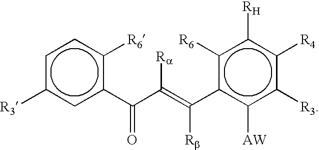

182. A compound according to claim 181 with the general formula

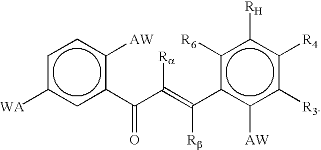

183. A compound according to claim 182 wherein AW is OR_{H}.

184. A compound according to claim 183 wherein R₄ and R₆ are hydrogen.

185. A compound according to claim 184 selected from
2,2′,5′-trimethoxy-5-ethylchalcone,
2,2′,5′-triethoxy-5-ethylchalcone,
2,2′,5′-tripropoxy-5-ethylchalcone,
2,2′,5′-tributoxy-5-ethylchalcone,
2,2′,5′-triisobutoxy-5-ethylchalcone,
2,2′,5′-tripentoxy-5-ethylchalcone,
2,2′,5′-tri-(1-methylbutyl)oxy-5-ethylchalcone,
2,2′,5′-tri-(1-ethylpropyl)oxy-5-ethylchalcone,
2,2′,5′-trihexoxy-5-ethylchalcone,
2,2′,5′-tri-(1-methylpentyl)oxy-5-ethylchalcone,
2,2′,5′-tri-(1-ethylbutyl)oxy-5-ethylchalcone, and
2,2′,5′-tri(-propylpropyl)oxy-5-ethylchalcone.

186. A compound according to claim 176 with the general formula

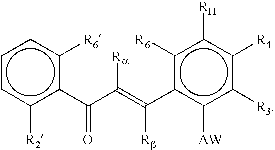

187. A compound according to claim 186 with the general formula

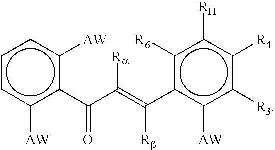

188. A compound according to claim 187 wherein AW is OR_{H}.

189. A compound according to claim 188 wherein R₃, R₄ and R₆ are hydrogen.

190. A compound according to claim 189 selected from
2,2′,6′-trimethoxy-5-ethylchalcone,
2,2′,6′-triethoxy-5-ethylchalcone,
2,2′,6′-tripropoxy-5-ethylchalcone,
2,2′,6′-tributoxy-5-ethylchalcone,
2,2′,6′-triisobutoxy-5-ethylchalcone,
2,2′,6′-tripentoxy-5-ethylchalcone,
2,2′,6′-tri-(1-methylbutyl)oxy-5-ethylchalcone,
2,2′,6′-tri-(1-ethylpropyl)oxy-5-ethylchalcone,
2,2′,6′-trihexoxy-5-ethylchalcone,
2,2′,6′-tri-(1-methylpentyl)oxy-5-ethylchalcone,
2,2′,6′-tri-(1-ethylbutyl)oxy-5-ethylchalcone, and
2,2′,6′-tri-(1-propylpropyl)oxy-5-ethylchalcone.

191. A compound according to claim 186 with the general formula

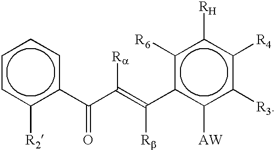

192. A compound according to claim 191 with the general formula

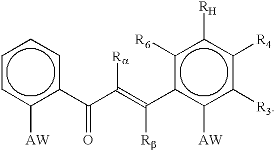

193. A compound according to claim 192 wherein AW is OR_{H}.

194. A compound according to claim 193 wherein R₃, R₄, and R₆ are hydrogen.

195. A compound according to claim 194 selected from
2,2′-dimethoxy-5ethylchalcone,
2,2′-diethoxy-5-ethylchalcone,
2,2′-dipropoxy-5-ethylchalcone,
2,2′-dibutoxy-5-ethylchalcone,
2,2′-diisobutoxy-5-ethylchalcone,
2,2′-dipentoxy-5-ethylchalcone,
2,2′-di-(1-methylbutyl)oxy-5-ethylchalcone,
2,2′-di-(1-ethylpropyl)oxy-5-ethylchalcone,
2,2′-dihexoxy-5-ethylchalcone,
2,2′-di-(1-methylpentyl)oxy-5-ethylchalcone,
2,2′-di-(1-ethylbutyl)oxy-5-ethylchalcone, and
2,2′-di-(1-propylpropyl)oxy-5-ethylchalcone.

196. A compound according to claim 176 with the general formula

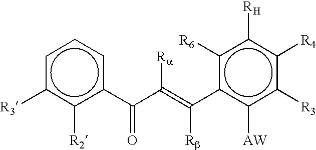

197. A compound according to claim 196 with the general formula

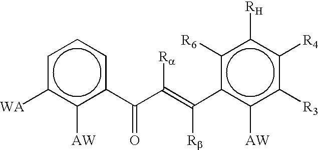

198. A compound according to claim 197 wherein AW is OR_{H}.

199. A compound according to claim 198 wherein R₃, R₄, and R₆ are hydrogen.

200. A compound according to claim 199 selected from
2,2′,3′-trimethoxy-5-ethylchalcone,
2,2′,3′-triethoxy-5-ethylchalcone,
2,2′,3′-tripropoxy-5-ethylchalcone,
2,2′,3′-tributoxy-5-ethylchalcone,
2 2′,3′-triisobutoxy-5-ethylchalcone,
2,2′,3′-tripentoxy-5-ethylchalcone,
2,2′,3′-tri-(1-methylbutyl)oxy-5ethylchalcone,
2,2′,3′-tri-(1-ethylpropyl)oxy-5-ethylchalcone,
2,2′,3′-trihexoxy-5-ethylchalcone,
2,2′,3′-tri-(1-methylpentyl)oxy-5-ethylchalcone,
2,2′,3′-tri-(1-ethylbutyl)oxy-5-ethylchalcone, and
2,2′,3′-tri-(1-propylpropyl)oxy-5-ethylchalcone.

201. A compound according to claim 196 with the general formula

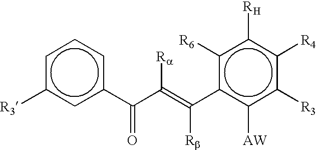

202. A compound according to claim 201 with the general formula

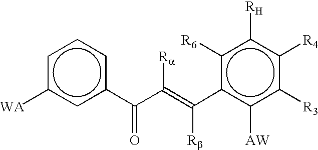

203. A compound according to claim 202 wherein AW is OR_{H}.

204. A compound according to claim 203 wherein R₃, R₄, and R₆ are hydrogen.

205. A compound according to claim 204 selected from
2,3′-dimethoxy-5-ethylchalcone,
2,3′-diethoxy-5-ethylchalcone,
2,3′-dipropoxy-5-ethylchalcone,
2,3′-dibutoxy-5-ethylchalcone,
2,3′-diisobutoxy-5-ethylchalcone,
2,3′-dipentoxy-5-ethylchalcone,
2,3′-di-(1-methylbutyl)oxy-5-ethylchalcone,
2,3′-di-(1-ethylpropyl)oxy-5-ethylchalcone,
2,3′-dihexoxy-5-ethylchalcone, and
2,3′-di-(1-methylpentyl)oxy-5-ethylchalcone.

206. A compound according to any of claims 157-164 with the general formula

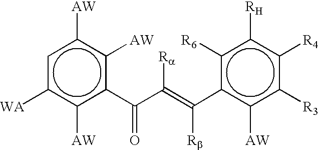

207. A compound according to claim 206 wherein AW is OR_{H}.

208. A compound according to claim 207 wherein R₃, R₄, and R₆ are hydrogen.

209. A compound according to claim 208 selected from
2,2′,3′,5′,6′-pentamethoxy-5-ethylchalcone,
2,2′,3′,5′,6′-pentaethoxy-5-ethylchalcone,
2,2′,3′,5′,6′-pentapropoxy-5-ethylchalcone,
2,2′,3′,5′,6′-pentabutoxy-5-ethylchalcone,
2,2′,3′,5′,6′-pentaisobutoxy-5-ethylchalcone,
2,2′,3′,5′,6′-pentapentoxy-5-ethyichalcone,
2,2′,3′,5′,6′-penta-(1-methylbutyl)oxy-5-ethylchalcone,
2,2′,3′,5′,6′-penta-(1-ethylpropyl)oxy-5-ethylchalcone,
2,2′,3′,5′,6′-pentahexoxy-5-ethylchalcone,
2,2′,3′,5′,6′-penta-(1-methylpentyl)oxy-5-ethylchalcone,
2,2′,3′,5′,6′-penta-(1-ethylbutyl)oxy-5-ethylchalcone, and
2,2′,3′,5′,6′-penta-(1-propylpropyl)oxy-5-ethylchalcone.

210. A compound of the general formula 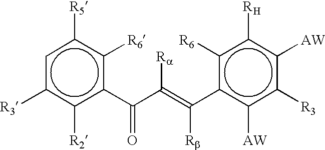
wherein R₃ and R₆ each independently is selected from hydrogen, R_{H}, cyano, nitro, nitroso, amino, and halogen; R_{α}, R_{β}, R′₂, R′₃, R′₅, and R′₆ each independently is selected from hydrogen, cyano, nitro, nitroso, halogen, R_{H}, and AW, wherein each A independently is selected from —O—, —S—, —NH—, and —NR_{H}—, preferably —O— and —NR_{H}—, and each W independently is selected from hydrogen, R_{H}, and R_{H}CO—, preferably R_{H}, wherein R_{H} is selected from C₁₋₆ straight, branched and cyclic aliphatic hydrocarbyl which may be saturated or may contain one or more unsaturated bonds selected from double bonds and triple bonds, which hydrocarbyl may be substituted with one or more substituents selected from C₁₋₆ alkoxy, hydroxy, halogen, amino, and amino which is optionally alkylated with one or two C₁₋₆ alkyl groups.

211. A compound according to claim 210, wherein each of R_{α} and R_{β} independently is selected from C₁₋₃ alkyl, cyano, and halogen.

212. A compound according to claim 210, wherein R_{α} is hydrogen and R_{β} is as defined in claim 211.

213. A compound according to claim 210, wherein R_{β} is hydrogen and R_{α} is as defined in claim 211.

214. A compound according to claim 210, wherein R_{α} and R_{β} are both hydrogen.

215. A compound according to any of claims 210-214, wherein R_{H} is selected from C₁₋₆ straight, branched and cyclic aliphatic hydrocarbyl which may be saturated or may contain one or more unsaturated bonds selected from double bonds and triple bonds.

216. A compound according to claim 215, wherein R_{H} is selected from methyl, ethyl, propyl, isopropyl, butyl, isobutyl, secbutyl, tertbutyl, pentyl, 1-methylbutyl, 1,1-dimethylpropyl, hexyl, 1-methylpentyl, 1-ethylbutyl, cyclopentyl, cyclohexyl, prop-2-enyl, 1,1-dimethyl-prop-2-enyl, and 3-methyl-but-2-enyl.

217. A compound according to any of claims 210-216, wherein R₃ and R₆ each independently is selected from hydrogen and R_{H}.

218. A compound according to any of claims 210-217, wherein R′₂, R′₃, R′₅, and R′₆ each independently is selected from hydrogen, R_{H}, and AW.

219. A compound according to any of claims 210-217 with the general formula

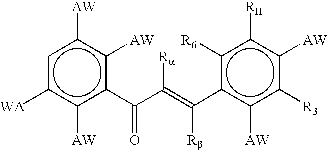

220. A compound according to claim 219 wherein AW is OR_{H}.

221. A compound according to claim 220 wherein R₃ and R₆ are hydrogen.

222. A compound according to any of claims 210-218 with the general formula

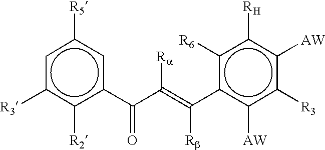

223. A compound according to claim 222 with the general formula

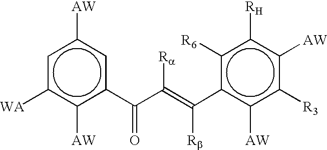

224. A compound according to claim 223 wherein AW is OR_{H}.

225. A compound according to claim 224 wherein R₃ and R₆ are hydrogen.

226. A compound according to claim 222 with a general formula

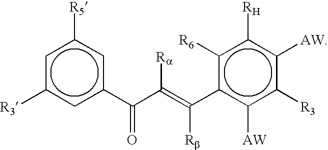

227. A compound according to claim 226 with the general formula

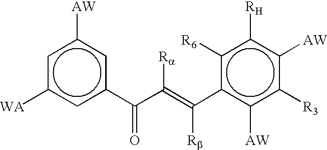

228. A compound according to claim 227 wherein AW is OR_{H}.

229. A compound according to claim 228 wherein R₃ and R₆ are hydrogen.

230. A compound according to claim 222 with the general formula

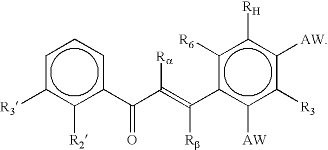

231. A compound according to claim 230 with the general formula

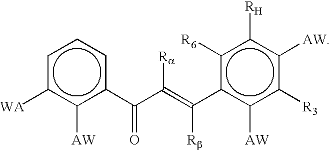

232. A compound according to claim 231 wherein A is OR_{H}.

233. A compound according to claim 232 wherein R₃ and R₆ are hydrogen.

234. A compound according to claim 230 with the general formula

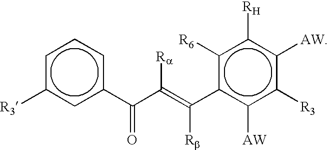

235. A compound according to claim 234 with the general formula

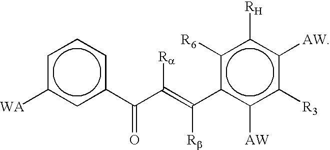

236. A compound according to claim 235 wherein AW is OR_{H}.

237. A compound according to claim 236 wherein R₃ and R₆ are hydrogen.

238. A compound according to claim 222 with the general formula

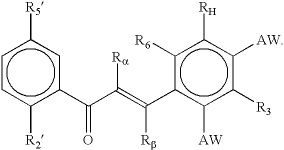

239. A compound according to claim 238 with the general formula

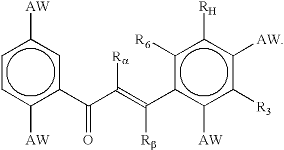

240. A compound according to claim 239 wherein AW is OR_{H}.

241. A compound according to claim 240 wherein R₃ and R₆ are hydrogen.

242. A compound according to claim 238 with the general formula

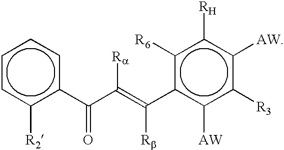

243. A compound according to claim 242 with the general formula

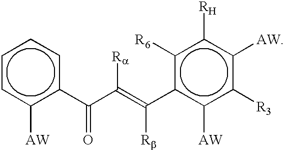

244. A compound according to claim 243 wherein AW is OR_{H}.

245. A compound according to claim 244 wherein R₃ and R₆ are hydrogen.

246. A compound according to any of claims 210-218 with the general formula

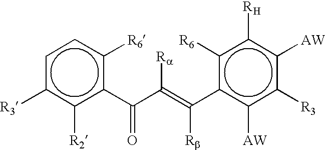

247. A compound according to claim 246 with the general formula

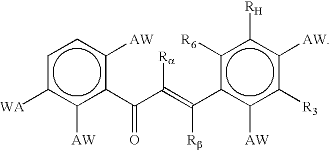

248. A compound according to claim 247 wherein AW is OR_{H}.

249. A compound according to claim 248 wherein R₃ and R₆ are hydrogen.

250. A compound according to claim 246 with the general formula

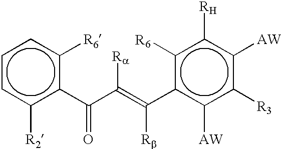

251. A compound according to claim 250 with the general formula

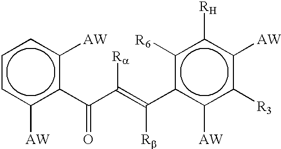

252. A compound according to claim 251 wherein AW is OR_{H}.

253. A compound according to claim 252 wherein R₃ and R₆ are hydrogen.

254. A compound of the general formula 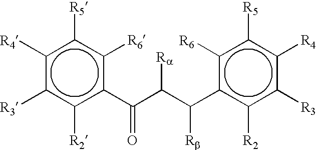
wherein R_{α}, R_{β}, R₂, R₃, R₄, R₅, R₆, R₂′, R₃′, R₄′, R₅′, and R₆′ each independently is selected from hydrogen, amino, halogen, R_{H}, and AW, wherein each A independently is selected from —O—, —S—, —NH—, and —NR_{H}—, preferably —O— and —NR_{H}—, and each W independently is selected from hydrogen, R_{H}, and R_{H}CO—, preferably R_{H}, wherein R_{H} is selected from C₁₋₆ straight, branched and cyclic aliphatic hydrocarbyl which may be saturated or may contain one or more unsaturated bonds selected from double bonds and triple bonds, which hydrocarbyl may be substituted with one or more substituents selected from C₁₋₆ alkoxy, hydroxy, halogen, amino, and amino which is optionally alkylated with one or two C₁₋₆ alkyl groups,
with the first proviso that one of R_{α} and R_{β} is hydrogen, and
with the second proviso that
a) the substituents R₂ and R₄ are both different from hydrogen, or
b) the substituents R₂ and R₅ are both different from hydrogen, or
c) the substituents R₂ and R₆ are both different from hydrogen, or
d) the substituents R₃ and R₅ are both different from hydrogen; and
with the third proviso that the compound is not one of:
1,3-bis(2,5dihydroxyphenyl)-1-propanone,
3-(2,5-dimethoxyphenyl)-1-(2-hydroxy-6-methoxyphenyl)-1-propanone,
3-(4-hydroxy-2-methoxyphenyl)-1-phenyl-1-propanone,
α,β-dihydrolicochalcone A,
1,3-bis[4-methoxy-2-methyl-5-(1-methylethyl)phenyl]-1-propanone,
3-[2-(3,7-dimethyl-2,6-octadienyl)-3-hydroxy-4-methoxyphenyl]-1-(2-hydroxy-4-methoxyphenyl)-1-propanone,
3-(2,4-dichlorophenyl)-1-(2,4-dimethoxyphenyl)-1-propanone,
3-[2-hydroxy-5-]3-(2-hydroxy-4-methoxyphenyl)-1-(4-hydroxyphenyl)propyl]-4-methoxyphenyl]-1-(4-hydroxyphenyl)-1-propanone,
3-(2,4-dihydroxyphenyl)-1-(2-hydroxyphenyl)-1-propanone,
3-(4-hydroxy-2,6-dimethoxyphenyl)-1-(4-hydroxyphenyl)-1-propanone,
3-(2-hydroxy-4,6-dimethoxyphenyl)-1-(4-hydroxyphenyl)-1-propanone1-phenyl-3-(2,4,6-trihydroxyphenyl)-1-propanone,
3-(5-hydroxy-4-methoxy-2-methylphenyl)-1-(2,4,6-trihydroxyphenyl)-1-propanone,
3-[2-bromo-4-methoxy-5-(phenylmethoxy)phenyl]-1-[2,4,6-tris(phenylmethoxy)phenyl]-1-propanone,
3-(2-amino-5-hydroxy-4-methoxyphenyl)-1-[2,4,6-trihydroxyphenyl]-1-propanone,
3-(2,5-dihydroxy-4-methoxyphenyl)-1-(2,4,6-trihydroxyphenyl)-1-propanone,
3-(2,5-dihydroxy-4-methoxyphenyl)-1-(2,4,6-trihydroxyphenyl)-1-propanone,
3-(2-bromo-5-hydroxy-4-methoxyphenyl)-1-(2,4,6-trihydroxyphenyl)-1-propanone,
3-(2,3,4-trihydroxyphenyl)-1-(2,4,6-trihydroxyphenyl)-1-propanone,
3-[4-hydroxy-2-methyl-5-(1-methylethyl)phenyl]-1-phenyl-1-propanone,
1-(2,4-dihydroxyphenyl)-3-[2-(3,7-dimethyl-2,6-octadienyl)-3,4-dimethoxyphenyl]-1-propanone,
3-[2-[3-[(2,2-dimethylpropyl)amino]-2-hydroxypropyl]-4-methoxyphenyl]-1-phenyl-1-propanone,
3-(2,4-dihydroxy-6-methoxyphenyl)-1-(4-hydroxyphenyl)-1-propanone (loureirin D),
1-(4-hydroxyphenyl)-3-(2,4-6-trimethoxyphenyl)-1-propanone (loureirin B),
3-(2,4-dimethoxyphenyl)-1-(4-hydroxyphenyl)-1-propanone (loureirin A),
3-(4-hydroxy-2-methoxyphenyl)-1-(4-hydroxyphenyl)-1-propanone,
3-(2-bromo-3,4,5-trimethoxyphenyl)-3′,4′-dimethoxy-1-propiophenone,
1,3-dimesityl-1-propanone,
3-(2,4-dimethoxyphenyl)-1-(3,4-dimethoxyphenyl)-1-propanone,
3-(2-hydroxy-4-methoxyphenyl)-1-(4-hydroxyphenyl)-1-propanone,
3-(2,4-dihydroxyphenyl)-1-(4-methoxyphenyl)-1-propanone,
1-phenyl-3-(2,4,6-trimethylphenyl)-1-chalcone,
1-(3,4-dimethoxyphenyl)-1-(2,4,6-trihydroxyphenyl)-1-propanone,
1-[1,1′-biphenyl-4-yl-3-(2,4-dimethoxyphenyl)-1-propanone,
3-[2,4-bis[(3-methyl-2-butenyl)oxy]phenyl]-1-[4-[(3,7-dimethyl-2,6-octadienyl)oxy]-2-hydroxyphenyl]-2-hydroxyphenyl]-1-propanone,
1-(4-hydroxyphenyl)-3-(2,4,6-trihydroxyphenyl)-1-propanone,
3-(2,4-dichlorophenyl)-1-[2-[2-hydroxy-3-(propylamino)propoxy]phenyl]-1-propanone,
3-(2,4-dichlorophenyl)-1-(2-hydroxyphenyl)-1-propanone,
3-(2,4-dichlorophenyl)-1-[2-[2-hydroxy-3-(propylamino)propoxy]phenyl]-1-propanone,
3-[4-(3-bromopropoxy)-2,6-bis(phenylmethoxy)phenyl]-1-[4-methoxy-3-(phenylmethoxy)phenyl]-1-propanone,
3-[4-hydroxy-2,6-bis(phenylmethoxy)phenyl]-1-[4-methoxy-3-(phenylmethoxy)phenyl]-1-propanone,
3-[4-(3-aminopropoxy)-2,6-dihydroxyohenyl]-1-(3-hydroxy-4-methoxyphenyl)-1-propanone,
3-[2,6-dihydroxy-4-[2-(2-methoxyethoxy)ethoxy]phenyl]-1-(3-hydroxy-4-methoxyphenyl)-1-propanone,
1,3-bis(2,4,6-trimethoxyphenyl)-1-propanone,
1-(2-hydroxy-4,6-dimethoxyphenyl)-3-(2,4,6-trimethoxyphenyl)-1-propanone,
3-[5-(1,1-dimethoxypropyl)-4-hydroxy-2-methoxyphenyl]-1-(4-hydroxyphenyl)-1-propanone,
3-(4-methoxy-2-methylphenyl)-1-phenyl-1-propanone,
3-(4-hydroxy-2-methylphenyl)-1-phenyl-1-propanone1-(2,4-dihydroxy-6-methylphenyl)-3-(2,4,5-trimethoxyphenyl)-1-propanone,
3-(5-chloro-2,4-dimethoxyphenyl)-1-phenyl-1-propanone,
3-(3,5-dimethoxyphenyl)-1-[2-hydroxy-4,6-dimethoxy-3-[5-methyl-2-(1-methylethyl)hexyl]phenyl]-1-propanone,
1,3-bis(2,4,5-trimethoxyphenyl)-1-propanone,
3-(2-ethoxy-4-methoxyphenyl)-1-phenyl-1-propanone,
3-(2,4,6-trimethoxyphenyl)-1-(3,4,5-trimethoxyphenyl)-1-propanone,
3-(2-(hydroxy-4,6-dimethoxyphenyl)-1-(3,4,5-trimethoxyphenyl)-1-propanone,
3-(4-hydroxy-2,6-dimethoxyphenyl)-1-(trimethoxyphenyl)-1-propanone,
1-(4-methoxyphenyl)-3-(2,4,6-trimethoxyphenyl)-1-propanone,
3-(4-hydroxy-2,6-dimethoxyphenyl)-1-(4-methoxyphenyl)-1-propanone,
1-(3,4-dimethoxyphenyl)-3-(2-ethoxy-4,6-dimethoxyphenyl)-1-propanone,
3-(2-hydroxy-4,6-dimethoxyphenyl)-3′,4′-diethoxypropiophenone,
1-(3,4-dimethoxyphenyl)-3-(4-ethoxy-2,6-dimethoxyphenyl)-1-propanone,
1-(3,4-dimethoxyphenyl)-3-(2,4,6-trimethoxyphenyl)-1-propanone,
1-(3,4-dimethoxyphenyl)-3-(4-hydroxy-2,6-dimethoxyphenyl)-1-propanone,
3-(2,3,4,5-tetrachloro-6-methoxyphenyl)-1-phenyl-1-propanone,
3-(2,3,4,5-tetrachloro-6-hydroxyphenyl)-1-phenyl)-1-propanone,
3-(4-ethoxy-2-methoxyphenyl)-1-phenyl-1-propanone, Hexahydrokuraridin,
1,3-bis(2,4-dimethoxyphenyl)-1-propanone,
3-pentafluorophenyl-1-phenyl-1-propanone,
3-(2-hydroxy-4,6-dimethoxyphenyl)-1-(4-methoxyphenyl)-1-propanone,
3-(2,4-dihydroxyphenyl)-1-(4-hydroxyphenyl)-1-propanone,
3-(2-hydroxy-4-methoxyphenyl)-1-(4-methoxyphenyl)-1-propanone,
2′,3′,4′-trimethoxy-3-(2,3,4-trimethoxyphenyl)propiophenone,
3-(2,5-dimethoxyphenyl)-1-(2-hydroxy-6-methoxyphenyl)-1-propanone,
3-(6-hydroxy-m-tolyl)-propiophenone,
3-(2,5-dihydroxyphenyl)-1-(2-hydroxy-4,6-dimethoxyphenyl)-1-propanone,
1,3-bis[5-(1,1-dimethylethyl)-2-methoxyphenyl]-1-propanone,
1,3-bis-[5-(1,1-dimethylethyl)-2-methylphenyl]-1-propanone,
3-(3,5-dichloro-2-hydroxyphenyl)-1-propiophenone,
3-(3-amino-2,5-dimethoxyphenyl)-2′,5′-dihydroxypropiophenone,
3-(5-chloro-2-hydroxyphenyl)-1-(4-chlorophenyl)-1-propanone,
3-(3-amino-2,5-dimethoxyphenyl)-2′,5′-dihydroxy-1-propiophenone,
3-(2,5-dimethoxyphenyl)-1-phenyl-1-propanone,
3-(2,5-dimethylphenyl)-1-(2-hydroxyphenyl)-1-propanone,
2′-[2-(diethylaminoethoxy]-3-(2,5-xylyl)-1-propiophenone,
3-(2,5-dihydroxyphenyl)-2′,5′-dihydroxypropiophenone,
3-(2,5-dimethoxyphenyl)-2′,5′-dimethoxypropiophenone,
1-(2,4,6-trihydroxyphenyl)-3-(3,4,5-trimethoxyphenyl)-1-propanone,
3-[3,4-dihydroxy-5-(3-methyl-2-butenyl)phenyl]-1-[2,4,6-trihydroxy-3-(3-methyl-2-butenyl)phenyl]-1-propanone,
3-[3,4-dihydroxy-5-(3-methyl-2-butenyl)phenyl]-1-[3-(3,5-dimethyl-2,6-octadienyl)-2,4,6-trihydroxyphenyl]-1-propanone,
3-[3,5-bis(trifluoromethyl)phenyl]-1-phenyl-1-propanone,
3-(3,5-dimethoxyphenyl)-1-phenyl-1-propanone,
1-((3-hydroxyphenyl)-3-(3,4,5-trimethoxyphenyl)-1-propanone,
3-[3-hydroxy-4-methoxy-5-(3-methyl-2-butenyl)phenyl]-1-[2,4,6-trihydroxy-3-(3-methyl-2-butenyl)phenyl]-1-propanone,
1-(3-hydroxy-4-methoxyphenyl)-3-(3,4,5-trimethoxyphenyl)-1-propanone,
3-[4-hydroxy-3,5-bis(3-methyl-2-butenyl)phenyl]-1-(2,4,6-trihydroxyphenyl)-1-propanone,
3-[3-(3,7-dimethyl-2,6-octadienyl)-4-hydroxy-5-(3-methyl-2-butenyl)phenyl]-1-(2,4,6-trihydroxyphenyl)-1-propanone,
1-(2,6-dihydroxy-4-methoxyphenyl)-3-(4-hydroxy-3,5-dimethylphenyl)-1-propanone,
3-[3,5-bis(trifluoromethyl)phenyl]-1-(4-methoxyphenyl)-1-propanone,
1-(2-hydroxyphenyl)-3-(3,4,5-trimethoxyphenyl)-1-propanone,
1-(4-fluorophenyl)-3-(4-hydroxy-3,5-dimethylphenyl)-1-propanone1-(2,4,6-trihydroxyphenyl)-3-(3,4,5-trihydroxyphenyl)-1-propanone,
3-(4-hydroxy-3,5-dimethylphenyl)-1-phenyl-1-propanone,
3-(4-hydroxy-3,5-dimethoxyphenyl)-1-[2,4,6-trihydroxy-3-(3-methylbutyl)phenyl]-1-propanone-3′,4′-dimethoxy-3-(3,4,5-trimethoxyphenyl)-propiophenone,
3′-methoxy-3-(3,4,5-trimethoxyphenyl)propiophenone,
4′-methoxy-3-(3,4,5-trimethoxyphenyl)propiophenone,
3-(2-bromo-3,4,5-trimethoxyphenyl)-3′,4′-dimethoxypropiophenone,
3-(3,5-dichloro-2-hydroxyphenyl)propiophenone,
3(3-amino-2,5-dimethoxyphenyl)-2′,5′-dihydroxypropiophenone,
1-[2-[2-hydroxy-3-propylamino)propoxyl]phenyl]-3-(3,4,5-trimethoxyphenyl)-1-propanone,
3-[3,5-bis(1,1-dimethylethyl)-4-hydroxyphenyl]-1-phenyl-1-propanone,
3-(3-hydroxy-4,5-dimethoxyphenyl)-1-[4-methoxy-3-(phenylmethoxy)phenyl-1-propanone,
1,3-bis(3,4,5-trimethoxyphenyl)-1-pentanone,
1-(3,4-dimethoxyphenyl)-3-(3,4,5-trimethoxyphenyl)-1-pentanone,
3-(3,5-dihydroxy-4-methoxyphenyl)-1-(2,4-dihydroxyphenyl)-3-hydroxypropanone,
1,3-bis(2-chloro-4-hydroxy-5-methoxyphenyl)-3-hydroxy-1-propanone,
1,3-bis(2,4,5-trimethoxyphenyl)-4-penten-1-one,
1-[5-[3-[2,4-dimethoxyphenyl)-1-methyl-3-oxopropyl]-2,4-dimethoxyphenyl]-2-butenone,
3-(2-amino-5-chlorophenyl)-3-hydroxy-1-(4-methylphenyl)-1-propanone,
1-[2,4,6-trimethoxy-3-[1-methyl-3-oxo-3-(2,4,6-trimethoxyphenyl)propyl]phenyl]-2-buten-1-one,
3-(2,4,5-trimethoxyphenyl)-1,5-bis(3,4,5-trimethoxyphenyl)-1,5-pentadienone,
2-methyl-1-phenyl-3-(2,4,6-trimethylphenyl)-1-propanone,
2-hydroxy-3-(2-hydroxy-4,6-dimethoxyphenyl)-3′,4′-dimethoxypropiophenone,
1,3-bis(2-chloro-4-hydroxy-5-methoxyphenyl)-3-hydroxy-1-propanone,
1,3-bis(2,4,5-trimethoxyphenyl)-4-penten-1-one,
1-[5-[3-[2,4-dimethoxyphenyl)-1-methyl-3-oxopropyl]-2,4-dimethoxyphenyl]-2-butenone,
1-[2,4,6-trimethoxy-3-[1-methyl-3-oxo-3-(2,4,6-trimethoxyphenyl)propyl]phenyl]-2-buten-1-one,
1,3-bis(2,4,6-trimethoxyphenyl)-1-butanone,
3-(2,4,5-trimethoxyphenyl)-1,5-bis(3,4,5-trimethoxyphenyl)-1,5-pentanedione,
1,3-bis(2,6-dichlorophenyl)-3-hydroxy-1-propanone,
1-[2,4,6-trimethoxy-3-[1-methyl-3-oxo-3-(2,4,6-trimethoxyphenyl)propyl]phenyl]-2-buten-1-one,
1,3-bis(2,4,6-trimethoxyphenyl)-1-butanone,
4-chloro-1-(4-fluorophenyl)-3-(2,4,6-trimethoxyphenyl)-1-butanone,
2-methyl-1-phenyl-3-(2,4,6-trimethylphenyl)-1-propanone,
2-hydroxy-3-(2-hydroxy-4,6-dimethoxyphenyl)-3′,4′-dimethoxy-propiophenone,
2-hydroxy-3-(2-hydroxy-4,6-dimethoxyphenyl)-1-(4-methoxyphenyl)-1-propanone,
3-(3-amino-2,5-dimethoxyphenyl)-2′,5′-dihydroxypropiophenone,
6′-(2-m-anisoylethyl)-2′,3′,4′-trimethoxyacetanilide,
3-[2-(acetyloxy)-4,6-dimethoxyphenyl]-1-(4-methoxyphenyl)-1-propanone,
3-[4-(acetyloxy)-2,6-dimethoxyphenyl]-1-(3,4-dimethoxyphenyl)-1-propanone,
3-[2-(acethyloxy)-4,6-dimethoxyphenyl)-3′,4′-dimethoxypropiophenone,
3-[4-(acetyloxy)-2,6-dimethoxyphenyl]-1-(3,4,5-trimethoxyphenyl)-1-propanone,
3-(2,5-dimethoxy-3,4,6-trimethylphenyl)1-phenyl-1-propanone,
3-(4-benzoyloxy-2-methyl)-1-phenyl-1-propanone,
3-[2,4-bis(acetyloxy)phenyl]-1-3,4-bis(acetylxoy)phenyl]-1-propanone,
6′-(2-m-anisoylethyl)-2′,3′,4′-trimethoxyacetanilide,
3-(2-acetyloxy-4-methoxyphenyl)-4′-methoxy-propiophenone,
3-(acetyloxy)-3-[3,5-bis(acetyloxy)-4-methoxyphenyl]-1-[2,4-bis(acetyloxy)phenyl]-1-propanone,
2-acetyloxy-3-(2-acetyloxy-4,6-dimethoxyphenyl)-3′,4′-dimethoxy-1-propiophenone,
1,3-bis[4-acetyloxy)-2-chloro-5-methoxyphenyl]-3-hydroxy-1-propanone,
2-acetyloxoxy-3-(2-acetyloxy-4,6-dimethoxyphenyl)-3′,4′-dimethoxypropiophenone, or
2,3′,4′-triacetyloxy-3-(2,4,6-trihydroxyphenyl)propiophenone.

255. A compound according to claim 254, wherein R_{α} is hydrogen and R_{β} is selected from C₁₋₃ alkyl, cyano, and halogen.

256. A compound according to claim 254, wherein R_{β} is hydrogen and R is selected from C₁₋₃ alkyl, cyano, and halogen.

257. A compound according to claim 254, wherein R_{α} and R_{β} are both hydrogen.

258. A compound according to any of claims 254-257, wherein R₂, R₃, R₄, R₅, R₆, R₂′, R₃′, R₄′, R₅′, and R₆′ each independently is selected from hydrogen, R_{H}, and AW.

259. A compound according to any of claims 254-258, wherein at least one of R₂′, R₃′, R₄′, R₅′, and R₆′ is not hydrogen, preferably at least two of R₂′, R₃′, R₄′, R₅′, and R₆′ are not hydrogen.

260. A compound according to any of claims 254-259, wherein at least one of R₂′, R₃′, R₄′, R₅′, and R₆′ is AW, preferably at least two of R₂′, R₃′, R₄′, R₅′, and R₆′ are AW.

261. A compound according to any of claims 254-260, wherein at least three of R₂, R₃, R₄, R₅, and R₆ are not hydrogen.

262. A compound according to any of claims 254-261, wherein at least one of R₂, R₃, R₄, R₅, and R₆ is AW, preferably at least two of R₂, R₃, R₄, R₅, and R₆ are AW.

263. A compound according to any of claims 254-262, wherein R_{H} is selected from C₁₋₆ straight, branched, and cyclic hydrocarbyl which may be saturated or may contain one or more unsaturated bonds selected from double and triple bonds.

264. A compound according to claim 263, wherein R_{H} is selected from methyl, ethyl, propyl, isopropyl, butyl, isobutyl, secbutyl, tertbutyl, pentyl, 1-methylbutyl, 1,1-dimethylpropyl, hexyl, 1-methylpentyl, 1-ethylbutyl, cyclopentyl, cyclohexyl, prop-2-enyl, 1,1-dimethyl-prop-2-enyl, and 3-methyl-but-2-enyl.

265. A compound according to any of the claims 254-265, wherein AW is OW.

266. A compound according to any of claims 254-265, wherein AW is OR_{H}.

267. A compound according to any of claims 254-266, wherein R₄′ is hydrogen.

268. A compound according to any of claims 254-267, wherein R₂ and R₄ both are different from hydrogen.

269. A compound according to claim 268 having the general formula

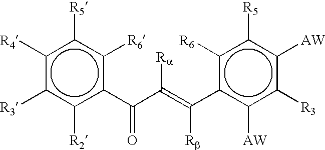

270. A compound according to claim 269, wherein AW is OW.

271. A compound according to claim 269, wherein R_{4′} is hydrogen.

272. A compound according to claim 269 or 270, with the general formula

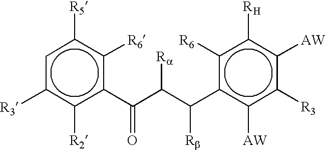

273. A compound according to any of claims 254-267, wherein R₃ and R₅ both are different from hydrogen.

274. A compound according to claim 273 having the general formula

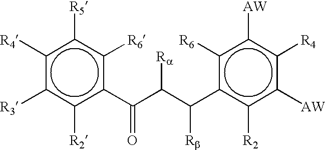

275. A compound according to claim 273, wherein AW is OW.

276. A compound according to claim 273, wherein R_{4′} is hydrogen.

277. A compound according to claim 274 or 275, with the general formula

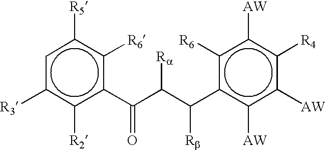

278. A compound according to any of claims 254-267, wherein R₂ and R₅ both are different from hydrogen.

279. A compound according to claim 278 having with the general formula

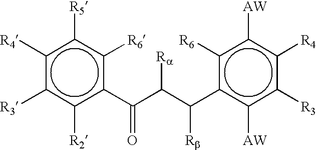

280. A compound according to claim 279, wherein AW is OW.

281. A compound according to claim 279, wherein R_{4′} is hydrogen.

282. A compound according to claim 278 having the general formula

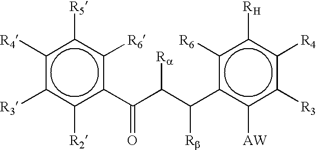

283. A compound according to claim 282, wherein AW is OW.

284. A compound according to claim 282 or 283, wherein R_{4′} is hydrogen.

285. A compound according to any of claims 254-267, wherein R₂ and R₆ both are different from hydrogen.

286. A compound according to claim 285 having the general formula

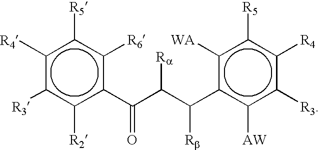

287. A compound according to claim 286 wherein AW is OW.

288. A compound according to claim 286 or 287, wherein R_{4′} is hydrogen.

289. A compound according to claims 254-288 selected from
2,5,2′,3′,5′,6′-hexamethoxy-α,β-dihydrochalcone,
2,5,2′,3′,5′,6′-hexaethoxy-α,β-dihydrochalcone,
2,5,2′,3′,5′,6′-hexapropoxy-α,β-dihydrochalcone,
2,5,2′,3′,5′,6′-hexa-isopropoxy-α,β-dihydrochalcone,
2,5,2′,3′,5′,6′-hexabutoxy-α,β-dihydrochalcone,
2,5,2′,3′,5′,6′-hexa-isobutoxy-α,β-dihydrochalcone,
2,5,2′,3′,5′,6′-hexa-tertbutoxy-α,β-dihydrochalcone,
2,5,2′,3′,5′,6′-hexapentoxy-α,β-dihydrochalcone,
2,5,2′,3′,5′,6′-hexa-(1-methylbutyl)oxy-α,β-dihydrochalcone,
2,5,2′,3′,5′,6′-hexahexoxy-α,β-dihydrochalcone,
2,5,2′,3′,5′,6′-hexa-(1-methylpentyl)oxy-α,β-dihydrochalcone,
2,5,2′,3′,5′,6′-hexa-(1-ethylbutyl)oxy-α,β-dihydrochalcone,
2,5,2′,3′,5′,6′-hexacyclohexoxy-α,β-dihydrochalcone,
2,5,2′,3′,6′-pentamethoxy-α,β-dihydrochalcone,
2,5,2′,3′,6′-pentaethoxy-α,β-dihydrochalcone,
2,5,2′,3′,6′-pentapropoxy-α,β-dihydrochalcone,
2,5,2′,3′,6′-penta-isopropoxy-α,β-dihydrochalcone,
2,5,2′,3′,6′-pentabutoxy-α,β-dihydrochalcone,
2,5,2′,3′,6′-penta-isobutoxy-α,β-dihydrochalcone,
2,5,2′,3′,6′-penta-tertbutoxy-α,β-dihydrochalcone,
2,5,2′,3′,6′-pentapentoxy-α,β,-dihydrochalcone,
2,5,2′,3′,6′-penta-(1-methylbutyl)oxy-α,β-dihydrochalcone,
2,5,2′,3′,6′-pentahexoxy-α,β-dihydrochalcone,
2,5,2′,3′,6′-penta-(1-methylpentyl)oxy-α,β-dihydrochalcone,
2,5,2′,3′,6′-penta-(1-ethylbutyl)oxy-α,β-dihydrochalcone,
2,5,2′,3′,6′-pentacyclohexoxy-α,β-dihydrochalcone,
2,5,2′,3′,5′-pentamethoxy-α,β-dihydrochalcone,
2,5,2′,3′,5′-pentaethoxy-α,β-dihydrochalcone,
2,5,2′,3′,5′-pentapropoxy-α,β-dihydrochalcone,
2,5,2′,3′,5′-penta-isopropoxy-α,β-dihydrochalone,
2,5,2′,3′,5′-pentabutoxy-α,β-dihydrochalcone,
2,5,2′,3′,5′-penta-isobutoxy-α,β-dihydrochalcone,
2,5,2′,3′,5′-penta-tertbutoxy-α,β-dihydrochalcone,
2,5,2′,3′,5′-pentapentoxy-α,β-dihydrochalcone,
2,5,2′,3′,5′-penta-(1-methylbutyl)oxy-α,β-dihydrochalcone,
2,5,2′,3′,5′-pentahexoxy-α,β-dihydrochalcone,
2,5,2′,3′,5′-penta-(1methylpentyl)oxy-α,β-dihydrochalcone,
2,5,2′,3′,5′-penta-(1-ethylbutyl)oxy-α,β-dihydrochalcone,
2,5,2′,3′,5′-pentacyclohexoxy-α,β-dihydrochalcone,
2,5,2′,3′-tetramethoxy-α,β-dihydrochalcone,
2,5,2′,3′-tetraethoxy-α,β-dihydrochalcone,
2,5,2′,3′-tetrapropoxy-α,β-dihydrochalcone,
2,5,2′,3′-tetra-isopropoxy-α,β-dihydrochalcone,
2,5,2′,3′-tetrabutoxy-α,β-dihydrochalcone,
2,5,2′,3′-tetra-isobutoxy-α,β-dihydrochalcone,
2,5,2′,3′-tetra-tertbutoxy-α,β-dihydrochalcone,
2,5,2′,3′-tetrapentoxy-α,β-dihydrochalcone,
2,5,2′,3′-tetra-(1-methylbutyl)oxy-α,β-dihydrochalcone,
2,5,2′,3′-tetrahexoxy-α,β-dihydrochalcone,
2,5,2′,3′-tetra-(1-methylpentyl)oxy-α,β-dihydrochalcone,
2,5,2′,3′-tetra-(1-ethylbutyl)oxy-α,β-dihydrochalcone,
2,5,2′,3′-tetracyclohexoxy-α,β-dihydrochalcone,
2,5,3′-trimethoxy-α,β-dihydrochalcone,
2,5,3′-triethoxy-α,β-dihydrochalcone,
2,5,3′-tripropoxy-α,β-dihydrochalcone,
2,5,3′-tri-isopropoxy-α,β-dihydrochalcone,
2,5,3′-tributoxy-α,β-dihydrochalcone,
2,5,3′-tri-isobutopoy-α,β-dihydrochalcone,
2,5,3′-tri-tertbutoxy-α,β-dihydrochalcone,
2,5,3′-tripentoxy-α,β-dihydrochalcone,
2,5,3′-tri-(1-methylbutyl)oxy-α,β-dihydrochalcone,
2,5,3′-trihexoxy-α,β-dihydrochalcone,
2,5,3′-tri(1-methylpentyl)oxy-α,β-dihydrochalcone,
2,5,3′-tri-(1-ethylbutyl)oxy-α,β-dihydrochalcone,
2,5,3′-tricyclohexoxy-α,β-dihydrochalcone,
2,5,2′-trimethoxy-α,β-dihydrochalcone,
2,5,2′-triethoxy-α,β-dihydrochalcone,
2,5,2′-tripropoxy-α,β-dihydrochalcone,
2,5,2′-tri-isopropoxy-α,β-dihydrochalcone,
2,5,2′-tributoxy-α,β- dihydrochalcone,
2,5,2′-tri-isobutoxy-α,β-dihydrochalcone,
2,5,2′-tri-tertbutoxy-α,β-dihydrochalcone,
2,5,2′-tripentoxy-α,β-dihydrochalcone,
2,5,2′-tri-(1-methylbutyl)oxy-α,β-dihydrochalcone,
2,5,2′-trihexoxy-α,β-dihydrochalcone,
2,5,2′-tri-(1-methylpentyl)oxy-α,β-dihydrochalcone,
2,5,2′-tri-(1-ethylbutyl)oxy-α,β-dihydrochalcone,
2,5,2′-tricyclohexoxy-α,β-dihydrochalcone,
3-methoxy-2,5,2′,3′,5′,6′-hexamethoxy-α,β-dihydrochalcone,
3-methoxy-2,5,2′,3′,5′,6′-hexaethoxy-α,β-dihydrochalcone,
3-methoxy-2,5,2′,3′,5′,6′-hexapropoxy-α,β-dihydrochalcone,
3-methoxy-2,5,2′,3′,5′,6′-hexa-isopropoxy-α,β-dihydrochalcone,
3-methoxy-2,5,2′,3′,5′,6′-hexabutoxy-α,β-dihydrochalcone,
3-methoxy-2,5,2′,3′,5′,6′-hexa-isobutoxy-α,β-dihydrochalcone,
3-methoxy-2,5,2′,3′,5′,6′-hexa-tertbutoxy-α,β-dihydrochalcone,
3-methoxy-2,5,2′,3′,5′,6′-hexapentoxy-α,β-dihydrochalcone,
3-methoxy-2,5,2′,3′,5′,6′-hexa-(1-methylbutyl)oxy-α,β-dihydrochalcone,
3-methoxy-2,5,2′,3′,5′,6′-hexahexoxy-α,β-dihydrochalcone,
3-methoxy-2,5,2′,3′,5′,6′-hexa-(1-methylpentyl)oxy-α,β-dihydrochalcone,
3-methoxy-2,5,2′,3′,5′,6′-hexa-(1-ethylbutyl)oxy-α,β-dihydrochalcone,
3-methoxy-2,5,2′,3′,5′,6′-hexacyclohexoxy-α,β-dihydrochalcone,
3-methoxy-2,5,2′,3′,6′-pentamethoxy-α,β-dihydrochalcone,
3-methoxy-2,5,2′,3′,6′-pentaethoxy-α,β-dihydrochalcone,
3-methoxy-2,5,2′,3′,6′-pentapropoxy-α,β-dihydrochalcone,
3-methoxy-2,5,2′,3′,6′-penta-isopropoxy-α,β-dihydrochalcone,
3-methoxy-2,5,2′,3′,6′-pentabutoxy-α,β-dihydrochalcone,
3-methoxy-2,5,2′,3′,6′-penta-isobutoxy-α,β-dihydrochalcone,
3-methoxy-2,5,2′,3′,6′-penta-tertbutoxy-α,β-dihydrochalcone,
3-methoxy-2,5,2′,3′,6′-pentapentoxy-α,β-dihydrochalcone,
3-methoxy-2,5,2′,3′,6′-penta-(1-methylbutyl)oxy-α,β-dihydrochalcone,
3-methoxy-2,5,2′,3′,6′-pentahexoxy-α,β-dihydrochalcone,
3-methoxy-2,5,2′,3′,6′-penta-(1-methylpentyl)oxy-α,β-dihydrochalcone,
3-methoxy-2,5,2′,3′,6′-penta-(1-ethylbutyl)oxy-α,β-dihydrochalcone,
3-methoxy-2,5,2′,3′,6′-pentacyclohexoxy-α,β-dihydrochalcone,
3-methoxy-2,5,2′,3′,5′-pentamethoxy-α,β-dihydrochalcone,
3-methoxy-2,5,2′,3′,5′-pentaethoxy-α,β-dihydrochalcone,
3-methoxy-2,5,2′,3′,5′-pentapropoxy-α,β-dihydrochalcone,
3-methoxy-2,5,2′,3′,5′-penta-isopropoxy-α,β-dihydrochalcone,
3-methoxy-2,5,2′,3′,5′-pentabutoxy-α,β-dihydrochalcone,
3-methoxy-2,5,2′,3′,5′-penta-isobutoxy-α,β-dihydrochalcone,
3-methoxy-2,5,2′,3′,5′-penta-tertbutoxy-α,β-dihydrochalcone,
3-methoxy-2,5,2′,3′,5′-pentapentoxy-α,β-dihydrochalcone,
3-methoxy-2,5,2′,3′,5′-penta-(1-methylbutyl)oxy-α,β-dihydrochalcone,
3-methoxy-2,5,2′,3′,5′-pentahexoxy-α,β-dihydrochalcone,
3-methoxy-2,5,2′,3′,5′-penta-(1-methylpentyl)oxy-α,β-dihydrochalcone,
3-methoxy-2,5,2′,3′,5′-penta-(1-ethylbutyl)oxy-α,β-dihydrochalcone,
3-methoxy-2,5,2′,3′,5′-pentacyclohexoxy-α,β-dihydrochalcone,
3-methoxy-2,5,2′-trimethoxy-α,β-dihydrochalcone
3-methoxy-2,5,2′-triethoxy-α,β-dihydrochalcone,
3-methoxy-2,5,2′-tripropoxy-α,β-dihydrochalcone,
3-methoxy-2,5,2′-tri-isopropoxy-α,β-dihydrochalcone,
3-methoxy-2,5,2′-tributoxy-α,β-dihydrochalcone,
3-methoxy-2,5,2′-tri-isobutoxy-α,β-dihydrochalcone,
3-methoxy-2,5,2′-tri-tertbutoxy-α,β-dihydrochalcone,
3-methoxy-2,5,2′-tripentoxy-α,β-dihydrochalcone,
3-methoxy-2,5,2′-tri-(1-methylbutyl)oxy-α,β-dihydrochalcone,
3-methoxy-2,5,2′-trihexoxy-α,β-dihydrochalcone,
3-methoxy-2,5,2′-tri-(1-methylpentyl)oxy-α,β-dihydrochalcone,
3-methoxy-2,5,2′-tri-(1-ethylbutyl)oxy-α,β-dihydrochalcone,
3-methoxy-2,5,2′-tricyclohexoxy-α,β-dihydrochalcone,
3-methoxy-2,5,2′,3′-tetramethoxy-α,β-dihydrochalcone,
3-methoxy-2,5,2′,3′-tetraethoxy-α,β-dihydrochalcone,
3-methoxy-2,5,2′,3′-tetrapropoxy-α,β-dihydrochalcone,
3-methoxy-2,5,2′,3′-tetra-isopropoxy-α,β-dihydrochalcone,
3-methoxy-2,5,2′,3′-tetrabutoxy-α,β-dihydrochalcone,
3-metboxy-2,5,2′,3′-tetra-isobutoxy-α,β-dihydrochalcone,
3-methoxy-2,5,2′,3′-tetra-tertbutoxy-α,β-dihydrochalcone,
3-methoxy-2,5,2′,3′-tetrapentoxy-α,β-dihydrochalcone,
3-methoxy-2,5,2′,3′-tetra-(1-methylbutyl)oxy-α,β-dihydrochalcone,
3-methoxy-2,5,2′,3′-tetrahexoxy-α,β-dihydrochalcone,
3-methoxy-2,5,2′,3′-tetra-(1-methylpentyl)oxy-α,β-dihydrochalcone,
3-methoxy-2,5,2′,3′-tetra-(1-ethylbutyl)oxy-α,β-dihydrochalcone,
3-methoxy-2,5,2′,3′-tetracyclohexoxy-α,β-dihydrochalcone,
3-methoxy-2,5,3′-trimethoxy-α,β-dihydrochalcone,
3-methoxy-2,5,3′-triethoxy-α,β-dihydrochalcone,
3-methoxy-2,5,3′-tripropoxy-α,β-dihydrochalcone,
3-methoxy-2,5,3′-tri-isopropoxy-α,β-dihydrochalcone,
3-methoxy-2,5,3′-tributoxy-α,β-dihydrochalcone,
3-methoxy-2,5,3′-tri-isobutoxy-α,β-dihydrochalcone,
3-methoxy-2,5,3′-tri-tertbutoxy-α,β-dihydrochalcone,
3-methoxy-2,5,3′-tripentoxy-α,β-dihydrochalcone,
3-methoxy-2,5,3′-tri-(1-methylbutyl)oxy-α,β-dihydrochalcone,
3-methoxy-2,5,3′-trihexoxy-α,β-dihydrochalcone,
3-methoxy-2,5,3′-tri-(1-methylpentyl)oxy-α,β-dihydrochalcone,
3-methoxy-2,5,3′-tri-(1-ethylbutyl)oxy-α,β-dihydrochalcone,
3-methoxy-2,5,3′-tricyclohexoxy-α,β-dihydrochalcone,
2,2′,3′,5′-tetramethoxy-5-ethyl-α,β-dihydrochalcone,
2,2′,3′,5′-tetraethoxy-5-ethyl-α,β-dihydrochalcone,
2,2′,3′,5′-tetrapropoxy-5-ethyl-α,β-dihydrochalcone,
2,2′,3′,5′-tetrabutoxy-5-ethyl-α,β-dihydrochalcone,
2,2′,3′,5′-tetraisobutoxy-5-ethyl-α,β-dihydrochalcone,
2,2′,3′,5′-tetrapentoxy-5-ethyl-α,β-dihydrochalcone,
2,2′,3′,5′-tetra-(1-methylbutyl)oxy-5-ethyl-α,β-dihydrochalcone,
2,2′,3′,5′-tetra-(1-ethylpropyl)oxy-5-ethyl-α,β-dihydrochalcone,
2,2′,3′,5′-tetrahexoxy-5-ethyl-α,β-dihydrochalcone,
2,2′,3′,5′-tetra-(1-methylpentyl)oxy-5-ethyl-α,β-dihydrochalcone,
2,2′,3′,5′-tetra-(1-ethylbutyl)oxy-5-ethyl-α,β-dihydrochalcone,
2,2′,3′,5′-tetra-(1-propylpropyl)oxy-5-ethyl-α,β-dihydrochalcone,
2,3′,5′-trimethoxy-5-ethyl-α,β-dihydrochalcone,
2,3′,5′-triethoxy-5-ethyl-α,β-dihydrochalcone,
2,3′,5′-tripropoxy-5-ethyl-α,β-dihydrochalcone,
2,3′,5′-tributoxy-5-ethyl-α,β-dihydrochalcone,
2,3′,5′-triisobutoxy-5-ethyl-α,β-dihydrochalcone,
2,3′,5′-tripentoxy-5-ethyl-α,β-dihydrochalcone,
2,3′,5′-tri-(1-methylbutyl)oxy-5-ethyl-α,β-dihydrochalcone,
2,3′,5′-tri-(1-ethylpropyl)oxy-5-ethyl-α,β-dihydrochalcone,
2,3′,5′-trihexoxy-5-ethyl-α,β-dihydrochalcone,
2,3′,5′-tri-(1-methylpentyl)oxy-5-ethyl-α,β-dihydrochalcone,
2,3′,5′-tri-(1-ethylbutyl)oxy-5-ethyl-α,β-dihydrochalcone,
2,3′,5′-tri-(1-propylpropyl)oxy-5-ethyl-α,β-dihydrochalcone,
2,2′,3′,6′-tetramethoxy-5-ethyl-α,β-dihydrochalcone,
2,2′,3′,6′-tetraethoxy-5-ethyl-α,β-dihydrochalcone,
2,2′,3′,6′-tetrapropoxy-5-ethyl-α,β-dihydrochalcone,
2,2′,3′,6′-tetrabutoxy-5-ethyl-α,β-dihydrochalcone,
2,2′,3′,6′-tetraisobutoxy-5-ethyl-α,β-dihydrochalcone,
2,2′,3′,6′-tetrapentoxy-5-ethyl-α,β-dihydrochalcone,
2,2′,3′,6′-tetra-(1-methylbutyl)oxy-5-ethyl-α,β-dihydrochalcone,
2,2′,3′,6′-tetra-(1-ethylpropyl)oxy-5-ethyl-α,β-dihydrochalcone,
2,2′,3′,6′-tetrahexoxy-5-ethyl-α,β-dihydrochalcone,
2,2′,3′,6′-tetra-(1-methylpentyl)oxy-5-ethyl-α,β-dihydrochalcone,
2,2′,3′,6′-tetra-(1-ethylbutyl)oxy-5-ethyl-α,β-dihydrochalcone,
2,2′,3′,6′-tetra-(1-propylpropyl)oxy-5-ethyl-α,β-dihydrochalcone,
2,2′,5′-trimethoxy-5-ethyl-α,β-dihydrochalcone,
2,2′,5′-triethoxy-5-ethyl-α,β-dihydrochalcone,
2,2′,5′-tripropoxy-5-ethyl-α,β-dihydrochalcone,
2,2′,5′-tributoxy-5-ethyl-α,β-dihydrochalcone,
2,2′,5′-triisobutoxy-5-ethyl-α,β-dihydrochalcone,
2,2′,5′-tripentoxy-5-ethyl-α,β-dihydrochalcone,
2,2′,5′-tri-(1-methylbutyl)oxy-5-ethyl-α,β-dihydrochalcone,
2,2′,5′-tri-(1-ethylpropyl)oxy-5-ethyl-α,β-dihydrochalcone,
2,2′,5′-trihexoxy-5-ethyl-α,β-dihydrochalcone,
2,2′,5′-tri-(1-methylpentyl)oxy-5-ethyl-α,β-dihydrochalcone,
2,2′,5′-tri-(1-ethylbutyl)oxy-5-ethyl-α,β-dihydrochalcone,
2,2′,5′-tri-(1-propylpropyl)oxy-5-ethyl-α,β-dihydrochalcone,
2,2′,6′-trimethoxy-5-ethyl-α,β-dihydrochalcone,
2,2′,6′-triethoxy-5-ethyl-α,β-dihydrochalcone,
2,2′,6′-tripropoxy-5-ethyl-α,β-dihydrochalcone,
2,2′,6′-tributoxy-5-ethyl-α,β-dihydrochalcone,
2,2′,6′-triisobutoxy-5-ethyl-α,β-dihydrochalcone,
2,2′,6′-tripentoxy-5-ethyl-α,β-dihydrochalcone,
2,2′,6′-tri-(1-methylbutyl)oxy-5-ethyl-α,β-dihydrochalcone,
2,2′,6′-tri-(1-ethylpropyl)oxy-5-ethyl-α,β-dihydrochalcone,
2,2′,6′-trihexoxy-5-ethyl-α,β-dihydrochalcone,
2,2′,6′-tri-(1-methylpentyl)oxy-5-ethyl-α,β-dihydrochalcone,
2,2′,6′-tri-(1-ethylbutyl)oxy-5-ethyl-α,β-dihydrochalcone,
2,2′,6′-tri-(1-propylpropyl)oxy-5-ethyl-α,β-dihydrochalcone,
2,2′-dimethoxy-5-ethyl-α,β-dihydrochalcone,
2,2′-diethoxy-5-ethyl-α,β-dihydrochalcone,
2,2′-dipropoxy-5-ethyl-α,β-dihydrochalcone,
2,2′-dibutoxy-5-ethyl-α,β-dihydrochalcone,
2,2′-disobutoxy-5-ethyl-α,β-dihydrochalcone,
2,2′-dipentoxy-5-ethyl-α,β-dihydrochalcone,
2,2′-di-(1-methylbutyl)oxy-5-ethyl-α,β-dihydrochalcone,
2,2′-di-(1-ethylpropyl)oxy-5-ethyl-α,β-dihydrochalcone,
2,2′-dihexoxy-5-ethyl-α,β-dihydrochalcone,
2,2′-di-(1-methylpentyl)oxy-5-ethyl-α,β-dihydrochalcone,
2,2′-di-(1-ethylbutyl)oxy-5-ethyl-α,β-dihydrochalcone,
2,2′-di-(1-propylpropyl)oxy-5-ethyl-α,β-dihydrochalcone,
2,2′,3′-trimethoxy-5-ethyl-α,β-dihydrochalcone,
2,2′,3′-triethoxy-5-ethyl-α,β-dihydrochalcone,
2,2′,3′-tripropoxy-5-ethyl-α,β-dihydrochalcone,
2,2′,3′-tributoxy-5-ethyl-α,β-dihydrochalcone,
2,2′,3′-triisobutoxy-5-ethyl-α,β-dihydrochalcone,
2,2′,3′-tripentoxy-5-ethyl-α,β-dihydrochalcone,
2,2′,3′-tri-(1-methylbutyl)oxy-5-ethyl-α,β-dihydrochalcone,
2,2′,3′-tri-(1-ethylpropyl)oxy-5-ethyl-α,β-dihydrochalcone,
2,2′,3′-trihexoxy-5-ethyl-α,β-dihydrochalcone,
2,2′,3′-tri-(1-methylpentyl)oxy-5-ethyl-α,β-dihydrochalcone,
2,2′,3′-tri-(1-ethylbutyl)oxy-5-ethyl-α,β-dihydrochalcone,
2,2′,3′-tri-(1-propylpropyl)oxy-5-ethyl-α,β-dihydrochalcone,
2,3′-dimethoxy-5-ethyl-α,β-dihydrochalcone,
2,3′-diethoxy-5-ethyl-α,β-dihydrochalcone,
2,3′-dipropoxy-5-ethyl-α,β-dihydrochalcone,
2,3′-dibutoxy-5-ethyl-α,β-dihydrochalcone,
2,3′-diisobutoxy-5-ethyl-α,β-dihydrochalcone,
2,3′-dipentoxy-5-ethyl-α,β-dihydrochalcone,
2,3′-di-(1-methylbutyl)oxy-5-ethyl-α,β-dihydrochalcone,
2,3′-di-(1-ethylpropyl)oxy-5-ethyl-α,β-dihydrochalcone,
2,3′-dihexoxy-5-ethyl-α,β-dihydrochalcone,
2,3′-di-(1-methylpentyl)oxy-5-ethyl-α,β-dihydrochalcone,
2,2′,3′,5′,6′-pentamethoxy-5-ethyl-α,β-dihydrochalcone,
2,2′,3′,5′,6′-pentaethoxy-5-ethyl-α,β-dihydrochalcone,
2,2′,3′,5′,6′-pentapropoxy-5-ethyl-α,β-dihydrochalcone,
2,2′,3′,5′,6′-pentabutoxy-5-ethyl-α,β-dihydrochalcone,
2,2′,3′,5′,6′-pentaisobutoxy-5-ethyl-α,β-dihydrochalcone,
2,2′,3′,5′,6′-pentapentoxy-5-ethyl-α,β-dihydrochalcone,
2,2′,3′,5′,6′-penta-(1-methylbutyl)oxy-5-ethyl-α,β-dihydrochalcone,
2,2′,3′,5′,6′-penta-(1-ethylpropyl)oxy-5-ethyy-α,β-dihydrochalcone,
2,2′,3′,5′,6′-pentahexoxy-5-ethyl-α,β-dihydrochalcone,
2,2′,3′,5′,6′-penta-(1-methylpentyl)oxy-5-ethyl-α,β-dihydrochalcone,
2,2′,3′,5′,6′-penta-(1-ethylbutyl)oxy-5-ethyl-α,β-dihydrochalcone, and
2,2′,3′,5′,6′-penta-(1-propylpropyl)oxy-5-ethyl-α,β-dihydrochalcone.

290. The use of a compound according to any of the claims 57-289 as a drug substance.

291. A pharmaceutical composition comprising a compound according to any of the claims 57-289.

292. The use of a bis-aromatic compound according to any of the claims 57-289 for the preparation of a pharmaceutical composition or a medicated feed, food or drinking water for the treatment or prophylaxis of a disease caused by a microorganism or a parasite in an animal, including a vertebrate, such as a bird, a fish or a mammal, including a human, the microorganism or parasite being selected from parasitic protozoa, in particular tissue and blood protozoa such as Leishmania, Trypanosoma, Toxoplasma, Plasmodium, Pneumocystis, Babesia and Theileria; intestinal protozoan flagellates such as Trichomonas and Giardia; intestinal protozoan Coccidia such as Eimeria, Isospora, Cryptosporidium; Cappilaria, Microsporidium, Sarcocystis, Trichodina, Trichodinella, Dacthylogurus, Pseudodactylogurus, Acantocephalus, Ichthyophtherius, Botrecephalus; and intracellular bacteria, in particular Mycobacterium, Legionella species, Listeria, and Salmonella.

293. The use according to claim 292, wherein the aromatic compound, in a concentration in which it causes less than 50% reduction of the thymidine uptake by human lymphocytes in the Lymphocyte Proliferation Assay using phytobemagglutinin (PHA), meets at least one of the following criteria:
a) the aromatic compound is capable of inhibiting in vitro the growth or multiplication of Leishmania major promastigotes by at least 80%, as determined by uptake of tritiated thymidine,
b) the aromatic compound is capable of inhibiting in vitro the growth or multiplication of Plasmodium falciparum by at least 80%, as determined by uptake of tritiated hypoxanthine,
c) the aromatic compound is capable of inhibiting in vitro the growth or multiplication of Eimeria tenella in chicken fibroblast cell cultures by at least 70%, as determined by counting the parasites,
d) the aromatic compound is capable of inhibiting in vitro the growth or multiplication of Mycobacterium tuberculosis or Legionella pneumophila by at least 50%, as determined by colony counts.

294. The use according to claim 293, wherein the aromatic compound, in a concentration in which it causes less than 40% reduction, preferably less than 30% reduction, more preferably less than 20% reduction, of the thymidine uptake by human lymphocytes in the Lymphocyte Proliferation Assay using PHA, meets at least one of the criteria a) to d).

295. The use according to claim 292, wherein the pharmaceutical composition is a composition for the treatment or prophylaxis of diseases caused by Leishmania in humans or dogs, and the aromatic compound is capable of inhibiting in vitro the growth of Leishmania major promastigotes by at least 80%, as determined by uptake of tritiated thymidine, in a concentration of the compound in which it causes less than 50% reduction, preferably less than 40% reduction, more preferably less than 30% reduction, most preferably less than 20% reduction, of the thymidine uptake by human lymphocytes in the Lymphocyte Proliferation Assay using PHA.

296. The use according to claim 292, wherein the pharmaceutical composition is a composition for the treatment or prophylaxis of diseases caused by Leishmania in humans or dogs, and the aromatic compound, or the prodrug, when administered intraperitoneally in the in vivo test described in Example 5 herein in a dose of up to 20 mg per kg body weight, preferably in a dose of up to 10 mg per kg body weight, once daily for 40 days to female BALB/c mice which have been infected with L. major ((10⁷/mouse), the administration being initiated one week after infection, is capable of preventing increase in lesion size by at least 60%, preferably at least 80%, more preferably at least 90%.

297. The use according to claim 292, wherein the pharmaceutical composition is a composition for the treatment or prophylaxis of diseases caused by Leishmania in humans or dogs, and the aromatic compound, or the prodrug, when administered intraperitoneally in the in vivo test described in Example 6 herein in a dose of up to 20 mg per kg body weight, preferably in a dose of up to 10 mg per kg body weight, two times daily for 7 days to male Syrian golden hamsters which have been infected with L. donovani promastigotes (2×10⁷/hamster), the administration being initiated one day after infection, is capable of reducing the parasite load in the liver of the hamsters by at least 60%, preferably by at least 80%, and more preferably by at least 90%.

298. The use according to claim 292, wherein the pharmaceutical composition is a composition for the treatment or prophylaxis of malaria caused by Plasmodium spp. in humans, and the aromatic compound is capable of inhibiting in vitro the growth of Plasmodium falciparum by at least 80%, as measured by uptake of tritiated hypoxanthine, in a concentration of the compound in which it causes less than 50% reduction, preferably less than 40% reduction, more preferably less than 20% reduction, of the thymidine uptake by human lymphocytes, as measured by the Lymphocyte Proliferation Assay using PHA.

299. The use according to claim 292, wherein the pharmaceutical composition is a composition for the treatment or prophylaxis of diseases caused by Plasmodium spp. in humans, and the aromatic compound, when administered intraperitoneally in the Plasmodium Assay described herein in a dose of up to 20 mg per kg body weight two times daily for 6 days to female BALB/c mice which have been infected with malaria P. yoelii (2×10⁵/mouse), the administration being initiated one day after infection, is able to prevent increase in the parasitemia during the administration period.

300. The use according to claim 299, wherein the aromatic compound, or the prodrug, when administered intraperitoneally in the Plasmodium Assay described herein in a dose of up to 20 mg per kg body weight, preferably in a dose of up to 5 mg per kg body weight, most preferably in a dose of up to 2.5 mg per kg body weight, two times daily for 8 days to 8 weeks old female BALB/c mice which have been infected with malaria P. yoelii strain YM (1×10⁶/mouse), the administration being initiated one day after infection, is capable of clearing the parasite from the mice within at the most 23 days, preferably within at the most 17 days.

301. The use according to claim 292 of an aromatic compound, or a prodrug thereof, which aromatic compound contains an alkylating site and which aromatic compound is capable of alkylating the thiol group in N-acetyl-L-cysteine at physiological pH, for the preparation of a pharmaceutical composition or a medicated feed or drinking water for the treatment or prophylaxis of diseases caused by Coccidia in poultry such as chickens or turkeys, wherein the aromatic compound, or the prodrug, when administered to chickens with the feed in a concentration of up to 400 ppm, preferably in a concentration of up to 120 ppm, more preferably in a concentration of up to 60 ppm, for at most 28 days in the in vivo test described in the Anticoccidial Assay described herein, is capable of controlling infection by Eimeria tenella in at least 60%, preferably at least 80%, of the chickens and preventing pathological alterations in at least 50%, preferably in at least 65%, of the chickens.

302. The use according to claim 292, wherein the pharmaceutical composition is a composition for the treatment or prophylaxis of diseases caused by intracellular bacteria such as Mycobacteria in humans or animals such as cattle, and the aromatic compound is one which is capable of inhibiting the growth and multiplication of Mycrobacteria Assay described herein at a mean MIC of up to 20 μg per ml, preferably up to 10 μg per ml, more preferably up to 5 μg per ml, and, in the same concentration, causes less than 50% reduction, preferably less than 40% reduction, more preferably less than 20% reduction, of the thymidine uptake of human lymphocytes as measured by The Lymphocyte Proliferation Assay.

303. The use according to claim 292, wherein the pharmaceutical composition is a composition for the treatment or prophylaxis of diseases caused by intracellular bacteria such as Legionella in humans, and the aromatic compound is one which is capable of inhibiting the growth and multiplication of Legionella pneumophila in vitro in the Legionella Assay described herein at a mean MIC of up to 20 μg per ml, preferably up to 10 μg per ml, more preferably up to 5 μg per ml, and, in the same concentration, causes less than 50% reduction, preferably less than 40% reduction, more preferably less than 20% reduction, of the thymidine uptake of human lymphocytes as measured by The Lymphocyte Proliferation Assay.

304. The use according to any of claims 292-294, wherein the disease is human leishmaniasis caused by Leishmania donovani, L. infantum, L. aethiopica, L. major, L. tropica, L. mexicana complex, or L. braziliensis complex or human malaria caused by Plasmodium falciparum, P. ovale, P. vivax, or P. malariae.

305. The use according to any of claims 292-294, wherein the disease is a parasitic disease in livestock, such as Babesia in cattle, or a parasitic disease in birds, such as a disease caused by Coccidia such as Eimeria tenella in poultry such as chicken or turkey, or a parasitic disease in fish, such as Pseudodactylogurus or Trichodina.

306. A method for producing a β-substituted chalcone derivative of the general formula (III) 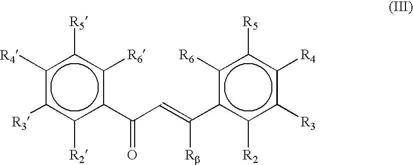
wherein R_{β} is selected from C₁₋₆ straight, branched and cyclic aliphatic hydrocarbyl which may be saturated or may contain one or more unsaturated bonds selected from double bonds and triple bonds, which hydrocarbyl may be substituted with one or more substituents selected from C₁₋₆ alkoxy, hydroxy, halogen, amino, and amino which is optionally alkylated with one or two C₁₋₆ alkyl groups; R₂, R₃, R₄, R₅, R₆, R₂′, R₃′, R₄′, R₅′, and R₆′ each independently is selected from hydrogen, cyano, nitro, nitroso, amino, halogen, R_{H}, and AW, wherein each A independently is selected from —O—, —S—, —NH—, and —NR_{H}—, and each W independently is selected from hydrogen, R_{H}, and R_{H}CO—, wherein R_{H} is selected from C₁₋₆ straight, branched and cyclic aliphatic hydrocarbyl which may be saturated or may contain one or more unsaturated bonds selected from double bonds and triple bonds, which hydrocarbyl may be substituted with one or more substituents selected from C₁₋₆ alkoxy, hydroxy, halogen, amino, and amino which is optionally alkylated with one or two C₁₋₆ alkyl groups, or salts or hydrates thereof,
comprising the steps of
1) reacting a compound of the general formula (IV) 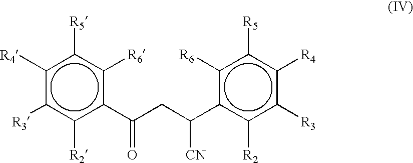
wherein R₂, R₃, R₄, R₅, R₆, R₂′, R₃′, R₄′, R₅′, and R₆′ are as defined above, with i) a base, and ii) a hydrocarbyl halide, R₆₂—X₁, wherein X₁ is selected from fluoro, chloro, bromo, or iodo, to obtain a compound of the general formula (V) 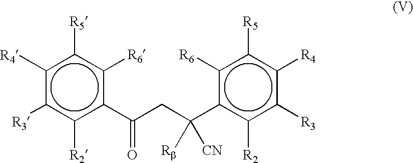
wherein R_{β}, R₂, R₃, R₄, R₅, ,R₂′, R₃′, R₄′, R₅′, and R₆′ are as defined above,
2) subjecting a compound of the general formula (V) to elimination conditions to obtain a compound of the general formula (III).

307. A method according to claim 306, wherein the base is selected from a basic salt of a dialkylamine and NaH.

308. A method according to claim 307, wherein the base is lithium diisopropylamide.

309. A method according to any of claims 306-308, wherein the elimination conditions comprises the use of a reagent selected from NaH and a basic salt of a dialkylamine.

310. A method according to claim 309, wherein the elimination condition comprises the use of NaH.

311. A method for producing an α-substituted chalcone derivative of the general formula (VI) 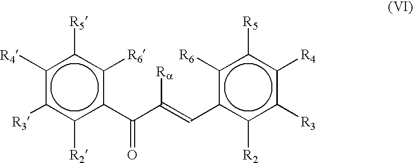
wherein R_{α} is selected from C₁₋₆ straight, branched and cyclic aliphatic hydrocarbyl which may be saturated or may contain one or more unsaturated bonds selected from double bonds and triple bonds, which hydrocarbyl may be substituted with one or more substituents selected from C₁₋₆ alkoxy, hydroxy, halogen, amino, and amino which is optionally alkylated with one or two C₁₋₆ alkyl groups; R₂, R₃, R₄, R₅, R₆, R₂′, R₃′, R₄′, R₅′, and R₆′ each independently is selected from hydrogen cyano, nitro, nitroso, amino, halogen, R_{H}, and AW, wherein each A independently is selected from —O—, —S—, —NH—, and —NR_{H}—, and each W independently is selected from hydrogen, R_{H}, and R_{H}CO—, wherein R_{H} is selected from C₁₋₆ straight, branched and cyclic aliphatic hydrocarbyl which may be saturated or may contain one or more unsaturated bonds selected from double bonds and triple bonds, which hydrocarbyl may be substituted with one or more substituents selected from C₁₋₆ alkoxy, hydroxy, halogen, amino, and amino which is optionally alkylated with one or two C₁₋₆ alkyl groups, or salts or hydrates thereof,
comprising the steps of
1) reacting a compound of the general formula (VII) 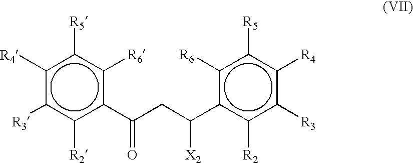
wherein R₂, R₃, R₄, R₅, R₆, R₂′, R₃′, R₄′, R₅′, and R₆′ are as defined above, and X₂ is selected from cyano and halogen, with a trialkylsilyl halide to obtain a compound of the general formula (VIII) 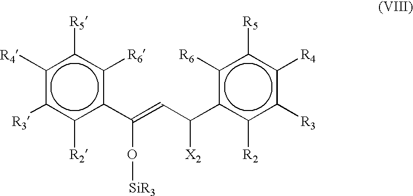
wherein each R independently is selected from C₁₋₆ straight, branched and cyclic alkyl, and X₂, R₂, R₃, R₄, R₅, R₆, R₂′, R₃′, R₄′, R₅′, and R₆′ are as defined above,
2) reacting a compound of the general formula (VIII) with a hydrocarbyl halide, R_{α}—X₃, wherein X₃ is selected from fluoro, chloro, bromo, or iodo, in the presence of a fluoride-donating agent, to obtain a compound of the general formula (IX) 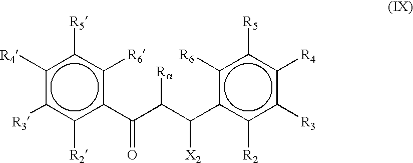
wherein R_{α}, X₂, R₂, R₃, R₄, R₅, R₆, R₂′, R₃′, R₄′, R₅′, and R₆′ are as defined above, and
3) subjecting a compound of the general formula (IX) to elimination conditions to obtain a compound of the general formula (VI).

312. A method according to claim 311, wherein X₂ is cyano.

313. A method according to claim 311 or claim 312, wherein step 1) is carried out in the presence of NaH.

314. A method according to any of claims 311-313, wherein the fluoride-donating agent is selected from LiF, NaF, KF, RbF, CsF, HF, (C₄H₉)₄NF, and (C₂H₅)₃NHF.

315. A method according to claim 314, wherein the fluoride-donating agent is CsF.

316. A method according to any of claims 311-315, wherein the trialkylsilyl halide is selected from tertbutyldimethylsilyl chloride, tertbutyldimethylsilyl bromide, and tertbutyldimethylsilyl iodide.

317. A method according to claim 316, wherein the trialkylsilyl halide is tertbutyldimethylsilyl chloride.

318. A method according to any of claims 311-317, wherein the elimination condition comprises the use of a compound selected from NaH, a basic salt of a dialkylamine, and an alkali metal alkoxylate.

319. A method according to claim 318, wherein the elimination condition comprises the use of NaH.

320. A method for the isolation and purification of Leishmania fumarate reductase comprising steps substantially as the steps for isolation and purification described in Example 4.

321. Leishmania fumarate reductase obtainable by the method defined in claim 320.

322. The use of a compound according to any of the claims 57-289 for the preparation of a pharmaceutical composition for the treatment of prophylaxis of a number of diseases including i) conditions relating to harmful effects of inflammatory cytokines, ii) conditions involving infections by Helicobacter species, iii) conditions involving injections by viruses, and iv) neoplastic disorders.

323. A compound according to any of claims 57-289 which, according to a QSAR analysis, has a predicted IC₅₀ value in the Cytokine inhibition Assay defined herein of at the most 100 μg/ml, such at the most, e.g., 75 μg/ml, 60 μg/ml, 50 μg/ml, 40 μg/ml, 30 μg/ml, 20 μg/ml, or 10 μg/ml.

324. A compound according to claim 323, which, according to a QSAR analysis, at the predicted IC₅₀ concentration in the Cytokine inhibition Assay, has a predicted reduction of the thymidine uptake by human lymphocytes in the Lymphocyte Proliferation Assay, as defined herein, using phytomemagglutinin (PHA), of less than 50%, preferably less than 40%, especially less than 30%, in particular less than 20%.

325. A compound according to claim 323 or claim 324, for which the ratio between the predicted IC₅₀ value in the Cytokine inhibition Assay and the predicted IC₅₀ value in the Lymphocyte Proliferation Assay for the compound in question is equal to or less than 1, such as less than, e.g., 0.9, 0.8, 0.7, 0.6, 0.5, 0.4, 0.3, or 0.2.

326. A compound according to any of claims 57-254 which, according to a QSAR analysis, has a predicted MIC value in the Helicobacter pylori Assay of at the most 200 μg/ml, such as at the most, erg., 100 μg/ml, 75 μg/ml, 50 μg/ml, 40 μg/ml, 30 μg/ml, 20 μg/ml, or 10 μg/ml.

327. A compound according to claim 326, which, according to a QSAR analysis, at the predicted MIC concentration in the Helicobacter species Assay, has a predicted reduction of the thymidine uptake by human lymphocytes in the Lymphocyte Proliferation Assay, as defined herein, using phytomemagglutinin (PHA), of less than 50%, preferably less than 40%, especially less than 30%, in particular less than 20%.

328. A compound according to claim 326 or 327, for which the ratio between the predicted MIC value in the Helicobacter species Assay and the predicted IC₅₀ value in the Lymphocyte Proliferation Assay for the compound in question is less than 2, such as less than, e.g., 1.5, 1.2, 1.0, 0.9, 0.8, 0.7, 0.6, 0.5, 0.4, 0.3, or 0.2.

329. A compound according to any of claims 57-254 which, according to a QSAR analysis, has a predicted IC₅₀ value in the Virus plaque formation and/or Virus cytopathic Assay of at the most 50 μg/ml, such as at the most, e.g. 40 μg/ml, 30 μg/ml, 20 μg/ml, 10 μg/ml, or 5 μg/ml.

330. A compound according to claim 329, which, according to a QSAR analysis, at the predicted IC₅₀ concentration in the Virus plaque formation and/or Virus cytopathic Assay, has a predicted reduction of the thymidine uptake by human lymphocytes in the Lymphocyte Proliferation Assay, as defined herein, using phytomemagglutinin (PHA), of less than 50%, preferably less than 40%, especially less than 30%, in particular less than 20%.

331. A compound according to claim 329 or 330, for which the ratio between the predicted IC₅₀ value in the Virus plaque formation and/or Virus cytopathic Assay and the predicted IC₅₀ value in the Lymphocyte Proliferation Assay for the compound in question is equal to or less than 1, such as less than, e.g., 0.9, 0.8, 0.7, 0.6, 0.5, 0.4, 0.3, or 0.2.

332. A compound according to any of claims 57-254 which, according to a QSAR analysis, has a predicted IC₅₀ value in the Anti-cancer Assay of at the most 100 μg/ml, such at the most, e.g., 75 μg/ml, 60 μg/ml, 50 μg/ml, 40 μg/ml, 30 μg/ml, 20 μg/ml, or 10 μg/ml.

333. A method for selecting, among compounds selected from molecular types of 1,3-bis-aromatic-prop-2-en-1-ones, 1,3-bis-aromatic-propan-1-ones, and 1,3-bis-aromatic-prop-2-yn-1-ones, such compounds which have an IC₅₀ value in the Cytokine inhibition Assay defined herein of at the most 100 μg/ml, such at the most, e.g., 75 μg/ml, 60 μg/ml, 50 μg/ml, 40 μg/ml, 30 μg/ml, 20 μg/ml, or 10 μg/ml, the method comprising establishing a panel of compounds of the molecular type in question and having a variability of variables suitable for the QSAR analysis, including varying activities in the assay, subjecting the compounds with their assay data to QSAR analysis, constructing compounds which, according to the output of the QSAR analysis, are predicted to have an improved activity in the assay, providing, e.g. synthesising, the compounds constructed, testing the compounds constructed in the assay and, optionally or if necessary, including one or more of the synthesised and tested compounds with their data in a further QSAR analysis, and, optionally or if necessary repeating these operations, and finally selecting compounds having IC₅₀ data of the desired maximum value in the assay.

334. A method for selecting, among compounds selected from molecular types of 1,3-bis-aromatic-prop-2-en-1-ones, 1,3-bis-aromatic-propan-1-ones, and 1,3-bis-aromatic-prop-2-yn-1-ones, such compounds which have an MIC value in the Helicobacter pylori Assay defined herein of at the most 200 μg/ml, such as at the most, e.g., 100 μg/ml, 75 μg/ml, 50 μg/ml, 40 μg/ml, 30 μg/ml, 20 μg/ml, or 10 μg/ml, the method comprising establishing a panel of compounds of the molecular type in question and having a variability of variables suitable for the QSAR analysis, including varying activities in the assay, subjecting the compounds with their assay data to QSAR analysis, constructing compounds which, according to the output of the QSAR analysis, are predicted to have improved activity in the assay, providing, e.g. synthesising, the compounds constructed, testing the compounds constructed in the assay and, optionally or if necessary, including one or more of the thus synthesised and tested compounds with their data in a further QSAR analysis, and, optionally or if necessary repeating these operations, and finally selecting compounds having MIC data of the desired miaximum value in the assay.

335. A method for selecting, among compounds selected from molecular types of 1,3-bis-aromatic-prop-2-en-1-ones, 1,3-bis-aromatic-propan-1-ones, and 1,3-bis-aromatic-prop-2-yn-1-ones, such compounds which have an IC₅₀ in the Virus plaque formation and/or Virus cytopathic Assay of at the most 50 μg/ml, such as at the most, e.g. 40 μg/ml, 30 μg/ml, 20 μg/ml, 10 μg/ml, or 5 μg/m, the method comprising establishing a panel of compounds of the molecular type in question and having a variability of variables suitable for the QSAR analysis, including varying activities in the assay, subjecting the compounds with their assay data to QSAR analysis, constructing compounds which,
